SEQUENCE LISTING (69 sequences)

SEQ ID NO: 1 - protein, 239 aa, synthetic construct
MVSKGEELFTGVVPILVELDGDVNGHKFSVSGEGEGDATYGKLTLKFICTTGKLPVPWPTLVTTFTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 2 - protein, 113 aa, synthetic construct
MQVQLVETGGGLVQDGDSLRLSCAASGFTFSNYYMSWVRQAPGKGLEWVSGITSGGTYTKYADAVKDRFTISRDNAKNTLYLQMNSLKPEDTAVYYCAKGGTSVAVRGFYDYR

SEQ ID NO: 3 - protein, 119 aa, synthetic construct
MAQVQLVESGGRLVQAGDSLRLSCAASGRTFSTSAMAWFRQAPGREREFVAAITWTVGNTILGDSVKGRFTISRDRAKNTVDLQMDNLEPEDTAVYYCSARSRGYVLSVLRSVDSYDYW

SEQ ID NO: 4 - protein, 623 aa, Lotus japonicus
MKLKTGLLLFFILLLGHVCFHVESNCLKGCDLALASYYILPGVFILQNITTFMQSEIVSSNDAITSYNKDKILNDINIQSFQRLNIPFPCDCIGGEFLGHVFEYSASKGDTYETIANLYYANLTTVDLLKRFNSYDPKNIPVNAKVNVTVNCSCGNSQVSKDYGLFITYPIRPGDTLQDIANQSSLDAGLIQSFNPSVNFSKDSGIAFIPGRYKNGVYVPLYHRTAGLASGAAVGISIAGTFVLLLLAFCMYVRYQKKEEEKAKLPTDISMALSTQDGNASSSAEYETSGSSGPGTASATGLTSIMVAKSMEFSYQELAKATNNFSLDNKIGQGGFGAVYYAELRGKKTAIKKMDVQASTEFLCELKVLTHVHHLNLVRLIGYCVEGSLFLVYEHIDNGNLGQYLHGSGKEPLPWSSRVQIALDAARGLEYIHEHTVPVYIHRDVKSANILIDKNLRGKVADFGLTKLIEVGNSTLQTRLVGTFGYMPPEYAQYGDISPKIDVYAFGVVLFELISAKNAVLKTGELVAESKGLVALFEEALNKSDPCDALRKLVDPRLGENYPIDSVLKIAQLGRACTRDNPLLRPSMRSLVVALMTLSSLTEDCDDESSYESQTLINLLSVR

SEQ ID NO: 5 - protein, 620 aa, Medicago truncatula
MNLKNGLLLFILFLDCVFFKVESKCVKGCDVALASYYIIPSIQLRNISNFMQSKIVLTNSFDVIMSYNRDVVFDKSGLISYTRINVPFPCECIGGEFLGHVFEYTTKEGDDYDLIANTYYASLTTVELLKKFNSYDPNHIPVKAKINVTVICSCGNSQISKDYGLFVTYPLRSDDTLAKIATKAGLDEGLIQNFNQDANFSIGSGIVFIPGRDQNGHFFPLYSRTGIAKGSAVGIAMAGIFGLLLFVIYIYAKYFQKKEEEKTKLPQTSRAFSTQDASGSAEYETSGSSGHATGSAAGLTGIMVAKSTEFTYQELAKATNNFSLDNKIGQGGFGAVYYAELRGEKTAIKKMDVQASSEFLCELKVLTHVHHLNLVRLIGYCVEGSLFLVYEHIDNGNLGQYLHGIGTEPLPWSSRVQIALDSARGLEYIHEHTVPVYIHRDVKSANILIDKNLRGKVADFGLTKLIEVGNSTLHTRLVGTFGYMPPEYAQYGDVSPKIDVYAFGVVLYELITAKNAVLKTGESVAESKGLVQLFEEALHRMDPLEGLRKLVDPRLKENYPIDSVLKMAQLGRACTRDNPLLRPSMRSIVVALMTLSSPTEDCDDDSSYENQSLINLLSTR

SEQ ID NO: 6 - protein, 599 aa, Phaseolus vulgaris
MELKKGLLLLFLLLEYVCCNVEAKCLKGCGAALASYYVSPGISTLDDITHLMKSSVVSNSDDIVSYNKDRIFNKNVLFFYRINIPFPCECIRDEFLGHVFEYSAAAGDTYDSIAKVTYANLTTVELLRRFNSYGQNDIPTNAKVNVTVNCSCGNSQVSQDYGLFITYPLRPGNNLHDIANETQLDAQLLQNYNPGVNFSQESGIVFIPGRGLSRGAAAGISIAGICGLLLLVICIYAKFFQKKEGEKSKLPTMVFSTQYASGSAEYETSGSSGTATATGLTGIMVAKSMEFTYQELAKATNNFSLENKIGQGGFGAVYYAELRGEKTAIKKMDVQASTEFLCELKVLTHVHHLNLVRLIGYCVEGSLFLVYEYIDNGNLGQYLHGIGKDPLPWSSRVQIALDSARGLEYIHEHTVPVYIHRDVKSANILIDKNFRGKVADFGLTKLIEVGGSTLHTRLVGTFGYMPPEYAQYGDVSPKVDVYAFGVVLYELISAKNAVLKTGEYVAESKGLVALFEEALNQSNPSEVIRKLVDPRLGENYPIDSVLKIAQLGRACTRDNPLLRPSMRSIVVALMTLSSPTEDLDDSYENQTLINLLSVR

SEQ ID NO: 7 - protein, 633 aa, Arachis hypogaea
MKLKKSFLLFFLLLECSCFINLESKCLKGCDLAIASYFVYPKVMLGSIASFMHSNVVPNSNVIISYNKDKMPNNLPVSFTRINIPFPCDCINGEFLGHVFEYSAIEGDTYDLIANMRYSDLTTVEILQRFNNYDPNHVPVNAKVNVTVNCSCGNSNVSKDYGLFVTFPLSEGNTLLQIANQTKLDPKLLQGYNPGVNFNQTRGIVFIPGRDQNGVYVPLYPRLAGLAKGVAVGISIAGTSGVLLLLICACVRYFQKKEEEKIKLPTEDFISSSTQVGNASSSGEYETSGSTAASASGLPAIMAAKSMEFSYQELAKATNNFSSDNKIGQGGFGAVYFAELRGQKAAIKKMDVQASTEFLSELKVLTHVHHLNLVRLIGYCVEGSLFLVYEYIENGNLGQYLHGTEREPLAWSTRVQIALDSARGLEYIHEHTVPVYIHRDVKPANILIDKDFRGKVADFGLTKLREVGSSLSLHTRLVGTFGYMPPEYAQYGDVSPKVDVYAFGVVLYELISAKNAVLKTGETVAESSKGLVTLFEEALNKSDPSKAIRKLVDPRLGENYPIDSVLKVAELGRACTRDNPLLRPSMRSVVVALMTLLASSTSIEDCDDNDDDDDDDDDNSYENRTLIDQLSVR

SEQ ID NO: 8 - protein, 631 aa, Arachis hypogaea
MKLKKSFLLFFLLLECSCFINLESKCLKGCDLAIASYFVYPRVKLGSIASFMHSNVVPNSNVIISYNKDKMPNNLPVSFTTINIPFPCDCINGEFLGHVFEYSAIEGDTYYLIANMRYSDLTTVEILQRFNNYDPNHVPVNAKVNVTVNCSCGNSHVSKDYGLFVTFPLSEGNTLLQIANQTKLDPKLLQSYNPGVNFNQTRGIVFIPGRDQNGVYVPLYPRLAGLAKDVAVGISIAGTSGVLLLLICVCVRYFQKKEEEKIKLPTEDFISSSTQDGNASSSGEYETSGSTAASASGLPAIMAAKSMEFSYQELAKATNNFSSDNKIGQGGFGAVYFAELRGQKAAIKKMDVQASTEFLSELKVLTHVHHLNLVRLIGYCVEGSLFLVYEYIENGNLGQYLHGTGREPLAWLTRVQIALDSARGLEYIHEHTVPVYIHRDVKPANILIDKDFRGKVADFGLTKLREVGSSLSLHTRLVGTFGYMPPEYAQYGDVSPKVDVYAFGVVLYELISAKNAVLKTGETVAESSKGLVTLFEEALNKNDPSKAIRKLVDPRLGENYPIDSVLKVAELGRACTRDNPLLRPSMRSVVVALMTLLASSTSIEDCDDNDDDDDDDNSYENRTLIDQLSVR

SEQ ID NO: 9 - protein, 632 aa, Cajanus cajan
MFLSYPMELKKGLLFLLLLECVFCDVESMCVKECDVALASYYILPGYLTVENVTGWLESSVLSNSDVIKSYNKDKIIKDNLPSFDRINVPFPCDCIHEEFLGHVFEYSAAAGDTYDSIAKVTYANLTTVELLTRFNSYGHDIPQNAKINVTVKCSCGNSQVSKDYGLFITYPLRPGNNLHDIANEVHLDAQLLEKYNPGVNFSKDSGIVFIPGRDQNGDYVPLYPRKAGGIKKKISAKDAAVGVSIAGICGLLLLVVCIYVKYLQKKEGEEVKLPTESSMAFSTQDGTASGSSEYETSGSSGPTTASATGLTGIMVAKSMEFSYQELAKATNNFSLENKIGQGGFGAVYYAELRGEKTAIKKMDVQASTEFLCELKVLTHVHHLNLVRLIGYCVEGSLFLVYEYIDNGNLGQYLHGTGKDPLPWSSRVQIALDSARGLEYIHEHTVPVYIHRDVKSANILIDKNFRGKVADFGLTKLIEVGGSTLHTRLVGTFGYMPPEYAQYGDVSPKVDVYAFGVVLYELISAKNAILRTGESVAETKGLVALFEEALNQSNPGEGIRKLVDPRLGENYPIDSVLKIAQLGRACTRDNPLLRPSMRSIVVALMTLSSPTEDCEDGASYENQTLINLLSVR

SEQ ID NO: 10 - protein, 616 aa, Cicer arietinum
MKLKNIFLFFFFMFLECVFFKVESKCVKGCDLALASYYIKPPFEFTNITNFMQSNSDIIISYNKQLVSNNGKLFSFSRINIPFQCECIQGEFLGHMFEYTTNEGDTYDLIANSYYASLTTIDLLQKFNKYDHNQTLPSKVKVNVTVNCSCGNSQISKEYGLFLTYPLRSSDTLQIIAIESKVDEVLIQNYNPNVNFSRGSGIVFIPGRDKNGDYVPLYPRTGLAKGAAVGISMAGIFGFLLFVICIYVKYFQKNEEQNTKLLETSIALSTQSASGSGEYETSGSSGHGTSSAAGLTGIMVAKSTEFSYQELAKATNNFSLDNKIGQGGFGAVYYAELRGEKTAIKKMDVQASTEFLCELKVLTHVHHLNLVRLIGYCVEGSLFLVYEHIDNGNLGQYLHGIGTEPLPWSSRVQIALDSARGLEYIHEHTVPVYIHRDVKSANILIDKNLRGKVADFGLTKLIEVGNSTLHTRLVGTFGYMPPEYAQYGDVSPKIDVYAFGVVIYELISAKNAVLKTGESVAESKGLVALFEEALNKSDPCEGLRKLVDPRLGENYPIDSVLKMAQLGRACTRDNPLLRPSMRSIVVALMTLSSPTEDCDDDTSYENQTLINLLSVR

SEQ ID NO: 11 - protein, 620 aa, Abrus precatorius
MKPANGFLLFLLLLEYVCCNVESKCMKGCHVALASYYVRPKFLSLDNIMRLMQSKILSTSDVIRSYNKDKILNDNVPSFSRVNIPFPCDCIGDEFLGHVFEYSTAAGDTYDLIAKVKYANLTTVELLQRFNSYDQDDIPANSKLNVTVNCYCGNSQISKDYGMFITYPLRPGNTLQDISNETNLDAHLLQSYNPGVNFSSESGIVFIPGRDQNGDYVPLYPRSGLATGAAVGITIAAICGLILLVIFMYVKYFQKKEGEKAKLPSESSVAFSTQDASGSGEYETSGSTGPANASAAGLTGIMVAKSTEFSYQELAKATNNFSLDNKIGQGGFGAVYYAELRGEKTAIKKMDVQASTEFLCELKVLTHVHHLNLVRLIGYCVEGSLFLVYEYIDNGNLGQYLHGTGKDPLPWSSRVQIALDSARGLEYIHEHTVPVYIHRDVKSANILIDKHFRGKVADFGLTKLIEVGSSTLHTRLVGTFGYMPPEYAQYGDVSPKIDVYAFGVVLYELISAKNAVLKTGESVAESKGLVALFEEALNQSNPLEGIHKLVDPRLGENYPIDSVLKIAQLGRACTRDNPLLRPSMRSIVVALMTLASPTEDCDDDTISYENQTLINLLSVR

SEQ ID NO: 12 - protein, 611 aa, Glycine max
MELKKGLLVFFLLLECVCYNVESKCVKGCDVAFASYYVSPDLSLENIARLMESSIEVIISFNEDNISNGYPLSFYRLNIPFPCDCIGGEFLGHVFEYSASAGDTYDSIAKVTYANLTTVELLRRFNGYDQNGIPANARVNVTVNCSCGNSQVSKDYGMFITYPLRPGNNLHDIANEARLDAQLLQRYNPGVNFSKESGTVFIPGRDQHGDYVPLYPRKTGLARGAAVGISIAGICSFLLLVICLYGKYFQKKEGEKTKLPTENSMAFSTQDVSGSAEYETSGSSGTASATGLTGIMVAKSMEFSYQELAKATNNFSLENKIGQGGFGAVYYAELRGEKTAIKKMDVQASTEFLCELKVLTHVHHFNLVRLIGYCVEGSLFLVYEYIDNGNLGQYLHGTGKDPLPWSGRVQIALDSARGLEYIHEHTVPVYIHRDVKSANILIDKNIRGKVADFGLTKLIEVGGSTLHTRLVGTFGYMPPEYAQYGDISPKVDVYAFGVVLYELISAKNAVLKTGESVAESKGLVALFEEALNQSNPSESIRKLVDPRLGENYPIDSVLKIAQLGRACTRDNPLLRPSMRSIVVALMTLSSPTEDCDTSYENQTLINLLSVR

SEQ ID NO: 13 - protein, 618 aa, Glycine max
MELKKWLLFFLLLEYVCCNAESKCVKGCDVALASYYVSPGYLLLENITRLMESIVLSNSDVIIYNKDKIFNENVLAFSRLNIPFPCGCIDGEFLGHVFEYSASAGDTYDSIAKVTYANLTTVELLRRFNSYDQNGIPANATVNVTVNCSCGNSQVSKDYGLFITYLLRPGNNLHDIANEARLDAQLLQSYNPGVNFSKESGDIVFIPGKDQHGDYVPLYPRKTGLATSASVGIPIAGICVLLLVICIYVKYFQKKEGEKAKLATENSMAFSTQDVSGSAEYETSGSSGTASTSATGLTGIMVAKSMEFSYQELAKATNNFSLENKIGQGGFGIVYYAELRGEKTAIKKMDVQASTEFLCELKVLTHVHHLNLVRLIGYCVEGSLFLVYEYIDNGNLGQYLHGTGKDPFLWSSRVQIALDSARGLEYIHEHTVPVYIHRDVKSANILIDKNFRGKVADFGLTKLIEVGGSTLQTRLVGTFGYMPPEYAQYGDISPKVDVYAFGVVLYELISAKNAVLKTVESVAESKGLVALFEEALNQSNPSESIRKLVDPRLGENYPIDSVLKIAQLGRACTRDNPLLRPSMRSIVVALLTLSSPTEDCYDDTSYENQTLINLLSVR

SEQ ID NO: 14 - protein, 621 aa, Lupinus angustifolius
MKLKLVFLLLLKYVCFIVESKCIKGCDLALASYYVPVWPIVSLGNITSFMHSNVLTNPNVVTSYNKDKVFNGDVMLALYRTNVPFPCDCIGGEFLGHVFEYSAVEGDTYGLIAMKRYSNLTTVEILKRFNSYDPNHIPVNAKVNVTVKCSCGNSQISKDYGLFITYPLRPGNNLQELSKETKIDAKLLQSYNPGVNFSQENGIVFIPGKDQNGVYVPLYPRTGGVAKGVAVGISIAATCGLVLLVICIYDRYFKKKEGEKAKLSIENSIGFSTQDAYGSGEYETSGSSVHASALTGIMVAKSLEFSYQELAKATNNFSLDNKIGQGGFGAVYYAELRGEKTAIKKMDVQASSEFLAELKVLTHVHHLNLVRLIGYCVEGSLFLVYEYIDNGNLGQYLHGKGKDPLPWSTRLQIALDSARGLEYIHEHTVPLYIHRDVKSANILIDKNLRGKVADFGLTKLIEVGTSSLHTRLVGTFGYMPPEYAQYGDISPKIDVYAFGVVLYELISAKNAVLKTGETVAESKGLVNLFEEALNQINPLEPLTTLVDPRLGDNYPIQSLLKIAELGRACTRDNPLLRPNMKSIVVALMTLSSSNEDNTTSSYDNQTLINLLLDEGFRGITF

SEQ ID NO: 15 - protein, 624 aa, Lupinus angustifolius
MKLKFVFFLLLECVCFIVESKCIKGCDLALASYYVSVWPSISLGNITNFMHSNVLTNSDVIISYNKGKIFNGDVLLSLTRTNVPFPCDCIGGEFLGHVFQYSSVAGDTYDTIAMKSYSNLTTVEFLKRFNSYDPNHIPVNSKVNVTINCSCGNSLISKDYGLFTTYPLRPGNNLQELSKETNIDAKLLQSYNPGANFSQESRIVFIPGRDQNGVYVPLYPRIGGLARGAAVGISIAATCGLVLLIICIYDRYFKKKEGEKTKLSTEDSIGLSTEDGTGSGSGEYEASGSSGHAVGLTSIMVAKSLEFSYQELAKATNNFSLDNKIGQGGFGAVYYAELRGEKTAIKKMDVQASSEFLAELKVLTHIHHSNLVRLIGYCVEGSLFLVYEYIDNGNLGQYLHGKGRDPLPWSTRLQIALDSARGLEYIHEHTVPLYIHRDVKSANILIDKNLRGKVADFGLTKLIEVGTSSFHTRLVGTFGYMPPEYAQYGDISSKIDVYAFGVVLYELISAKSAILKTGETVSESKGLVTLFEGALNQINPLEALPKLVDPRIGDNYPIESVLKIAQLGRACTRDNPLLRPNMKSIVVALMTLSSSAEHSTSYDNQTLINLLLDEGFREIITPTS

SEQ ID NO: 16 - protein, 617 aa, Pisum sativum
MKLKNGLLLFFMFLDCIFFKVESKCVIGCDIALASYYVMPLVELLNITTFMQSKLVTNSSEVIVRYNRDIVFSNDNLFSYFRINIPFPCECIGGEFLGHVFEYTANEGDTYDLIANTYYASLTTVEVLKKFNSYDPNHIPAKAKVNVTVNCSCGNSQISKDYGLFITYPLRPRDTLEKIASHSKLDEGVIQSYNLGVNFSKGSGIVFFPGRDKNGEYVPLYPRAGLGKGAAAGISIAGIFALLLFVICIYIKYFQKKEEEKTKLPQISTALSAQDASGSGEYETSGSSGHGTGSTAGLTGIMVAKSTEFSYQELAKATNNFSLDNKIGQGGFGAVYYAVLRGEKTAIKKMDVQASTEFLCELQVLTHVHHLNLVRLIGYCVEGSLFLVYEHIDNGNLGQYLHGIDKAPLPWPSRVQIALDSARGLEYIHEHTVPVYIHRDVKSANILIDKNLHGKVADFGLTKLIEVGNSTLHTRLVGTFGYMPPEYAQYGDVSPKIDVYAFGVVLYELISAKNAILKTGESAVESKGLVALFEEALNQIDPLEALRILVDPRLKENYPIDSVLKMAQLGRACTRDNPLLRPSMRSLVVALMTLLSHTDDDDTFYENQSLTNLLSVR

SEQ ID NO: 17 - protein, 618 aa, Vigna angularis
MELRKGLLLLFLLLEYVCCNVEAKCLKGCDLALASYYVSSGLTLDDITHLMKSSVVSNSDDIISYNKDKTFNKNIFLFDRINVPFPCDCIHDEFLGHVFEYSAAEGDTYDSIAKVEYADLTTVELLRRFNSYGQNGIPKNAKVNVTVNCSCGNSQVSQEYGLFITYPLRPDNNLHDIANEAHLDAQLLQNYNPGVNFSKESGIVFIPGRDQFGDYVPLHPRKTGLATGAVAGISIAGICGLLLFIICTYVKFFKKMEGEKPKLPTEKSTAFSIQDASGSADYETSGSSGTATATGLTGIMVAKSMEFTYQELAKATNNFSLENKIGQGGFGAVYYAELRGEKTAIKKMDVQASTEFLCELKVLTHVHHLNLVRLIGYCVEGSLFLVYEHIDNGNLGQYLHGTGKDPLTWSSRVQIALDSARGLEYIHEHTVPVYIHRDVKSANILIDKNFRGKVADFGLTKLIEVGSSTLHTRLVGTFGYMPPEYAQYGDVSPKVDVYAFGVVLYELISAKNAVLKTGEYVAESKGLVALFEEALNQSNPSEAIRKLVDPRLGENYPIDSVLKIAQLGRACTRDNPLLRPSMRSIVVALMTLSSPTEDFDDETASYENQTLINLLSVR

SEQ ID NO: 18 - protein, 618 aa, Vigna radiata
  source 1..618: note = var. radiata
MELRKGLLLLFLLLEYVCCNVEAKCLKGCDLALASYYVNRGLSLDNITHLMKSSVVSNSDDIISYNKNKIFNKNVFMFNRYNVPFPCDCIRDEFLGHVFEYSAAEGDTYDSIAKVEYADLTTVELLRRFNSYGQNGIPKNAKVNVTVNCSCGNSQVSQEYGLFITYPLRPDNNLHDIANEAHLDAQLLQNYNPGVNFSKESGIVFIPGRDQFGDYVPLHPRKTGLATGAVAGISIAGICGLLLLVICIYVKFFKKMEGEKPKLPTEKSTASSIQDASGSAEYETSGSSGTATATGLTGIMVAKSMEFTYQELAKATNNFSLENKIGQGGFGAVYYAELRGEKTAIKKMDVQASTEFLCELKVLTHVHHLNLVRLIGYCVEGSLFLVYEHIDNGNLGQYLHGTGKDPLTWSSRVQIALDSARGLEYIHEHTVPVYIHRDVKSANILIDKNFRGKVADFGLTKLIEVGSSTLHTRLVGTFGYMPPEYAQYGDVSPKVDVYAFGVVLYELISAKNAVLKTGEFVAESKGLVALFEEALNQSNPSEAIRKLVDPRLGENYPIDSVLKIAQLGRACTRDNPLLRPSMRSIVVALMTLSSPTEDFDDETASYENQTLINLLSVR

SEQ ID NO: 19 - protein, 618 aa, Vigna unguiculata
MELRKGLLLLFLLLEYVCCNVEAKCLKGCNVALGSYYVNTGMSLDVITPLMKSSVVSNSDDIISYNKDKIFNKNVFYFDRINVPFPCDCISDEFLGHVFEYSAAEGDTYDSIAKVEYADLTTVELLRRFNSYGQNGIPKNAKVNVTVNCSCGNSQVSQDYGLFITYPLRPGNNLHDIANEAHLDAQLLQNYNPGVNFSKESGIVFIPGRDQFGEYVPLHPRNTGLAKGDVAGISIAGICGLLLLVICIYVKFFKKKEGEKPKLPTENSTAFSTQDASGSAEYEISGSSGTTTAAGLTGIMVAKSMEFTYQELAKATNNFSLENKIGQGGFGAVYYAELRGEKTAIKKMDVQASTEFLCELKVLTHVHHLNLVRLIGYCVEGSLFLVYEHIDNGNLGQYLHGTGKDPLPWSSRVQIALDSARGLEYIHEHTVPVYIHRDVKSANILIDKNFRGKVADFGLTKLIEVGGSTLHTRLVGTFGYMPPEYAQYGDVSPKVDVYAFGVVLYELISAKNAVLKTGEFVAESKGLVALFEEALNQSNPSEAIRKLVDPRLGENYPIDSVLKIAQLGRACTRDNPLLRPSMRSIVVALMTLSSPTEDFEDETASYENQTLINLLSVR

SEQ ID NO: 20 - protein, 629 aa, Arachis duranensis
MKLKRSFLLFFLLLECSCFINLESKCLKGCDLAIASYFVYPGVMLGSIASFMHSNVVPNSNVIISYNKDKMPNNLPVSFSRINIPFPCDCINGEFLGHVFEYSAIEGDTYDLIANLRYSDLTTVEILQRFNNYDPNHVPVNAKVNVTVNCSCGNSNVSKDYGLFVTFPLSEGNTLLQIANQTKLDPKLLQSYNPGVNFNQTRGIVFIPARDQNGVYVPLYPRTGLAKGVAVGISIAGTSGVLLLLICACVRYFQKKEEEKIKLPTEDFISSSTQDASSSGEYETSGSTAASASGLPAIMAAKSMEFSYQELSKATNNFSSDNKIGQGGFGAVYFAELRGQKAAIKKMDVQASTEFLSELKVLTHVHHLNLVRLIGYCVEGSLFLVYEYIENGNLGQYLHGTGREPLAWLTRVQIALDSARGLEYIHEHTVPVYIHRDVKPANILIDKDFRGKVADFGLTKLREVGSSLSLHTRLVGTFGYMPPEYAQYGDVSPKVDVYAFGVVLYELISAKNAVLKTGETVAESSKGLVTLFEEALDKSDPSKAIRKLVDPRLGENYSIDSVLKVAELGRACTRDNPLLRPSMRSVVVALMTLLASSTSIENCDDDDDDDDDDDNSYENRTLIDQLSVR

SEQ ID NO: 21 - protein, 630 aa, Arachis ipaensis
MKLKKSFLLFFLLLECSCFINLESKCLKGCDLAIASYFVYPKVMLGSIASFMHSNVVPNSNVIISYNKDKMPNNLPVSFTRINIPFPCDCINGEFLGHVFEYSAIEGDTYDLIANMRYSDLTTVEILQRFNNYDPNHVPVNAKVNVTVNCSCGNSNVSKDYGLFVTFPLSEGNTLLQIANQTKLDPKLLQGYNPGVNFNQTRGIVFIPGRDQNGVYVPLYPRTGLAKGVAVGISIAGTSGVLLLLICACVRYFQKKEEEKIKLPTEDFISSSTQVASSSGEYETSGSTAASASGLPAIMAAKSMEFSYQELAKATNNFSSDNKIGQGGFGAVYFAELRGQKAAIKKMDVQASTEFLSELKVLTHVHHLNLVRLIGYCVEGSLFLVYEYIENGNLGQYLHGTEREPLAWSTRVQIALDSARGLEYIHEHTVPVYIHRDVKPANILIDKDFRGKVADFGLTKLREVGSSLSLHTRLVGTFGYMPPEYAQYGDVSPKVDVYAFGVVLYELISAKNAVLKTGETVAESSKGLVTLFEEALNKSDPSKAIRKLVDPRLGENYPIDSVLKVAELGRACTRDNPLLRPSMRSVVVALMTLLASSTSIEDCDDNDDDDDDDDDNSYENRTLIDQLSVR

SEQ ID NO: 22 - protein, 550 aa, Chamaecrista fasciculata
MKPLLALALLFLLLETAFLNVESMCIKGCDLALASYYIPLFFTLQNLTILLQSKVLSDYQVIASFNKDSIFNGVNIKSYQSINVPFPCDCIDGDFLGHVFEYTIANGDTYERIASRKYAMLTSVELLKKYNNYSDPNHLPINGKLNVTVNCYCGDRQISKDYGLFVTYPLRDGDSLQTIAEKTNLDLGLLERYNQGMNFSQGSGLVFYPGKGLRGSAITGMSIGGMLGSLLVAIYIYLRYYRKKAVKVALTTEDSTDLSTQDASGSAGYDTSGSGNSAAGLKGIVVSKSVEFSYQEIAKATNNFSFDNKIGQGGFGAVYYAELRGEKTAIKKMDIEASQEFLAELKVLTHVHHLNLVSHLSMKGERQCHGLRGCKLLWIQQEVADFGLSKLIEVGNASLNTRLVGTFGYMSPEYAQYGITSPKIDVYAFGVVLYELISAKNAVLKTGECVGVAESKGLVTLFEEALRWPDPIEGLRKLVDPKLGENYPIDSIYKMAQLARACTQEDPQLRPSMRSIVVALMTLSSYGEDCDHVDSNLYDNQTLINMLSER

SEQ ID NO: 23 - protein, 594 aa, Mimosa pudica
MQPKSHNALVLAVFFQILLLETTFLINNVESKCHKGCPLALASYLCHQNITLSNITHLLKSNLVSLDFQIISSYNKEGIVPNNIQFFTRINIPFPCECINNDDFLGHVFEYTISKGDTYHSIATQTYSSLTSVDLLRRFNNYSDPNQLPPNGKLNVTVKCSCGNRQISKDYGLFITYPLGFEDSLESIVNQTGIDALLLQSYNPGANFSQQSGVVFIPGRADQTGNYMPLDYIRSGRLRGTAIAGISVAATFVFLLLAAYVFVRYFRKKENAKLALFSENSAELSSQEGSSGVASTTDLRGIMEEKSMEFSYQEIAKATNNFSSDNRIGQGGFGTVYYAELRGQKTAIKKMDIKASQGFLSELKVLTHVHHLNLCFFHYINVNIYNDFDQEKIYNKIYCLNVLPWSTRVQIALDSARGLEYIHEHTVPTYIHRDVKSLNILIDKTYHAKVVADFGLSKLIEVGNASLNTRRLVGTFGYMSPEYAQYGIQSPKIDVYAFGVVLYELISAKNAVLKITNEFVAESKGLVALFEKCLRRPDAVEDLRNLVDPRLGQNYPIESVYKVQKLFEYESGLDSLNFEILKFFIKNSKRKVVT

SEQ ID NO: 24 - protein, 626 aa, Lupinus albus
MKLKFVFLLLLEYVCFTVESKCIKGCDIALASYYVPVWPTVSLGNITNYMYSNVLTKSNVIISYNKDKVFNGDVMLALYRTNIPIPCDCIGGEFLGHVFEYTAVAGDTYDLIAMKRYSNLTTVEFLKRFNSYDPNHIPVNAKVNVTVNCSCGNSLISKDYGLFITYPLRPGNNLQELSKETNIDAKLLQSYNPSVNFSQENGIVFIPGRDQNGVYVPLYPRTEGLARGVAVGISIAASCGLVLLVICIYDRYFKKKEGEKAKLSIENSLVLSTQDASGSSEYETSGSSVHASGLTGIMVAKSLEFSYQELAKATNNFSLDNKIGQGGFGAVYYAELRGEKTAIKKMDVQASSEFLAELKVLTHVHHLNLVRLIGYCVEGSLFLVYEHIDNGNLGQYLHGKGKDPLPWSTRLQIALDSARGLEYIHEHTVPLYIHRDVKSANILIDKNLRGKVADFGLTKLIEVGTSSLHTRLVGTFGYMPPEYAQYGDISPKIDVYAFGVVLYELISAKNAVLKTGETVAESKGLVSLFEGALNHINPLEALSTLVDPRLGDNYPIESVLKIAQVGRACTRDNPILRPNMKSIVVALMTLSSSTHQDHTSSSSYDHQTLINLLLDEGFRGIITPTF

SEQ ID NO: 25 - protein, 644 aa, Spatholobus suberectus
MFLLFGLRDPMKLKKGLLFFLLLEYVCRSVESKCVKGCDVALASYYVSPGYFPLENITRLMESSVLSNSDVVTSYNKDKIFNDMVQSFVRLNIPFSCGCIGGEFLGHVFEYSAAAGDTYDSIAKVTYANLTTVELLRRFNSYDQNDIPANAKLNVTVNCSCGNSQVSKDYGLFITYPLRPGNNLHDIANETQLDAQLLQSYNPGVNFSQESGIVFIPGRGILSFSHHVHLVHSSLYFDQNGDYVPLYPRKTGLARGAAVGISIAGICGLLLLVIFVYVRYFQNKEGKKDKLPTENSMVFSTQDGTEYETSGSSGLATASAAGLTGIMVAKSMEFSYQELAKATNNFSLENKIGQGGFGAVYYAELGGEKTAIKKMDVQASTEFLCELKVLTHVHHLNLVRLIGYCVEGSLFLVYEYIDNGNLGQYLHGTGKDPLPWSSRVQIALDSARGLEYIHEHTVPVYIHRDVKSANILIDKNLRGKVADFGLTKLIEVGGSTLHTRLVGTFGYMPPEYAQYGDISPKVDVYAFGVVLYELISAKNAVLKTGESVAETKGLVALFEEALSQSNPSEGIRKLVDPRLGENYPVDSVLKIAQLGRACTRDNPLLRPSMRSIVVALMTLSSPTEDCDDDASYENQTLINLLSVR

SEQ ID NO: 26 - protein, 629 aa, Prosopis alba
MQPKSNSLVLVFFLLLLETSLLNNVESKCLQGCPLALASYYAYKNNLGNITKFLRSDLVSDFGVVSSYNEGKVSNNGMYVQSLTRINIPFPCDCINNGDFLGHVFEYTIAEGDTYDSIATQTYSSLTTVDLLRRFNSYKDPNQLPLNGKLNVTVNCSCGDRQISKDYGLFLTYPLRFGDSLESIVDQTGIDATLLQKYNLRVNFSQESGVVFIPGRDQNGNYVPLDPRSRGLSEVAIAGICVGATFVFLLLSAYVFVKHFRRKTAKVALITENSTDLSSNQDGMIITSTNYETSGSSGSASATGLRGIMVGKSMEFSYQEIAKATNNFSSDNKIGQGGFGAVYYAELRGEKTAIKKMDIEASQEFLAELKVLTHVHHLNLVRLIGYCVEGSLFLVYEYINNGNLSQHLRSSELDALPWCTRVQIALDSARGLEYIHEHTVPVYIHRDVKSLNILIDESFHAKVADFGLSKLIEVGNASLNTRLVGTFGYMSPEYAQYGITSPKIDVYAFGVVLYELISAKNAVLKTGEFVAESKGLVALFEKSLGRPDAVEDLRKLVDPRLGQNYPIDSVFKMAQLARACTQDDPQLRPTMRSIVVALMTLSSNADDEDCDVGSAFSDNHTIINLLSGR

SEQ ID NO: 27 - DNA, 1317 bp, Lotus japonicus
  source 1..1317: mol_type = unassigned DNA
ggacatgagattgaagctccaaaattagctcttttttctgatgaatacttaatgctttgttgtattcacttgattaagtgctagaaatcatctttgcatgatcatagattaaatgaatttccagttggtgtgtggagagctattttgttatgctgacatctgcaatttgcagggcatctaatgattgtcatttcttaaattattattggttgtttccgtttctttaattatctgttttaatcttgcaggtcatacaaattaaaatactagccaccacccaagacatactaaatggggtagtagagggaagggtaaggtcgataaggatgactttttattctataaaatttaggagaatttgagcttaagtggcaaggcaaacgacattactatacgaattggctttgtaccagaaacagggaacaaataatattttacaaataagctattatcatgtcagctcatttgttcaactttgatttgattaaaaattaaatgaagttgaatttgttgagctgctttattatatatgccactggatgtttccgcattctaagtgcatgtttgaaaacatttctacaattgattacgaaggaaaaattaatcatggagagaagcttatgtgcgtagcttctgtatttctgaattgattctatctgtacagtagcatttagataatgaatgatcttggttctcgctaagcatcaaaccaatctctacccttttaaaattgcaagaattataagtcatgcattgacccaaatccttctgtggttatgccccttaaaaatccggcaagacatcaagttagttggtcattagggttccaccagctagctgacaccttgtacaacaactggccgtcctaaagttgggtaagcattacaatactaaatgccattttattatattttgcgcatggttatatacctaagtaggatttgtccacagtttctttgattcggaaaggaaaaaatatttagttgacactgacagaagcagattttatatacatatattatgaaatgactcctacatgagatacacgaatctcatccccatgagttgcagtttgacagagtacacacttatcaacttgctggaatataggaaagtctaaccaatgatgtcgatccgtattgccttaattttggtaaatttagtattacatgatcattattgatatactaaaccacaggatattttattgacaatgtgaatgttccatattttcaacaatgctgattccctctgataaagaacaagttccttttctctttccctgttaactatcatttgttccccacttcacaaaca

SEQ ID NO: 28 - DNA, 2000 bp, Medicago truncatula
  source 1..2000: mol_type = unassigned DNA
aaataatgtgtctaaaattaacaaagatattttatggttaatttattgagcttagttaaagtcattttctaaaacgagacattgtagttgttagataaataatttcctttaattttcatcaaagtttgtaaataattatagggcaggacacatatgtccatcaattgatatatatatatcttttgtaataacgatcttatagaagaatcaatatctacaaagaaagttatcacttgtctcaaattatgctttatgcaactggtattgtcatgtagattccattttggtttatatgatattgaagtcaacgaatcacagctgcttaggcgtggttcataatttaaaattgaaactatggtgtttgttggaattatgagaggacagactgtgaagtggttgcacagctaccgacagggttgccattactgatgcaaaggattaaacaatagaacattttcaatcaacttaatctatgtttgacatcacgctcgatgtgtcagaatcacgacgacccactgtaatttttaaaaaaattacattttataatttttgtagaatcacggtgtatcacagtgattttgacatacccactgtaataccaaacatgcacttgcattctttcttccgtttgatgccaaaaatggtattgtttataatacacttaaaataattcatatccaagttgttgtataaaaaaataaacaatgtgatttcccaaaaaaaaaaaataataagtgattggcatgcaacatcagattttgttgtccaagtggtcacatttgaaatatttgagtttacatttttctcactaatatctttatattgaacttcaatctgtctagctataatcaaatggttgactaaaatggttaataagcttcaattaaattcaaattatttgaaagaacttgaatttaaatcttaacttgaacataaccccgaagtggatgatttacccaaaaaaattacctaactactctaataatcattttaacaataagagtattttactcaaagaaatttatataatcacaaaatcctagtatactcaatgattttcttaaaaaactattataaccagaaatgacaatatgagacaaatataattattttaataaaaatatctaaattagaattatttgaaaatgtaaaactatattattttttgtagttttccaaactttattaagcatcgtttaattgcacacaaaatttactccaattttttttagtctctgaaagttgctttttggtccatgttttagttttggtaattataattaagtttgttcatattaattagttctaatgtgtgtactatatagatcatactaattttgttcccaaactcatcttagttaggatctttgtatctactataagattaatccttgtgccgatataagaatatttgtcatgttatcaactatcacaatattgtattggtgagatcatataagatttgatgtcaacatcttcgtaaaggtgtcagattcgattctccccggtatcaatttaagtgagctaatttagcttcttaaaaaataaaatcaaacaacttttacataaactcagtgaaaacttggatataaagtatccttatactactctttagtcttgattagtctctgcaaagatatttatatgtactttgtattatcataagaacattcattgacattttaagttaatgaattactaacatgtcaactcttattctagccaacagttactttgttccctccacattctctttgaaatagtcaaacgtatccaatcatgcatgtctgttctgatcataacagcaaaagcatgtgtatagaaaattgatagttgaattagagtcattttccataaaaaaatattcaataagtgtgacattatttttcgtatgaattaatccattttttgctgatttgagattctttctttctttgcttcttgctttccttcatcagccattttttttgttttctctttctctctctcttcttgattca

SEQ ID NO: 29 - DNA, 1135 bp, Medicago truncatula
  source 1..1135: mol_type = unassigned DNA
cgatccttcgttgtattcacttggtcaagtgcaagaaatcatctttacacaatcatataatgaatggacctttggttgatgtgttgagagctacattcagtcctctgctgacttctgcaacttgaggggaatctaatgattgtcttattgcaattaacaattccttttaattttttgacagaaagagttggttacaaaatctcatctaacatattgttgagttcctcttttttttttttttttttttttttgataaaagggtacatctgttcgcagctctgaagattaatccctcgaatagggttggataacatgaacgacaaatttgtttcttaagagttttaacactgtcatattcttatggcagaattcgaactcgaacccactggttaagctagaagtaagaagtgacccatgccatctcattcaatcgttcttggcttgttttcggtttagatgaatatgaagtacaaaaagctacgctcaagcaaaggagagcaggtaagaagggcatcttatttttttaatatattagatgtcaagcttacgtggcgatgtaaacactgtttctatccgaattagctatgtactagaaatagggaacaaataatataaatggtgagtttgtcaaggtgttttaatatattgaaaccaatgtaactggaagtgtcaacattgaaagctgttcattgcctgtattatattataattgcatatatataatggcattagcctttgttaaacgttaaaccatatcttttgctagtttgccccttaaaaatttggtaaaggcatcaagttagatggtctttaggtaccagccagctagttgacatcgtgtatggacaccaaattgggttataataccatattattcttaccatctttattatattctgcgcatgatttcatacttgtttgggatttgccaacagtgtctaagatttgaaaaagaaaaatagtagaactaatgacagagacatcagcatatatttttaatatcaaaccaaaagatatttgatgtccaaagaagagataaatataaagttagaagtatgacaataagtcttggttgttatttgccataagaaactctcttttctcttcccctcataatttgcatttcctcacaaca

SEQ ID NO: 30 - protein, 627 aa, Vigna angularis
MFLLLITDPMELRKGLLLLFLLLEYVCCNVEAKCLKGCDLALASYYVSSGLTLDDITHLMKSSVVSNSDDIISYNKDKTFNKNIFLFDRINVPFPCDCIHDEFLGHVFEYSAAEGDTYDSIAKVEYADLTTVELLRRFNSYGQNGIPKNAKVNVTVNCSCGNSQVSQEYGLFITYPLRPDNNLHDIANEAHLDAQLLQNYNPGVNFSKESGIVFIPGRDQFGDYVPLHPRKTGLATGAVAGISIAGICGLLLFIICTYVKFFKKMEGEKPKLPTEKSTAFSIQDASGSADYETSGSSGTATATGLTGIMVAKSMEFTYQELAKATNNFSLENKIGQGGFGAVYYAELRGEKTAIKKMDVQASTEFLCELKVLTHVHHLNLVRLIGYCVEGSLFLVYEHIDNGNLGQYLHGTGKDPLTWSSRVQIALDSARGLEYIHEHTVPVYIHRDVKSANILIDKNFRGKVADFGLTKLIEVGSSTLHTRLVGTFGYMPPEYAQYGDVSPKVDVYAFGVVLYELISAKNAVLKTGEYVAESKGLVALFEEALNQSNPSEAIRKLVDPRLGENYPIDSVLKIAQLGRACTRDNPLLRPSMRSIVVALMTLSSPTEDFDDETASYENQTLINLLSVR

SEQ ID NO: 31 - protein, 627 aa, Vigna unguiculata
MFLLLITNPMELRKGLLLLFLLLEYVCCNVEAKCLKGCNVALGSYYVNTGMSLDVITPLMKSSVVSNSDDIISYNKDKIFNKNVFYFDRINVPFPCDCISDEFLGHVFEYSAAEGDTYDSIAKVEYADLTTVELLRRFNSYGQNGIPKNAKVNVTVNCSCGNSQVSQDYGLFITYPLRPGNNLHDIANEAHLDAQLLQNYNPGVNFSKESGIVFIPGRDQFGEYVPLHPRNTGLAKGDVAGISIAGICGLLLLVICIYVKFFKKKEGEKPKLPTENSTAFSTQDASGSAEYEISGSSGTTTAAGLTGIMVAKSMEFTYQELAKATNNFSLENKIGQGGFGAVYYAELRGEKTAIKKMDVQASTEFLCELKVLTHVHHLNLVRLIGYCVEGSLFLVYEHIDNGNLGQYLHGTGKDPLPWSSRVQIALDSARGLEYIHEHTVPVYIHRDVKSANILIDKNFRGKVADFGLTKLIEVGGSTLHTRLVGTFGYMPPEYAQYGDVSPKVDVYAFGVVLYELISAKNAVLKTGEFVAESKGLVALFEEALNQSNPSEAIRKLVDPRLGENYPIDSVLKIAQLGRACTRDNPLLRPSMRSIVVALMTLSSPTEDFEDETASYENQTLINLLSVR

SEQ ID NO: 32 - protein, 615 aa, Mimosa pudica
MEPRNISPVFIIIIFFISSVFPITESSCSKSCDLALASYYVWPFSNLTFISDQMKSQILTDPNNIVSYNKDQTISKDYVQSTTRIDIPFPCDCLQDGSLGHLFQYKISEGDTYKTIAETYYSNLTTVESLRKLNGNTIPSSGTINVPVTCYCGDSLVSKLYGLFITWPLRPEDSLASIAQSENLDAELLQRYNPGVNFSQGSGLVFIPGKXXXAGLSGGVIAGISVGVVAGLLLLAVSIYFGCFWKKTDDTRPLSQDFQEFSVNEEKGKNLFSCYNLSSNIESEILGYGGPTGITVDKSVEFSYEELAAATNNFSMANKIGQGGFGAVYYAELRGEVAIKKMDMQATREFLAELKVLTNVHHLNLVRLIGYCTENSLFLVYEFIDNGNLSQHLRGTGAGRDPLPWPTRVHIALDSARGLEYIHEHTVPVYIHRDIKSANILIDNSFRGKVADFGLTKLTEVGNSSLPTGNLVGTFGYMPPEYAQYGDVSPKVDVYAFGVVLFELISAKEAIVKTTQSDVDSKGLVMQFHGVLNQPDPSEDLRKLVDPRLGDDYPTDSVLKMAQLAKACTQENPQLRPTMRSIVVALMTLYSSTADDWDVGAFYNNPNLMNLMSGR

SEQ ID NO: 33 - protein, 591 aa, Lotus japonicus
MTSFFLFTNTLFLALMMFFSTTHHILAQLSHTNGTNFSCPVDSPPSCDTYVTYFAQSPNFLTLTSISDLFDTSPLSIARASNIKDENQNLVPGQLLLVPVTCACSGSNSFSNISHMIKEGESYYYLSTTSYENLTNWETVQDSNPNYNPYLLPVGIKVVIPLFCKCPSNYHLNKGIEYLITYVWHNNDNVSLVASKFGVSTQDIISENNFSHQNFTAATNFPILIPVTQLPSLSQSYSSSERKRSNHIHIIISIGISLGSTLLIALLVLVSVTCLRKRKSSENKSLLSVEIAGKKLISGVSNYVSKSILYEFRLIMEATLNLNEQCKIGESVYKAKLDGQVLAVKKVKEDVTEEVMILQKVNHLNLVKLMGVSSGHDGNHFLVYEFAENGSLHNWLFSNSSTGSRFLTWSQRISIAVDVAMGLQYMHEHTQPSIVHRDITSSNILLDSNFKAKIANFSVARTSINPMILKVDVFGYGVVLLELLSGKKSLTNNEINHIREIFDLKEKREERIRRWMDPKIESLYPIDDALSLAFLAMNCTSEKPLSRPTMGEVVLSLSLLMTQHSPTTLERSWTCGLDVDVTEMQTLIAAR

SEQ ID NO: 34 - protein, 596 aa, Abrus precatorius
MAFFFLSYHTLFLALMFFTTTHILAQLPLTNGTNFSCPVNSIPSCDTYVAYFAQSPNFLTLTSISDIFDTSPLSIARASNMKSEYEDLIPGQLLLVPVNCSCNGNLSFATISHEIRQGDSYYSLSTTSYENLTTWEKVKDLNPHLDPYQLHAGIKIVIPLFCKCSSRYHLDKGIEYLITYVWQQNDNVTLVAAKFGASPVDIITENNISQNFNVATNLPVFIPVTQLPALSQIYSPHERKGSNHLSIVIISIGVSIVLFTLLLLVYVYCLRKKKTSSENRSGPSVETADKLISGVSCYVSKPTMYEVGVIMEATMNLNEQCRIGESMYMAKMDGQVLAVKKVKEDVTKEVMILQKVNHANLVKLTGVSLGNDGNFFLVYEYAENGSLHNWLFSKSSNVSSSMGFLNWSQRISIAIDVAMGLQYLHEHIQPCIVHMDITTSNILLDSNFKAKIANFSAARTSTNPMITKLDVFGYGMVLLELLTGKESLANNENGELAMVWSKVKGIFDQEVKREERFRKLMDPMLGEFYSLDDALSIAFWAVNCTAEKPLSRPTMGEVVLSLSLLTQHSPSTLERSWTHGLEVEVTRTLLTPVTAR

SEQ ID NO: 35 - protein, 603 aa, Cicer arietinum
MVSSFLPSHTLFFALMFFSATHILLQLPQINGTNFSHPLNSPPSCHTYAAYHAHSPNFLTLTSISDIFDTSPLSIARASNIKDENKELIPGQLLLVPITCACSGNGNYSSANISHFIKQGESYYYLSTISFQNLTNWQTVEDSNPNLNPYLLKLGTKIVIPLLCKCPSNYQFEKGIEYVITYVWQPNDNVTLVASKFGALPHDIITANANNFGQNFTTATNLPVFIPVTNLPVISQSYYYSSERKRSNHFPIIIAIGMSLVCTLLISLSLMVCVYCWRKRKACENRCVPSVEIANKLISEISNYVSKPKVYQVDIIMEATMNLNEQCKIGESVYKGKIDGLVLAVKNVKEKVTVTEELMILQKVNHTNLVKLIGVSSGYDGNHFLVYEYAENGSLYNWLFSEFSTSSSSGVFLTWSQRLSIAVDVAMGLQYMHEHTQPRIVHRDITSSNILLNSNFKAKIANFSAARTTKDPMITKVDVFAYGVVLLELLTGKKSLSYSENGEVNMSWKDIREIFDIEEKRDERLRRWIDPLLGRFFNVVEVLSLASIAVNCTKEQPLSRPTMGEVVLSLSLLTQHSTTTLERSWNFGLDLEDTGMVTPITAR

SEQ ID NO: 36 - protein, 590 aa, Medicago truncatula
MVSSFFHTLIFFSATHILLQLPQANGKNFSCTLNSSPSCDTYVAYFANSPNFLTLTAISDIFDTSPQSIARASNIKDENMNLIHGQLLLIPITCGCNGNGNYSFANISHLIKESESYYYLSTISYQNLTNWQTVEDSNPNLNPYLLKIGTKINIPLFCRCPSNYFAKGIEYLITYVWQPNDNLTLVASKLGASPKDIITANTNNFGQNFTVAINLPVFIPVKNLPALSQSYYSSSERKRINHFSIIISIGICLGCTILISLLLLLFYVYCLRKRKACENKCVPSVEITDKLISEVSNYVSKPTVYEVGMIMKATMNLNEMCKIGKSVYKAKIDGLVLAVKNVKGHITVTEELMILQKVNHANLVKLVGVSSGYDGNHFLVYEYAENGSLYNWLLSEFCTLSWSQRLSIAVDIAIGLQYLHEHTQPCIVHRNIKSSNILLDSKFKAKIANFSVARTTKNPMITKVDVLGYGMVLMELITGKKFLSYSEHSEVNMLWKDFKCVFDTEQKREEIVRRWMDPKLGRFYNVVEALSLFTLAVNCIEEQPLLRPTMGEVVLSLSLLTQPSPTLLEVSWTYGLDVEVAEMVTPIIAR

SEQ ID NO: 37 - protein, 595 aa, Lotus japonicus
MAVFFLTSGSLSLFLALTLLFTNIAARSEKISGPDFSCPVDSPPSCETYVTYTAQSPNLLSLTNISDIFDISPLSIARASNIDAGKDKLVPGQVLLVPVTCGCAGNHSSANTSYQIQLGDSYDFVATTLYENLTNWNIVQASNPGVNPYLLPERVKVVFPLFCRCPSKNQLNKGIQYLITYVWKPNDNVSLVSAKFGASPADILTENRYGQDFTAATNLPILIPVTQLPELTQPSSNGRKSSIHLLVILGITLGCTLLTAVLTGTLVYVYCRRKKALNRTASSAETADKLLSGVSGYVSKPNVYEIDEIMEATKDFSDECKVGESVYKANIEGRVVAVKKIKEGGANEELKILQKVNHGNLVKLMGVSSGYDGNCFLVYEYAENGSLAEWLFSKSSGTPNSLTWSQRISIAVDVAVGLQYMHEHTYPRIIHRDITTSNILLDSNFKAKIANFAMARTSTNPMMPKIDVFAFGVLLIELLTGRKAMTTKENGEVVMLWKDMWEIFDIEENREERIRKWMDPNLESFYHIDNALSLASLAVNCTADKSLSRPSMAEIVLSLSFLTQQSSNPTLERSLTSSGLDVEDDAHIVTSITAR

SEQ ID NO: 38 - protein, 595 aa, Medicago truncatula
MSAFFLPSSSHALFLVLMLFFLTNISAQPLYISETNFTCPVDSPPSCETYVAYRAQSPNFLSLSNISDIFNLSPLRIAKASNIEAEDKKLIPDQLLLVPVTCGCTKNHSFANITYSIKQGDNFFILSITSYQNLTNYLEFKNFNPNLSPTLLPLDTKVSVPLFCKCPSKNQLNKGIKYLITYVWQDNDNVTLVSSKFGASQVEMLAENNHNFTASTNRSVLIPVTSLPKLDQPSSNGRKSSSQNLALIIGISLGSAFFILVLTLSLVYVYCLKMKRLNRSTSSSETADKLLSGVSGYVSKPTMYEIDAIMEGTTNLSDNCKIGESVYKANIDGRVLAVKKIKKDASEELKILQKVNHGNLVKLMGVSSDNDGNCFLVYEYAENGSLEEWLFSESSKTSNSVVSLTWSQRITIAMDVAIGLQYMHEHTYPRIIHRDITTSNILLGSNFKAKIANFGMARTSTNSMMPKIDVFAFGVVLIELLTGKKAMTTKENGEVVILWKDFWKIFDLEGNREERLRKWMDPKLESFYPIDNALSLASLAVNCTADKSLSRPTIAEIVLCLSLLNQPSSEPMLERSLTSGLDAEATHVVTSVIAR

SEQ ID NO: 39 - protein, 597 aa, Cajanus cajan
MAVFSCFLPLCSQILCLALVLFSTNIASQSQQANGTNTNFSCPADSPPSCETYVTYIAQSPNFLSLTSISNLFDTSPLSIARASNLDPDNNKLIPDQILLIPVTCGNRSFANISYEINQGDTFSFLSTTSYENLTNWHVVDDLNPDLNPNMLPIGIQVVFPLFCKCPSKNQLDKGIKYLITYVWQPSDNVSLVSDKFDASPEDILSENNYGQNLTAANNLPVLIPVTRLPVLARSPSDGRKGRNHLPVIIGISLGCTLLVVVSMLLLVYVYCLKMKSLNRSASSAETADRLLSGVSGYVSKPTVYETNVIMEGTMNLSEQCKIGESVYKANIEGKVLAIKRFKEDVVEQLKILQKVNHGNLVKLMGVSSDNDGNCFVVYEYAENGSLDEWLFSKSSSETSNSMASLTWCQRISIAVDVAMGLQYMHEHAYPRIVHRDITSSNILLDSNFKAKIANFSMARTFTNPMMTKIDVFAFGVVLIELLTGRKAITTKENGEVVMLWKDIWKIFDQEEDREERLRRWMDPKLESYYPIDYALSLASLAVNCTSDKSLSRPTIAEIVLSLSLLTQPSPATLERSLTSSGLDVDATQIVTSVVAR

SEQ ID NO: 40 - protein, 597 aa, Phaseolus vulgaris
MAVFFVSLTLGAQILYVVLMFFTCIEAQSQQTNGTNFSCPSNSPPSCETYVTYISQSPNFLSLTSVSNIFDTSPLSIARASNLQHEEDKLIPGQVLLIPVTCGCTGNRSFANISYEINQGDSFYFVATTLYQNLTNWHAVMDLNPGLSPFTLPIGIQVVIPLFCKCPSKNQLDRGIKYLITHVWQPNDNVSFVSNKLGASPQDILSENNYGQNFTAASNLPVLIPVTLLPDLIQSPSDGRKHRIGLPVIIGISLGCTLLVVVSAILLVCVCCLKMKSLNRSASSAETADKLLSGVSGYVSKPTMYETGAILEATMNLSEQCKIGESVYKANIEGKVLAVKRFKEDVTEELKILQKVNHGNLVKLMGVSSDNDGNCFVVYEYAENGSLQEWLFAKSCSETLNSRTSLTWCQRISIAVDVSMGLQYMHEHAYPRIVHRDITSSNILLDSNFKAKIANFSMARTFTNPMMSKIDVFAFGVVLIELLTGRKAMTTKENGEVVMLWTDIWKIFDQEENREERLRKWMDPKLDNYYPIDYALSLASLAMNCTADKSLSRPTIAEIVLSLSLLTQPSPATLERSLTSSGLDVEATQIVTSISAR

SEQ ID NO: 41 - protein, 590 aa, Abrus precatorius
MASRSHSLCLALMLFFTNIAAQSQQTNGTNFSCPADSPPSCETYVTYIAQSPNFLSLTNISNIFDTSPLSIARASNLEQEDDKLIPDQVLLIPVTCGCTGTLSYANISYEINQGDSYSFVATTLYENLTNWHAVMDLNPGLSPNMLPVGVKVIVPLFCRCPSKNQLDKGIQYLITYVWQPNDNVSLVSAKFGASLQNILTENNYGQNFTSATNLPVLIPVTHLPDLIQPHLDVKKGSIHLSVILGISLGCTVLTVVLMVLLVYVYCLKMKSLNRSASSAETADKLLSGVSGYLRKPTVYEIDVIIEATMSLSEQCKIGESVYKANIKDQVLAVKKIKEDASEELKILQKLNHGNLVKLMGVSSDNDGNCFVVYEYAENGSLDDWLFSKSSSDTSSSMASLTWCQRISIAVDVAMGLQYMHEHAYPRIVHRDITSSNILLDSNFKAKIANFSMARTFTNPMMPKIDVFAFGVVLIELLTGRKAITTKENGEVVMLWKDIWKIFDQEENREEMLRKWMDPKLESFYPIDYALSLASLAVNCTADKSLSRPTIAEIVLSLSLFTQPSPATLERSLTSGLDVEVTQIVTSIAAR

SEQ ID NO: 42 - protein, 595 aa, Cicer arietinum
MSVFFLPSRSHVLFLALMLFLTNISAQSQHLSGTNFSCPVDSPPSCETYVTYIAQSPNFLSLTNISDLFDISPLSIARASNIDDEDKELIPGQVLLVPVTCGCTKHRSFANNTYTIKLGDSYILVSTTSYQNLTNYLEMEDSNPGLNPNLIPPFIKVVVPIFCRCPSKTQLNKGIKYLITYVWHANDNVSTVSSKFGASQVDILTENNYNQNFASAANLPVLIPVTRLPILAQPSSNGRKRSIQLPVIIGISIGSAFFVTVLTVSLVYLYCLKMKRLNRTASLSETADKLLSGVSGYVSKPTMYEMDVIMEATMNLSDQCKIGESVYKANIDGKVLAVKKTKKDASEELKILQKVNHGNLVKLMGVSSDNEGNCFLVYEYAENGSLDEWLFLESSKTSDSTVSLTWSQRIGIAVDVAVGLQYMHEHTYPRIIHRDITTSNILLDANFKAKIANFSMARTSTNPMMPKIDVFAFGVVLIELLTGKKGVTTKENGEVVIMWKDFWMIFDLEGNKEERLRKWMDPKLENFYPIDNALSLASLAVNCTADKSLSRPTIEEIVLCLNLLNQPSSEPTLERSLTFGLDVEDTQIVTSIAAR

SEQ ID NO: 43 - protein, 578 aa, Lupinus angustifolius
MLFFTNIAVQSQQINATDFSCPVDSPPSCETYVTYIAHSPNFLSVVSISNIFDTSPLSIARASNLEAEDNTLIPDQVLLVPVTCSCIGNRSFANMSYVIKLGDTYELVVETIYEHLTNWLVVADLNPGLVPTLLTVGVEVIFPLFCRCPSKNQLNKGIKNLITYVWQPNDTVSTVSSKFGASSADILSENNYGQNFTAAIHQPFLIPVTQLPHLSQPEPSNARKSSINLMLIIGISLGCTLLVAALAVLLVYVCYLKKSVLKRKASFVETADRLLTGVSGYVSKPTVYEIDAIMEATMNLSELCKIGESVYKAKMDGRILAVKKAKEDVSEELKILQKVNHGNLVKLMGVSSDNDGNFFLVYEYAENGCLDEWMFSKSTSNSMVSLTWSQRISIAVDIAMGLQYMHEHTHPRIVHRDITTSNILIDSNFKAKISNFSMARTSTNPMMPKIDVFAFGVVLLELLTGKKALTTKENGEVVMLWKDIRKVFDLEEDNKEVMLQRWMDPKLENLYPVDYALHLASLAVNCTADKPLSRPTMAEIVLTLSLLAQPPPASLERSLTSSGLDAEATQTIAPVAAR

SEQ ID NO: 44 - protein, 595 aa, Arachis hypogaea
MAFFLPSLSSSIFLAFMLFSVTSIPTQSQQVNGTDFSCPVDSPSSCGTYVTYIAKSPNFLSLSNISDIFDTSPLSIARASNIKNEGDKLVPGQVLLIPVTCGCTQNQSFANITYELRQGDVYDIVSKTTYENLTNWRAVNNSNPDLNPVLLPIGVKVLFPLFCRCPSKKQLQKGIEYMITYVWQNNDNVSSVAAKFGASPVDILSENNYGGNFTAATYLPVLIPVTKLPVLTQPEASHGRKRSIQIPVIISISLGFTLVVAVIVISMVYAYLYQRKRTLNRRDLSAGTADKLLSGVSGYVSKPTVYEANEVIKATMNLSEQCKLGGTVYKAKIEGQVLAVKKVNQVVSEELNILQKVNHGNLVKLMGVSSDSDGNHFLVYEYADNGSLDEWLFSKLSLKASLTWYQRINIALDVAMGLQYLHEHTYPRIVHRDITTSNILLDSNFKAKIGNFSMVRTTTNPMISKIDVFAFGVVLIELLTGKKAMTTKADGEVVMLWKDIRKMFEVEDEKEKEECLRRWMDPKLECLYPVDYALSLVTLAANCTADVSLSRPTMAEVVLGLSLLTQPSQAALERSLTSSALEAEVTHVATPIAAR

SEQ ID NO: 45 - protein, 594 aa, Arachis ipaensis
MAFFLPSLSSSIFLVFMFSITSIPTQSQQVNGTDFSCPVDSPSSCGTYVTYIAKSPNFLSLSNISDIFDTSPLSIARASNIKNEGDKLVPGQVLLIPVTCGCTQNQSFANITYELRQGDMYDFVSKTTYENLTNWRAVNDSNPDLNPVLLPVGVKVLFPLFCRCPSKKQLQKGIEYMITYVWQNNDNVSSVAAKFGASAVDILSENNYGGNFTAATYLPVLIPVTKLPVLTQPEPSHGRKRSIQIPVIISISLGFTLVVAVIVISMVYAYLYQRKRTLNRRDSSAGTADKLLSGVSGYVSKPTVYEANEVIKATMNLSEQCKLGGTVYKAKIEGQVLAVKKVNQVVSEELNILQKVNHGNLVKLMGVSSDSDGNHFLVYEYADNGSLDGWLFSKLSLKASLTWYQRINIALDVAMGLQYLHEHTYPRIVHRDITTSNILLDSNFKAKIGNFSMVRTTTNPMISKIDVFAFGVVLIELLTGRKAMTTKADGEVVMLWKDIRKMFEVEDEKEKEECLRRWMDPKLECLYPVDYALSLATLAANCTADVSLSRPTMAEVVLGLSLLTQPSQAALERSLTSSALEAEVTHVATPIAAR

SEQ ID NO: 46 - DNA, 2496 bp, Lotus japonicus
  source 1..2496: mol_type = unassigned DNA
tcgactatataaggggttcctccatacctaataagacatgtttgtttcatgtcgatctacttactctattttatggttttctcgtgagcaatttacatataaaatacacattaaattgcacccaataactaactagttacaagattagagcacataaatcatggaatgaaacaatagcctatgacttgggtattcggtccacaacatctctagaagttcgtacaacaacgatcaaagagaaagtactgactagtgatatgagaaatttggtcaggaacaatgggctggacacattacgaacttgagcctgaatagtagcagcatatgtctgccttcgcaatgacgaagaaggttggacggagctaaagttaaagctggaagggtgtgtcgtggcatttgaaatttagggttcaactgtagctttgtttgtctaactacccttctagctttaataaaaaaacgtaaatcaattaattaaagagaaatcacgccaacctgtctcaaacccgctctaacttgttggttaaggttatgtgttgtatttcaatttgcttgttgcttcaacttgactccttacctcctttcatgttttttcttgaaaattgtgtaaactcagttttttttttttgagaatgtatgagaatgtgatacaaattaaaatacaaacatacattgaatgagcggggctaaaatgactcaaggtacatatgagttaagtttttaccaacccatctctttgatgagttgggtttaattgggttagctcacgtgttaactatttcgacaattctaactttttggaataatgccatccaaccctaggtaaaactcaacatgacttctcttaaagaaatgaagagtgaaagtctacataaatcttaaacacttaaaacactacaacttggtagatcattaaaatatgctccagaaataccataatacttgaaaataaataatttataaatgtaaaatataaatttgttgctgcacccaagtctgagtgaggaatgaataaagataaataaaatatatatatgcgttgatactaacatatgatatgatgtgatagatgtcatatataatgaacagaatgatagaaattgttgagcgcatggaagcttcttgttcaataattcaagagctgcttgtggagggctgctgcagggttgttatggatgcaattatcaaatatcttgctggaaattgatccaaaagttgactattaccttatcaatgtccggtgtccttcaagccaccattcaattagtcaataaccttttgtttttgtttttttataattttggtgccaaataattttaaccatttatttttatcatatatattatataagatggcaagtctataatcatttaaaattaaattagtctaaatttttctaatatgctgtataaatttattatatataagaataaataatattaaaaatattgtttaattgctaccagttcaatcattaattaactattgactgaaaagaaaaatgtctaacaaattttgttacattctgtttgttttgagaaaaaaaaaattgtttttcattttctgaaaatatttatttttaaatttattaaatttagaaattatgtttgtttttacgctttgttttcatcttcaccgtcaccaccatcgctaccactgtcgtcactattactatataccactaccatcgttgttgccgttgtcattgtcaacaccattgctgtcgtcgttgttattatcgccccacaaccactatcatcatcactgctatcgtcatagctaccacgaacaccaccatcacttttcattgaaaatgaaaaatgtttatttatttattttgattatattttaaaaaatatttttaatcaaccaaatttctttttaactttttttttcttatgaaaaaaaataatatgaaaagaacaaaccaaacagatccttatattcttgtttccattctttatcacccatgagagtagctgtagcgcgttaactttctaatagaggcgggaatttaccgttaaccgttgggtcatcactcagcttactgagtcatcttcttcctactgtaggcggaacccacacgccactgcctctctattattacatcaaattctattttttttacatcatcatcaagataaataaattgcattattcagaaattaattcctgaatctccctatcttaactcaggtgtctctagctattaccacttaagctattattagaagtctacgtcaaattctataataaacacatattcttattcttattattatactaatacaaaccttgatttgaagccaaaaatgataaaacaattgcagactcgttcaaacaacagacaacatgtaccattaaattccaccccaaattctcaaagcaaattctgaattctccaactagtttccattctcctagtcaactcaactcaaaacccagatgagaatgagataattccaactttaacaaacacacacaattcatcattca

SEQ ID NO: 47 - protein, 595 aa, Medicago truncatula; identical to SEQ ID NO: 38
  REGION 147: note = hydrophobic patch residue
  REGION 151..152: note = hydrophobic patch residue
  REGION 154: note = hydrophobic patch residue
  REGION 156..158: note = hydrophobic patch residue
  REGION 146: note = residue immediately adjacent to hydrophobic patch residue
  REGION 148: note = residue immediately adjacent to hydrophobic patch residue
  REGION 150: note = residue immediately adjacent to hydrophobic patch residue
  REGION 153: note = residue immediately adjacent to hydrophobic patch residue
  REGION 155: note = residue immediately adjacent to hydrophobic patch residue
  REGION 159: note = residue immediately adjacent to hydrophobic patch residue

SEQ ID NO: 48 - protein, 594 aa, Pisum sativum
  REGION 146: note = hydrophobic patch residue
  REGION 150..151: note = hydrophobic patch residue
  REGION 154..159: note = hydrophobic patch residue
  REGION 145: note = residue immediately adjacent to hydrophobic patch residue
  REGION 147: note = residue immediately adjacent to hydrophobic patch residue
  REGION 149: note = residue immediately adjacent to hydrophobic patch residue
  REGION 152..153: note = residue immediately adjacent to hydrophobic patch residue
  REGION 160: note = residue immediately adjacent to hydrophobic patch residue
MAIFFLPSSSHALFLALMFFVTNISAQPLQLSGTNFSCPVDSPPSCETYVTYFARSPNFLSLTNISDIFDMSPLSIAKASNIEDEDKKLVEGQVLLIPVTCGCTRNRYFANFTYTIKLGDNYFIVSTTSYQNLTNYVEMENFNPNLSPNLLPPEIKVVVPLFCKCPSKNQLSKGIKHLITYVWQANDNVTRVSSKFGASQVDMFTENNQNFTASTNVPILIPVTKLPVIDQPSSNGRKNSTQKPAFIIGISLGCAFFVVVLTLSLVYVYCLKMKRLNRSTSLAETADKLLSGVSGYVSKPTMYEMDAIMEATMNLSENCKIGESVYKANIDGRVLAVKKIKKDASEELKILQKVNHGNLVKLMGVSSDNDGNCFLVYEYAENGSLDEWLFSESSKTSNSVVSLTWSQRITVAVDVAVGLQYMHEHTYPRIIHRDITTSNILLDSNFKAKIANFSMARTSTNSMMPKIDVFAFGVVLIELLTGKKAITTMENGEVVILWKDFWKIFDLEGNREESLRKWMDPKLENFYPIDNALSLASLAVNCTADKSLSRPSIAEIVLCLSLLNQSSSEPMLERSLTSGLDVEATHVVTSIVAR

SEQ ID NO: 49 - protein, 598 aa, Glycine max
  REGION 144: note = hydrophobic patch residue
  REGION 147..148: note = hydrophobic patch residue
  REGION 151..153: note = hydrophobic patch residue
  REGION 155..161: note = hydrophobic patch residue
  REGION 143: note = residue immediately adjacent to hydrophobic patch residue
  REGION 145..146: note = residue immediately adjacent to hydrophobic patch residue
  REGION 149..150: note = residue immediately adjacent to hydrophobic patch residue
  REGION 154: note = residue immediately adjacent to hydrophobic patch residue
  REGION 162: note = residue immediately adjacent to hydrophobic patch residue
MAVFFPFLPLHSQILCLVIMLFSTNIVAQSQQDNRTNFSCPSDSPPSCETYVTYIAQSPNFLSLTNISNIFDTSPLSIARASNLEPMDDKLVKDQVLLVPVTCGCTGNRSFANISYEINQGDSFYFVATTSYENLTNWRAVMDLNPVLSPNKLPIGIQVVFPLFCKCPSKNQLDKEIKYLITYVWKPGDNVSLVSDKFGASPEDIMSENNYGQNFTAANNLPVLIPVTRLPVLARSPSDGRKGGIRLPVIIGISLGCTLLVLVLAVLLVYVYCLKMKTLNRSASSAETADKLLSGVSGYVSKPTMYETDAIMEATMNLSEQCKIGESVYKANIEGKVLAVKRFKEDVTEELKILQKVNHGNLVKLMGVSSDNDGNCFVVYEYAENGSLDEWLFSKSCSDTSNSRASLTWCQRISMAVDVAMGLQYMHEHAYPRIVHRDITSSNILLDSNFKAKIANFSMARTFTNPMMPKIDVFAFGVVLIELLTGRKAMTTKENGEVVMLWKDIWKIFDQEENREERLKKWMDPKLESYYPIDYALSLASLAVNCTADKSLSRPTIAEIVLSLSLLTQPSPATLERSLTSSGLDVEATQIVTSIAAR

SEQ ID NO: 50 - protein, 557 aa, Cicer arietinum
  REGION 146: note = hydrophobic patch residue
  REGION 150..151: note = hydrophobic patch residue
  REGION 155..159: note = hydrophobic patch residue
  REGION 145: note = residue immediately adjacent to hydrophobic patch residue
  REGION 147: note = residue immediately adjacent to hydrophobic patch residue
  REGION 149: note = residue immediately adjacent to hydrophobic patch residue
  REGION 152: note = residue immediately adjacent to hydrophobic patch residue
  REGION 154: note = residue immediately adjacent to hydrophobic patch residue
  REGION 160: note = residue immediately adjacent to hydrophobic patch residue
MSVFFLPSRSHVLFLALMLFLTNISAQSQHLSGTNFSCPVDSPPSCETYVTYIAQSPNFLSLTNISDLFDISPLSIARASNIDDEDKELIPGQVLLVPVTCGCTKHRSFANNTYTIKLGDSYILVSTTSYQNLTNYLEMEDSNPGLNPNLIPPFIKVVVPIFCRCPSKTQLNKGIKYLITYVWHANDNVSTVSSKFGASQVDILTENNYNQNFASAANLPVLIPVTRLPILAQPSSNGRKRSIQLPVIIDKLLSGVSGYVSKPTMYEMDVIMEATMNLSDQCKIGESVYKANIDGKVLAVKKTKKDASEELKILQKVNHGNLVKLMGVSSDNEGNCFLVYEYAENGSLDEWLFLESSKTSDSTVSLTWSQRIGIAVDVAVGLQYMHEHTYPRIIHRDITTSNILLDANFKAKIANFSMARTSTNPMMPKIDVFAFGVVLIELLTGKKGVTTKENGEVVIMWKDFWMIFDLEGNKEERLRKWMDPKLENFYPIDNALSLASLAVNCTADKSLSRPTIEEIVLCLNLLNQPSSEPTLERSLTFGLDVEDTQIVTSIAAR

SEQ ID NO: 51 - protein, 595 aa, Arachis hypogaea
  source 1..595: note = subsp. hypogaea
  REGION 146: note = hydrophobic patch residue
  REGION 149..151: note = hydrophobic patch residue
  REGION 153..159: note = hydrophobic patch residue
  REGION 161..162: note = hydrophobic patch residue
  REGION 145: note = residue immediately adjacent to hydrophobic patch residue
  REGION 147..148: note = residue immediately adjacent to hydrophobic patch residue
  REGION 152: note = residue immediately adjacent to hydrophobic patch residue
  REGION 160: note = residue immediately adjacent to hydrophobic patch residue
  REGION 163: note = residue immediately adjacent to hydrophobic patch residue
MAFFLPSLSSSIFLAFMLFSVTSIPTQSQQVNGTDFSCPVDSPSSCGTYVTYIAKSPNFLSLSNISDIFDTSPLSIARASNIKNEGDKLVPGQVLLIPVTCGCTQNQSFANITYELRQGDVYDIVSKTTYENLTNWRAVNNSNPDLNPVLLPIGVKVLFPLFCRCPSKKQLQKGIEYMITYVWQNNDNVSSVAAKFGASPVDILSENNYGGNFTAATYLPVLIPVTKLPVLTQPEASHGRKRSIQIPVIISISLGFTLVVAVIVISMVYAYLYQRKRTLNRGDLSAGTADKLLSGVSGYVSKPTVYEANEVIKATMNLSGQCKLGGTVYKAKIEGQVLAVKKVNQVVSEELNILQKVNHGNLVKLMGVSSDSDGNHFLVYEYADNGSLDGWLFSKLSLKASLTWYQRINIALDVAMGLQYLHEHTYPRIVHRDITTSNILLDSNFKAKIGNFSMVRTTTNPMISKIDVFAFGAVLIELLTGMKAMTTKADGEVVMLWKDIRKMFEVEDEKEKEECLRRWMDPKLECLYPVDYALSLATLAANCTADVSLSRPTMAEVVLGLSLLTQPSQAALERSLTSSALEAEVTHVATSITAR

SEQ ID NO: 52 - protein, 613 aa, Parasponia andersonii
  REGION 136..138: note = hydrophobic patch residue
  REGION 142..143: note = hydrophobic patch residue
  REGION 145..151: note = hydrophobic patch residue
  REGION 135: note = residue immediately adjacent to hydrophobic patch residue
  REGION 139: note = residue immediately adjacent to hydrophobic patch residue
  REGION 141: note = residue immediately adjacent to hydrophobic patch residue
  REGION 144: note = residue immediately adjacent to hydrophobic patch residue
  REGION 152: note = residue immediately adjacent to hydrophobic patch residue
MAISLYLLLFFITHISAQSPPTLATNFSCSTNSSQPSCKTYVAYFAQPPLFMDLKSISNLFGVSPSSISEASNLVSESTKLTRGQLLLIPLSCSCNGSHYFSNVTYNITMGDSYYLVSIHSFENLTNWPLVRDTNPTLNPNLLQIGTKVIFPLYCGCPSKSHSKNGIKYLITYVWQPSDDIYRVSAMFNASEVDIIIENNYQDFKAAVGYPVLIPVSRMPALSQPPYPSHSHHRSQLKHRWFLIAVISSAGALLILFLATFLVHSIGLYEKKKNLSHEESSLETTDLIQVKNFSKSDTLELQAKHDKLLPGVSVYLGKPIMYEIKMIMEATMNFNDQYKIGGSVYRAMINGSFLAVKKAKENVTEELHILQKVNHGNLVKLMGISLDRDGNCFFVYEYAENGSLDKWLNPQSSTSTSSSVGILSWSQRLNIALDVANGLQYMHEHTQPSIVHKEIRTSNILLDSRFKAKIANFSMARSAASAGMTKVDVFAFGVVLLKLLSGRKAMATRENGEIVMLWKEAKAVLEEEEKRAEKVREWIDPKLESFYPIDGALSLMTLAKACTQEKASARPSIGEVVFSLCVLTQSFSETLEPSWTCTLEGEDVVQITSPIVA

SEQ ID NO: 53 - protein, 582 aa, Parasponia andersonii
  REGION 144..159: note = hydrophobic patch residue
  REGION 143: note = residue immediately adjacent to hydrophobic patch residue
  REGION 160: note = residue immediately adjacent to hydrophobic patch residue
MADSYFPFQAIFLLLLLFSTLNMAASQLNNSATDFSCSDSPPSCEAYVAYFSQPPNYMNVGNISDLFGISQALIAKSSNLVSKDSPLIPQQLLLIPLTCTCTGNHYFANITYQVEPGDTYHYLSTLLFENLTNSQVMKKMNPEISPEYVLPYIDIIIPVFCRCPSKSHLKSEIQQFITYVWQPNDQVSNVSAKFNTSASEIVNENKYNNFSSAVGLPVLIPVSKLPVLARVKPPKSVRSKKQWILIGVESLGGIVLITLFATLLVYSNRLLKKRRKILEARRLEPRIIIQDKLLSGVSEYLGRPIMYDNKMVVEGTMDFSEQCRIGGSVYRGEIYGEVFAVKKTKQDITDELNLLQKVNHVNLVNLMGASYDTDGNRFLVYEYVENGSLERWLDLKPSSLAAASSSSSVQFLSWSQRIQIALDVANGLQYLHEHTQPNIAHWNIRTSTILLDSKFRAKIANFEVARPVGNPAMLKVDIFAFGIVLLALVSGKKALQTIENGEVIMLWKDLAKEVFEVEEKKEDRLRKWMDPNLQSFYPIDGALSLSSLARACIREKSSARPKMAEIVFSLSVLAQSSSPGTP

SEQ ID NO: 54 - protein, 620 aa, Malus domestica
  REGION 145..158: note = hydrophobic patch residue
  REGION 144: note = residue immediately adjacent to hydrophobic patch residue
  REGION 159: note = residue immediately adjacent to hydrophobic patch residue
MAISFLCSKPLCILLLLLFFTARILAQSTPSNSSTSFSCSVDAPPSCDTYVSYFARPQFMSLENISHLFGVSPLSIAKASNLVSEHIRLIAGQLLLVPISCGCSGNSYFSNITYEIKSGDSFYLVSINSFENLTDWHEVLNMNPTLDPSLLQIGQKVIFPLFCKCPSKMYTENGIKYHITYIWQPNDDISRVSSRFNVSTLDISSANNLHNDSAAVELPVVIPVSRLPALVQPKPPQGRNIFKQRWWLILIIILGGVLLVSSLLAIFAVYTRHQHKVKKALDGPGSSLESAEWFKMKEGKIDENFDLKFIQDKLLPGVSSYLGKPIMYEVKTIMEATMNLNEHCRIGGSVYRAIVDGQVLAVKNTKEDVTEELNILQKVNHANLVKLMGVSSETDGSRFLVYEYAANGSLDKWLYSKSSATSSSAELLTWNQRLSIALDIANGLQYMHEHTQRSIVHMDIRTSNILLDSKFKAKIANFSMARAAANDVTPKVDVFAFGVVLLALLSGKKGMEAKENGEAIMLWKDVRWVLEAEEEKVERLRKWMDPNLENFYPIDGALSLTALARACTQEKPSTRPSMGEVVFNLSVLTHSSSQSTLERSWTSALEAEEVLETISPIAAR

SEQ ID NO: 55 - protein, 624 aa, Fragaria vesca
  source 1..624: note = subsp. vesca
  REGION 149..162: note = hydrophobic patch residue
  REGION 148: note = residue immediately adjacent to hydrophobic patch residue
  REGION 163: note = residue immediately adjacent to hydrophobic patch residue
MAVSFLCSSMVCILLLFFFTSQILAQPAPQSNSTTSFSCAVDAPSSCETYVAYFVESPGYMNLENISDLFGVSVSSISQASNLASSYTGQTRLVAGQLLLVPITCGCTGNRSFANITYSIKRGDSYYVVSMYTFENLTRWPLVVEMNPALVPSLLQIGVKVIFPLFCKCPSKMYSDLGIKYLLTYVWQTNDDIFRVSAKFNISALNISGANNFDNGSPVVGQPVLIPLTKLPALSQPLPPHGKHIFKHRLMLIVIICLGVALSVASLLAIFLVHTHRLRKRQKLLNDKSLSLESAEWFRMKEGKSEEKIEMKFIQDKLLPGVSSYLGKAILYDVKTIMEATMNLNDHCGIGGSVYRAVIDGKVLAVKKTKEDVTEELNILQKVNHANLVKLMGISSEIDGVRFLVYEYAENGSLDKWLYHKTSTNSSSGAFLTWSQRLSIALDVANGLQYLHEHTQPSIVHMDIRTSNILLDSKYKAKIANFSMARTAANSVTPKVDVFSFGVILLSLLSGKKGMETTDNGEVIMLWKDVRGVLEAEEKKQEKLRAWMDPTLESFYPIDGALSLTALASACTQEKSSARPSMAEVVFNLSVLTHSSSESTLERSWNSALEVEEVLQTISPIKAR

SEQ ID NO: 56 - protein, 626 aa, Hordeum vulgare
MEAPLHSLLLLLLLAAAAGPKTAAAVGDGCSRGCDLALASYYIAPNQNVTYIASLFGFSEYRVLGQYNPGVNNLDYVVAGDRLNVSLTCKCLASLSAPASTFLAASIPYKVATGETYLRIADNYNNLTTADWLVATNTYPANNIPDVATVNATVNCSCGDAGISTDYGLFLTYPLRDRETLASVAANHGFSSPEKMDLLKKYNPGMDGVTGSGIVYIPAKDPNGSYRPLESPGKKSSAGAIAGGVVAGVVALVLGVVLFLFYRRRKAKKDALLPSSEESTRLASAISMQKVTPSTSQADGASPAAGITVDKSVEFSYEELFNATEGFNIIHKIGQGGFGAVYYAELRGEKAAIKKMDMQATQEFLAELKVLTHVHHLNLVRLIGYCTESSLFLVYEFIENGNLSQHLRGTGYEPLSWVERVQIALDSARGLEYIHEHTVPVYIHRDIKSANILIDKNTRAKVADFGLTKLTEVGGGTSLQTRVVGTFGYMPPEYARYGDVSPKVDVYAFGVVLYELISAKDAIVRSAESTSDSKGLVYLFEEALSAPDPKEGIRRLMDPKLGDDYPIDAILKMTHLANACTQEDPKLRPTMRSVVVALMTLSSTSEFWDMNALYENPGLVNLMSGR

SEQ ID NO: 57 - protein, 633 aa, Hordeum vulgare
  REGION 144..157: note = hydrophobic patch residue
  REGION 143: note = residue immediately adjacent to hydrophobic patch residue
  REGION 158: note = residue immediately adjacent to hydrophobic patch residue
MEPRRFLCCCLVAVLAVASRRCDAQGGAGNGTGRFACVVPAPCDTFVLYRTQSPGSLDLGAISDLFGVSRAMIAAANNLSLIDEDAALLPDQPLLVPVRCGCTGNRSFVNVTYPIHSGDTFYALALTGYENLTTPDVIQELNPQAVFNKLNVSQLVTVPLFCRCPTPAERSAGVLQQITYMWRPVDTMSRVSKLMGSDASAIAAANNVSADFTSTTMLPMLIPVARPPVLPPLRYGPSATTGDPGATKRFSGATVAASIAGSLVAVAALCVAIFGYRRYRRKKATVHSASRFASPRFCFNQNAYGIQSSSSIARMINGGDKLLTSVSQFIDKPVIFGTAEIMEATMNLDERCRIGSSYYRAKLEGEVFAVKPAKGDVSAELRMMQMVNHANLIRLAGISIGADGDYTFLVYEFAEKGSLDKWLYQKPPSSLPSSSSSVDTLSWNQRLGIALDVANGLLYMHEHTQPSMVHGDVRARNILLTADFRARISNFSVATPAMADAAATSSDVFAFGLLVLELLSGRTAMEARVGAEIGMLWRDIRAVLEAGDKRDAKLRKWMDPALGDEYYLDAALSLAGMARACTEEDAARRPKMADVVFSLSMLVQPLPVGDAFEKLWQPSSEENIRIVNEVAAR

SEQ ID NO: 58 - protein, 622 aa, Lotus japonicus
MEHPRLGFPITLLLFSFILLPSTSQSKCTHGCALAQASYYLLNGSNLTYISEIMQSSLLTKPEDIVSYNQDTIASKDSVQAGQRINVPFPCDCIEGEFLGHTFQYDVQKGDRYDTIAGTNYANLTTVEWLRRFNSYPPDNIPDTGTLNVTVNCSCGDSGVGDYGLFVTYPLRPGETLGSVASNVKLDSALLQKYNPNVNFNQGSGIVYIPAKDQNGSYVLLGSSSGGLAGGAIAGIAAGVAVCLLLLAGFIYVGYFRKKRIQKEELLSQETRAIFPQDGKDENPRSTVNETPGPGGPAAMAGITVDKSVEFSYDELATATDNFSLANKIGQGGFGSVYYAELRGERAAIKKMDMQASKEFLAELKVLTRVHHLNLVRLIGYSIEGSLFLVYEFIENGNLSQHLRGSGRDPLPWATRVQIALDSARGLEYIHEHTVPVYIHRDIKSANILIDKNYRGKVADFGLTKLTEVGSSSLPTGRLVGTFGYMPPEYAQYGDVSPKVDVYAFGVVLYELISAKDAIVKTSESITDSKGLVALFEGVLSQPDPTEDLRKLVDQRLGDNYPVDSVRKMAQLAKACTQDNPQLRPSMRSIVVALMTLSSTTDDWDVGSFYENQNLVNLMSGR

SEQ ID NO: 59 - protein, 670 aa, Hordeum vulgare
MAAPPGRRGLAFGTAALALLAILAVARGQQQYEANAQTNCYGRNGSSVLGYVCNATAAAAPCATYVVFRSSPPYYGTAVSISYLLGSDPEAVADANGVPTVSPLADSRLVLAPVPCGCSPRGYYQHNSSHTIELRGETYFIIANNTYQGLTTCQALLAQNPRHGSRDLVAGNNLTVPIRCACPTPAQAAAGVRHLLTYLVTWGDSVSAIADRFRVDAQAVFQANNLTAREIIFPFTTLLIPLKSAPTPDMLVSPAPPPAPAPPQAQQPPASGSGKWIAVGVGVGVGVLALASLIGLMLLCVRRRRTRQGVRERGRLSKVVLDVPSSADYNALASGKHASSATTTSASSSALVSSDARAAVESLTVYKYSELEKATAGFSEDRRVKNASVYRAEINGDAAAVKRVAGDVSGEVGILKRVNHSSLVRLSGLCVHHGETYLVFEFAENGALSDWLHGGGATLVWKQRVQAAFDVADGLNYLHHYTNPPCVHKNLKSSNVLLDANLRAKVSSFALARSVPTGADGGDAQLTRHVVGTQGYLAPEYLEHGLITPKLDVFAFGVILLELLSGKEAMFNGGDKRGETLLWESAEGLVVDNEDARGKVRPFMDPRLHGDYPLDLAVAVASLAVRCVAREPRRRPSIDVVFATLSAVYNSTLDWDPSDDGNSRSSIVGR

SEQ ID NO: 60 - protein, 650 aa, Hordeum vulgare
  REGION 152..165: note = hydrophobic patch residue
  REGION 151: note = residue immediately adjacent to hydrophobic patch residue
  REGION 166: note = residue immediately adjacent to hydrophobic patch residue
MAPLTRRRRLLATLLCLCALPAPARSQNASATPAPASVEGFNCSANGTYPCQAYALYRAGLAGVPPDLSAAGDLFGVSRFMLAHANNLSTSAAPAAGQPLLVPLQCGCPSGSPNAYAPTQYQISSGDTFWIVSVTKLQNLTQYQAVERVNPTVVPTKLEVGDMVTFPIFCQCPTAAQNATALVTYVMQQGDTYASIAAAFAVDAQSLVSLNGPEQGTQLFSEILVPLRRQVPKWLPPIVTRNDASATPPSPSPPPTTTPGPSDVADNRDGVVTGLAVGLGVVGGLWLLQLLLLGCLWRRLKAKGRRGDAVASGEGGEGGRSAKTASASGGVGGERFLVTDISEWLDKYRVFKVEELERGTDGFDDAHLIQGSVYKANIGGEVFAVKKMKWDACEELKILQKVNHSNLVKLEGFCINTATGDCFLVYEYVENGSLDLCLLDRGRARRLDWRTRLHIALDLAHGLQYIHEHTWPRVVHKDVKSSNVLLDARMRAKIANFGLAKTGHNAVTTHIVGTQGYIAPEYLVDGLVTTKMDVFAYGVVLLELVSGREAAGDGGDLLLADAEERVFRGREDRLEARAAAWMDPVLAEQTCPPGSVATVMGVARACLQRDPSKRPSMVDVAYTLSRADEYFADYSGESVSVDGSGEIAAR

SEQ ID NO: 61 - protein, 622 aa, Hordeum vulgare
MGAPLPLLLPFLLLLLLLVASNTATAAGDGCNMGCDLALGSYYVTPNVNVSYIASLFGFSDYRELAKYNRGFPSLDYVAAGNRLDVPFTCKCLTLPSDRASTYLAASFPHKVDTGETYVSIASKYSNLTTADWLQATNTNTYPPNNIPANTILNVIVNCTCGDARISADYGLFRTFPVKDWQVLASISEFSPDQKALLTIYNPAIHSGTGSGIAYIPAKDPDGSYRPLKSQAGKKVPAGAIAGSVAAGLVAPVLGVLLFLFYRRRKAKQGALLPSSNESTRLASTILIQKLSPSTTEADVASLAAGITVDKSVEFTYQELFNATEGFHITHKIGQGGFGAVYYAELLGEKAAIKKMDMQATQEFLAELKVLTHVHHLNLVRLIGYCTESSLFLVYEFVENGNLSQHLHGTGYEPLSWAERVRIALDSARGLEYIHEHTVPVYIHRDIKSANILIDKNTRAKVADFGLTKLTEVGGASLLTRVVGTFGYMPPEYVRYGDVSRKVDVYAFGVVLYELISAKDAIVRSTDGSASGSRGLVYLFEEALTGLDPKEGLQKLIDPKLGDDYPVDAILMMTHLANACTEEDPKLRPTMRSVVVALMTLSSMTEFWDMKNPGLVNLMSGR

SEQ ID NO: 62 - protein, 681 aa, Hordeum vulgare
MATPTRWRGLAAVGRAALAFLVLLAVAAPWCPVARGQQEYEANAQNNCYGNNGSSVLGYTCNATAAVRPCASYVVFRSSPPYESPITISYLLNTTPAALADANAVPTVSSVAASRLVLAPLNCGCAPGGYYQHNASYTLQFSNETYFITANITYQGLTTCQALMAQNPNHDSRNLVVGNNLTVPIRCACPSPAQAASGVRHLLTYLVASGDTIADIATRFRVDAQAVLRANRLTDSENIYPFTTLLIPLKSAPTPDMLVSPAPPPAPVPPQAQQPLPTGGSGSGKGVAIGVGVGVGVLALAGLLGLMFLCVRRRRRLRPGVGENGHPGKVVIDVPSSADYDPLASGKHTSSATTTSSSSSAFVSSDARAAVESLTVYKYSELEKATAGFSEDRRVKDASVYRAVINGDTAAVKRVAGDVSGEVGILKRVNHSSLVRLSGLCVHHGDTYLVFEFAENGALSDWLHGGGATLVWKQRVQAAFDVADGLNYLHHYSTPPCVHKNLKSSNVLLDADLRAKVSSFALARSVPTGAEGGDAQLTRHVVGTQGYLAPEYLEHGLITPKLDVFAFGVILLELLSGKEATFNGGDKRGEKLLWESAEGLVVDGEDARSKVRAFMDPQLSGDYPLDLAVAVASLALRCVAREPRGRPSMYEVFVTLSAVYNSTLDWDPSDYSNSRSSIVGR

SEQ ID NO: 63 - protein, 631 aa, Hordeum vulgare
MVPGFLPWGRRVMQAPLPLVLPLFLLVASKTATVAGDGCRGCSLAFGSYYVTKDTNLTFISQLFGLSDYRDLAKYNRGLPNLDNAAAGDRVDVPFPCECLTRPSHPASTYLAASIPYKVATGETYVSIASNYNNLTTADWLQATNTYPPNDIPDVGVVNITVNCSCGDARISTDYGLFRTFPLRDWETLDSVAATRDLSSPERMDQLRRYNPGMEGATGSGIVYIPAQDPYGSYLPLKSPAGKKVSAGAIAGSVVAGVVAPVLLVLLFLFYKGRKAKQNALLPSSKDSTRLASTILMQKVKPSTAQADVASLAADITVDKSVEFTYQELFNATEGFNITHKIGQGGFGAVYYAELKGEKAAVKKMDMQATQEFLAELKVLTHVHHLNLVRLIGYCTDSSLFLVYEFVENGNLSQHLRGTGYEPLSWPERVRIALDSARGLEYIHEHTVPVYIHRDIKSANILIDKNTRAKVADFGLTKLTEVGGASLQTRVVGTFGYMPPEYVRYGDVSRKVDVYAFGVVLYELISAKDAIVRSTDGSASGSRGLVYLFEEALTGLDPKEGLQKLIDPKLGDDYPVDAILMMTHLANACTEEDPKLRPTMRSVVVALMTLSSMTEFWDMKNPGLVNLMSGR

SEQ ID NO: 64 - protein, 670 aa, Hordeum vulgare; identical to SEQ ID NO: 59

SEQ ID NO: 65 - protein, 650 aa, Hordeum vulgare; identical to SEQ ID NO: 60

SEQ ID NO: 66 - protein, 121 aa, synthetic construct
MQVQLVETGGGLVQDGDSLRLSCAASGFTFSNYYMSWVRQAPGKGLEWVSGITSGGTYTYDAVKDRFTISRDNAKNTLYLQMNSLKPEDTAVYYCAKGGTSVAVRGFYDYRGQGTQVTVSS

SEQ ID NO: 67 - protein, 129 aa, synthetic construct
MAQVQLVESGGRLVQAGDSLRLSCAASGRTFSTSAMAWFRQAPGREREFVAAITWTVGNTILGDSVKGRFTISRDRAKNTVDLQMDNLEPEDTAVYYCSARSRGYVLSVLRSVDSYDYWGQGTQVTVSS

SEQ ID NO: 68 - protein, 129 aa, synthetic construct
MAQVQLVESGGRLVQAGDSLRLSCAASGRTWSTSAMAWFRQAPGREREFVAAITWWVGNTILGDSVKGRFTISRDRAKNTVDLQMDNLEPEDTAVYYCSARSRWYVLSVLRSVDSYDYWGQGTQVTVSS

SEQ ID NO: 69 - DNA, 4171 bp, Lotus japonicus
  source 1..4171: mol_type = unassigned DNA
gcatgcatatagctctatttctttagtaatgttacacctgcacgatgtccataataatagaagtcataatacatatacagattaaaattaaataaacaatttctaatcaaatttaaaaatgtcaacttaatttcattattaaaatataacaatatgaataaccaaaaataaattaagacattcacccccccccccccgaaaagaaatttaagacaattacaattttttggtatatatattaaagacttccaattatggacataggatctcaacttagtaatcttcactttaggaaagtcttttccccacaagtcacaaccatctattaatatcaatacaaaatgaagtcaactcaataaaaagatccttttataggaaattgatgaataaaactgatatatatttcagttaaaattgttcaaacattagtgcaatggacagaagtatcctttgtgccctcatttgccaacaactggctcatcaagcaataaattaattcgccatttccaaacttttgcagttttaagtagaagatatccattcgttgaaactttcttcacaccaccaatttcctcctaaatgggttaacaaatgtgcaatgatcgaaatatatagttgaaacgatcaagatcctctcaatggtaaaagaatttgaccacgctaagttttattatctcactagctattaatttaattatcatttatcttttcaattattaaacacacaaataatcaatcctaaaatgatgaaacttgacatggtctatttttacaataacttaaccaaaaacttataagttagcaactttcaaaaacaggttttcccttgttaagaatagacaaatcaaatggagtgtgttaaatattgtgtttaaaatagtgtgttgcaagcatttctcttataaaaaatcagtataaatatgtttggaactgtttattttagtttatcttatattataaatacaaaacaagtgtttggtaaagctaatgaaaataacttaaaacatacctatggtttgtcggatgtgtcgcgggtggagctccttgccattttgtgtggcctttgtatgtgttgagatatggccatgggttattgtagaatccttcttcttttggattcttctttgttcctgtctctcattcaacgctcatgttacccatttcatccatgccaagttttttttatacatgcatctaaattttttccgccatatcttaattttgtttttattaaaattaaaaagaatatgattgaatgtcaatgtaaattttttttacacagacaatgcatatccattaaggtttgttagaattacactccaccccatttttatctaaaatctacatcccaccccattttatatagaggcaaatttagtgacgaaaaatattcttcattaataattagttattatttaaattgttaatcaataatttcaaaaaaaaattcaatataatccaataaatttaaaaatgaaaacatcacaatcctcctcattctctcaatcgcgttttacctccgtaaatttacaatgcaaattatgcaatagcacacctgcccgatttacaaaccatatttcgaacatagtgaaacatgcttgtgttttcatatttggtgataattcaattttaatcaaaataatctctttatacctccaattttcaaaattgggttgtaggccaaaaaagcaacacaaatgggtgaagaaaatagagaaacaaaattatgaaaatatgaagtggatctgaggttattagagcccaacgaggcggtggagcatcgtttttaacaaaatccaacaatatctttaggggtgaaatccaccaaccgagcgttcgctcacaacttaaaggggtgaaattcacaagaagtagtttaaaggggtgaaattcacaagaagtagttgaacaagtgactttaggaatgtgcgattcacgttctggggttcaggtcgcaacaaaactttgaggcatggtggtgccatgtggtttcacattgtcggacagtggagccgtgttaaagggagtaaaggcttggtggtggccgtttgtggtgaaaaatatgatttggagatagatgggacgtggacttaacagacagagtggatggtttttttttaaatttaattcagtaaaattatttttataaattaatgtatgatagtgtatatgcattaaatttatttaaatttttactaattagtaatttgtttttagtagtgacgaatttgtttttgtcactaaattttgcctttataaaaaatggggtggagtgtagatttttaaaaagaatggggtggagtgtcattcttgcaaatcttgaggggggtgagtgtattttactcaactcttaaaaaaattaggaattaattagttgtaaattataaaagtttatttcattgaataacataacaaattaaaggcaaaaaaatacaaaacttcattttatatgtatttcagaaaaattgcctactttcaattatgagaaactaaaattatgtttagtttaaaatgagcatagattcaaaaattaataaataatatatatagcaggatacatgcctatcaattaacatatcgtttgtccacgatgatgatcttattggaggatcaatatcttcaaattaacaaagttatcacttggctcttattggtcataatgcaataaaaaaattgcaattagtatcaaatcaaactgaaatttgcaactatatgctgctggtgttgtcgcgcagattcctttttgatttttatgggaatgaagtcaatgaagcaacagtttcacaggcgtgcttaaaaataaaaaaattggaaatttgatgtttgttaggattatgagaggacacaatgggaggatgtttcacaagctgcagacagggttgccacttcagatgcaaaggattaaataaacaaagccaaggtttgcaatcaacaagattccatcgtcgttttgcttcctttaatcgtattaatcaaaagcacaccaagtaaagcatcaatatataacatccaagaaatcacaacatgatagttgctcgtgtcgtctattaactatgatgtcaggagttcgatccccgctcatgtgaatggaagtcatttcgttgttagatgtttaccgtttaatgcaaatactcgcggtgagataataagtcattgttgtgggcgaataccctaaaataagaataaaattaaatatagcatccaagttattgcccaaatatataaacaatggtattgttgacattattaggcataaaagcagtaggtaagtgtattatatttatttaattttttaaaattttgaaattaattaataattgttaacataagtaaaccatttttagcaaaaactctacacttctattaccttaacaagtacatttttgatggtacaccttaacaattaacaagtcatatgattgacaaacatattttatatgctttacaatttattctaaaatcaaagtttatgggaagaagctcataaaagtagttcctgggtgttttttagaatagagaagttgatcatgttagaaattaagttaaaaatgagttgaaagtgatttatgtttgattatatttatgagaaaaatgaattgtctgatgtaatattgtaaaatctaacaattaattaagtaccacagaaactagaatttatagcttcaccttagaattgattttggagttaaaatcaattattaaaggagcaattattaaaggagacatccaaatacactagttaattttgacaatcaattctaacacttgcaaatgtgtaaccaaacttactatcagtaagtgaactaatgattcccaagtcaacttttgttctagctagccaaccgttactatgttccctccacaatacattctccttgaaactgtcaagtgtcaactgcacccaaacatccttgtttgtgatgaaaagatcgaaaacgtgtgcttatgaatttacatgtttacattcaccaaaaatcaaaagttacacctctatacttatcacatatgtttgagtcactttccatataaaatcccatagtctattaattatcagagtaagtgtgacattagtttcaagagaaaaataaatgatcaaaacctggtagagagtcctagaaattcaatgttctgatttctttcattcatctctgctgccattttgatttgcaca

(19) United States
(12) Patent Application Publication
(10) Pub. No.: US 2024/0200085 A1
(43) Pub. Date: Jun. 20, 2024

(54) SYNTHETIC ACTIVATION OF MULTIMERIC TRANSMEMBRANE RECEPTORS

(71) Applicant: Aarhus Universitet, Aarhus (DK)

(72) Inventors: Kasper Røjkjær ANDERSEN, Aarhus (DK); Elena Simona RADUTOIU, Aarhus (DK); Henriette RÜBSAM, Aarhus (DK); Christina KRÖNAUER, Aarhus (DK); Jens Stougaard JENSEN, Aarhus (DK)

(73) Assignee: Aarhus Universitet, Aarhus (DK)

(21) Appl. No.: 18/541,719

(22) Filed: Dec. 15, 2023

Related U.S. Application Data
(60) Provisional application No. 63/387,674, filed on Dec. 15, 2022.

(51) Int. Cl. C12N 15/82; C07K 14/415; G01N 33/50

(52) U.S. Cl. CPC C12N 15/8205; C07K 14/415; G01N 33/5097; G01N 2333/415

(57) ABSTRACT

The present disclosure relates to synthetic approaches based on affinity peptides to drive assembly and activation of transmembrane receptor complexes. The present disclosure further relates to the core NFR1-NFR5 receptor complex initiating the cortical root nodule organogenesis program and the epidermal program, as well as the identification of barley receptor complexes that function in root nodule symbiosis.

### CROSS-REFERENCE TO RELATED APPLICATIONS

This application claims the benefit of U.S. Provisional Application No. 63/387,674, filed Dec. 15, 2022, which is hereby incorporated by reference in its entirety.

### REFERENCE TO AN ELECTRONIC SEQUENCE LISTING

The content of the electronic sequence listing (794542002100SEQLIST.xml; Size: 135,817 bytes; and Date of Creation: Dec. 8, 2023) is herein incorporated by reference in its entirety.

### TECHNICAL FIELD

The present disclosure relates to synthetic approaches using affinity polypeptides and optionally tag polypeptides to drive assembly and activation of transmembrane receptor complexes (i.e., multimeric transmembrane receptors). The present disclosure further relates to activation of the core Nod-factor transmembrane receptor complex (e.g., NFR1-NFR5) to initiate the cortical root nodule organogenesis program as well as the epidermal program important for infection, as well as the identification of barley receptor complexes that function in root nodule symbiosis.

### BACKGROUND

Living cells constantly need to monitor their environment to integrate changes and to communicate with neighboring cells and microorganisms. Transmembrane receptor complexes allow cells to perceive external signals and convert stimuli into intracellular responses. Transmembrane receptor complexes can be readily identified by signature transmembrane domains and have been estimated to make up 30% of the proteome (Wallin E & von Heijne G (1998) Genome-wide analysis of integral membrane proteins from eubacterial, archaean, and eukaryotic organisms. Protein Sci 7, 1029-1038; Lomize A L, Hage J M, Pogozheva I D (2018) Membranome 2.0: database for proteome-wide profiling of bitopic proteins and their dimers. Bioinformatics 34(6), 1061-1062; and membranome [dot]org/about). While transmembrane receptor complex subunits can be readily identified in genome sequencing, determining the role of a receptor complex or partners that interact with a receptor complex is quite difficult. Further, there is a limited understanding and lack of methodology for exploring how receptor subunits form signaling-competent complexes that control and activate specific intracellular pathways. Thus, there is a need for new tools to assess the function of transmembrane receptor complexes to aid in assigning roles to those receptor complexes.

Plants interact with a wealth of microbes and need to distinguish between those that pose a risk and those that offer a potential benefit. To this end, plants use pattern-recognition transmembrane receptor complexes, including lysin motif (LysM) receptors, that perceive microbial-derived carbohydrate signals and mount an intracellular response. Plant LysM receptors recognize conserved cell-wall components such as chitin from pathogenic fungi (Kaku et al. (2006) PNAS 103, 11086-11091; Willmann et al. (2011) PNAS 108, 19824-19829), Myc factors in the context of arbuscular mycorrhizal (i.e., fungal) symbiosis (He et al. (2019) Molecular Plant 12, 1561-1576; Feng et al. (2019) Nature Communications 10: 5047) and Nod factors in the context of nitrogen-fixing symbiosis with bacteria (rhizobia) (Limpens et al. (2003) Science 302, 630-633; Radutoiu et al. (2003) Nature 425, 585-592; Broghammer et al. (2012) PNAS 109, 13859-13864; Bozsoki et al. (2020) Science 369, 663-670; Gysel et al. (2021) PNAS 118, e2111031118). Genetic studies in the model legume Lotus japonicus (Lotus) have identified two Nod-factor receptors, NFR1 and NFR5 (Radutoiu et al. (2003) Nature 425, 585-592), and the symbiosis receptor-like kinase SYMRK (Stracke et al. (2002) Nature 417, 959-962) that initiate and control the two developmental programs leading to nodule organogenesis and infection (intracellular accommodation), respectively. Loss-of-function mutations in these three receptors render plants incapable of establishing root nodule symbiosis, but precisely how the receptors collaborate during signaling, as well as what constitutes the active receptor complexes in this process, have remained enigmatic.

There exists a need to answer the long-standing question of the composition of the core receptor complex that induces signaling in root nodule symbiosis. More broadly, there is a need to develop generally applicable synthetic approaches that can be used to interrogate signaling processes involving transmembrane receptor complexes in vivo. Finally, there is more generally a need for compositions and methods for signal-independent activation of transmembrane receptor complexes in vivo through genetic engineering.

### BRIEF SUMMARY

The present disclosure provides generally applicable synthetic approaches using affinity polypeptides and optionally tag polypeptides to drive assembly and activation of cell-surface receptor complexes (i.e., single-pass transmembrane cell-surface receptor complexes) from within the cell. This novel approach is able to tackle questions relating to signaling processes, and can be used in both a sequence-directed way (e.g., by designing targeted affinity polypeptides, such as heavy-chain variable domain (VHHs)) as well as a sequence-agnostic way (e.g., by using tag polypeptides and commercially available affinity polypeptides directed to those tags). The receptor complexes that govern plant symbiosis with nitrogen-fixing bacteria were manipulated using these approaches, which allowed the core NFR1-NFR5 receptor complex initiating the cortical root nodule organogenesis program as well as the epidermal program important for infection to be defined. Moreover, these approaches were used to characterize barley receptor complexes that function in root nodule symbiosis.

An aspect of the disclosure includes a genetically modified cell including: a transmembrane (TM) receptor complex including a first subunit polypeptide and a second subunit polypeptide, wherein the first subunit polypeptide includes an affinity polypeptide that binds to the second subunit polypeptide intracellularly inducing oligomerization; and wherein oligomerization of the first subunit polypeptide and the second subunit polypeptide activates TM receptor signaling, wherein the affinity polypeptide is heterologous to the first subunit polypeptide. In an additional embodiment of this aspect, the TM receptor is a single-pass TM (SPTM) receptor, or the TM receptor is a SPTM receptor including an intracellular kinase domain (SPTM-kinase). In a further embodiment of this aspect, which may be combined with any of the preceding embodiments, the affinity polypeptide binds directly to the second subunit polypeptide. In yet another embodiment of this aspect, which may be combined with any of the preceding embodiments, the second subunit polypeptide includes a tag polypeptide, and the affinity polypeptide binds to the tag polypeptide. In still another embodiment of this aspect, the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, or a ubiquitin tag polypeptide. In a further embodiment of this aspect the tag polypeptide is a fluorescent protein polypeptide selected from a green fluorescent protein (GFP) polypeptide, a red fluorescent polypeptide (RFP) (e.g., mCherry), or a blue fluorescent polypeptide (BFP); wherein the tag polypeptide is a luminescence polypeptide selected from a luciferase polypeptide; or wherein the tag polypeptide is a combination of a fluorescent protein polypeptide and a luminescence polypeptide. In yet another embodiment of this aspect, which may be combined with any of the preceding embodiments, the affinity polypeptide is selected from a heavy-chain variable domain (VHH), a single-chain variable fragment (scFV), a designed ankyrin repeat protein (DARPins), a human fibronectin III domain 3 monobody, an Affibody, or an anticalin, or a synthetic version of any of the foregoing. In an additional embodiment of this aspect, the affinity polypeptide is a heavy-chain variable domain (VHH). In a further embodiment of this aspect, the affinity polypeptide is a VHH_{LaG16}.

In still another embodiment of this aspect, which may be combined with any of the preceding embodiments, the cell is a eubacterial cell, an archaeal cell, or a eukaryotic cell. In another embodiment of this aspect, the eukaryotic cell is a plant cell, an animal cell, or a fungal cell. In yet another embodiment of this aspect, which may be combined with any of the preceding embodiments, the cell is a plant cell and the first subunit polypeptide and/or the second subunit polypeptide are selected from a LysM receptor, a Leucine rich repeat (LRR) receptor, a Malectin like receptor, a plant TM receptor, a plant SPTM (pSPTM) receptor, and a plant SPTM receptor including an intracellular kinase domain (pSPTM-kinase). In a further embodiment of this aspect, the first subunit polypeptide and/or the second subunit polypeptide is a LysM receptor. In an additional embodiment of this aspect, the first subunit polypeptide and/or the second subunit polypeptide lacks an ectodomain and/or a transmembrane domain. In another embodiment of this aspect, which may be combined with any of the preceding embodiments where the first subunit polypeptide and/or the second subunit polypeptide is a LysM receptor, the first subunit polypeptide is a NFR1 polypeptide and the second subunit polypeptide is a NFR5 polypeptide, wherein the first subunit polypeptide is a NFR5 polypeptide and the second subunit is a NFR1 polypeptide, wherein the first subunit polypeptide is a LYK3 polypeptide and the second subunit polypeptide is a NFP polypeptide, wherein the first subunit polypeptide is a NFP polypeptide and the second subunit polypeptide is a LYK3 polypeptide, wherein the first subunit polypeptide is a RLK4 receptor polypeptide and the second subunit polypeptide is a RLK10 receptor polypeptide, or wherein the first subunit polypeptide is a RLK10 polypeptide and the second subunit polypeptide is a RLK4 polypeptide. In a further embodiment of this aspect, the NFR1, LYK3, or RLK4 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 56, and wherein the NFR5, NFP, or RLK10 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, and SEQ ID NO: 57. In yet another embodiment of this aspect, which may be combined with any of the preceding embodiments, the affinity polypeptide binding to the second subunit polypeptide is regulated by a small molecule.

An additional aspect of the disclosure includes a bispecific affinity polypeptide including: a first affinity polypeptide that binds to an intracellular portion of a first subunit polypeptide of a TM receptor; and a second affinity polypeptide that binds to an intracellular portion of a second subunit polypeptide of the TM receptor; wherein binding of the first affinity polypeptide to the first subunit polypeptide and of the second affinity polypeptide to the second subunit polypeptide induces oligomerization; and wherein oligomerization of the first subunit polypeptide and the second subunit polypeptide activates TM receptor signaling. In a further embodiment of this aspect, the TM receptor is a single-pass TM (SPTM) receptor, or the TM receptor is a SPTM including an intracellular kinase domain (SPTM-kinase). In yet another embodiment of this aspect, which may be combined with any of the preceding embodiments, the first affinity polypeptide binds directly to the first subunit polypeptide and/or the second affinity polypeptide binds directly to the second subunit polypeptide. In an additional embodiment of this aspect, which may be combined with any of the preceding embodiments, the first subunit polypeptide includes a tag polypeptide, and the first affinity polypeptide binds to the tag polypeptide, and/or the second subunit polypeptide includes a tag polypeptide, and the second affinity polypeptide binds to the tag polypeptide. In still another embodiment of this aspect, the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, or a ubiquitin tag polypeptide. In a further embodiment of this aspect, the tag polypeptide is a fluorescent protein polypeptide selected from a green fluorescent protein (GFP) polypeptide, a red fluorescent polypeptide (RFP) (e.g., mCherry), or a blue fluorescent polypeptide (BFP); wherein the tag polypeptide is a luminescence polypeptide selected from a luciferase polypeptide; or wherein the tag polypeptide is a combination of a fluorescent protein polypeptide and a luminescence polypeptide. In yet another embodiment of this aspect, which may be combined with any of the preceding embodiments, the affinity polypeptide is selected from a heavy-chain variable domain (VHH), a single-chain variable fragment (scFV), a designed ankyrin repeat protein (DARPins), a human fibronectin III domain 3 monobody, an Affibody, or an anticalin, or a synthetic version of any of the foregoing. In an additional embodiment of this aspect, the affinity polypeptide is a heavy-chain variable domain (VHH). In a further embodiment of this aspect, the affinity polypeptide is a VHH_{LaG16}. In an additional embodiment of this aspect, which may be combined with any of the preceding embodiments, the first subunit polypeptide and/or the second subunit polypeptide are selected from a LysM receptor, a Leucine rich repeat (LRR) receptor, a Malectin like receptor, a plant TM receptor, a plant SPTM (pSPTM) receptor, and a plant SPTM receptor including an intracellular kinase domain (pSPTM-kinase). In yet another embodiment of this aspect, the first subunit polypeptide and/or the second subunit polypeptide is a LysM receptor. In an additional embodiment of this aspect, the first subunit polypeptide and/or the second subunit polypeptide lacks an ectodomain and/or a transmembrane domain. In another embodiment of this aspect, which may be combined with any of the preceding embodiments where the first subunit polypeptide and/or the second subunit polypeptide is a LysM receptor, the first subunit polypeptide is a NFR1 polypeptide and the second subunit polypeptide is a NFR5 polypeptide, wherein the first subunit polypeptide is a NFR5 polypeptide and the second subunit is a NFR1 polypeptide, wherein the first subunit polypeptide is a LYK3 polypeptide and the second subunit polypeptide is a NFP polypeptide, wherein the first subunit polypeptide is a NFP polypeptide and the second subunit polypeptide is a LYK3 polypeptide, wherein the first subunit polypeptide is a RLK4 receptor polypeptide and the second subunit polypeptide is a RLK10 receptor polypeptide, or wherein the first subunit polypeptide is a RLK10 polypeptide and the second subunit polypeptide is a RLK4 polypeptide. In a further embodiment of this aspect, the NFR1, LYK3, or RLK4 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 56, and wherein the NFR5, NFP, or RLK10 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 57. In yet another embodiment of this aspect, which may be combined with any of the preceding embodiments, the affinity polypeptide binding to the second subunit polypeptide is regulated by a small molecule.

A further aspect of the disclosure includes methods of screening transmembrane (TM) receptor including: (a) providing a cell expressing a first subunit polypeptide of a TM receptor; (b) expressing a second subunit polypeptide of a TM receptor in the cell; and (c) assaying the cell for a TM receptor phenotype; and wherein (i) the presence of the TM receptor phenotype indicates the first subunit polypeptide and the second subunit polypeptide oligomerize to form the TM receptor; or (ii) the absence of the TM receptor phenotype indicates the first subunit polypeptide and the second subunit polypeptide do not oligomerize to form a TM receptor, and wherein (1) the second subunit polypeptide includes a tag polypeptide, the first subunit polypeptide includes an affinity polypeptide that binds to the tag polypeptide, and the affinity polypeptide is heterologous to the first subunit polypeptide, or (2) the first subunit polypeptide includes the tag polypeptide, the second subunit polypeptide includes the affinity polypeptide that binds to the tag polypeptide, and the affinity polypeptide is heterologous to the second subunit polypeptide. In an additional embodiment of this aspect, the TM receptor is a single-pass TM (SPTM) receptor, or the TM receptor is a SPTM including an intracellular kinase domain (SPTM-kinase). In yet another embodiment of this aspect, which may be combined with any of the preceding embodiments, the binding partner for the first subunit polypeptide is unknown and (b) is repeated using two or more candidate second subunit polypeptides to identify the second subunit polypeptide that is the binding partner for the first subunit polypeptide. In a further embodiment of this aspect, the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, or a ubiquitin tag polypeptide. In a further embodiment of this aspect, the tag polypeptide is a fluorescent protein polypeptide selected from a green fluorescent protein (GFP) polypeptide, a red fluorescent polypeptide (RFP) (e.g., mCherry), or a blue fluorescent polypeptide (BFP); wherein the tag polypeptide is a luminescence polypeptide selected from a luciferase polypeptide; or wherein the tag polypeptide is a combination of a fluorescent protein polypeptide and a luminescence polypeptide. In yet another embodiment of this aspect, which may be combined with any of the preceding embodiments, the affinity polypeptide is selected from a heavy-chain variable domain (VHH), a single-chain variable fragment (scFV), a designed ankyrin repeat protein (DARPins), a human fibronectin III domain 3 monobody, an Affibody, or an anticalin, or a synthetic version of any of the foregoing. In an additional embodiment of this aspect, the affinity polypeptide is a heavy-chain variable domain (VHH). In a further embodiment of this aspect, the affinity polypeptide is a VHH_{LaG16}. In still another embodiment of this aspect, which may be combined with any of the preceding embodiments, the cell is a eubacterial cell, an archaeal cell, or a eukaryotic cell. In another embodiment of this aspect, the eukaryotic cell is a plant cell, an animal cell, or a fungal cell. In yet another embodiment of this aspect, which may be combined with any of the preceding embodiments, the cell is a plant cell and the first subunit polypeptide and/or the second subunit polypeptide are selected from a LysM receptor, a Leucine rich repeat (LRR) receptor, a Malectin like receptor, a plant TM receptor, a plant SPTM (pSPTM) receptor, and a plant SPTM receptor including an intracellular kinase domain (pSPTM-kinase). In a further embodiment of this aspect, the first subunit polypeptide and/or the second subunit polypeptide is a LysM receptor. In an additional embodiment of this aspect, the first subunit polypeptide and/or the second subunit polypeptide lacks an ectodomain and/or a transmembrane domain. In another embodiment of this aspect, which may be combined with any of the preceding embodiments where the first subunit polypeptide and/or the second subunit polypeptide is a LysM receptor, the first subunit polypeptide is a NFR1 polypeptide and the second subunit polypeptide is a NFR5 polypeptide, wherein the first subunit polypeptide is a NFR5 polypeptide and the second subunit is a NFR1 polypeptide, wherein the first subunit polypeptide is a LYK3 polypeptide and the second subunit polypeptide is a NFP polypeptide, wherein the first subunit polypeptide is a NFP polypeptide and the second subunit polypeptide is a LYK3 polypeptide, wherein the first subunit polypeptide is a RLK4 receptor polypeptide and the second subunit polypeptide is a RLK10 receptor polypeptide, or wherein the first subunit polypeptide is a RLK10 polypeptide and the second subunit polypeptide is a RLK4 polypeptide. In a further embodiment of this aspect, the NFR1, LYK3, or RLK4 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 56, and wherein the NFR5, NFP, or RLK10 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 57. In yet another embodiment of this aspect, which may be combined with any of the preceding embodiments, the affinity polypeptide binding to the second subunit polypeptide is regulated by a small molecule.

Some aspects of the disclosure are related to a genetically modified plant or part thereof including the genetically modified plant cell of any one of the preceding embodiments. An additional embodiment of this aspect further includes the TM receptor including the first subunit polypeptide and the second subunit polypeptide, wherein the first subunit polypeptide includes the affinity polypeptide that binds to the second subunit polypeptide intracellularly inducing oligomerization, and wherein oligomerization of the first subunit polypeptide and the second subunit polypeptide activates TM receptor signaling. In a further embodiment of this aspect, the affinity polypeptide binds directly to the second subunit polypeptide or wherein the second subunit polypeptide includes a tag polypeptide, and wherein the affinity polypeptide binds to the tag polypeptide. In yet another embodiment of this aspect, which may be combined with any of the preceding embodiments, the genetically modified plant cell is selected from a root epidermal cell, a root cortex cell, a root endodermis cell, a root pericycle cell, a root primordia cell, a xylem cell, a phloem cell, a meristem cell, a leaf cell, a stem cell, a flower cell, or a fruit cell. In still another embodiment of this aspect, the genetically modified plant cell is a root epidermal cell, a root cortex cell, a root endodermis cell, a root pericycle cell, or a root primordia cell.

Some aspects of the disclosure are related to a genetically modified plant or part thereof including the bispecific affinity polypeptide of any one of the preceding embodiments. Another embodiment of this aspect further includes the first affinity polypeptide that binds to the intracellular portion of the first subunit polypeptide of the TM receptor and the second affinity polypeptide that binds to the intracellular portion of the second subunit polypeptide of the TM receptor, wherein binding of the first affinity polypeptide to the first subunit polypeptide and of the second affinity polypeptide to the second subunit polypeptide induces oligomerization; and wherein oligomerization of the first subunit polypeptide and the second subunit polypeptide activates TM receptor signaling. In an additional embodiment of this aspect, the first affinity polypeptide binds directly to the first subunit polypeptide and/or wherein the second affinity polypeptide binds directly to the second subunit polypeptide; or wherein the first subunit polypeptide includes the tag polypeptide, and wherein the first affinity polypeptide binds to the tag polypeptide, and/or wherein the second subunit polypeptide includes the tag polypeptide, and wherein the second affinity polypeptide binds to the tag polypeptide.

In yet another embodiment of this aspect, which may be combined with any preceding embodiment that has a genetically modified plant or part thereof, the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, or a ubiquitin tag polypeptide. In a further embodiment of this aspect, the tag polypeptide is a fluorescent protein polypeptide selected from a green fluorescent protein (GFP) polypeptide, a red fluorescent polypeptide (RFP) (e.g., mCherry), or a blue fluorescent polypeptide (BFP); wherein the tag polypeptide is a luminescence polypeptide selected from a luciferase polypeptide; or wherein the tag polypeptide is a combination of a fluorescent protein polypeptide and a luminescence polypeptide. In still another embodiment of this aspect, which may be combined with any preceding embodiment that has a genetically modified plant or part thereof, the affinity polypeptide is selected from a heavy-chain variable domain (VHH), a single-chain variable fragment (scFV), a designed ankyrin repeat protein (DARPins), a human fibronectin III domain 3 monobody, an Affibody, or an anticalin, or a synthetic version of any of the foregoing. In a further embodiment of this aspect, the affinity polypeptide is a heavy-chain variable domain (VHH). In an additional embodiment of this aspect, the affinity polypeptide is a VHH_{LaG16}. In yet another embodiment of this aspect, which may be combined with any preceding embodiment that has a genetically modified plant or part thereof, the first subunit polypeptide and/or the second subunit polypeptide are selected from a LysM receptor, a Leucine rich repeat (LRR) receptor, a Malectin like receptor, a plant TM receptor, a plant SPTM (pSPTM) receptor, and a plant SPTM receptor including an intracellular kinase domain (pSPTM-kinase). In another embodiment of this aspect, the first subunit polypeptide and/or the second subunit polypeptide is a LysM receptor. In a further embodiment of this aspect, the first subunit polypeptide and/or the second subunit polypeptide lacks an ectodomain and/or a transmembrane domain. In yet another embodiment of this aspect, the first subunit polypeptide is a NFR1 polypeptide and the second subunit polypeptide is a NFR5 polypeptide, wherein the first subunit polypeptide is a NFR5 polypeptide and the second subunit is a NFR1 polypeptide, wherein the first subunit polypeptide is a LYK3 polypeptide and the second subunit polypeptide is a NFP polypeptide, wherein the first subunit polypeptide is a NFP polypeptide and the second subunit polypeptide is a LYK3 polypeptide, wherein the first subunit polypeptide is a RLK4 receptor polypeptide and the second subunit polypeptide is a RLK10 receptor polypeptide, or wherein the first subunit polypeptide is a RLK10 polypeptide and the second subunit polypeptide is a RLK4 polypeptide. In an additional embodiment of this aspect, the NFR1, LYK3, or RLK4 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 56, and wherein the NFR5, NFP, or RLK10 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 57. In a further embodiment of this aspect, which may be combined with any preceding embodiment that has a genetically modified plant or part thereof, the TM receptor is localized to a plant cell membrane. In still another embodiment of this aspect, which may be combined with any preceding embodiment that has a genetically modified plant or part thereof, the plant part is a leaf, a stem, a root, a root primordia, a flower, a seed, a fruit, a kernel, a grain, a cell, or a portion thereof. In yet another embodiment of this aspect, which may be combined with any preceding embodiment that has a genetically modified plant or part thereof, the plant is selected from the group of cassava, corn, cowpea, rice, barley, wheat, Trema spp., apple, pear, plum, apricot, peach, almond, walnut, strawberry, raspberry, blackberry, red currant, black currant, melon, cucumber, pumpkin, squash, grape, bean, soybean, pea, chickpea, cowpea, pigeon pea, lentil, Bambara groundnut, lupin, pulses, Medicago spp., Lotus spp., forage legumes, indigo, legume trees, or hemp.

Further aspects of the present disclosure relate to methods of making the genetically modified plant of any of the preceding embodiments that have a genetically modified plant including a genetically modified cell including a TM receptor complex, including introducing a genetic alteration to the plant cell including a first nucleic acid sequence encoding a heterologous first subunit polypeptide including an affinity polypeptide; and/or introducing a genetic alteration to the plant cell including a second nucleic acid sequence encoding a heterologous second subunit polypeptide optionally including a tag polypeptide. In an additional embodiment of this aspect, the first nucleic acid sequence is operably linked to a promoter and/or wherein the second nucleic acid sequence is operably linked to a promoter. In a further embodiment of this aspect, the promoter is a root specific promoter, an inducible promoter, a constitutive promoter, or a combination thereof. In yet another embodiment of this aspect, which may be combined with any preceding embodiment that has a promoter, the promoter is a root specific promoter, and wherein the promoter is selected from the group of a NFR1 promoter, a NFR5/NFP promoter, a LYK3 promoter, a CERK6 promoter, a NFR5/NFP promoter, a Lotus japonicus NFR5 promoter (SEQ ID NO: 27), a Lotus japonicus NFR1 promoter (SEQ ID NO: 69), a Lotus japonicus CERK6 promoter (SEQ ID NO: 46), a Medicago truncatula NFP promoter (SEQ ID NO: 29), a Medicago truncatula LYK3 promoter (SEQ ID NO: 28), a maize metallothionein promoter, a chitinase promoter, a maize ZRP2 promoter, a tomato LeExtl promoter, a glutamine synthetase soybean root promoter, a RCC3 promoter, a rice antiquitin promoter, a LRR receptor kinase promoter, or an Arabidopsis pCO2 promoter. In still another embodiment of this aspect, which may be combined with any preceding embodiment that has a promoter, the promoter is a constitutive promoter, and wherein the promoter is selected from the group of a CaMV35S promoter, a derivative of the CaMV35S promoter, a maize ubiquitin promoter, a polyubiquitin promoter, a vein mosaic cassava virus promoter, or an Arabidopsis UBQ10 promoter. In a further embodiment of this aspect, the first nucleic acid sequence is inserted into the genome of the plant so that the nucleic acid sequence is operably linked to an endogenous promoter, and/or wherein the second nucleic acid sequence is inserted into the genome of the plant so that the nucleic acid sequence is operably linked to an endogenous promoter. In an additional embodiment of this aspect, the endogenous promoter is a root specific promoter.

Additional aspects of the present disclosure relate to methods of making the genetically modified plant of any of the preceding embodiments that have a genetically modified plant including a genetically modified cell including a TM receptor complex, including genetically modifying the plant cell by transforming the plant cell with one or more gene editing components that target a first endogenous nuclear genome sequence encoding the first subunit polypeptide, wherein the first subunit polypeptide is genetically modified to include the affinity polypeptide; and/or genetically modifying the plant cell by transforming the plant cell with one or more gene editing components that target a second endogenous nuclear genome sequence encoding the second subunit polypeptide, wherein the endogenous second subunit polypeptide is genetically modified to include a tag polypeptide. In another embodiment of this aspect, the one or more gene editing components include a ribonucleoprotein complex that targets the first and/or second nuclear genome sequence; a vector including a TALEN protein encoding sequence, wherein the TALEN protein targets the first and/or second nuclear genome sequence; a vector including a ZFN protein encoding sequence, wherein the ZFN protein targets the first and/or second nuclear genome sequence; an oligonucleotide donor (OND), wherein the OND targets the first and/or second nuclear genome sequence; or a vector CRISPR/Cas enzyme encoding sequence and a targeting sequence, wherein the targeting sequence targets the first and/or second nuclear genome sequence. In a further embodiment of this aspect, genetically modifying the first subunit to include the affinity polypeptide includes inserting a first nucleic acid sequence encoding a heterologous first subunit polypeptide including an affinity polypeptide into the first endogenous nuclear genome sequence; and wherein genetically modifying the second subunit to include the tag polypeptide includes inserting a second nucleic acid sequence encoding a heterologous second subunit polypeptide including a tag polypeptide into the second endogenous nuclear genome sequence.

Further aspects of the present disclosure relate to methods of making the genetically modified plant or part thereof of any one of the preceding embodiments that have a genetically modified plant including a genetically modified cell including a bispecific affinity polypeptide, including introducing a genetic alteration to the plant cell including a first nucleic acid sequence encoding a heterologous first subunit polypeptide including an affinity polypeptide; and/or introducing a genetic alteration to the plant cell including a second nucleic acid sequence encoding a heterologous second subunit polypeptide including a tag polypeptide. In a further embodiment of this aspect, the first nucleic acid sequence is operably linked to a promoter and/or wherein the second nucleic acid sequence is operably linked to a promoter. In an additional embodiment of this aspect, the promoter is a root specific promoter, a constitutive promoter, or a combination thereof. In yet another embodiment of this aspect, which may be combined with any of the preceding embodiments that has a promoter, the promoter is a root specific promoter, and wherein the promoter is selected from the group of a NFR1 promoter, a NFR5 promoter, a LYK3 promoter, a CERK6 promoter, a NFP promoter, a Lotus japonicus NFR5 promoter (SEQ ID NO: 27), a Lotus japonicus NFR1 promoter (SEQ ID NO: 69), a Lotus japonicus CERK6 promoter (SEQ ID NO: 46), a Medicago truncatula NFP promoter (SEQ ID NO: 29), a Medicago truncatula LYK3 promoter (SEQ ID NO: 28), a maize metallothionein promoter, a chitinase promoter, a maize ZRP2 promoter, a tomato LeExtl promoter, a glutamine synthetase soybean root promoter, a RCC3 promoter, a rice antiquitin promoter, a LRR receptor kinase promoter, or an Arabidopsis pCO2 promoter. In still another embodiment of this aspect, which may be combined with any of the preceding embodiments that has a promoter, the promoter is a constitutive promoter, and wherein the promoter is selected from the group of a CaMV35S promoter, a derivative of the CaMV35S promoter, a maize ubiquitin promoter, a polyubiquitin promoter, a vein mosaic cassava virus promoter, or an Arabidopsis UBQ10 promoter. In another embodiment of this aspect, the first nucleic acid sequence is inserted into the genome of the plant so that the nucleic acid sequence is operably linked to an endogenous promoter, and/or wherein the second nucleic acid sequence is inserted into the genome of the plant so that the nucleic acid sequence is operably linked to an endogenous promoter. In a further embodiment of this aspect, the endogenous promoter is a root specific promoter.

Additional aspects of the present disclosure relate to methods of making the genetically modified plant or part thereof of any one of the preceding embodiments that have a genetically modified plant including a genetically modified cell including a bispecific affinity polypeptide, including genetically modifying the plant cell by transforming the plant cell with one or more gene editing components that target a first nuclear genome sequence encoding an endogenous first subunit polypeptide, wherein the endogenous first subunit polypeptide is genetically modified to include an affinity polypeptide; and/or genetically modifying the plant cell by transforming the plant cell with one or more gene editing components that target a second nuclear genome sequence encoding an endogenous second subunit polypeptide to include a tag polypeptide, wherein the endogenous second subunit polypeptide is genetically modified to include an affinity polypeptide. In a further embodiment of this aspect, the one or more gene editing components include a ribonucleoprotein complex that targets the first and/or second nuclear genome sequence; a vector including a TALEN protein encoding sequence, wherein the TALEN protein targets the first and/or second nuclear genome sequence; a vector including a ZFN protein encoding sequence, wherein the ZFN protein targets the first and/or second nuclear genome sequence; an oligonucleotide donor (OND), wherein the OND targets the first and/or second nuclear genome sequence; or a vector CRISPR/Cas enzyme encoding sequence and a targeting sequence, wherein the targeting sequence targets the first and/or second nuclear genome sequence.

A further aspect of the present disclosure includes an expression vector or isolated DNA molecule including one or more nucleotide sequences encoding a first subunit polypeptide of a TM receptor including an affinity polypeptide, wherein the affinity polypeptide is heterologous to the first subunit polypeptide, and wherein the one or more nucleotide sequences are operably linked to at least one expression control sequence.

Yet another aspect of the present disclosure includes an expression vector or isolated DNA molecule including one or more nucleotide sequences encoding a second subunit polypeptide of a TM receptor optionally including a tag polypeptide, wherein the one or more nucleotide sequences are operably linked to at least one expression control sequence.

Still another aspect of the present disclosure includes an expression vector or isolated DNA molecule including one or more nucleotide sequences encoding: (a) a first subunit polypeptide of a transmembrane (TM) receptor complex including an affinity polypeptide, wherein the affinity polypeptide is heterologous to the first subunit polypeptide; and/or (b) a second subunit polypeptide of a TM receptor optionally including a tag polypeptide, wherein the one or more nucleotide sequences are operably linked to at least one expression control sequence.

Still another aspect of the present disclosure includes an expression vector or isolated DNA molecule including one or more nucleotide sequences encoding a bispecific affinity polypeptide including a first affinity polypeptide that binds to an intracellular portion of a first subunit polypeptide of a TM receptor and a second affinity polypeptide that binds to an intracellular portion of a second subunit polypeptide of a TM receptor, wherein the one or more nucleotide sequences are operably linked to at least one expression control sequence. In an additional embodiment of this aspect, the first affinity polypeptide binds directly to the first subunit polypeptide and/or wherein the second affinity polypeptide binds directly to the second subunit polypeptide. In yet another embodiment of this aspect, the first subunit polypeptide includes a tag polypeptide, and wherein the first affinity polypeptide binds to the tag polypeptide, and/or wherein the second polypeptide includes a tag polypeptide, and wherein the second affinity polypeptide binds to the tag polypeptide. In still another embodiment of this aspect, which may be combined with any preceding embodiments and aspects that have a tag polypeptide, the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, or a ubiquitin tag polypeptide. In a further embodiment of this aspect, the tag polypeptide is a fluorescent protein polypeptide selected from a green fluorescent protein (GFP) polypeptide, a red fluorescent polypeptide (RFP) (e.g., mCherry), or a blue fluorescent polypeptide (BFP); wherein the tag polypeptide is a luminescence polypeptide selected from a luciferase polypeptide; or wherein the tag polypeptide is a combination of a fluorescent protein polypeptide and a luminescence polypeptide. In yet another embodiment of this aspect, which may be combined with any preceding embodiments that have a first affinity polypeptide and a second affinity polypeptide, the first affinity polypeptide and the second affinity polypeptide are selected from a heavy-chain variable domain (VHH), a single-chain variable fragment (scFV), a designed ankyrin repeat protein (DARPins), a human fibronectin III domain 3 monobody, an Affibody, or an anticalin, or a synthetic version of any of the foregoing. In still another embodiment of this aspect, the first affinity polypeptide and/or the second affinity polypeptide is a heavy-chain variable domain (VHH). In a further embodiment of this aspect, the affinity polypeptide is a VHH_{LaG16}. In an additional embodiment of this aspect, which may be combined with any of the preceding embodiments and aspects that have an expression vector or isolated DNA molecule, the first subunit polypeptide and/or the second subunit polypeptide are selected from a LysM receptor, a Leucine rich repeat (LRR) receptor, a Malectin like receptor, a single-pass TM (SPTM) receptor, a plant TM receptor, a plant SPTM (pSPTM) receptor, a SPTM receptor including an intracellular kinase domain (SPTM-kinase), and a plant SPTM receptor including an intracellular kinase domain (pSPTM-kinase). In a further embodiment of this aspect, the first subunit polypeptide and/or the second subunit polypeptide is a LysM receptor. In another embodiment of this aspect, the first subunit polypeptide and/or the second subunit polypeptide lacks an ectodomain and/or a transmembrane domain. In still another embodiment of this aspect, which may be combined with any of the preceding embodiments and aspects that have a LysM receptor, the first subunit polypeptide is a NFR1 polypeptide and the second subunit polypeptide is a NFR5 polypeptide, wherein the first subunit polypeptide is a NFR5 polypeptide and the second subunit is a NFR1 polypeptide, wherein the first subunit polypeptide is a LYK3 polypeptide and the second subunit polypeptide is a NFP polypeptide, wherein the first subunit polypeptide is a NFP polypeptide and the second subunit polypeptide is a LYK3 polypeptide, wherein the first subunit polypeptide is a RLK4 receptor polypeptide and the second subunit polypeptide is a RLK10 receptor polypeptide, or wherein the first subunit polypeptide is a RLK10 polypeptide and the second subunit polypeptide is a RLK4 polypeptide. In yet another embodiment of this aspect, the NFR1, LYK3, or RLK4 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 56, and wherein the NFR5, NFP, or RLK10 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 57. In an additional embodiment of this aspect, which may be combined with any of the preceding embodiments and aspects that have an expression vector or isolated DNA molecule, the at least one expression control sequence includes a promoter selected from the group of a root specific promoter, a constitutive promoter, or a combination thereof. In a further embodiment of this aspect, the promoter is a root specific promoter, and wherein the promoter is selected from the group of a NFR1 promoter, a NFR5/NFP promoter, a LYK3 promoter, a CERK6 promoter, a NFR5/NFP promoter, a Lotus japonicus NFR5 promoter (SEQ ID NO: 27), a Lotus japonicus NFR1 promoter (SEQ ID NO: 69), a Lotus japonicus CERK6 promoter (SEQ ID NO: 46), a Medicago truncatula NFP promoter (SEQ ID NO: 29), a Medicago truncatula LYK3 promoter (SEQ ID NO: 28), a maize metallothionein promoter, a chitinase promoter, a maize ZRP2 promoter, a tomato LeExtl promoter, a glutamine synthetase soybean root promoter, a RCC3 promoter, a rice antiquitin promoter, a LRR receptor kinase promoter, or an Arabidopsis pCO2 promoter. In another embodiment of this aspect, the promoter is a constitutive promoter, and wherein the promoter is selected from the group of a CaMV35S promoter, a derivative of the CaMV35S promoter, a maize ubiquitin promoter, a polyubiquitin promoter, a vein mosaic cassava virus promoter, or an Arabidopsis UBQ10 promoter.

Some aspects of the present disclosure relate to a bacterial cell or an Agrobacterium cell including the expression vector or isolated DNA molecule of any one of the preceding embodiments.

Additional aspects of the present disclosure relate to a genetically modified plant, plant part, plant cell, or seed including the expression vector or isolated DNA molecule of any one of the preceding embodiments.

Further aspects of the present disclosure relate to a kit including the expression vector or isolated DNA molecule of any one of the preceding embodiments of the bacterial cell or the Agrobacterium cell of the preceding embodiments.

Still further aspects of the present disclosure relate to methods of activating a target transmembrane (TM) receptor complex or inducing organogenesis including: introducing a genetic alteration via an expression vector or isolated DNA molecule of any one of the preceding embodiments to a cell. In an additional embodiment of this aspect, activating the target TM receptor complex or inducing organogenesis is in the absence of a native, an endogenous, or exogenous stimulus (e.g., Nod). In a further embodiment of this aspect, which may be combined with any preceding embodiments, the method further includes knocking out a native target TM receptor complex or subunits thereof in the cell. In still another embodiment of this aspect, which may be combined with any preceding embodiments, the cell is a plant cell.

Another aspect of the disclosure includes a genetically modified plant cell including: an NFR1-NFR5 receptor complex including a first subunit polypeptide and a second subunit polypeptide, wherein the first subunit polypeptide includes an affinity polypeptide that binds to the second subunit polypeptide inducing oligomerization, and wherein the affinity polypeptide is heterologous to the first subunit polypeptide; and wherein oligomerization of the first subunit polypeptide and the second subunit polypeptide activates NFR1-NFR5 receptor complex signaling and wherein (i) the first subunit polypeptide is an NFR1 polypeptide and the second subunit polypeptide is an NFR5 polypeptide, or (ii) the first subunit polypeptide is the NFR5 polypeptide and the second subunit polypeptide is the NFR1 polypeptide. In an additional embodiment of this aspect, the NFR1 polypeptide and/or the NFR5 polypeptide lacks an ectodomain and/or a transmembrane domain. In a further embodiment of this aspect, which may be combined with any of the preceding embodiments, the NFR1 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 56, and wherein the NFR5 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 57. In yet another embodiment of this aspect, which may be combined with any of the preceding embodiments, the affinity polypeptide binds directly to an intracellular portion of the second subunit polypeptide. In still another embodiment of this aspect, which may be combined with any of the preceding embodiments, the second subunit polypeptide includes a tag polypeptide fused to an intracellular portion of the second subunit polypeptide, and wherein the affinity polypeptide binds to the tag polypeptide. In a further embodiment of this aspect, the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, or a ubiquitin tag polypeptide. In an additional embodiment of this aspect, the tag polypeptide is a fluorescent protein polypeptide selected from a green fluorescent protein (GFP) polypeptide, a red fluorescent polypeptide (RFP) (e.g., mCherry), or a blue fluorescent polypeptide (BFP); wherein the tag polypeptide is a luminescence polypeptide selected from a luciferase polypeptide; or wherein the tag polypeptide is a combination of a fluorescent protein polypeptide and a luminescence polypeptide. In yet another embodiment of this aspect, which may be combined with any of the preceding embodiments, the affinity polypeptide is selected from a heavy-chain variable domain (VHH), a single-chain variable fragment (scFV), a designed ankyrin repeat protein (DARPins), a human fibronectin III domain 3 monobody, an Affibody, or an anticalin, or a synthetic version of any of the foregoing. In still another embodiment of this aspect, the affinity polypeptide is a heavy-chain variable domain (VHH). In a further embodiment of this aspect, the affinity polypeptide is VHH_{LaG16}. In another embodiment of this aspect, which may be combined with any of the preceding embodiments, the affinity polypeptide binding to the second subunit polypeptide is regulated by a small molecule.

A further aspect of the disclosure includes a bispecific affinity polypeptide for activation of an NFR1-NFR5 receptor including: a first affinity polypeptide that binds to an intracellular portion of an NFR1 polypeptide, wherein the first affinity polypeptide is heterologous to the NFR1 polypeptide; a second affinity polypeptide that binds to an intracellular portion of an NFR5 polypeptide, wherein the affinity polypeptide is heterologous to the NFR5 receptor subunit polypeptide; and wherein binding of the first affinity polypeptide to the NFR1 polypeptide and of the second affinity to the NFR5 polypeptide induces dimerization; and wherein dimerization of the NFR1 polypeptide and the NFR5 polypeptide activates the NFR1-NFR5 receptor. In an additional aspect of this aspect. the NFR1 polypeptide and/or the NFR5 polypeptide lacks an ectodomain and/or a transmembrane domain. In yet another embodiment of this aspect, which may be combined with any of the preceding embodiments, the NFR1 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 56, and wherein the NFR5 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 57. In still another embodiment of this aspect, which may be combined with any of the preceding embodiments, the first affinity polypeptide binds directly to the NFR1 polypeptide and/or wherein the second affinity polypeptide binds directly to the NFR5 polypeptide. In an additional embodiment of this aspect, which may be combined with any of the preceding embodiments, the NFR1 polypeptide includes a tag polypeptide fused to an intracellular portion of the NFR1 polypeptide, and wherein the first affinity polypeptide binds to the tag polypeptide; and/or wherein the NFR5 polypeptide includes a tag polypeptide fused to an intracellular portion of the NFR5 polypeptide, and wherein the second affinity polypeptide binds to the tag polypeptide. In a further embodiment of this aspect, the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a luciferase tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, or a ubiquitin tag polypeptide. In another embodiment of this aspect, the tag polypeptide is a fluorescent protein polypeptide selected from a green fluorescent protein (GFP) polypeptide, a red fluorescent polypeptide (RFP) (e.g., mCherry), or a blue fluorescent polypeptide (BFP); wherein the tag polypeptide is a luminescence polypeptide selected from a luciferase polypeptide; or wherein the tag polypeptide is a combination of a fluorescent protein polypeptide and a luminescence polypeptide. In an additional embodiment of this aspect, which may be combined with any of the preceding embodiments, the first affinity polypeptide and the second affinity polypeptide are selected from a heavy-chain variable domain (VHH), a single-chain variable fragment (scFV), a designed ankyrin repeat protein (DARPins), a human fibronectin III domain 3 monobody, an Affibody, or an anticalin, or a synthetic version of any of the foregoing. In a further embodiment of this aspect, the first affinity polypeptide and/or the second affinity polypeptide is a heavy-chain variable domain (VHH). In an additional embodiment of this aspect, the affinity polypeptide is VHH_{LaG16}. In yet another embodiment of this aspect, which may be combined with any of the preceding embodiments, the first affinity polypeptide binding to the NFR1 polypeptide is regulated by a small molecule and/or the second affinity polypeptide binding to the NFR5 polypeptide is regulated by a small molecule.

Some aspects of the disclosure relate to a genetically modified plant or part thereof including the genetically modified plant cell of any of the preceding embodiments including a NFR1-NFR5 receptor complex. Another embodiment of this aspect further includes the NFR1-NFR5 receptor complex including the first subunit polypeptide and the second subunit polypeptide, wherein the first subunit polypeptide includes an affinity polypeptide that binds to the second subunit polypeptide inducing oligomerization; and wherein oligomerization of the first subunit polypeptide and the second subunit polypeptide activates NFR1-NFR5 receptor complex signaling. In an additional embodiment of this aspect, the affinity polypeptide binds directly to the second subunit polypeptide or wherein the second subunit polypeptide includes a tag polypeptide fused to an intracellular portion of the second subunit polypeptide, and wherein the affinity polypeptide binds to the tag polypeptide. In yet another embodiment of this aspect, which may be combined with any of the preceding embodiments, the genetically modified plant cell is selected from a root epidermal cell, a root cortex cell, a root endodermis cell, a root pericycle cell, a root primordia cell, a xylem cell, a phloem cell, a meristem cell, a leaf cell, a stem cell, a flower cell, or a fruit cell. In a further embodiment of this aspect, the genetically modified plant cell is a root epidermal cell, a root cortex cell, a root endodermis cell, a root pericycle cell, or a root primordia cell.

Further aspects of the disclosure relate to a genetically modified plant or part thereof including the bispecific affinity polypeptide of any of the preceding embodiments including a NFR1-NFR5 receptor complex. Another embodiment of this aspect further includes the first affinity polypeptide that binds to an intracellular portion of the NFR1 polypeptide and the second affinity polypeptide that binds to the intracellular portion of the NFR5 polypeptide, wherein binding of the first affinity polypeptide to the NFR1 polypeptide and of the second affinity polypeptide to the NFR5 polypeptide induces oligomerization; and wherein oligomerization of the NFR1 polypeptide and the NFR5 polypeptide activates NFR1-NFR5 receptor complex signaling. In a further embodiment of this aspect, the first affinity polypeptide binds directly to the NFR1 polypeptide and/or wherein the second affinity polypeptide binds directly to the NFR5 polypeptide; or wherein the NFR1 polypeptide includes a tag polypeptide fused to the intracellular portion of the NFR1 polypeptide, and wherein the first affinity polypeptide binds to the tag polypeptide, and/or wherein the NFR5 polypeptide includes a tag polypeptide fused to the intracellular portion of the NFR5 polypeptide, and wherein the second affinity polypeptide binds to the tag polypeptide. In yet another embodiment of this aspect, which may be combined with any of the preceding embodiments, the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, or a ubiquitin tag polypeptide. In a further embodiment of this aspect, the tag polypeptide is a fluorescent protein polypeptide selected from a green fluorescent protein (GFP) polypeptide, a red fluorescent polypeptide (RFP) (e.g., mCherry), or a blue fluorescent polypeptide (BFP); wherein the tag polypeptide is a luminescence polypeptide selected from a luciferase polypeptide; or wherein the tag polypeptide is a combination of a fluorescent protein polypeptide and a luminescence polypeptide. In still another embodiment of this aspect, which may be combined with any of the preceding embodiments, the affinity polypeptide is selected from a heavy-chain variable domain (VHH), a single-chain variable fragment (scFV), a designed ankyrin repeat protein (DARPins), a human fibronectin III domain 3 monobody, an Affibody, or an anticalin, or a synthetic version of any of the foregoing. In another embodiment of this aspect, the affinity polypeptide is a heavy-chain variable domain (VHH). In an additional embodiment of this aspect, the affinity polypeptide is a VHH_{LaG16}. In yet another embodiment of this aspect, which may be combined with any of the preceding embodiments, the NFR1-NFR5 receptor complex is localized to a plant cell membrane. In a further embodiment of this aspect, which may be combined with any of the preceding embodiments, the plant part is a leaf, a stem, a root, a root primordia, a flower, a seed, a fruit, a kernel, a grain, a cell, or a portion thereof. In another embodiment of this aspect, which may be combined with any of the preceding embodiments, the plant is selected from the group of cassava, corn, cowpea, rice, barley, wheat, Trema spp., apple, pear, plum, apricot, peach, almond, walnut, strawberry, raspberry, blackberry, red currant, black currant, melon, cucumber, pumpkin, squash, grape, bean, soybean, pea, chickpea, cowpea, pigeon pea, lentil, Bambara groundnut, lupin, pulses, Medicago spp., Lotus spp., forage legumes, indigo, legume trees, or hemp.

Additional aspects of the disclosure relate to methods of making the genetically modified plant cell of any of the preceding embodiments including a NFR1-NFR5 receptor complex, including introducing a genetic alteration to the plant cell including a first nucleic acid sequence encoding a heterologous NFR1 polypeptide including an affinity polypeptide; and/or introducing a genetic alteration to the plant cell including a second nucleic acid sequence encoding a heterologous NFR5 polypeptide optionally including a tag polypeptide. In a further embodiment of this aspect, the NFR1 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 56, and wherein the NFR5 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 57. In an additional embodiment of this aspect, which may be combined with any of the preceding embodiments, the first nucleic acid sequence is operably linked to a promoter and/or wherein the second nucleic acid sequence is operably linked to a promoter. In another embodiment of this aspect, the promoter is a root specific promoter, a constitutive promoter, or a combination thereof. In a further embodiment of this aspect, the promoter is a root specific promoter, and wherein the promoter is selected from the group of a NFR1 promoter, a NFR5/NFP promoter, a LYK3 promoter, a CERK6 promoter, a NFR5/NFP promoter, a Lotus japonicus NFR5 promoter (SEQ ID NO: 27), a Lotus japonicus NFR1 promoter (SEQ ID NO: 69), a Lotus japonicus CERK6 promoter (SEQ ID NO: 46), a Medicago truncatula NFP promoter (SEQ ID NO: 29), a Medicago truncatula LYK3 promoter (SEQ ID NO: 28), a maize metallothionein promoter, a chitinase promoter, a maize ZRP2 promoter, a tomato LeExtl promoter, a glutamine synthetase soybean root promoter, a RCC3 promoter, a rice antiquitin promoter, a LRR receptor kinase promoter, or an Arabidopsis pCO2 promoter. In yet another embodiment of this aspect, the promoter is a constitutive promoter, and wherein the promoter is selected from the group of a CaMV35S promoter, a derivative of the CaMV35S promoter, a maize ubiquitin promoter, a polyubiquitin promoter, a vein mosaic cassava virus promoter, or an Arabidopsis UBQ10 promoter. In still another embodiment of this aspect, the first nucleic acid sequence is inserted into the genome of the plant so that the nucleic acid sequence is operably linked to an endogenous promoter, and/or wherein the second nucleic acid sequence is inserted into the genome of the plant so that the nucleic acid sequence is operably linked to an endogenous promoter. In a further embodiment of this aspect, the endogenous promoter is a root specific promoter.

Further aspects of the disclosure relate to methods of making the genetically modified plant cell of any of the preceding embodiments including a NFR1-NFR5 receptor complex, including genetically modifying the plant cell by transforming the plant cell with one or more gene editing components that target a first nuclear genome sequence encoding an endogenous NFR1 polypeptide, wherein the endogenous NFR1 polypeptide is genetically modified to include an affinity polypeptide; and/or genetically modifying the plant cell by transforming the plant cell with one or more gene editing components that target a second nuclear genome sequence encoding an endogenous NFR5 polypeptide to include a tag polypeptide, wherein the endogenous NFR5 polypeptide is genetically modified to include a tag polypeptide. In a further embodiment of this aspect, the NFR1 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 56, and wherein the NFR5 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 57. In an additional embodiment of this aspect, which may be combined with any of the preceding embodiments, the one or more gene editing components include a ribonucleoprotein complex that targets the first and/or second nuclear genome sequence; a vector including a TALEN protein encoding sequence, wherein the TALEN protein targets the first and/or second nuclear genome sequence; a vector including a ZFN protein encoding sequence, wherein the ZFN protein targets the first and/or second nuclear genome sequence; an oligonucleotide donor (OND), wherein the OND targets the first and/or second nuclear genome sequence; or a vector CRISPR/Cas enzyme encoding sequence and a targeting sequence, wherein the targeting sequence targets the first and/or second nuclear genome sequence. In yet another embodiment of this aspect, which may be combined with any of the preceding embodiments, genetically modifying the endogenous NFR1 polypeptide to include the affinity polypeptide includes inserting a first nucleic acid sequence encoding a heterologous NFR1 polypeptide including an affinity polypeptide into the endogenous NFR1 nuclear genome sequence; and wherein genetically modifying the endogenous NFR5 polypeptide to include the tag polypeptide includes inserting a second nucleic acid sequence encoding a heterologous NFR5 polypeptide including a tag polypeptide into the endogenous NFR5 nuclear genome sequence.

Yet further aspects of the disclosure relate to methods of making the genetically modified plant cell of any of the preceding embodiments including a NFR1-NFR5 receptor complex, including introducing a genetic alteration to the plant cell including a first nucleic acid sequence encoding a heterologous NFR1 polypeptide including an affinity polypeptide; and/or introducing a genetic alteration to the plant cell including a second nucleic acid sequence encoding a heterologous NFR5 polypeptide optionally including a tag polypeptide. In a further embodiment of this aspect, the NFR1 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 56, and wherein the NFR5 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 57. In an additional embodiment of this aspect, which may be combined with any of the preceding embodiments, the first nucleic acid sequence is operably linked to a promoter and/or wherein the second nucleic acid sequence is operably linked to a promoter. In another embodiment of this aspect, the promoter is a root specific promoter, a constitutive promoter, or a combination thereof. In a further embodiment of this aspect, the promoter is a root specific promoter, and wherein the promoter is selected from the group of a NFR1 promoter, a NFR5/NFP promoter, a LYK3 promoter, a CERK6 promoter, a NFR5/NFP promoter, a Lotus japonicus NFR5 promoter (SEQ ID NO: 27), a Lotus japonicus NFR1 promoter (SEQ ID NO: 69), a Lotus japonicus CERK6 promoter (SEQ ID NO: 46), a Medicago truncatula NFP promoter (SEQ ID NO: 29), a Medicago truncatula LYK3 promoter (SEQ ID NO: 28), a maize metallothionein promoter, a chitinase promoter, a maize ZRP2 promoter, a tomato LeExtl promoter, a glutamine synthetase soybean root promoter, a RCC3 promoter, a rice antiquitin promoter, a LRR receptor kinase promoter, or an Arabidopsis pCO2 promoter. In yet another embodiment of this aspect, the promoter is a constitutive promoter, and wherein the promoter is selected from the group of a CaMV35S promoter, a derivative of the CaMV35S promoter, a maize ubiquitin promoter, a polyubiquitin promoter, a vein mosaic cassava virus promoter, or an Arabidopsis UBQ10 promoter. In still another embodiment of this aspect, the first nucleic acid sequence is inserted into the genome of the plant so that the nucleic acid sequence is operably linked to an endogenous promoter, and/or wherein the second nucleic acid sequence is inserted into the genome of the plant so that the nucleic acid sequence is operably linked to an endogenous promoter. In a further embodiment of this aspect, the endogenous promoter is a root specific promoter.

A further aspect of the present disclosure includes an expression vector or isolated DNA molecule including one or more nucleotide sequences encoding a NFR1 polypeptide including an affinity polypeptide, wherein the affinity polypeptide is heterologous to the NFR1 polypeptide, wherein the one or more nucleotide sequences are operably linked to at least one expression control sequence.

Yet another aspect of the present disclosure includes an expression vector or isolated DNA molecule including one or more nucleotide sequences encoding a NFR5 polypeptide optionally including a tag polypeptide, wherein the one or more nucleotide sequences are operably linked to at least one expression control sequence.

Still another aspect of the present disclosure includes an expression vector or isolated DNA molecule including one or more nucleotide sequences encoding: (a) a NFR1 polypeptide including an affinity polypeptide, wherein the affinity polypeptide is heterologous to the first subunit polypeptide; and/or (b) a NFR5 polypeptide optionally including a tag polypeptide, wherein the one or more nucleotide sequences are operably linked to at least one expression control sequence.

An additional aspect of the present disclosure includes an expression vector or isolated DNA molecule including one or more nucleotide sequences encoding a bispecific affinity polypeptide including a first affinity polypeptide that binds to an intracellular portion of a NFR1 polypeptide and a second affinity polypeptide that binds to an intracellular portion of a NFR5 polypeptide, wherein the one or more nucleotide sequences are operably linked to at least one expression control sequence, and wherein the first affinity polypeptide is heterologous to the NFR1 polypeptide and the second affinity polypeptide is heterologous to the NFR5 polypeptide. In a further embodiment of this aspect, the first affinity polypeptide binds directly to the NFR1 polypeptide and/or wherein the second subunit polypeptide binds directly to the NFR5 polypeptide. In another embodiment of this aspect, which may be combined with any of the preceding embodiments, the NFR1 polypeptide includes a tag polypeptide, and wherein the first affinity polypeptide binds to the tag polypeptide and/or wherein the wherein the NFR5 polypeptide includes a tag polypeptide, and wherein second affinity polypeptide binds to the tag polypeptide. In a further embodiment of this aspect, which may be combined with any of the preceding embodiments and aspects, the NFR1 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 56, and wherein the NFR5 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 57. In yet another embodiment of this aspect, which may be combined with any of the preceding embodiments and aspects including an expression vector or isolated DNA molecule, the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a luciferase tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, or a ubiquitin tag polypeptide. In a further embodiment of this aspect, the tag polypeptide is a fluorescent protein polypeptide selected from a green fluorescent protein (GFP) polypeptide, a red fluorescent polypeptide (RFP) (e.g., mCherry), or a blue fluorescent polypeptide (BFP); wherein the tag polypeptide is a luminescence polypeptide selected from a luciferase polypeptide; or wherein the tag polypeptide is a combination of a fluorescent protein polypeptide and a luminescence polypeptide. In still another embodiment of this aspect, which may be combined with any of the preceding embodiments, the affinity polypeptide is selected from a heavy-chain variable domain (VHH), a single-chain variable fragment (scFV), a designed ankyrin repeat protein (DARPins), a human fibronectin III domain 3 monobody, an Affibody, or an anticalin, or a synthetic version of any of the foregoing. In another embodiment of this aspect, the affinity polypeptide is a heavy-chain variable domain (VHH). In an additional embodiment of this aspect, the affinity polypeptide is a VHH_{LaG16}. In still another embodiment of this aspect, which may be combined with any of the preceding embodiments and aspects including an expression vector or isolated DNA molecule, the NFR1 polypeptide and/or the NFR5 polypeptide lacks an ectodomain and/or a transmembrane domain. In yet another embodiment of this aspect, which may be combined with any of the preceding embodiments and aspects including an expression vector or isolated DNA molecule, the at least one expression control sequence includes a promoter selected from the group of a root specific promoter, a constitutive promoter, or a combination thereof. In a further embodiment of this aspect, the promoter is a root specific promoter, and wherein the promoter is selected from the group of a NFR1 promoter, a NFR5/NFP promoter, a LYK3 promoter, a CERK6 promoter, a NFR5/NFP promoter, a Lotus japonicus NFR5 promoter (SEQ ID NO: 27), a Lotus japonicus NFR1 promoter (SEQ ID NO: 69), a Lotus japonicus CERK6 promoter (SEQ ID NO: 46), a Medicago truncatula NFP promoter (SEQ ID NO: 29), a Medicago truncatula LYK3 promoter (SEQ ID NO: 28), a maize metallothionein promoter, a chitinase promoter, a maize ZRP2 promoter, a tomato LeExtl promoter, a glutamine synthetase soybean root promoter, a RCC3 promoter, a rice antiquitin promoter, a LRR receptor kinase promoter, or an Arabidopsis pCO2 promoter. In yet another embodiment of this aspect, the promoter is a constitutive promoter, and wherein the promoter is selected from the group of a CaMV35S promoter, a derivative of the CaMV35S promoter, a maize ubiquitin promoter, a polyubiquitin promoter, a vein mosaic cassava virus promoter, or an Arabidopsis UBQ10 promoter.

Some aspects of the present disclosure relate to a bacterial cell or an Agrobacterium cell including the expression vector or isolated DNA molecule of any one of the preceding embodiments.

Additional aspects of the present disclosure relate to a genetically modified plant, plant part, plant cell, or seed including the expression vector or isolated DNA molecule of any one of the preceding embodiments.

Further aspects of the present disclosure relate to a kit including the expression vector or isolated DNA molecule of any one of the preceding embodiments of the bacterial cell or the Agrobacterium cell of the preceding embodiments.

Still further aspects of the present disclosure relate to methods of activating a target NFR1-NFR5 receptor complex or inducing organogenesis including: introducing a genetic alteration via an expression vector or isolated DNA molecule of any one of the preceding embodiments to a cell. In an additional embodiment of this aspect, activating the target NFR1-NFR5 receptor complex or inducing organogenesis is in the absence of a native, an endogenous, or exogenous stimulus (e.g., Nod). In a further embodiment of this aspect, which may be combined with any preceding embodiments, the method further includes knocking out a native target NFR1-NFR5 receptor complex or subunits thereof in the cell. In still another embodiment of this aspect, which may be combined with any preceding embodiments, the cell is a plant cell.

### ENUMERATED EMBODIMENTS

1. A genetically modified cell comprising:
   a transmembrane (TM) receptor complex comprising a first subunit polypeptide and a second subunit polypeptide,
   wherein the first subunit polypeptide comprises an affinity polypeptide that binds to the second subunit polypeptide intracellularly inducing oligomerization; and
   wherein oligomerization of the first subunit polypeptide and the second subunit polypeptide activates TM receptor signaling, wherein the affinity polypeptide is heterologous to the first subunit polypeptide.
2. The genetically modified cell of embodiment 1, wherein the TM receptor is a single-pass TM (SPTM) receptor, or wherein the TM receptor is a SPTM receptor comprising an intracellular kinase domain (SPTM-kinase).
3. The genetically modified cell of embodiment 1 or embodiment 2, wherein the affinity polypeptide binds directly to the second subunit polypeptide.
4. The genetically modified cell of embodiment 1 or embodiment 2, wherein the second subunit polypeptide comprises a tag polypeptide, and wherein the affinity polypeptide binds to the tag polypeptide.
5. The genetically modified cell of embodiment 4, wherein the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, and a ubiquitin tag polypeptide.
6. The genetically modified cell of embodiment 5, wherein the tag polypeptide is a fluorescent protein polypeptide selected from a green fluorescent protein (GFP) polypeptide, a red fluorescent polypeptide (RFP) (e.g., mCherry), and a blue fluorescent polypeptide (BFP); wherein the tag polypeptide is a luminescence polypeptide selected from a luciferase polypeptide; or wherein the tag polypeptide is a combination of a fluorescent protein polypeptide and a luminescence polypeptide.
7. The genetically modified cell of any one of embodiments 1-6, wherein the affinity polypeptide is selected from a heavy-chain variable domain (VHH), a single-chain variable fragment (scFV), a designed ankyrin repeat protein (DARPins), a human fibronectin III domain 3 monobody, an Affibody, and an anticalin, and a synthetic version of any of the foregoing.
8. The genetically modified cell of embodiment 7, wherein the affinity polypeptide is a heavy-chain variable domain (VHH).
9. The genetically modified cell of embodiment 8, wherein the affinity polypeptide is a VHH_{LaG16}.
10. The genetically modified cell of any one of embodiments 1-9, wherein the cell is a eubacterial cell, an archaeal cell, or a eukaryotic cell.
11. The genetically modified cell of embodiment 10, wherein the eukaryotic cell is a plant cell, an animal cell, or a fungal cell.
12. The genetically modified cell of any one of embodiments 1-11, wherein the cell is a plant cell and the first subunit polypeptide and/or the second subunit polypeptide are selected from a LysM receptor, a Leucine rich repeat (LRR) receptor, a Malectin like receptor, a plant TM receptor, a plant SPTM (pSPTM) receptor, and a plant SPTM receptor comprising an intracellular kinase domain (pSPTM-kinase).
13. The genetically modified cell of embodiment 12, wherein the first subunit polypeptide and/or the second subunit polypeptide is a LysM receptor.
14. The genetically modified cell of embodiment 13, wherein the first subunit polypeptide and/or the second subunit polypeptide lacks an ectodomain and/or a transmembrane domain.
15. The genetically modified cell of embodiment 13 or embodiment 14, wherein the first subunit polypeptide is a NFR1 polypeptide and the second subunit polypeptide is a NFR5 polypeptide, wherein the first subunit polypeptide is a NFR5 polypeptide and the second subunit is a NFR1 polypeptide, wherein the first subunit polypeptide is a LYK3 polypeptide and the second subunit polypeptide is a NFP polypeptide, wherein the first subunit polypeptide is a NFP polypeptide and the second subunit polypeptide is a LYK3 polypeptide, wherein the first subunit polypeptide is a RLK4 receptor polypeptide and the second subunit polypeptide is a RLK10 receptor polypeptide, or wherein the first subunit polypeptide is a RLK10 polypeptide and the second subunit polypeptide is a RLK4 polypeptide.
16. The genetically modified cell of embodiment 15, wherein the NFR1, LYK3, or RLK4 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 56, and wherein the NFR5, NFP, or RLK10 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, and SEQ ID NO: 57.
17. The genetically modified cell of any one of embodiments 1-16, wherein the affinity polypeptide binding to the second subunit polypeptide is regulated by a small molecule.
18. A bispecific affinity polypeptide comprising:
   a first affinity polypeptide that binds to an intracellular portion of a first subunit polypeptide of a TM receptor; and
   a second affinity polypeptide that binds to an intracellular portion of a second subunit polypeptide of the TM receptor;
   wherein binding of the first affinity polypeptide to the first subunit polypeptide and of the second affinity polypeptide to the second subunit polypeptide induces oligomerization; and
   wherein oligomerization of the first subunit polypeptide and the second subunit polypeptide activates TM receptor signaling.
19. The bispecific affinity polypeptide of embodiment 18, wherein the TM receptor is a single-pass TM (SPTM) receptor, or wherein the TM receptor is a SPTM comprising an intracellular kinase domain (SPTM-kinase).
20. The bispecific affinity polypeptide of embodiment 18 or embodiment 19, wherein the first affinity polypeptide binds directly to the first subunit polypeptide and/or wherein the second affinity polypeptide binds directly to the second subunit polypeptide.
21. The bispecific affinity polypeptide of any one of embodiments 18-20, wherein the first subunit polypeptide comprises a tag polypeptide, and wherein the first affinity polypeptide binds to the tag polypeptide, and/or wherein the second subunit polypeptide comprises a tag polypeptide, and wherein the second affinity polypeptide binds to the tag polypeptide.
22. The bispecific affinity polypeptide of embodiment 21, wherein the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, and a ubiquitin tag polypeptide.
23. The bispecific affinity polypeptide of embodiment 22, wherein the tag polypeptide is a fluorescent protein polypeptide selected from a green fluorescent protein (GFP) polypeptide, a red fluorescent polypeptide (RFP) (e.g., mCherry), and a blue fluorescent polypeptide (BFP); wherein the tag polypeptide is a luminescence polypeptide selected from a luciferase polypeptide; or wherein the tag polypeptide is a combination of a fluorescent protein polypeptide and a luminescence polypeptide.
24. The bispecific affinity polypeptide of any one of embodiments 18-23, wherein the first affinity polypeptide and the second affinity polypeptide are selected from a heavy-chain variable domain (VHH), a single-chain variable fragment (scFV), a designed ankyrin repeat protein (DARPins), a human fibronectin III domain 3 monobody, an Affibody, and an anticalin, and a synthetic version of any of the foregoing.
25. The bispecific affinity polypeptide of embodiment 24, wherein the first affinity polypeptide and/or the second affinity polypeptide is a heavy-chain variable domain (VHH).
26. The bispecific affinity polypeptide of embodiment 25, wherein the first affinity polypeptide and/or the second affinity polypeptide is a VHH_{LaG16}.
27. The bispecific affinity polypeptide of any one of embodiments 18-26, wherein the first subunit polypeptide and/or the second subunit polypeptide are selected from a LysM receptor, a Leucine rich repeat (LRR) receptor, a Malectin like receptor, a plant TM receptor, a plant SPTM (pSPTM) receptor, and a plant SPTM receptor comprising an intracellular kinase domain (pSPTM-kinase).
28. The bispecific affinity polypeptide of embodiment 27, wherein the first subunit polypeptide and/or the second subunit polypeptide is a LysM receptor.
29. The bispecific affinity polypeptide of embodiment 28, wherein the first subunit polypeptide and/or the second subunit polypeptide lacks an ectodomain and/or a transmembrane domain.
30. The bispecific affinity polypeptide of embodiment 28 or embodiment 29, wherein the first subunit polypeptide is a NFR1 polypeptide and the second subunit polypeptide is a NFR5 polypeptide, wherein the first subunit polypeptide is a NFR5 polypeptide and the second subunit is a NFR1 polypeptide, wherein the first subunit polypeptide is a LYK3 polypeptide and the second subunit polypeptide is a NFP polypeptide, wherein the first subunit polypeptide is a NFP polypeptide and the second subunit polypeptide is a LYK3 polypeptide, wherein the first subunit polypeptide is a RLK4 receptor polypeptide and the second subunit polypeptide is a RLK10 receptor polypeptide, or wherein the first subunit polypeptide is a RLK10 polypeptide and the second subunit polypeptide is a RLK4 polypeptide.
31. The bispecific affinity polypeptide of embodiment 30, wherein the NFR1, LYK3, or RLK4 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 56, and wherein the NFR5, NFP, or RLK10 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, and SEQ ID NO: 57.
32. The bispecific affinity polypeptide of any one of embodiments 18-31, wherein the first affinity polypeptide binding to the first subunit polypeptide is regulated by a small molecule and/or the second affinity polypeptide binding to the second subunit polypeptide is regulated by a small molecule.
33. A method of screening transmembrane (TM) receptor complexes comprising:
   (a) providing a cell expressing a first subunit polypeptide of a TM receptor;
   (b) expressing a second subunit polypeptide of a TM receptor in the cell; and
   (c) assaying the cell for a TM receptor phenotype; and
   wherein (i) the presence of the TM receptor phenotype indicates the first subunit polypeptide and the second subunit polypeptide oligomerize to form the TM receptor; or (ii) the absence of the TM receptor phenotype indicates the first subunit polypeptide and the second subunit polypeptide do not oligomerize to form a TM receptor, and wherein (1) the second subunit polypeptide comprises a tag polypeptide, the first subunit polypeptide comprises an affinity polypeptide that binds to the tag polypeptide, and the affinity polypeptide is heterologous to the first subunit polypeptide, or (2) the first subunit polypeptide comprises the tag polypeptide, the second subunit polypeptide comprises the affinity polypeptide that binds to the tag polypeptide, and the affinity polypeptide is heterologous to the second subunit polypeptide.
34. The method of embodiment 33, wherein the TM receptor is a single-pass TM (SPTM) receptor, or wherein the TM receptor is a SPTM comprising an intracellular kinase domain (SPTM-kinase).
35. The method of embodiment 33 or embodiment 34, wherein the binding partner for the first subunit polypeptide is unknown and step (b) is repeated using two or more candidate second subunit polypeptides to identify the second subunit polypeptide that is the binding partner for the first subunit polypeptide.
36. The method of any one of embodiments 33-35, wherein the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, and a ubiquitin tag polypeptide.
37. The method of embodiment 36, wherein the tag polypeptide is a fluorescent protein polypeptide selected from a green fluorescent protein (GFP) polypeptide, a red fluorescent polypeptide (RFP) (e.g., mCherry), and a blue fluorescent polypeptide (BFP); wherein the tag polypeptide is a luminescence polypeptide selected from a luciferase polypeptide; or wherein the tag polypeptide is a combination of a fluorescent protein polypeptide and a luminescence polypeptide.
38. The method of any one of embodiments 33-37, wherein the affinity polypeptide is selected from a heavy-chain variable domain (VHH), a single-chain variable fragment (scFV), a designed ankyrin repeat protein (DARPins), a human fibronectin III domain 3 monobody, an Affibody, and an anticalin, and a synthetic version of any of the foregoing.
39. The method of embodiment 38, wherein the affinity polypeptide is a heavy-chain variable domain (VHH).
40. The method of embodiment 39, wherein the affinity polypeptide is a VHH_{LaG16}.
41. The method of any one of embodiments 33-40, wherein the cell is a eubacterial cell, an archaeal cell, or a eukaryotic cell.
42. The method of embodiment 41, wherein the eukaryotic cell is a plant cell, an animal cell, or a fungal cell.
43. The method of any one of embodiments 33-42, wherein the cell is a plant cell and the first subunit polypeptide and/or the second subunit polypeptide are selected from a LysM receptor, a Leucine rich repeat (LRR) receptor, a Malectin like receptor, a plant TM receptor, a plant SPTM (pSPTM) receptor, and a plant SPTM receptor comprising an intracellular kinase domain (pSPTM-kinase).
44. The method of embodiment 43, wherein the first subunit polypeptide and/or the second subunit polypeptide is a LysM receptor.
45. The method of embodiment 44, wherein the first subunit polypeptide and/or the second subunit polypeptide lacks an ectodomain and/or a transmembrane domain.
46. The method of embodiment 44 or embodiment 45, wherein the first subunit polypeptide is a NFR1 polypeptide and the second subunit polypeptide is a NFR5 polypeptide, wherein the first subunit polypeptide is a NFR5 polypeptide and the second subunit is a NFR1 polypeptide, wherein the first subunit polypeptide is a LYK3 polypeptide and the second subunit polypeptide is a NFP polypeptide, wherein the first subunit polypeptide is a NFP polypeptide and the second subunit polypeptide is a LYK3 polypeptide, wherein the first subunit polypeptide is a RLK4 receptor polypeptide and the second subunit polypeptide is a RLK10 receptor polypeptide, or wherein the first subunit polypeptide is a RLK10 polypeptide and the second subunit polypeptide is a RLK4 polypeptide.
47. The method of embodiment 46, wherein the NFR1, LYK3, or RLK4 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 56, and wherein the NFR5, NFP, or RLK10 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, and SEQ ID NO: 57.
48. The method of any one of embodiments 33-47, wherein the affinity polypeptide binding to the tag polypeptide is regulated by a small molecule.
49. A genetically modified plant or part thereof comprising the genetically modified plant cell of any one of embodiments 1-17.
50. The genetically modified plant or part thereof of embodiment 49, further comprising the TM receptor comprising the first subunit polypeptide and the second subunit polypeptide, wherein the first subunit polypeptide comprises the affinity polypeptide that binds to the second subunit polypeptide intracellularly inducing oligomerization, and wherein oligomerization of the first subunit polypeptide and the second subunit polypeptide activates TM receptor signaling.
51. The genetically modified plant or part thereof of embodiment 50, wherein the affinity polypeptide binds directly to the second subunit polypeptide or wherein the second subunit polypeptide comprises a tag polypeptide, and wherein the affinity polypeptide binds to the tag polypeptide.
52. The genetically modified plant or part thereof of any one of embodiments 49-51, wherein the genetically modified plant cell is selected from a root epidermal cell, a root cortex cell, a root endodermis cell, a root pericycle cell, a root primordia cell, a xylem cell, a phloem cell, a meristem cell, a leaf cell, a stem cell, a flower cell, and a fruit cell.
53. The genetically modified plant or part thereof of embodiment 52, wherein the genetically modified plant cell is a root epidermal cell, a root cortex cell, a root endodermis cell, a root pericycle cell, or a root primordia cell.
54. A genetically modified plant or part thereof comprising the bispecific affinity polypeptide of any one of embodiments 18-32.
55. The genetically modified plant or part thereof of embodiment 54, further comprising the first affinity polypeptide that binds to the intracellular portion of the first subunit polypeptide of the TM receptor and the second affinity polypeptide that binds to the intracellular portion of the second subunit polypeptide of the TM receptor, wherein binding of the first affinity polypeptide to the first subunit polypeptide and of the second affinity polypeptide to the second subunit polypeptide induces oligomerization; and wherein oligomerization of the first subunit polypeptide and the second subunit polypeptide activates TM receptor signaling.
56. The genetically modified plant or part thereof of embodiment 55, wherein the first affinity polypeptide binds directly to the first subunit polypeptide and/or wherein the second affinity polypeptide binds directly to the second subunit polypeptide; or wherein the first subunit polypeptide comprises the tag polypeptide, and wherein the first affinity polypeptide binds to the tag polypeptide, and/or wherein the second subunit polypeptide comprises the tag polypeptide, and wherein the second affinity polypeptide binds to the tag polypeptide.
57. The genetically modified plant or part thereof of any one of embodiments 51-56, wherein the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, and a ubiquitin tag polypeptide.
58. The genetically modified plant or part thereof of embodiment 57, wherein the tag polypeptide is a fluorescent protein polypeptide selected from a green fluorescent protein (GFP) polypeptide, a red fluorescent polypeptide (RFP) (e.g., mCherry), and a blue fluorescent polypeptide (BFP); wherein the tag polypeptide is a luminescence polypeptide selected from a luciferase polypeptide; or wherein the tag polypeptide is a combination of a fluorescent protein polypeptide and a luminescence polypeptide.
59. The genetically modified plant or part thereof of any one of embodiments 51-58, wherein the affinity polypeptide is selected from a heavy-chain variable domain (VHH), a single-chain variable fragment (scFV), a designed ankyrin repeat protein (DARPins), a human fibronectin III domain 3 monobody, an Affibody, and an anticalin, and a synthetic version of any of the foregoing.
60. The genetically modified plant or part thereof of embodiment 59, wherein the affinity polypeptide is a heavy-chain variable domain (VHH).
61. The genetically modified plant or part thereof of embodiment 60, wherein the affinity polypeptide is a VHH_{LaG16}.
62. The genetically modified plant or part thereof of any one of embodiments 51-61, wherein the first subunit polypeptide and/or the second subunit polypeptide are selected from a LysM receptor, a Leucine rich repeat (LRR) receptor, a Malectin like receptor, a plant TM receptor, a plant SPTM (pSPTM) receptor, and a plant SPTM receptor comprising an intracellular kinase domain (pSPTM-kinase).
63. The genetically modified plant or part thereof of embodiment 62, wherein the first subunit polypeptide and/or the second subunit polypeptide is a LysM receptor.
64. The genetically modified plant or part thereof of embodiment 63, wherein the first subunit polypeptide and/or the second subunit polypeptide lacks an ectodomain and/or a transmembrane domain.
65. The genetically modified plant or part thereof of embodiment 63 or embodiment 64, wherein the first subunit polypeptide is a NFR1 polypeptide and the second subunit polypeptide is a NFR5 polypeptide, wherein the first subunit polypeptide is a NFR5 polypeptide and the second subunit is a NFR1 polypeptide, wherein the first subunit polypeptide is a LYK3 polypeptide and the second subunit polypeptide is a NFP polypeptide, wherein the first subunit polypeptide is a NFP polypeptide and the second subunit polypeptide is a LYK3 polypeptide, wherein the first subunit polypeptide is a RLK4 receptor polypeptide and the second subunit polypeptide is a RLK10 receptor polypeptide, or wherein the first subunit polypeptide is a RLK10 polypeptide and the second subunit polypeptide is a RLK4 polypeptide.
66. The genetically modified plant or part thereof of embodiment 65, wherein the NFR1, LYK3, or RLK4 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 56, and wherein the NFR5, NFP, or RLK10 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, and SEQ ID NO: 57.
67. The genetically modified plant or part thereof of any one of embodiments 51-66, wherein the TM receptor is localized to a plant cell membrane.
68. The genetically modified plant or part thereof of any one of embodiments 51-68, wherein the plant part is a leaf, a stem, a root, a root primordia, a flower, a seed, a fruit, a kernel, a grain, a cell, or a portion thereof.
69. The genetically modified plant or part thereof of any one of embodiments 51-68, wherein the plant is selected from the group consisting of cassava, corn, cowpea, rice, barley, wheat, Trema spp., apple, pear, plum, apricot, peach, almond, walnut, strawberry, raspberry, blackberry, red currant, black currant, melon, cucumber, pumpkin, squash, grape, bean, soybean, pea, chickpea, cowpea, pigeon pea, lentil, Bambara groundnut, lupin, pulses, Medicago spp., Lotus spp., forage legumes, indigo, legume trees, and hemp.
70. A method of making the genetically modified plant cell of any one of embodiments 1-17, comprising introducing a genetic alteration to the plant cell comprising a first nucleic acid sequence encoding a heterologous first subunit polypeptide comprising an affinity polypeptide; and/or introducing a genetic alteration to the plant cell comprising a second nucleic acid sequence encoding a heterologous second subunit polypeptide optionally comprising a tag polypeptide.
71. The method of embodiment 70, wherein the first nucleic acid sequence is operably linked to a promoter and/or wherein the second nucleic acid sequence is operably linked to a promoter.
72. The method of embodiment 71, wherein the promoter is a root specific promoter, an inducible promoter, a constitutive promoter, or a combination thereof.
73. The method of embodiment 71 or embodiment 72, wherein the promoter is a root specific promoter, and wherein the promoter is selected from the group consisting of a NFR1 promoter, a NFR5 promoter, a LYK3 promoter, a CERK6 promoter, a NFP promoter, a Lotus japonicus NFR5 promoter (SEQ ID NO: 27), a Lotus japonicus NFR1 promoter (SEQ ID NO: 69), a Lotus japonicus CERK6 promoter (SEQ ID NO: 46), a Medicago truncatula NFP promoter (SEQ ID NO: 29), a Medicago truncatula LYK3 promoter (SEQ ID NO: 28), a maize metallothionein promoter, a chitinase promoter, a maize ZRP2 promoter, a tomato LeExtl promoter, a glutamine synthetase soybean root promoter, a RCC3 promoter, a rice antiquitin promoter, a LRR receptor kinase promoter, and an Arabidopsis pCO2 promoter.
74. The method of embodiment 71 or embodiment 72, wherein the promoter is a constitutive promoter, and wherein the promoter is selected from the group consisting of a CaMV35S promoter, a derivative of the CaMV35S promoter, a maize ubiquitin promoter, a polyubiquitin promoter, a vein mosaic cassava virus promoter, and an Arabidopsis UBQ10 promoter.
75. The method of embodiment 70, wherein the first nucleic acid sequence is inserted into the genome of the plant so that the nucleic acid sequence is operably linked to an endogenous promoter, and/or wherein the second nucleic acid sequence is inserted into the genome of the plant so that the nucleic acid sequence is operably linked to an endogenous promoter.
76. The method of embodiment 75, wherein the endogenous promoter is a root specific promoter.
77. A method of making the genetically modified plant cell of any one of embodiments 1-17, comprising genetically modifying the plant cell by transforming the plant cell with one or more gene editing components that target a first endogenous nuclear genome sequence encoding the first subunit polypeptide, wherein the first subunit polypeptide is genetically modified to comprise the affinity polypeptide; and/or genetically modifying the plant cell by transforming the plant cell with one or more gene editing components that target a second endogenous nuclear genome sequence encoding the second subunit polypeptide, wherein the endogenous second subunit polypeptide is genetically modified to comprise a tag polypeptide.
78. The method of embodiment 77, wherein the one or more gene editing components comprise a ribonucleoprotein complex that targets the first and/or second nuclear genome sequence; a vector comprising a TALEN protein encoding sequence, wherein the TALEN protein targets the first and/or second nuclear genome sequence; a vector comprising a ZFN protein encoding sequence, wherein the ZFN protein targets the first and/or second nuclear genome sequence; an oligonucleotide donor (OND), wherein the OND targets the first and/or second nuclear genome sequence; or a vector CRISPR/Cas enzyme encoding sequence and a targeting sequence, wherein the targeting sequence targets the first and/or second nuclear genome sequence.
79. The method of embodiment 77 or embodiment 78, wherein genetically modifying the first subunit to comprise the affinity polypeptide comprises inserting a first nucleic acid sequence encoding a heterologous first subunit polypeptide comprising an affinity polypeptide into the first endogenous nuclear genome sequence; and wherein genetically modifying the second subunit to comprise the tag polypeptide comprises inserting a second nucleic acid sequence encoding a heterologous second subunit polypeptide comprising a tag polypeptide into the second endogenous nuclear genome sequence.
80. A method of making the genetically modified plant or part thereof of any one of embodiments 49-53, comprising introducing a genetic alteration to the plant cell comprising a first nucleic acid sequence encoding a heterologous first subunit polypeptide comprising an affinity polypeptide; and/or introducing a genetic alteration to the plant cell comprising a second nucleic acid sequence encoding a heterologous second subunit polypeptide comprising a tag polypeptide.
81. The method of embodiment 80, wherein the first nucleic acid sequence is operably linked to a promoter and/or wherein the second nucleic acid sequence is operably linked to a promoter.
82. The method of embodiment 81, wherein the promoter is a root specific promoter, a constitutive promoter, or a combination thereof.
83. The method of embodiment 81 or embodiment 82, wherein the promoter is a root specific promoter, and wherein the promoter is selected from the group consisting of a NFR1 promoter, a NFR5 promoter, a LYK3 promoter, a CERK6 promoter, a NFP promoter, a Lotus japonicus NFR5 promoter (SEQ ID NO: 27), a Lotus japonicus NFR1 promoter (SEQ ID NO: 69), a Lotus japonicus CERK6 promoter (SEQ ID NO: 46), a Medicago truncatula NFP promoter (SEQ ID NO: 29), a Medicago truncatula LYK3 promoter (SEQ ID NO: 28), a maize metallothionein promoter, a chitinase promoter, a maize ZRP2 promoter, a tomato LeExtl promoter, a glutamine synthetase soybean root promoter, a RCC3 promoter, a rice antiquitin promoter, a LRR receptor kinase promoter, and an Arabidopsis pCO2 promoter.
84. The method of embodiment 81 or embodiment 82, wherein the promoter is a constitutive promoter, and wherein the promoter is selected from the group consisting of a CaMV35S promoter, a derivative of the CaMV35S promoter, a maize ubiquitin promoter, a polyubiquitin promoter, a vein mosaic cassava virus promoter, and an Arabidopsis UBQ10 promoter.
85. The method of embodiment 80, wherein the first nucleic acid sequence is inserted into the genome of the plant so that the nucleic acid sequence is operably linked to an endogenous promoter, and/or wherein the second nucleic acid sequence is inserted into the genome of the plant so that the nucleic acid sequence is operably linked to an endogenous promoter.
86. The method of embodiment 85, wherein the endogenous promoter is a root specific promoter.
87. A method of making the genetically modified plant or part thereof of any one of embodiments 49-53, comprising genetically modifying the plant cell by transforming the plant cell with one or more gene editing components that target a first nuclear genome sequence encoding an endogenous first subunit polypeptide, wherein the endogenous first subunit polypeptide is genetically modified to comprise an affinity polypeptide; and/or genetically modifying the plant cell by transforming the plant cell with one or more gene editing components that target a second nuclear genome sequence encoding an endogenous second subunit polypeptide to comprise a tag polypeptide, wherein the endogenous second subunit polypeptide is genetically modified to comprise an affinity polypeptide.
88. The method of embodiment 87, wherein the one or more gene editing components comprise a ribonucleoprotein complex that targets the first and/or second nuclear genome sequence; a vector comprising a TALEN protein encoding sequence, wherein the TALEN protein targets the first and/or second nuclear genome sequence; a vector comprising a ZFN protein encoding sequence, wherein the ZFN protein targets the first and/or second nuclear genome sequence; an oligonucleotide donor (OND), wherein the OND targets the first and/or second nuclear genome sequence; or a vector CRISPR/Cas enzyme encoding sequence and a targeting sequence, wherein the targeting sequence targets the first and/or second nuclear genome sequence.
89. An expression vector or isolated DNA molecule comprising one or more nucleotide sequences encoding a first subunit polypeptide of a TM receptor comprising an affinity polypeptide, wherein the affinity polypeptide is heterologous to the first subunit polypeptide, and wherein the one or more nucleotide sequences are operably linked to at least one expression control sequence.
90. An expression vector or isolated DNA molecule comprising one or more nucleotide sequences encoding a second subunit polypeptide of a TM receptor optionally comprising a tag polypeptide, wherein the one or more nucleotide sequences are operably linked to at least one expression control sequence.
91. An expression vector or isolated DNA molecule comprising one or more nucleotide sequences encoding:
   (a) a first subunit polypeptide of a transmembrane (TM) receptor complex comprising an affinity polypeptide, wherein the affinity polypeptide is heterologous to the first subunit polypeptide; and/or
   (b) a second subunit polypeptide of a TM receptor optionally comprising a tag polypeptide, wherein the one or more nucleotide sequences are operably linked to at least one expression control sequence.
92. An expression vector or isolated DNA molecule comprising one or more nucleotide sequences encoding a bispecific affinity polypeptide comprising a first affinity polypeptide that binds to an intracellular portion of a first subunit polypeptide of a TM receptor and a second affinity polypeptide that binds to an intracellular portion of a second subunit polypeptide of a TM receptor, wherein the one or more nucleotide sequences are operably linked to at least one expression control sequence.
93. The expression vector or isolated DNA molecule of embodiment 92, wherein the first affinity polypeptide binds directly to the first subunit polypeptide and/or wherein the second affinity polypeptide binds directly to the second subunit polypeptide.
94. The expression vector or isolated DNA molecule of embodiment 92 or embodiment 93, wherein the first subunit polypeptide comprises a tag polypeptide, and wherein the first affinity polypeptide binds to the tag polypeptide, and/or wherein the second polypeptide comprises a tag polypeptide, and wherein the second affinity polypeptide binds to the tag polypeptide.
95. The expression vector or isolated DNA molecule of any one of embodiments 90, 91, and 94, wherein the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, and a ubiquitin tag polypeptide.
96. The expression vector or isolated DNA molecule of embodiment 95, wherein the tag polypeptide is a fluorescent protein polypeptide selected from a green fluorescent protein (GFP) polypeptide, a red fluorescent polypeptide (RFP) (e.g., mCherry), and a blue fluorescent polypeptide (BFP); wherein the tag polypeptide is a luminescence polypeptide selected from a luciferase polypeptide; or wherein the tag polypeptide is a combination of a fluorescent protein polypeptide and a luminescence polypeptide.
97. The expression vector or isolated DNA molecule of any one of embodiments 89 and 91-96, wherein the first affinity polypeptide and the second affinity polypeptide are selected from a heavy-chain variable domain (VHH), a single-chain variable fragment (scFV), a designed ankyrin repeat protein (DARPins), a human fibronectin III domain 3 monobody, an Affibody, and an anticalin, and a synthetic version of any of the foregoing.
98. The expression vector or isolated DNA molecule of embodiment 97, wherein the first affinity polypeptide and/or the second affinity polypeptide is a heavy-chain variable domain (VHH).
99. The expression vector or isolated DNA molecule of embodiment 98, wherein the affinity polypeptide is a VHH_{LaG16}.
100. The expression vector or isolated DNA molecule of any one of embodiments 89-99, wherein the first subunit polypeptide and/or the second subunit polypeptide are selected from a LysM receptor, a Leucine rich repeat (LRR) receptor, a Malectin like receptor, a single-pass TM (SPTM) receptor, a plant TM receptor, a plant SPTM (pSPTM) receptor, a SPTM receptor comprising an intracellular kinase domain (SPTM-kinase), and a plant SPTM receptor comprising an intracellular kinase domain (pSPTM-kinase).
101. The expression vector or isolated DNA molecule of embodiment 100, wherein the first subunit polypeptide and/or the second subunit polypeptide is a LysM receptor.
102. The expression vector or isolated DNA molecule of embodiment 101, wherein the first subunit polypeptide and/or the second subunit polypeptide lacks an ectodomain and/or a transmembrane domain.
103. The expression vector or isolated DNA molecule of embodiment 101 or embodiment 102, wherein the first subunit polypeptide is a NFR1 polypeptide and the second subunit polypeptide is a NFR5 polypeptide, wherein the first subunit polypeptide is a NFR5 polypeptide and the second subunit is a NFR1 polypeptide, wherein the first subunit polypeptide is a LYK3 polypeptide and the second subunit polypeptide is a NFP polypeptide, wherein the first subunit polypeptide is a NFP polypeptide and the second subunit polypeptide is a LYK3 polypeptide, wherein the first subunit polypeptide is a RLK4 receptor polypeptide and the second subunit polypeptide is a RLK10 receptor polypeptide, or wherein the first subunit polypeptide is a RLK10 polypeptide and the second subunit polypeptide is a RLK4 polypeptide.
104. The genetically modified cell of embodiment 103, wherein the NFR1, LYK3, or RLK4 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 56, and wherein the NFR5, NFP, or RLK10 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, and SEQ ID NO: 57.
105. The expression vector or isolated DNA molecule of any one of embodiments 89-104, wherein the at least one expression control sequence comprises a promoter selected from the group consisting of a root specific promoter, a constitutive promoter, and a combination thereof.
106. The expression vector or isolated DNA molecule of embodiment 105, wherein the promoter is a root specific promoter, and wherein the promoter is selected from the group consisting of a NFR1 promoter, a NFR5 promoter, a LYK3 promoter, a CERK6 promoter, a NFP promoter, a Lotus japonicus NFR5 promoter (SEQ ID NO: 27), a Lotus japonicus NFR1 promoter (SEQ ID NO: 69), a Lotus japonicus CERK6 promoter (SEQ ID NO: 46), a Medicago truncatula NFP promoter (SEQ ID NO: 29), a Medicago truncatula LYK3 promoter (SEQ ID NO: 28), a maize metallothionein promoter, a chitinase promoter, a maize ZRP2 promoter, a tomato LeExtl promoter, a glutamine synthetase soybean root promoter, a RCC3 promoter, a rice antiquitin promoter, a LRR receptor kinase promoter, and an Arabidopsis pCO2 promoter.
107. The expression vector or isolated DNA molecule of embodiment 105, wherein the promoter is a constitutive promoter, and wherein the promoter is selected from the group consisting of a CaMV35S promoter, a derivative of the CaMV35S promoter, a maize ubiquitin promoter, a polyubiquitin promoter, a vein mosaic cassava virus promoter, and an Arabidopsis UBQ10 promoter.
108. A bacterial cell or an Agrobacterium cell comprising the expression vector or isolated DNA molecule of any one of embodiments 89-107.
109. A genetically modified plant, plant part, plant cell, or seed comprising the expression vector or isolated DNA molecule of any one of embodiments 89-107.
110. A kit comprising the expression vector or isolated DNA molecule of any one of embodiments 89-107 or the bacterial cell or the Agrobacterium cell of embodiment 108.
111. A method of activating a target transmembrane (TM) receptor complex or inducing organogenesis comprising: introducing a genetic alteration via an expression vector or isolated DNA molecule of any one of embodiments 89-107 to a cell.
112. The method of embodiment 111, wherein activating the target TM receptor complex or inducing organogenesis is in the absence of a native, an endogenous, or exogenous stimulus (e.g., Nod).
113. The method of embodiment 111 or embodiment 112, further comprising knocking out a native target TM receptor complex or subunits thereof in the cell.
114. The method of any one of embodiments 111-113, wherein the cell is a plant cell.
115. A genetically modified plant cell comprising:
   an NFR1-NFR5 receptor complex comprising a first subunit polypeptide and a second subunit polypeptide,
   wherein the first subunit polypeptide comprises an affinity polypeptide that binds to the second subunit polypeptide inducing oligomerization, and wherein the affinity polypeptide is heterologous to the first subunit polypeptide; and
   wherein oligomerization of the first subunit polypeptide and the second subunit polypeptide activates NFR1-NFR5 receptor complex signaling and wherein (i) the first subunit polypeptide is an NFR1 polypeptide and the second subunit polypeptide is an NFR5 polypeptide, or (ii) the first subunit polypeptide is the NFR5 polypeptide and the second subunit polypeptide is the NFR1 polypeptide.
116. The genetically modified plant cell of embodiment 115, wherein the NFR1 polypeptide and/or the NFR5 polypeptide lacks an ectodomain and/or a transmembrane domain.
117. The genetically modified plant cell of embodiment 115 or embodiment 116, wherein the NFR1 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 56, and wherein the NFR5 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, and SEQ ID NO: 57.
118. The genetically modified plant cell of any one of embodiments 115-117, wherein the affinity polypeptide binds directly to an intracellular portion of the second subunit polypeptide.
119. The genetically modified plant cell of any one of embodiments 115-117, wherein the second subunit polypeptide comprises a tag polypeptide fused to an intracellular portion of the second subunit polypeptide, and wherein the affinity polypeptide binds to the tag polypeptide.
120. The genetically modified plant cell of embodiment 119, wherein the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, and a ubiquitin tag polypeptide.
121. The genetically modified plant cell of embodiment 120, wherein the tag polypeptide is a fluorescent protein polypeptide selected from a green fluorescent protein (GFP) polypeptide, a red fluorescent polypeptide (RFP) (e.g., mCherry), and a blue fluorescent polypeptide (BFP); wherein the tag polypeptide is a luminescence polypeptide selected from a luciferase polypeptide; or wherein the tag polypeptide is a combination of a fluorescent protein polypeptide and a luminescence polypeptide.
122. The genetically modified plant cell of any one of embodiments 115-121, wherein the affinity polypeptide is selected from a heavy-chain variable domain (VHH), a single-chain variable fragment (scFV), a designed ankyrin repeat protein (DARPins), a human fibronectin III domain 3 monobody, an Affibody, and an anticalin, and a synthetic version of any of the foregoing.
123. The genetically modified plant cell of embodiment 122, wherein the affinity polypeptide is a heavy-chain variable domain (VHH).
124. The genetically modified plant cell of embodiment 123, wherein the affinity polypeptide is VHH_{LaG16}.
125. The genetically modified plant cell of any one of embodiments 115-124, wherein the affinity polypeptide binding to the second subunit polypeptide is regulated by a small molecule.
126. A bispecific affinity polypeptide for activation of an NFR1-NFR5 receptor complex comprising:
   a first affinity polypeptide that binds to an intracellular portion of an NFR1 polypeptide; and
   a second affinity polypeptide that binds to an intracellular portion of an NFR5 polypeptide;
   wherein binding of the first affinity polypeptide to the NFR1 polypeptide and of the second affinity to the NFR5 polypeptide induces dimerization; and wherein dimerization of the NFR1 polypeptide and the NFR5 polypeptide activates NFR1-NFR5 receptor complex signaling.
127. The bispecific affinity polypeptide of embodiment 126, wherein the NFR1 polypeptide and/or the NFR5 polypeptide lacks an ectodomain and/or a transmembrane domain.
128. The bispecific affinity polypeptide of embodiment 126 or embodiment 127, wherein the NFR1 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 56, and wherein the NFR5 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, and SEQ ID NO: 57.
129. The bispecific affinity polypeptide of any one of embodiments 127-128, wherein the first affinity polypeptide binds directly to the NFR1 polypeptide and/or wherein the second affinity polypeptide binds directly to the NFR5 polypeptide.
130. The bispecific affinity polypeptide of any one of embodiments 127-128, wherein the NFR1 polypeptide comprises a tag polypeptide fused to an intracellular portion of the NFR1 polypeptide, and wherein the first affinity polypeptide binds to the tag polypeptide; and/or wherein the NFR5 polypeptide comprises a tag polypeptide fused to an intracellular portion of the NFR5 polypeptide, and wherein the second affinity polypeptide binds to the tag polypeptide.
131. The bispecific affinity polypeptide of embodiment 130, wherein the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a luciferase tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, and a ubiquitin tag polypeptide.
132. The bispecific affinity polypeptide of embodiment 131, wherein the tag polypeptide is a fluorescent protein polypeptide selected from a green fluorescent protein (GFP) polypeptide, a red fluorescent polypeptide (RFP) (e.g., mCherry), and a blue fluorescent polypeptide (BFP); wherein the tag polypeptide is a luminescence polypeptide selected from a luciferase polypeptide; or wherein the tag polypeptide is a combination of a fluorescent protein polypeptide and a luminescence polypeptide.
133. The bispecific affinity polypeptide of any one of embodiments 126-132, wherein the first affinity polypeptide and the second affinity polypeptide are selected from a heavy-chain variable domain (VHH), a single-chain variable fragment (scFV), a designed ankyrin repeat protein (DARPins), a human fibronectin III domain 3 monobody, an Affibody, and an anticalin, and a synthetic version of any of the foregoing.
134. The bispecific affinity polypeptide of embodiment 133, wherein the first affinity polypeptide and/or the second affinity polypeptide is a heavy-chain variable domain (VHH).
135. The bispecific affinity polypeptide of embodiment 134, wherein the affinity polypeptide is VHH_{LaG16}.
136. The bispecific affinity polypeptide of any one of embodiments 127-135, wherein the first affinity polypeptide binding to the NFR1 polypeptide is regulated by a small molecule and/or the second affinity polypeptide binding to the NFR5 polypeptide is regulated by a small molecule.
137. A genetically modified plant or part thereof comprising the genetically modified plant cell of any one of embodiments 115-125.
138. The genetically modified plant or part thereof of embodiment 137, further comprising the NFR1-NFR5 receptor complex comprising the first subunit polypeptide and the second subunit polypeptide, wherein the first subunit polypeptide comprises an affinity polypeptide that binds to the second subunit polypeptide inducing oligomerization; and wherein oligomerization of the first subunit polypeptide and the second subunit polypeptide activates NFR1-NFR5 receptor complex signaling.
139. The genetically modified plant or part thereof of embodiment 138, wherein the affinity polypeptide binds directly to the second subunit polypeptide or wherein the second subunit polypeptide comprises a tag polypeptide fused to an intracellular portion of the second subunit polypeptide, and wherein the affinity polypeptide binds to the tag polypeptide.
140. The genetically modified plant or part thereof of any one of embodiments 137-139, wherein the genetically modified plant cell is selected from a root epidermal cell, a root cortex cell, a root endodermis cell, a root pericycle cell, a root primordia cell, a xylem cell, a phloem cell, a meristem cell, a leaf cell, a stem cell, a flower cell, and a fruit cell.
141. The genetically modified plant or part thereof of embodiment 140, wherein the genetically modified plant cell is a root epidermal cell, a root cortex cell, a root endodermis cell, a root pericycle cell, or a root primordia cell.
142. A genetically modified plant or part thereof comprising the bispecific affinity polypeptide of any one of embodiments 126-136.
143. The genetically modified plant or part thereof of embodiment 142, further comprising the first affinity polypeptide that binds to an intracellular portion of the NFR1 polypeptide and the second affinity polypeptide that binds to the intracellular portion of the NFR5 polypeptide, wherein binding of the first affinity polypeptide to the NFR1 polypeptide and of the second affinity polypeptide to the NFR5 polypeptide induces oligomerization; and wherein oligomerization of the NFR1 polypeptide and the NFR5 polypeptide activates NFR1-NFR5 receptor complex signaling.
144. The genetically modified plant or part thereof of embodiment 143, wherein the first affinity polypeptide binds directly to the NFR1 polypeptide and/or wherein the second affinity polypeptide binds directly to the NFR5 polypeptide; or wherein the NFR1 polypeptide comprises a tag polypeptide fused to the intracellular portion of the NFR1 polypeptide, and wherein the first affinity polypeptide binds to the tag polypeptide, and/or wherein the NFR5 polypeptide comprises a tag polypeptide fused to the intracellular portion of the NFR5 polypeptide, and wherein the second affinity polypeptide binds to the tag polypeptide.
145. The genetically modified plant or part thereof of any one of embodiments 137-144, wherein the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, and a ubiquitin tag polypeptide.
146. The genetically modified plant or part thereof of embodiment 145, wherein the tag polypeptide is a fluorescent protein polypeptide selected from a green fluorescent protein (GFP) polypeptide, a red fluorescent polypeptide (RFP) (e.g., mCherry), and a blue fluorescent polypeptide (BFP); wherein the tag polypeptide is a luminescence polypeptide selected from a luciferase polypeptide; or wherein the tag polypeptide is a combination of a fluorescent protein polypeptide and a luminescence polypeptide.
147. The genetically modified plant or part thereof of any one of embodiments 137-146, wherein the affinity polypeptide is selected from a heavy-chain variable domain (VHH), a single-chain variable fragment (scFV), a designed ankyrin repeat protein (DARPins), a human fibronectin III domain 3 monobody, an Affibody, and an anticalin, and a synthetic version of any of the foregoing.
148. The genetically modified plant or part thereof of embodiment 147, wherein the affinity polypeptide is a heavy-chain variable domain (VHH).
149. The genetically modified plant or part thereof of embodiment 148, wherein the affinity polypeptide is a VHH_{LaG16}.
150. The genetically modified plant or part thereof of any one of embodiments 137-149, wherein the NFR1-NFR5 receptor complex is localized to a plant cell membrane.
151. The genetically modified plant or part thereof of any one of embodiments 137-150, wherein the plant part is a leaf, a stem, a root, a root primordia, a flower, a seed, a fruit, a kernel, a grain, a cell, or a portion thereof.
152. The genetically modified plant or part thereof of any one of embodiments 137-151, wherein the plant is selected from the group consisting of cassava, corn, cowpea, rice, barley, wheat, Trema spp., apple, pear, plum, apricot, peach, almond, walnut, strawberry, raspberry, blackberry, red currant, black currant, melon, cucumber, pumpkin, squash, grape, bean, soybean, pea, chickpea, cowpea, pigeon pea, lentil, Bambara groundnut, lupin, pulses, Medicago spp., Lotus spp., forage legumes, indigo, legume trees, and hemp.
153. A method of making the genetically modified plant cell of any one of embodiments 115-126, comprising introducing a genetic alteration to the plant cell comprising a first nucleic acid sequence encoding a heterologous NFR1 polypeptide comprising an affinity polypeptide; and/or introducing a genetic alteration to the plant cell comprising a second nucleic acid sequence encoding a heterologous NFR5 polypeptide optionally comprising a tag polypeptide.
154. The method of embodiment 153, wherein the NFR1 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 56, and wherein the NFR5 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, and SEQ ID NO: 57.
155. The method of embodiment 153 or embodiment 154, wherein the first nucleic acid sequence is operably linked to a promoter and/or wherein the second nucleic acid sequence is operably linked to a promoter.
156. The method of embodiment 155, wherein the promoter is a root specific promoter, a constitutive promoter, or a combination thereof.
157. The method of embodiment 156, wherein the promoter is a root specific promoter, and wherein the promoter is selected from the group consisting of a NFR1 promoter, a NFR5 promoter, a LYK3 promoter, a CERK6 promoter, a NFP promoter, a Lotus japonicus NFR5 promoter (SEQ ID NO: 27), a Lotus japonicus NFR1 promoter (SEQ ID NO: 69), a Lotus japonicus CERK6 promoter (SEQ ID NO: 46), a Medicago truncatula NFP promoter (SEQ ID NO: 29), a Medicago truncatula LYK3 promoter (SEQ ID NO: 28), a maize metallothionein promoter, a chitinase promoter, a maize ZRP2 promoter, a tomato LeExtl promoter, a glutamine synthetase soybean root promoter, a RCC3 promoter, a rice antiquitin promoter, a LRR receptor kinase promoter, and an Arabidopsis pCO2 promoter.
158. The method of embodiment 156, wherein the promoter is a constitutive promoter, and wherein the promoter is selected from the group consisting of a CaMV35S promoter, a derivative of the CaMV35S promoter, a maize ubiquitin promoter, a polyubiquitin promoter, a vein mosaic cassava virus promoter, and an Arabidopsis UBQ10 promoter.
159. The method of embodiment 153 or embodiment 154, wherein the first nucleic acid sequence is inserted into the genome of the plant so that the nucleic acid sequence is operably linked to an endogenous promoter, and/or wherein the second nucleic acid sequence is inserted into the genome of the plant so that the nucleic acid sequence is operably linked to an endogenous promoter.
160. The method of embodiment 159, wherein the endogenous promoter is a root specific promoter.
161. A method of making the genetically modified plant cell of any one of embodiments 115-125, comprising genetically modifying the plant cell by transforming the plant cell with one or more gene editing components that target a first nuclear genome sequence encoding an endogenous NFR1 polypeptide, wherein the endogenous NFR1 polypeptide is genetically modified to comprise an affinity polypeptide; and/or genetically modifying the plant cell by transforming the plant cell with one or more gene editing components that target a second nuclear genome sequence encoding an endogenous NFR5 polypeptide to comprise a tag polypeptide, wherein the endogenous NFR5 polypeptide is genetically modified to comprise a tag polypeptide.
162. The method of embodiment 161, wherein the NFR1 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 56, and wherein the NFR5 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, and SEQ ID NO: 57.
163. The method of embodiment 161 or embodiment 162, wherein the one or more gene editing components comprise a ribonucleoprotein complex that targets the first and/or second nuclear genome sequence; a vector comprising a TALEN protein encoding sequence, wherein the TALEN protein targets the first and/or second nuclear genome sequence; a vector comprising a ZFN protein encoding sequence, wherein the ZFN protein targets the first and/or second nuclear genome sequence; an oligonucleotide donor (OND), wherein the OND targets the first and/or second nuclear genome sequence; or a vector CRISPR/Cas enzyme encoding sequence and a targeting sequence, wherein the targeting sequence targets the first and/or second nuclear genome sequence.
164. The method of any one of embodiments 161-163, wherein genetically modifying the endogenous NFR1 polypeptide to comprise the affinity polypeptide comprises inserting a first nucleic acid sequence encoding a heterologous NFR1 polypeptide comprising an affinity polypeptide into the endogenous NFR1 nuclear genome sequence; and wherein genetically modifying the endogenous NFR5 polypeptide to comprise the tag polypeptide comprises inserting a second nucleic acid sequence encoding a heterologous NFR5 polypeptide comprising a tag polypeptide into the endogenous NFR5 nuclear genome sequence.
165. A method of making the genetically modified plant or part thereof of any one of embodiments 137-152, comprising introducing a genetic alteration to the plant cell comprising a first nucleic acid sequence encoding a heterologous NFR1 polypeptide comprising an affinity polypeptide; and/or introducing a genetic alteration to the plant cell comprising a second nucleic acid sequence encoding a heterologous NFR5 polypeptide optionally comprising a tag polypeptide.
166. The method of embodiment 165, wherein the NFR1 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 56, and wherein the NFR5 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, and SEQ ID NO: 57.
167. The method of embodiment 165 or embodiment 166, wherein the first nucleic acid sequence is operably linked to a promoter and/or wherein the second nucleic acid sequence is operably linked to a promoter.
168. The method of embodiment 167, wherein the promoter is a root specific promoter, a constitutive promoter, or a combination thereof.
169. The method of embodiment 168, wherein the promoter is a root specific promoter, and wherein the promoter is selected from the group consisting of a NFR1 promoter, a NFR5 promoter, a LYK3 promoter, a CERK6 promoter, a NFP promoter, a Lotus japonicus NFR5 promoter (SEQ ID NO: 27), a Lotus japonicus NFR1 promoter (SEQ ID NO: 69), a Lotus japonicus CERK6 promoter (SEQ ID NO: 46), a Medicago truncatula NFP promoter (SEQ ID NO: 29), a Medicago truncatula LYK3 promoter (SEQ ID NO: 28), a maize metallothionein promoter, a chitinase promoter, a maize ZRP2 promoter, a tomato LeExtl promoter, a glutamine synthetase soybean root promoter, a RCC3 promoter, a rice antiquitin promoter, a LRR receptor kinase promoter, and an Arabidopsis pCO2 promoter.
170. The method of embodiment 168, wherein the promoter is a constitutive promoter, and wherein the promoter is selected from the group consisting of a CaMV35S promoter, a derivative of the CaMV35S promoter, a maize ubiquitin promoter, a polyubiquitin promoter, a vein mosaic cassava virus promoter, and an Arabidopsis UBQ10 promoter.
171. The method of embodiment 165 or embodiment 166, wherein the first nucleic acid sequence is inserted into the genome of the plant so that the nucleic acid sequence is operably linked to an endogenous promoter, and/or wherein the second nucleic acid sequence is inserted into the genome of the plant so that the nucleic acid sequence is operably linked to an endogenous promoter.
172. The method of embodiment 171, wherein the endogenous promoter is a root specific promoter.
173. An expression vector or isolated DNA molecule comprising one or more nucleotide sequences encoding a NFR1 polypeptide comprising an affinity polypeptide, wherein the affinity polypeptide is heterologous to the NFR1 polypeptide, and wherein the one or more nucleotide sequences are operably linked to at least one expression control sequence.
174. An expression vector or isolated DNA molecule comprising one or more nucleotide sequences encoding a NFR5 polypeptide optionally comprising a tag polypeptide, wherein the one or more nucleotide sequences are operably linked to at least one expression control sequence.
175. An expression vector or isolated DNA molecule comprising one or more nucleotide sequences encoding:
   (a) a NFR1 polypeptide comprising an affinity polypeptide, wherein the affinity polypeptide is heterologous to the first subunit polypeptide; and/or
   (b) a NFR5 polypeptide optionally comprising a tag polypeptide,
      wherein the one or more nucleotide sequences are operably linked to at least one expression control sequence.
176. An expression vector or isolated DNA molecule comprising one or more nucleotide sequences encoding a bispecific affinity polypeptide comprising a first affinity polypeptide that binds to an intracellular portion of a NFR1 polypeptide and a second affinity polypeptide that binds to an intracellular portion of a NFR5 polypeptide, wherein the one or more nucleotide sequences are operably linked to at least one expression control sequence.
177. The expression vector or isolated DNA molecule of embodiment 176, wherein the first affinity polypeptide binds directly to the NFR1 polypeptide and/or wherein the second subunit polypeptide binds directly to the NFR5 polypeptide.
178. The expression vector or isolated DNA molecule of embodiment 176 or embodiment 177, wherein the NFR1 polypeptide comprises a tag polypeptide, and wherein the first affinity polypeptide binds to the tag polypeptide and/or wherein the wherein the NFR5 polypeptide comprises a tag polypeptide, and wherein second affinity polypeptide binds to the tag polypeptide.
179. The expression vector or isolated DNA molecule of any one of embodiments 173-178, wherein the NFR1 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 56, and wherein the NFR5 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, and SEQ ID NO: 57.
180. The expression vector or isolated DNA molecule of any one of embodiments 174, 175, and 178, wherein the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a luciferase tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, and a ubiquitin tag polypeptide.
181. The expression vector or isolated DNA molecule of embodiment 180, wherein the tag polypeptide is a fluorescent protein polypeptide selected from a green fluorescent protein (GFP) polypeptide, a red fluorescent polypeptide (RFP) (e.g., mCherry), and a blue fluorescent polypeptide (BFP); wherein the tag polypeptide is a luminescence polypeptide selected from a luciferase polypeptide; or wherein the tag polypeptide is a combination of a fluorescent protein polypeptide and a luminescence polypeptide.
182. The expression vector or isolated DNA molecule of any one of embodiments 173 and 175-178, wherein the first affinity polypeptide and the second affinity polypeptide are selected from a heavy-chain variable domain (VHH), a single-chain variable fragment (scFV), a designed ankyrin repeat protein (DARPins), a human fibronectin III domain 3 monobody, an Affibody, and an anticalin, and a synthetic version of any of the foregoing.
183. The expression vector or isolated DNA molecule of embodiment 182, wherein the first affinity polypeptide and/or the second affinity polypeptide is a heavy-chain variable domain (VHH).
184. The expression vector or isolated DNA molecule of embodiment 183, wherein the affinity polypeptide is VHH_{LaG16}.
185. The expression vector or isolated DNA molecule of any one of embodiments 173-184, wherein the NFR1 polypeptide and/or the NFR5 polypeptide lacks an ectodomain and/or a transmembrane domain.
186. The expression vector or isolated DNA molecule of any one of embodiments 173-185, wherein the at least one expression control sequence comprises a promoter selected from the group consisting of a root specific promoter, a constitutive promoter, and a combination thereof.
187. The expression vector or isolated DNA molecule of embodiment 186, wherein the promoter is a root specific promoter, and wherein the promoter is selected from the group consisting of a NFR1 promoter, a NFR5 promoter, a LYK3 promoter, a CERK6 promoter, a NFP promoter, a Lotus japonicus NFR5 promoter (SEQ ID NO: 27), a Lotus japonicus NFR1 promoter (SEQ ID NO: 69), a Lotus japonicus CERK6 promoter (SEQ ID NO: 46), a Medicago truncatula NFP promoter (SEQ ID NO: 29), a Medicago truncatula LYK3 promoter (SEQ ID NO: 28), a maize metallothionein promoter, a chitinase promoter, a maize ZRP2 promoter, a tomato LeExtl promoter, a glutamine synthetase soybean root promoter, a RCC3 promoter, a rice antiquitin promoter, a LRR receptor kinase promoter, and an Arabidopsis pCO2 promoter.
188. The expression vector or isolated DNA molecule of embodiment 187, wherein the promoter is a constitutive promoter, and wherein the promoter is selected from the group consisting of a CaMV35S promoter, a derivative of the CaMV35S promoter, a maize ubiquitin promoter, a polyubiquitin promoter, a vein mosaic cassava virus promoter, and an Arabidopsis UBQ10 promoter.
189. A bacterial cell or an Agrobacterium cell comprising the expression vector or isolated DNA molecule of any one of embodiments 173-188.
190. A genetically modified plant, plant part, plant cell, or seed comprising the expression vector or isolated DNA molecule of any one of embodiments 173-188.
191. A kit comprising the expression vector or isolated DNA molecule of any one of embodiments 173-188 or the bacterial cell or the Agrobacterium cell of embodiment 189.
192. A method of activating a target NFR1-NFR5 receptor complex or inducing organogenesis comprising: introducing a genetic alteration via an expression vector or isolated DNA molecule of any one of embodiments 173-188 to a cell.
193. The method of embodiment 192, wherein activating the target NFR-NFR5 receptor complex or inducing organogenesis is in the absence of a native, an endogenous, or exogenous stimulus (e.g., Nod).
194. The method of embodiment 192 or embodiment 193, further comprising knocking out the native target NFR1-NFR5 receptor complex or subunits thereof in the cell.
195. The method of any one of embodiments 192-194, wherein the cell is a plant cell.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1J show that VHH-driven assembly defines the core receptor complex for nodule organogenesis. FIG. 1A shows chromatogram profiles for the final size exclusion chromatography (SEC) purification step for NFR5-kinase (residues 276-563, HiLoad Superdex 75 16/600 pg) expressed in E. coli (left panel). The elution volume (Ve) fits the theoretical monomeric molecular weight (NFR5-kinase=33.2 kDa). SDS page analysis (right panel) of SEC fractions shows high purity (>95% pure). Fractions were pooled as indicated by the blue dashed lines in the chromatogram profiles (left panel) and the horizontal lines above fraction numbers on the SDS-PAGE (right panel). FIG. 1B shows chromatogram profiles for the final size exclusion chromatography (SEC) purification step for Nb_{NFR5} (Superdex 75 increase 10/300 GL) expressed in E. coli (left panel). The elution volume (Ve) fits the theoretical monomeric molecular weight (Nb_{NFR5}=14.43 kDa). SDS page analysis (right panel) of SEC fractions shows high purity (>95% pure). Fractions were pooled as indicated by the blue dashed lines in the chromatogram profiles (left panel) and the horizontal lines above fraction numbers on the SDS-PAGE (right panel). FIG. 1C shows that NFR5-kinase and VHH_{NFR5} (Nb_{NFR5}) form a stable complex as shown through SEC analysis. The left panel shows the SEC profile, and that NFR5-kinase+Nb_{NFR5} (red) shows a clear shift in elution volume, corresponding to larger particles as compared to the NFR5-kinase reference (grey). The right panel shows SDS-PAGE analysis of SEC fractions that show co-elution of NFR5-ID with Nb_{NFR5} (peak 1 from left panel), confirming complex formation. FIG. 1D shows the experimental design for VHH-induced complex formation to activate symbiotic signaling. Signal receptors NFR1 (white) and NFR5 (grey) are shown, with the intracellular components VHH_{NFR5} (Nb_{NFR5}; orange) and the NFR5 kinase domain (NFR5 kinase) are labelled. FIG. 1E shows schematics of constructs for expression in Lotus roots. The top schematic shows a Nfr1 promoter (pNfr1; white arrow), Nfr1 genomic sequence (white box), VHH_{NFR5} (Nb_{NFR5}; orange box), and Nfr1 terminator (tNfr1; white box with end bar). The bottom schematic shows a Nfr1 promoter (pNfr1; white arrow), Nfr1 genomic sequence (white box), VHH_{LaG16} (LaG16; teal box), Nfr1 terminator (tNFR1; white box with end bar), Nfr5 promoter (pNfr5; grey arrow), Nfr5 genomic sequence (grey box; equivalent to coding sequence as Nfr5 does not have introns), GFP coding sequence (green shape), and a Nfr5 terminator (tNfr5; grey box with end bar). FIG. 1F shows boxplots of the number of nodules formed on wild type Lotus plants (WT) and nfr1 mutant Lotus plants expressing the indicated constructs (protein schematics on x-axis) in the absence of rhizobia. Nodules were counted nine weeks after hairy root transformation; circles represent individual plants; and numbers below the boxplots specify number of nodulated plants out of total number of plants. In the protein schematics, NFR1 is white, NFR5 is grey, and Nb_{NFR5} is orange and attached to NFR1. Lowercase letters indicate significant differences between samples [analysis of variance (Kruskal-Wallis) and post-hoc analysis (Dunn's test), p<0.05]. FIG. 1G shows representative bright-field images of the nodulation phenotype of nfr1 nfr5 mutant Lotus roots transformed with NFR1 fused to the double VHH_{LaG16} and NFR5 fused to GFP in the absence of rhizobia; scale bar=1 mm. In the protein schematics shown at the right, NFR1 is white, NFR5 is grey, VHH_{LaG16} is teal and attached to NFR1, and GFP is green and attached to NFR5. FIG. 1H shows boxplots of the number of nodules formed on wild type Lotus plants (WT) and nfr1 nfr5 double mutant Lotus plants expressing the indicated constructs (protein schematics on x-axis) in the absence of rhizobia. Nodules were counted nine weeks after hairy root transformation; circles represent individual plants; and numbers below the boxplots specify number of nodulated plants out of total number of plants. In the protein schematics, NFR1 is white, NFR5 is grey, VHH_{LaG16} is teal, GFP is green, and “m” indicates the mutated VHH with no binding to GFP (i.e., VHH_{LaG16m}). Lowercase letters indicate significant differences between samples [analysis of variance (Kruskal-Wallis) and post-hoc analysis (Dunn's test), p<0.05]. FIG. 1I shows boxplots of the number of nodules formed on wild type Lotus plants (WT) or nfr1 nfr5 mutant Lotus plants expressing the indicated constructs (protein schematics on x-axis) in the absence of rhizobia. Nodules were counted nine weeks after hairy root transformation. Circles represent individual plants. Numbers below the boxplots specify number of nodulated plants out of total number of plants. In the protein schematics, NFR1 is white, NFR5 is grey, VHH_{LaG16} is teal, and GFP is green. Lowercase letters indicate significant differences between samples [analysis of variance (Kruskal-Wallis) and post-hoc analysis (Dunn's test), p<0.05]. Empty vector and complementation controls are identical to those in FIG. 1H. FIG. 1J shows co-immunoprecipitation of VHH_{LaG16}- and VHH_{LaG16m}-tagged mCherry and GFP-tagged NFR5 from N. benthamiana leaf extracts. Anti-GFP and anti-mCherry antibodies were used for immunoblotting. In the protein schematics below the blot, NFR5 is grey, GFP is green, VHH_{La}G16 is teal, the “m” indicates the mutated VHH with no binding to GFP (i.e., VHH_{LaG16m}), and mCherry is pink. Pluses and minuses above the blot indicate the respective presence or absence of the protein indicated to the left in the co-immunoprecipitation reaction.

FIGS. 2A-2C show that the core symbiotic signaling complex that activates organogenesis is conserved and operates via the known symbiotic pathway. FIG. 2A shows boxplots of the number of nodules formed on Medicago lyk3 nfp double mutant plants expressing the indicated constructs (protein schematics on x-axis) in the absence of rhizobia. In the protein schematics on the x-axis, MtLYK3 is light grey, MtNFP is dark grey, GFP is green, VHH_{LaG16} is teal, and the “m” indicates the mutated VHH with no binding to GFP (i.e., VHH_{LaG16m}). Lowercase letters indicate significant differences between samples [analysis of variance (Kruskal-Wallis) and post-hoc analysis (Dunn's test), p<0.05]. FIG. 2B shows representative bright-field (large image and top inset) and YFP fluorescence (transformation control; bottom inset) images of Medicago roots, showing the nodulation phenotype in the absence of rhizobia. Scale bar=1 mm. In the protein schematics below the images, MtLYK3 is light grey, MtNFP is dark grey, GFP is green, and VHH_{LaG16} is teal. FIG. 2C shows representative bright-field (top row) and YFP fluorescence (transformation control; bottom row) images of nfr1 nfr5 double mutant Lotus roots, nfr1 nfr5 symrk triple mutant Lotus roots, lhk1 mutant Lotus roots, nfr1 mutant Lotus roots, and nfr5 mutant Lotus roots, expressing NFR1-LaG16 with NFR5-GFP, NFR1-LaG16 with SYMRK-GFP, or NFR5-LaG16 with SYMRK-GFP in the absence of rhizobia. In the protein schematics below the images, NFR1 is white, NFR5 is grey, SYMRK is dark grey, GFP is green, VHH_{LaG16} is teal, and “kd” is the dead kinase domain. n=total number of plants; scale bars=1 mm.

FIGS. 3A-3H show that the cortical and epidermal programs are activated by VHH-mediated complex formation, but infection requires functional ectodomains. FIG. 3A shows boxplots of the number of nodules formed on wild type Lotus plants (WT) and nfr1 nfr5 double mutant Lotus plants expressing the indicated constructs (protein schematics on the x-axis) in the presence of M. loti. White boxplots show the number of white nodules, and pink boxplots show the number of pink nodules. Nodules were counted six weeks after inoculation with M. loti; numbers below the boxplots specify the number of plants with pink (infected) nodules out of total number of plants. In the protein schematics on the x-axis, NFR1 is white, NFR5 is grey, GFP is green, and VHH_{LaG16} is teal; constructs in the fourth through sixth columns shown as lacking the three overlapping pill-shapes at the top of the protein schematic were missing the ectodomain. Lowercase letters indicate significant differences between samples [analysis of variance (Kruskal-Wallis) and post-hoc analysis (Dunn's test), p<0.05]. FIG. 3B shows boxplots of the number of nodules formed on wild type Lotus plants (WT), nfr1 mutant Lotus plants, and nfr5 mutant Lotus plants expressing the indicated constructs (protein schematics on the x-axis) in the presence of M. loti. White boxplots show the number of white nodules, and pink boxplots show the number of pink nodules. Boxplots show the number of nodules formed on plants expressing the indicated constructs; nodules were counted six weeks after inoculation with M. loti; numbers below the boxplots specify the number of plants with pink (infected) nodules out of the total number of plants. In the protein schematics on the x-axis, NFR1 is white, NFR5 is grey, GFP is green, and VHH_{LaG16} is teal. Lowercase letters indicate significant differences between samples [analysis of variance (Kruskal-Wallis) and post-hoc analysis (Dunn's test), p<0.05]. FIG. 3C shows boxplots of the number of nodules formed on wild type Lotus plants (WT), nfr1 mutant, and symrk mutant Lotus plants expressing the indicated constructs (protein schematics on the x-axis) in the presence of M. loti. White boxplots show the number of white nodules, and pink boxplots show the number of pink nodules. Boxplots show the number of nodules formed on plants expressing the indicated constructs; nodules were counted six weeks after inoculation with M. loti; numbers below the boxplots specify the number of plants with pink (infected) nodules out of the total number of plants. In the protein schematics on the x-axis, NFR1 is white, SYMRK is dark grey, GFP is green, and VHH_{LaG16} is teal. Lowercase letters indicate significant differences between samples [analysis of variance (Kruskal-Wallis) and post-hoc analysis (Dunn's test), p<0.05]. FIG. 3D shows boxplots of the number of nodules formed on wild type Lotus plants (WT), nfr5 mutant, and symrk mutant Lotus plants expressing the indicated constructs (protein schematics on the x-axis) in the presence of M. loti. White boxplots show the number of white nodules, and pink boxplots show the number of pink nodules. Boxplots show the number of nodules formed on plants expressing the indicated constructs; nodules were counted six weeks after inoculation with M. loti; numbers below the boxplots specify the number of plants with pink (infected) nodules out of the total number of plants. In the protein schematics on the x-axis, NFR5 is light grey, SYMRK is dark grey, GFP is green, and VHH_{LaG16} is teal. Lowercase letters indicate significant differences between samples [analysis of variance (Kruskal-Wallis) and post-hoc analysis (Dunn's test), p<0.05]. FIG. 3E shows the number of nodules formed on nfr1 nfr5 double mutant Lotus plants expressing the indicated constructs (protein schematics on the x-axis) in the absence of rhizobia or the presence of IRBG74. Boxplots show the number of nodules formed on plants expressing the indicated constructs; nodules were counted four weeks after inoculation with IRBG74; black and red numbers below the boxplots specify the number of plants with white (non-infected) or pink (infected) nodules, respectively, out of the total number of plants. In the protein schematics on the x-axis, NFR1 is white, NFR5 is light grey, GFP is green, and VHH_{LaG16} is teal. Lowercase letters indicate significant differences between samples [analysis of variance (Kruskal-Wallis) and post-hoc analysis (Dunn's test), p<0.05]. FIG. 3F shows representative pictures of pNIN:GUS expression in wild type (WT) (note that WT is a transformed line carrying pNIN:GUS) roots inoculated with M. loti (top) and non-inoculated roots of stable Lotus lines expressing NFR1-LaG16 and NFR5-GFP (schematics shown at right). In the protein schematics on the right, NFR1 is white, NFR5 is light grey, GFP is green, and VHH_{LaG16} is teal. Triangles pinpoint cells expressing GUS. For the stable line root hair image, three pictures were overlaid. Scale bars=0.1 mm. FIG. 3G shows the number of infections threads (ITs) formed per centimeter of root at 10 days post-infection with M. loti in wild type (WT) and stable Lotus lines expressing NFR1-LaG16 and NFR5-GFP. Error bars represent standard deviation. A Kruskal-Wallis t-test was performed for statistical analysis; * indicates p<0.001. Numbers below the bars specify the number of plants with infection threads out of the total number of plants. In the protein schematics on the x-axis, NFR1 is white, NFR5 is light grey, GFP is green, and VHH_{LaG16} is teal. FIG. 3H shows that ectodomains of NFR1 and NFR5 are required for full efficiency of organogenesis activation. Boxplots show the number of nodules formed on Lotus WT or nfr1 nfr5 mutant plants expressing the indicated constructs (protein schematics on the x-axis). Nodules were counted nine weeks after hairy root transformation. Numbers below the boxplots specify number of nodulated plants out of total number of plants. In the protein schematics on the x-axis, NFR1 is white, NFR5 is light grey, GFP is green, and VHH_{LaG16} is teal; constructs in the fourth through sixth columns shown as lacking the three overlapping pill-shapes at the top of the protein schematic were missing the ectodomain. Lowercase letters indicate significant differences between samples [analysis of variance (Kruskal-Wallis) and post-hoc analysis (Dunn's test), p<0.05]. The empty vector and complementation controls are identical to those in FIG. 1H.

FIGS. 4A-4K show the identification of barley receptors capable of activating the symbiotic signaling pathway. FIG. 4A shows a phylogenetic tree of selected barley (Hv), Lotus (Lj), and Medicago (Mt) full-length LysM receptors. Yellow and teal shaded backgrounds highlight NFR1-type and NFR5-type receptors, respectively. FIG. 4B shows structural modelling on the left of barley (Hv) RLK4 (orange) and RLK10 (teal). The amino acid sequence identity compared to the intracellular parts of Lotus NFR1 and NFR5 is indicated. On the right, ribbon diagrams of the modelled ectodomains of barley RLK4 (top) and RLK10 (bottom) are superimposed over the crystal structures of Medicago (Mt) LYK3 (PDB-ID: 6XWE) and NFP (PDB-ID: 7AU7), respectively, and the sequence identity is indicated. C-α root-mean-square deviations (RMSD) are indicated in Angstrom (A). FIG. 4C shows the phylogenetic relationship of selected LysM receptors. The evolutionary relationship between Lotus (Lj), Medicago (Mt), and barley (Hv) LysM receptors is displayed. Yellow and teal shaded backgrounds highlight NFR1-type and NFR5-type receptors, respectively. FIG. 4D shows the number of white and pink nodules formed on nfr1 nfr5 double mutant Lotus plants expressing the indicated constructs (protein schematics on the x-axis) in the absence of rhizobia. White boxplots show the number of white nodules, and pink boxplots show the number of pink nodules; numbers below the boxplots specify the number of plants with pink (infected) nodules out of the total number of plants. In the protein schematics on the x-axis, NFR1 is tan, NFR5 is purple, barley RLK4 (HvRLK4) is dark teal, barley RLK10 (HvRLK10) is light teal, GFP is bright green, and the teal shape with the “m” indicates the mutated VHH with no binding to GFP (i.e., VHH_{LaG16m}). Lowercase letters indicate significant differences between samples [analysis of variance (Kruskal-Wallis) and post-hoc analysis (Dunn's test), p<0.05]. FIG. 4E shows the number of nodules formed on nfr1 nfr5 double mutant Lotus plants expressing the indicated constructs (protein schematics on the x-axis) in the absence of rhizobia. Boxplots show the number of nodules formed on plants expressing the indicated constructs; nodules were counted nine weeks after hairy root transformation; numbers below the boxplots indicate the number of nodulating plants out of the total number of plants. In the protein schematics on the x-axis, NFR1 is white, NFR5 is light grey, HvRLK4 is orange, HvRLK10 is dark teal, CERK6 is in pale green, GFP is bright green, and VHH_{LaG16} is teal. Lowercase letters indicate significant differences between samples [analysis of variance (Kruskal-Wallis) and post-hoc analysis (Dunn's test), p<0.05]. The complementation control is identical to that in FIG. 1I. FIG. 4F shows phenotypes of nodules formed in nfr1 nfr5 double mutant Lotus roots expressing barley RLK4-LaG16 with RLK10-GFP. YFP serves as a transformation control; the visible fluorescence signal in the YFP channel shows that the roots with nodules are transformed roots. Scale bars=1 mm. FIG. 4G shows the number of nodules formed on wild type Lotus plants (WT), nfr1 mutant Lotus plants, nfr5 mutant Lotus plants, and nfr1 nfr5 double mutant Lotus plants expressing the indicated constructs (protein schematics on the x-axis) in the presence of M. loti. White boxplots show the number of white nodules, and pink boxplots show the number of pink nodules. Boxplots show the number of nodules formed on plants expressing the indicated constructs; and nodules were counted six weeks inoculation with M. loti. In the protein schematics on the x-axis, NFR1 is white, NFR5 is light grey, HvRLK4 is dark teal, HvRLK10 is teal and shaped like NFR5, GFP is green, VHH_{LaG16} is teal and roughly circular, and the “m” indicates the mutated VHH with no binding to GFP (i.e., VHH_{LaG16m}). Lowercase letters indicate significant differences between samples [analysis of variance (Kruskal-Wallis) and post-hoc analysis (Dunn's test), p<0.05]. FIG. 4H shows representative bright-field and YFP fluorescence (transformation control) pictures of Medicago lyk3 nfp double mutant roots expressing RLK4-2xLaG16 and RLK10-GFP. Scale bar=1 mm. In the protein schematics to the right, HvRLK4 is orange, HvRLK10 is dark teal and labelled, GFP is green, and VHH_{LaG16} is teal. FIG. 4I shows the number of nodules formed on lyk3 nfp double mutant Medicago plants expressing the indicated constructs (protein schematics on the x-axis) in the absence of rhizobia. Boxplots shows the number of nodules formed on plants expressing the indicated constructs in the absence of rhizobia; nodules were counted nine weeks after hairy root transformation; numbers below the boxplots specify number of nodulated plants out of total number of plants. In the protein schematics on the x-axis, MtLYK3 is light grey, MtNFP is dark grey, HvRLK4 is orange, HvRLK10 is dark teal, GFP is green, VHH_{LaG16} is teal, and the “m” indicates the mutated VHH with no binding to GFP (i.e., VHH_{LaG16m}). Lowercase letters indicate significant differences between samples [analysis of variance (Kruskal-Wallis) and post-hoc analysis (Dunn's test), p<0.05]. FIG. 4J shows representative bright-field and YFP fluorescence (transformation control) pictures of Lotus nfr1 nfr5 double mutant roots expressing CERK6-2xLaG16 and NFR5-GFP. Scale bar=1 mm. In the protein schematics to the right, CERK6 is in pale green, NFR5 is in grey, GFP is bright green, and VHH_{LaG16} is teal. FIG. 4K shows the number of nodules formed on nfr1 nfr5 double mutant Lotus plants expressing the indicated constructs (protein schematics on the x-axis) in the absence of rhizobia. Boxplots show the number of nodules formed on plants expressing the indicated constructs in the absence of rhizobia; nodules were counted nine weeks after hairy root transformation; numbers below the boxplots specify total number of plants. In the protein schematics on the x-axis, NFR1 is in dark grey, NFR5 is in olive green, RLK10 is in yellow, CERK6 is in pale green, RLK4 is in dark brown, RLK1 and RLK2 are in pale grey, RLK7 is in brown with white labelling, RLK5 is in light brown with black lettering, GFP is in bright green, and VHH_{LaG16} is teal.

FIGS. 5A-5L show an amino acid sequence alignment of NFR1-type LysM receptor sequences from Mimosa pudica (Scaffold15743=SEQ ID NO: 32), Chamaecrista fasciculata (2879S20281=SEQ ID NO: 22), Prosopis alba (XP_0287539017.1=SEQ ID NO: 26), Arachis duranensis (XP_020982945.1=SEQ ID NO: 20), Arachis hypogaea (XP_029150476.1=SEQ ID NO: 7), Arachis ipaensis (XP_020962820.1=SEQ ID NO: 21), Lupinus angustifolius (XP_019434083.1=SEQ ID NO: 14; XP_019461629.1=SEQ ID NO: 15), Lupinus alba (Chr04g0249871=SEQ ID NO: 24), Cicer arietinum (XP_004491136.1=SEQ ID NO: 10), Medicago truncatula (Q6UD73_LYK3_MEDTR=SEQ ID NO: 5), Pisum sativum (ARX80051.1=SEQ ID NO: 16), Lotus japonicus (CAE02590.1=SEQ ID NO: 4), Cajanus cajan (XP_020213700.2=SEQ ID NO: 9), Abrus precatorius (XP_027332267.1=SEQ ID NO: 11), Phaseolus vulgaris (XP_007141617.1=SEQ ID NO: 6), Vigna angularis (KOM46748.1=SEQ ID NO: 30), Vigna unguiculata (CP+027939826.1=SEQ ID NO: 31), Spatholobus suberectus (TKY57029.1=SEQ ID NO: 25), and Glycine max (XP_006575588.1=SEQ ID NO: 12; XP_006595821.2=SEQ ID NO: 13). FIG. 5A shows the first portion of the alignment. FIG. 5B shows the second portion of the alignment. FIG. 5C shows the third portion of the alignment. FIG. 5D shows the fourth portion of the alignment. FIG. 5E shows the fifth portion of the alignment. FIG. 5F shows the sixth portion of the alignment. FIG. 5G shows the seventh portion of the alignment. FIG. 5H shows the eighth portion of the alignment. FIG. 5I shows the ninth portion of the alignment. FIG. 5J shows the tenth portion of the alignment. FIG. 5K shows the eleventh portion of the alignment. FIG. 5L shows the twelfth portion of the alignment.

FIGS. 6A-6F show an amino acid sequence alignment of NFR5-type LysM receptor sequences from Arachis hypogaea (XP_025698788.1=SEQ ID NO: 44), Arachis ipaensis (XP_016200640.1=SEQ ID NO: 45), Medicago truncatula (Medtr; Medtr8g078300.1=SEQ ID NO: 36; Medtr5g019040.1=SEQ ID NO: 38), Cicer arietinum (XP_004509233.1=SEQ ID NO: 35; XP_012574460.1=SEQ ID NO: 42), Lupinus angustifolius (XP_019420412.1=SEQ ID NO: 43), Lotus japonicus (AER51027.1 (NFR5)=SEQ ID NO: 37; BAI79275.1 (LYS11)=SEQ ID NO: 33), Abrus precatorius (XP_027347386.1=SEQ ID NO: 34; XP_027362672.1=SEQ ID NO: 41), Cajanus cajan (KYP66704.1=SEQ ID NO: 39), and Phaseolus vulgaris (XP_007156886.1=SEQ ID NO: 40). FIG. 6A shows the first portion of the alignment. FIG. 6B shows the second portion of the alignment. FIG. 6C shows the third portion of the alignment. FIG. 6D shows the fourth portion of the alignment. FIG. 6E shows the fifth portion of the alignment. FIG. 6F shows the sixth portion of the alignment.

FIG. 7 shows a schematic of spontaneous nodulation without rhizobia. At the top, the schematic shows that no LCO input from rhizobia is needed. On the middle right, a receptor with a 3×LysM ectodomain, a transmembrane helix, a juxtamembrane domain, and a kinase domain fused to GFP is shown. On the middle left, a receptor with a 3× LysM ectodomain, a transmembrane helix, a juxtamembrane domain, and a kinase domain fused to an anti-GFP affinity polypeptide is shown. At the bottom, the outcome of spontaneous nodulation without rhizobia is shown (Rubsam H, Krönauer C, Abel N B, Ji H, Lironi D, Hansen S B, Nadzieja M, Kolte M V, Abel D, de Jong N, Madsen L H, Liu H, Stougaard J, Radutoiu S, Andersen K R. Nanobody-driven signaling reveals the core receptor complex in root nodule symbiosis. Science. 2023 Jan. 20; 379(6629):272-277. doi: 10.1126/science.ade9204. Epub 2023 Jan. 19. PMID: 36656954).

### DETAILED DESCRIPTION

The following description sets forth exemplary methods, parameters, and the like. It should be recognized, however, that such description is not intended as a limitation on the scope of the present disclosure but is instead provided as a description of exemplary embodiments.

### Genetically Modified Cells

An aspect of the disclosure includes a genetically modified cell including: a transmembrane (TM) receptor complex including a first subunit polypeptide and a second subunit polypeptide, wherein the first subunit polypeptide includes an affinity polypeptide that binds to the second subunit polypeptide intracellularly inducing oligomerization; and wherein oligomerization of the first subunit polypeptide and the second subunit polypeptide activates TM receptor signaling, wherein the affinity polypeptide is heterologous to the first subunit polypeptide. In an additional embodiment of this aspect, the TM receptor is a single-pass TM (SPTM) receptor, or the TM receptor is a SPTM receptor including an intracellular kinase domain (SPTM-kinase). In a further embodiment of this aspect, which may be combined with any of the preceding embodiments, the affinity polypeptide binds directly to the second subunit polypeptide. In yet another embodiment of this aspect, which may be combined with any of the preceding embodiments, the second subunit polypeptide includes a tag polypeptide, and the affinity polypeptide binds to the tag polypeptide. In still another embodiment of this aspect, the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, or a ubiquitin tag polypeptide. In a further embodiment of this aspect the tag polypeptide is a fluorescent protein polypeptide selected from a green fluorescent protein (GFP) polypeptide, a red fluorescent polypeptide (RFP) (e.g., mCherry), or a blue fluorescent polypeptide (BFP); wherein the tag polypeptide is a luminescence polypeptide selected from a luciferase polypeptide; or wherein the tag polypeptide is a combination of a fluorescent protein polypeptide and a luminescence polypeptide. In yet another embodiment of this aspect, which may be combined with any of the preceding embodiments, the affinity polypeptide is selected from a heavy-chain variable domain (VHH), a single-chain variable fragment (scFV), a designed ankyrin repeat protein (DARPins), a human fibronectin III domain 3 monobody, an Affibody, or an anticalin, or a synthetic version of any of the foregoing. In an additional embodiment of this aspect, the affinity polypeptide is a heavy-chain variable domain (VHH). In a further embodiment of this aspect, the affinity polypeptide is a VHH_{LaG16}. In some embodiments, the VHH can be synthetic (e.g., a synthetic Nanobody®).

In still another embodiment of this aspect, which may be combined with any of the preceding embodiments, the cell is a eubacterial cell, an archaeal cell, or a eukaryotic cell. Single-pass transmembrane proteins in different cells are disclosed in, for example, Pogozheva and Lomize (2018), Evolution and adaptation of single-pass transmembrane proteins, BBA-Biomembranes, 1860(2):364-377. In another embodiment of this aspect, the eukaryotic cell is a plant cell, an animal cell, or a fungal cell. In yet another embodiment of this aspect, which may be combined with any of the preceding embodiments, the cell is a plant cell and the first subunit polypeptide and/or the second subunit polypeptide are selected from a LysM receptor, a Leucine rich repeat (LRR) receptor, a Malectin like receptor, a plant TM receptor, a plant SPTM (pSPTM) receptor, and a plant SPTM receptor including an intracellular kinase domain (pSPTM-kinase). In a further embodiment of this aspect, the first subunit polypeptide and/or the second subunit polypeptide is a LysM receptor. In an additional embodiment of this aspect, the first subunit polypeptide and/or the second subunit polypeptide lacks an ectodomain and/or a transmembrane domain. In another embodiment of this aspect, which may be combined with any of the preceding embodiments where the first subunit polypeptide and/or the second subunit polypeptide is a LysM receptor, the first subunit polypeptide is a NFR1 polypeptide and the second subunit polypeptide is a NFR5 polypeptide, wherein the first subunit polypeptide is a NFR5 polypeptide and the second subunit is a NFR1 polypeptide, wherein the first subunit polypeptide is a LYK3 polypeptide and the second subunit polypeptide is a NFP polypeptide, wherein the first subunit polypeptide is a NFP polypeptide and the second subunit polypeptide is a LYK3 polypeptide, wherein the first subunit polypeptide is a RLK4 receptor polypeptide and the second subunit polypeptide is a RLK10 receptor polypeptide, or wherein the first subunit polypeptide is a RLK10 polypeptide and the second subunit polypeptide is a RLK4 polypeptide. In a further embodiment of this aspect, the NFR1, LYK3, or RLK4 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 56, and wherein the NFR5, NFP, or RLK10 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, and SEQ ID NO: 57. Additional suitable LysM receptor polypeptides may include RLK1, RLK2, RLK5, RLK7, CERK6, and SYMRK. In yet another embodiment of this aspect, which may be combined with any of the preceding embodiments, the affinity polypeptide binding to the second subunit polypeptide is regulated by a small molecule.

Another aspect of the disclosure includes a genetically modified plant cell including: an NFR1-NFR5 receptor complex including a first subunit polypeptide and a second subunit polypeptide, wherein the first subunit polypeptide includes an affinity polypeptide that binds to the second subunit polypeptide inducing oligomerization, and wherein the affinity polypeptide is heterologous to the first subunit polypeptide; and wherein oligomerization of the first subunit polypeptide and the second subunit polypeptide activates NFR1-NFR5 receptor complex signaling and wherein (i) the first subunit polypeptide is an NFR1 polypeptide and the second subunit polypeptide is an NFR5 polypeptide, or (ii) the first subunit polypeptide is the NFR5 polypeptide and the second subunit polypeptide is the NFR1 polypeptide. In an additional embodiment of this aspect, the NFR1 polypeptide and/or the NFR5 polypeptide lacks an ectodomain and/or a transmembrane domain. In a further embodiment of this aspect, which may be combined with any of the preceding embodiments, the NFR1 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 56, and wherein the NFR5 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 57. Additional suitable LysM receptor polypeptides may include RLK1, RLK2, RLK5, RLK7, CERK6, and SYMRK. In yet another embodiment of this aspect, which may be combined with any of the preceding embodiments, the affinity polypeptide binds directly to an intracellular portion of the second subunit polypeptide. In still another embodiment of this aspect, which may be combined with any of the preceding embodiments, the second subunit polypeptide includes a tag polypeptide fused to an intracellular portion of the second subunit polypeptide, and wherein the affinity polypeptide binds to the tag polypeptide. In a further embodiment of this aspect, the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, or a ubiquitin tag polypeptide. In an additional embodiment of this aspect, the tag polypeptide is a fluorescent protein polypeptide selected from a green fluorescent protein (GFP) polypeptide, a red fluorescent polypeptide (RFP) (e.g., mCherry), or a blue fluorescent polypeptide (BFP); wherein the tag polypeptide is a luminescence polypeptide selected from a luciferase polypeptide; or wherein the tag polypeptide is a combination of a fluorescent protein polypeptide and a luminescence polypeptide. In yet another embodiment of this aspect, which may be combined with any of the preceding embodiments, the affinity polypeptide is selected from a heavy-chain variable domain (VHH), a single-chain variable fragment (scFV), a designed ankyrin repeat protein (DARPins), a human fibronectin III domain 3 monobody, an Affibody, or an anticalin, or a synthetic version of any of the foregoing. In still another embodiment of this aspect, the affinity polypeptide is a heavy-chain variable domain (VHH). In a further embodiment of this aspect, the affinity polypeptide is VHH_{LaG16}. In another embodiment of this aspect, which may be combined with any of the preceding embodiments, the affinity polypeptide binding to the second subunit polypeptide is regulated by a small molecule.

A further aspect of the disclosure includes a bispecific affinity polypeptide for activation of an NFR1-NFR5 receptor including: a first affinity polypeptide that binds to an intracellular portion of an NFR1 polypeptide, wherein the first affinity polypeptide is heterologous to the NFR1 polypeptide; a second affinity polypeptide that binds to an intracellular portion of an NFR5 polypeptide, wherein the affinity polypeptide is heterologous to the NFR5 receptor subunit polypeptide; and wherein binding of the first affinity polypeptide to the NFR1 polypeptide and of the second affinity to the NFR5 polypeptide induces dimerization; and wherein dimerization of the NFR1 polypeptide and the NFR5 polypeptide activates the NFR1-NFR5 receptor. In an additional aspect of this aspect. the NFR1 polypeptide and/or the NFR5 polypeptide lacks an ectodomain and/or a transmembrane domain. In yet another embodiment of this aspect, which may be combined with any of the preceding embodiments, the NFR1 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 56, and wherein the NFR5 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 57. Additional suitable LysM receptor polypeptides may include RLK1, RLK2, RLK5, RLK7, CERK6, and SYMRK. In still another embodiment of this aspect, which may be combined with any of the preceding embodiments, the first affinity polypeptide binds directly to the NFR1 polypeptide and/or wherein the second affinity polypeptide binds directly to the NFR5 polypeptide. In an additional embodiment of this aspect, which may be combined with any of the preceding embodiments, the NFR1 polypeptide includes a tag polypeptide fused to an intracellular portion of the NFR1 polypeptide, and wherein the first affinity polypeptide binds to the tag polypeptide; and/or wherein the NFR5 polypeptide includes a tag polypeptide fused to an intracellular portion of the NFR5 polypeptide, and wherein the second affinity polypeptide binds to the tag polypeptide. In a further embodiment of this aspect, the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a luciferase tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, or a ubiquitin tag polypeptide. In another embodiment of this aspect, the tag polypeptide is a fluorescent protein polypeptide selected from a green fluorescent protein (GFP) polypeptide, a red fluorescent polypeptide (RFP) (e.g., mCherry), or a blue fluorescent polypeptide (BFP); wherein the tag polypeptide is a luminescence polypeptide selected from a luciferase polypeptide; or wherein the tag polypeptide is a combination of a fluorescent protein polypeptide and a luminescence polypeptide. In an additional embodiment of this aspect, which may be combined with any of the preceding embodiments, the first affinity polypeptide and the second affinity polypeptide are selected from a heavy-chain variable domain (VHH), a single-chain variable fragment (scFV), a designed ankyrin repeat protein (DARPins), a human fibronectin III domain 3 monobody, an Affibody, or an anticalin, or a synthetic version of any of the foregoing. In a further embodiment of this aspect, the first affinity polypeptide and/or the second affinity polypeptide is a heavy-chain variable domain (VHH). In an additional embodiment of this aspect, the affinity polypeptide is VHH_{LaG16}. In yet another embodiment of this aspect, which may be combined with any of the preceding embodiments, the first affinity polypeptide binding to the NFR1 polypeptide is regulated by a small molecule and/or the second affinity polypeptide binding to the NFR5 polypeptide is regulated by a small molecule.

### Bispecific Affinity Polypeptides

An additional aspect of the disclosure includes a bispecific affinity polypeptide including: a first affinity polypeptide that binds to an intracellular portion of a first subunit polypeptide of a TM receptor; and a second affinity polypeptide that binds to an intracellular portion of a second subunit polypeptide of the TM receptor; wherein binding of the first affinity polypeptide to the first subunit polypeptide and of the second affinity polypeptide to the second subunit polypeptide induces oligomerization; and wherein oligomerization of the first subunit polypeptide and the second subunit polypeptide activates TM receptor signaling. In a further embodiment of this aspect, the TM receptor is a single-pass TM (SPTM) receptor, or the TM receptor is a SPTM including an intracellular kinase domain (SPTM-kinase). In yet another embodiment of this aspect, which may be combined with any of the preceding embodiments, the first affinity polypeptide binds directly to the first subunit polypeptide and/or the second affinity polypeptide binds directly to the second subunit polypeptide. In an additional embodiment of this aspect, which may be combined with any of the preceding embodiments, the first subunit polypeptide includes a tag polypeptide, and the first affinity polypeptide binds to the tag polypeptide, and/or the second subunit polypeptide includes a tag polypeptide, and the second affinity polypeptide binds to the tag polypeptide. In still another embodiment of this aspect, the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, or a ubiquitin tag polypeptide. In a further embodiment of this aspect, the tag polypeptide is a fluorescent protein polypeptide selected from a green fluorescent protein (GFP) polypeptide, a red fluorescent polypeptide (RFP) (e.g., mCherry), or a blue fluorescent polypeptide (BFP); wherein the tag polypeptide is a luminescence polypeptide selected from a luciferase polypeptide; or wherein the tag polypeptide is a combination of a fluorescent protein polypeptide and a luminescence polypeptide. In yet another embodiment of this aspect, which may be combined with any of the preceding embodiments, the affinity polypeptide is selected from a heavy-chain variable domain (VHH), a single-chain variable fragment (scFV), a designed ankyrin repeat protein (DARPins), a human fibronectin III domain 3 monobody, an Affibody, or an anticalin, or a synthetic version of any of the foregoing. In an additional embodiment of this aspect, the affinity polypeptide is a heavy-chain variable domain (VHH). In a further embodiment of this aspect, the affinity polypeptide is a VHH_{LaG16}. In an additional embodiment of this aspect, which may be combined with any of the preceding embodiments, the first subunit polypeptide and/or the second subunit polypeptide are selected from a LysM receptor, a Leucine rich repeat (LRR) receptor, a Malectin like receptor, a plant TM receptor, a plant SPTM (pSPTM) receptor, and a plant SPTM receptor including an intracellular kinase domain (pSPTM-kinase). In yet another embodiment of this aspect, the first subunit polypeptide and/or the second subunit polypeptide is a LysM receptor. In an additional embodiment of this aspect, the first subunit polypeptide and/or the second subunit polypeptide lacks an ectodomain and/or a transmembrane domain. In another embodiment of this aspect, which may be combined with any of the preceding embodiments where the first subunit polypeptide and/or the second subunit polypeptide is a LysM receptor, the first subunit polypeptide is a NFR1 polypeptide and the second subunit polypeptide is a NFR5 polypeptide, wherein the first subunit polypeptide is a NFR5 polypeptide and the second subunit is a NFR1 polypeptide, wherein the first subunit polypeptide is a LYK3 polypeptide and the second subunit polypeptide is a NFP polypeptide, wherein the first subunit polypeptide is a NFP polypeptide and the second subunit polypeptide is a LYK3 polypeptide, wherein the first subunit polypeptide is a RLK4 receptor polypeptide and the second subunit polypeptide is a RLK10 receptor polypeptide, or wherein the first subunit polypeptide is a RLK10 polypeptide and the second subunit polypeptide is a RLK4 polypeptide. In a further embodiment of this aspect, the NFR1, LYK3, or RLK4 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 56, and wherein the NFR5, NFP, or RLK10 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 57. Additional suitable LysM receptor polypeptides may include RLK1, RLK2, RLK5, RLK7, CERK6, and SYMRK. In yet another embodiment of this aspect, which may be combined with any of the preceding embodiments, the affinity polypeptide binding to the second subunit polypeptide is regulated by a small molecule.

A further aspect of the disclosure includes a bispecific affinity polypeptide for activation of an NFR1-NFR5 receptor including: a first affinity polypeptide that binds to an intracellular portion of an NFR1 polypeptide, wherein the first affinity polypeptide is heterologous to the NFR1 polypeptide; a second affinity polypeptide that binds to an intracellular portion of an NFR5 polypeptide, wherein the affinity polypeptide is heterologous to the NFR5 receptor subunit polypeptide; and wherein binding of the first affinity polypeptide to the NFR1 polypeptide and of the second affinity to the NFR5 polypeptide induces dimerization; and wherein dimerization of the NFR1 polypeptide and the NFR5 polypeptide activates the NFR1-NFR5 receptor. In an additional aspect of this aspect. the NFR1 polypeptide and/or the NFR5 polypeptide lacks an ectodomain and/or a transmembrane domain. In yet another embodiment of this aspect, which may be combined with any of the preceding embodiments, the NFR1 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 56, and wherein the NFR5 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 57. Additional suitable LysM receptor polypeptides may include RLK1, RLK2, RLK5, RLK7, CERK6, and SYMRK. In still another embodiment of this aspect, which may be combined with any of the preceding embodiments, the first affinity polypeptide binds directly to the NFR1 polypeptide and/or wherein the second affinity polypeptide binds directly to the NFR5 polypeptide. In an additional embodiment of this aspect, which may be combined with any of the preceding embodiments, the NFR1 polypeptide includes a tag polypeptide fused to an intracellular portion of the NFR1 polypeptide, and wherein the first affinity polypeptide binds to the tag polypeptide; and/or wherein the NFR5 polypeptide includes a tag polypeptide fused to an intracellular portion of the NFR5 polypeptide, and wherein the second affinity polypeptide binds to the tag polypeptide. In a further embodiment of this aspect, the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a luciferase tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, or a ubiquitin tag polypeptide. In another embodiment of this aspect, the tag polypeptide is a fluorescent protein polypeptide selected from a green fluorescent protein (GFP) polypeptide, a red fluorescent polypeptide (RFP) (e.g., mCherry), or a blue fluorescent polypeptide (BFP); wherein the tag polypeptide is a luminescence polypeptide selected from a luciferase polypeptide; or wherein the tag polypeptide is a combination of a fluorescent protein polypeptide and a luminescence polypeptide. In an additional embodiment of this aspect, which may be combined with any of the preceding embodiments, the first affinity polypeptide and the second affinity polypeptide are selected from a heavy-chain variable domain (VHH), a single-chain variable fragment (scFV), a designed ankyrin repeat protein (DARPins), a human fibronectin III domain 3 monobody, an Affibody, or an anticalin, or a synthetic version of any of the foregoing. In a further embodiment of this aspect, the first affinity polypeptide and/or the second affinity polypeptide is a heavy-chain variable domain (VHH). In an additional embodiment of this aspect, the affinity polypeptide is VHH_{LaG16}. In yet another embodiment of this aspect, which may be combined with any of the preceding embodiments, the first affinity polypeptide binding to the NFR1 polypeptide is regulated by a small molecule and/or the second affinity polypeptide binding to the NFR5 polypeptide is regulated by a small molecule.

### Methods of Screening

A further aspect of the disclosure includes methods of screening transmembrane (TM) receptor including: (a) providing a cell expressing a first subunit polypeptide of a TM receptor; (b) expressing a second subunit polypeptide of a TM receptor in the cell; and (c) assaying the cell for a TM receptor phenotype; and wherein (i) the presence of the TM receptor phenotype indicates the first subunit polypeptide and the second subunit polypeptide oligomerize to form the TM receptor; or (ii) the absence of the TM receptor phenotype indicates the first subunit polypeptide and the second subunit polypeptide do not oligomerize to form a TM receptor, and wherein (1) the second subunit polypeptide includes a tag polypeptide, the first subunit polypeptide includes an affinity polypeptide that binds to the tag polypeptide, and the affinity polypeptide is heterologous to the first subunit polypeptide, or (2) the first subunit polypeptide includes the tag polypeptide, the second subunit polypeptide includes the affinity polypeptide that binds to the tag polypeptide, and the affinity polypeptide is heterologous to the second subunit polypeptide. In an additional embodiment of this aspect, the TM receptor is a single-pass TM (SPTM) receptor, or the TM receptor is a SPTM including an intracellular kinase domain (SPTM-kinase). In yet another embodiment of this aspect, which may be combined with any of the preceding embodiments, the binding partner for the first subunit polypeptide is unknown and (b) is repeated using two or more candidate second subunit polypeptides to identify the second subunit polypeptide that is the binding partner for the first subunit polypeptide. In a further embodiment of this aspect, the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, or a ubiquitin tag polypeptide. In a further embodiment of this aspect, the tag polypeptide is a fluorescent protein polypeptide selected from a green fluorescent protein (GFP) polypeptide, a red fluorescent polypeptide (RFP) (e.g., mCherry), or a blue fluorescent polypeptide (BFP); wherein the tag polypeptide is a luminescence polypeptide selected from a luciferase polypeptide; or wherein the tag polypeptide is a combination of a fluorescent protein polypeptide and a luminescence polypeptide. In yet another embodiment of this aspect, which may be combined with any of the preceding embodiments, the affinity polypeptide is selected from a heavy-chain variable domain (VHH), a single-chain variable fragment (scFV), a designed ankyrin repeat protein (DARPins), a human fibronectin III domain 3 monobody, an Affibody, or an anticalin, or a synthetic version of any of the foregoing. In an additional embodiment of this aspect, the affinity polypeptide is a heavy-chain variable domain (VHH). In a further embodiment of this aspect, the affinity polypeptide is a VHH_{LaG16}. In still another embodiment of this aspect, which may be combined with any of the preceding embodiments, the cell is a eubacterial cell, an archaeal cell, or a eukaryotic cell. In another embodiment of this aspect, the eukaryotic cell is a plant cell, an animal cell, or a fungal cell. In yet another embodiment of this aspect, which may be combined with any of the preceding embodiments, the cell is a plant cell and the first subunit polypeptide and/or the second subunit polypeptide are selected from a LysM receptor, a Leucine rich repeat (LRR) receptor, a Malectin like receptor, a plant TM receptor, a plant SPTM (pSPTM) receptor, and a plant SPTM receptor including an intracellular kinase domain (pSPTM-kinase). In a further embodiment of this aspect, the first subunit polypeptide and/or the second subunit polypeptide is a LysM receptor. In an additional embodiment of this aspect, the first subunit polypeptide and/or the second subunit polypeptide lacks an ectodomain and/or a transmembrane domain. In another embodiment of this aspect, which may be combined with any of the preceding embodiments where the first subunit polypeptide and/or the second subunit polypeptide is a LysM receptor, the first subunit polypeptide is a NFR1 polypeptide and the second subunit polypeptide is a NFR5 polypeptide, wherein the first subunit polypeptide is a NFR5 polypeptide and the second subunit is a NFR1 polypeptide, wherein the first subunit polypeptide is a LYK3 polypeptide and the second subunit polypeptide is a NFP polypeptide, wherein the first subunit polypeptide is a NFP polypeptide and the second subunit polypeptide is a LYK3 polypeptide, wherein the first subunit polypeptide is a RLK4 receptor polypeptide and the second subunit polypeptide is a RLK10 receptor polypeptide, or wherein the first subunit polypeptide is a RLK10 polypeptide and the second subunit polypeptide is a RLK4 polypeptide. In a further embodiment of this aspect, the NFR1, LYK3, or RLK4 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 56, and wherein the NFR5, NFP, or RLK10 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 57. Additional suitable LysM receptor polypeptides may include RLK1, RLK2, RLK5, RLK7, CERK6, and SYMRK. In yet another embodiment of this aspect, which may be combined with any of the preceding embodiments, the affinity polypeptide binding to the second subunit polypeptide is regulated by a small molecule.

### Genetically Modified Plants and Related Methods

Some aspects of the disclosure are related to a genetically modified plant or part thereof including the genetically modified plant cell of any one of the preceding embodiments. An additional embodiment of this aspect further includes the TM receptor including the first subunit polypeptide and the second subunit polypeptide, wherein the first subunit polypeptide includes the affinity polypeptide that binds to the second subunit polypeptide intracellularly inducing oligomerization, and wherein oligomerization of the first subunit polypeptide and the second subunit polypeptide activates TM receptor signaling. In a further embodiment of this aspect, the affinity polypeptide binds directly to the second subunit polypeptide or wherein the second subunit polypeptide includes a tag polypeptide, and wherein the affinity polypeptide binds to the tag polypeptide. In yet another embodiment of this aspect, which may be combined with any of the preceding embodiments, the genetically modified plant cell is selected from a root epidermal cell, a root cortex cell, a root endodermis cell, a root pericycle cell, a root primordia cell, a xylem cell, a phloem cell, a meristem cell, a leaf cell, a stem cell, a flower cell, or a fruit cell. In still another embodiment of this aspect, the genetically modified plant cell is a root epidermal cell, a root cortex cell, a root endodermis cell, a root pericycle cell, or a root primordia cell.

Some aspects of the disclosure are related to a genetically modified plant or part thereof including the bispecific affinity polypeptide of any one of the preceding embodiments. Another embodiment of this aspect further includes the first affinity polypeptide that binds to the intracellular portion of the first subunit polypeptide of the TM receptor and the second affinity polypeptide that binds to the intracellular portion of the second subunit polypeptide of the TM receptor, wherein binding of the first affinity polypeptide to the first subunit polypeptide and of the second affinity polypeptide to the second subunit polypeptide induces oligomerization; and wherein oligomerization of the first subunit polypeptide and the second subunit polypeptide activates TM receptor signaling. In an additional embodiment of this aspect, the first affinity polypeptide binds directly to the first subunit polypeptide and/or wherein the second affinity polypeptide binds directly to the second subunit polypeptide; or wherein the first subunit polypeptide includes the tag polypeptide, and wherein the first affinity polypeptide binds to the tag polypeptide, and/or wherein the second subunit polypeptide includes the tag polypeptide, and wherein the second affinity polypeptide binds to the tag polypeptide.

In yet another embodiment of this aspect, which may be combined with any preceding embodiment that has a genetically modified plant or part thereof, the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, or a ubiquitin tag polypeptide. In a further embodiment of this aspect, the tag polypeptide is a fluorescent protein polypeptide selected from a green fluorescent protein (GFP) polypeptide, a red fluorescent polypeptide (RFP) (e.g., mCherry), or a blue fluorescent polypeptide (BFP); wherein the tag polypeptide is a luminescence polypeptide selected from a luciferase polypeptide; or wherein the tag polypeptide is a combination of a fluorescent protein polypeptide and a luminescence polypeptide. In still another embodiment of this aspect, which may be combined with any preceding embodiment that has a genetically modified plant or part thereof, the affinity polypeptide is selected from a heavy-chain variable domain (VHH), a single-chain variable fragment (scFV), a designed ankyrin repeat protein (DARPins), a human fibronectin III domain 3 monobody, an Affibody, or an anticalin, or a synthetic version of any of the foregoing. In a further embodiment of this aspect, the affinity polypeptide is a heavy-chain variable domain (VHH). In an additional embodiment of this aspect, the affinity polypeptide is a VHH_{LaG16}. In yet another embodiment of this aspect, which may be combined with any preceding embodiment that has a genetically modified plant or part thereof, the first subunit polypeptide and/or the second subunit polypeptide are selected from a LysM receptor, a Leucine rich repeat (LRR) receptor, a Malectin like receptor, a plant TM receptor, a plant SPTM (pSPTM) receptor, and a plant SPTM receptor including an intracellular kinase domain (pSPTM-kinase). In another embodiment of this aspect, the first subunit polypeptide and/or the second subunit polypeptide is a LysM receptor. In a further embodiment of this aspect, the first subunit polypeptide and/or the second subunit polypeptide lacks an ectodomain and/or a transmembrane domain. In yet another embodiment of this aspect, the first subunit polypeptide is a NFR1 polypeptide and the second subunit polypeptide is a NFR5 polypeptide, wherein the first subunit polypeptide is a NFR5 polypeptide and the second subunit is a NFR1 polypeptide, wherein the first subunit polypeptide is a LYK3 polypeptide and the second subunit polypeptide is a NFP polypeptide, wherein the first subunit polypeptide is a NFP polypeptide and the second subunit polypeptide is a LYK3 polypeptide, wherein the first subunit polypeptide is a RLK4 receptor polypeptide and the second subunit polypeptide is a RLK10 receptor polypeptide, or wherein the first subunit polypeptide is a RLK10 polypeptide and the second subunit polypeptide is a RLK4 polypeptide. In an additional embodiment of this aspect, the NFR1, LYK3, or RLK4 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 56, and wherein the NFR5, NFP, or RLK10 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 57. Additional suitable LysM receptor polypeptides may include RLK1, RLK2, RLK5, RLK7, CERK6, and SYMRK. In a further embodiment of this aspect, which may be combined with any preceding embodiment that has a genetically modified plant or part thereof, the TM receptor is localized to a plant cell membrane. In yet another embodiment of this aspect, which may be combined with any preceding embodiment that has a genetically modified plant or part thereof, the plant is selected from the group of cassava (e.g., manioc, yucca, Manihot esculenta), corn (e.g., maize, Zea mays), rice (e.g., indica rice, japonica rice, aromatic rice, glutinous rice, Oryza sativa, Oryza glaberrima), wild rice (e.g., Zizania spp., Porteresia spp.), barley (e.g., Hordeum vulgare), sorghum (e.g., Sorghum bicolor), millet (e.g., finger millet, fonio millet, foxtail millet, pearl millet, barnyard millets, Eleusine coracana, Panicum sumatrense, Panicum milaceum, Setaria italica, Pennisetum glaucum, Digitaria spp., Echinocloa spp.), teff (e.g., Eragrostis tej), oat (e.g., Avena sativa), triticale (e.g., X Triticosecale Wittmack, Triticosecale schlanstedtense Wittm., Triticosecale neoblaringhemii A. Camus, Triticosecale neoblaringhemii A. Camus), rye (e.g., Secale cereale, Secale cereanum), wheat (e.g., common wheat, spelt, durum, einkorn, emmer, kamut, Triticum aestivum, Triticum spelta, Triticum durum, Triticum urartu, Triticum monococcum, Triticum turanicum, Triticum spp.), Trema spp. (e.g., Trema cannabina, Trema cubense, Trema discolor, Trema domingensis, Trema integerrima, Trema lamarckiana, Trema micrantha, Trema orientalis, Trema philippinensis, Trema strigilosa, Trema tomentosa, Trema levigata), apple (e.g., Malus domestica, Malus pumila, Pyrus malus), pear (e.g., Pyrus communis, Pyrus x bretschneideri, Pyrus pyrifolia, Pyrus sinkiangensis, Pyrus pashia, Pyrus spp.), plum (e.g., Mirabelle, greengage, damson, Prunus domestica, Prunus salicina, Prunus mume), apricot (e.g., Prunus armeniaca, Prunus brigantine, Prunus mandshurica), peach (e.g., Prunus persica), almond (e.g., Prunus dulcis, Prunus amygdalus), walnut (e.g., Persian walnut, English walnut, black walnut, Juglans regia, Juglans nigra, Juglans cinerea, Juglans californica), strawberry (e.g., Fragaria x ananassa, Fragaria chiloensis, Fragaria virginiana, Fragaria vesca), raspberry (e.g., European red raspberry, black raspberry, Rubus idaeus L., Rubus occidentalis, Rubus strigosus), blackberry (e.g., evergreen blackberry, Himalayan blackberry, Rubus fruticosus, Rubus ursinus, Rubus laciniatus, Rubus argutus, Rubus armeniacus, Rubus plicatus, Rubus ulmifolius, Rubus allegheniensis, Rubus subgenus Eubatus sect. Moriferi & Ursini), red currant (e.g., white currant, Ribes rubrum), black currant (e.g., cassis, Ribes nigrum), gooseberry (e.g., Ribes uva-crispa, Ribes grossulari, Ribes hirtellum), melon (e.g., watermelon, winter melon, casabas, cantaloupe, honeydew, muskmelon, Citrullus lanatus, Benincasa hispida, Cucumis melo, Cucumis melo cantalupensis, Cucumis melo inodorus, Cucumis melo reticulatus), cucumber (e.g., slicing cucumbers, pickling cucumbers, English cucumber, Cucumis sativus), pumpkin (e.g., Cucurbita pepo, Cucurbita maxima), squash (e.g., gourd, Cucurbita argyrosperma, Cucurbita ficifolia, Cucurbita maxima, Cucurbita moschata), grape (e.g., Vitis vinifera, Vitis amurensis, Vitis labrusca, Vitis mustangensis, Vitis riparia, Vitis rotundifolia), bean (e.g., Phaseolus vulgaris, Phaseolus lunatus, Vigna angularis, Vigna radiate, Vigna mungo, Phaseolus coccineus, Vigna umbellate, Vigna acontifolia, Phaseolus acutifolius, Vicia faba, Vicia faba equine, Phaseolus spp., Vigna spp.), soybean (e.g., soy, soya bean, Glycine max, Glycine soja), pea (e.g., Pisum spp., Pisum sativum var. sativum, Pisum sativum var. arvense), pea (e.g., Pisum spp., Pisum sativum var. sativum, Pisum sativum var. arvense), chickpea (e.g., garbanzo, Bengal gram, Cicer arietinum), cowpea (e.g., Vigna unguiculata), pigeon pea (e.g., Arhar/Toor, cajan pea, Congo bean, gandules, Caganus cajan), lentil (e.g., Lens culinaris), Bambara groundnut (e.g., earth pea, Vigna subterranea), lupin (e.g., Lupinus spp.), pulses (e.g., minor pulses, Lablab purpureaus, Canavalia ensiformis, Canavalia gladiate, Psophocarpus tetragonolobus, Mucuna pruriens var. utilis, Pachyrhizus erosus), Medicago spp. (e.g., Medicago sativa, Medicago truncatula, Medicago arborea), Lotus spp. (e.g., Lotus japonicus), forage legumes (e.g., Leucaena spp., Albizia spp., Cyamopsis spp., Sesbania spp., Stylosanthes spp., Trifolium spp., Vicia spp.), indigo (e.g., Indigofera spp., Indigofera tinctoria, Indigofera suffruticosa, Indigofera articulata, Indigofera oblongifolia, Indigofera aspalthoides, Indigofera suffruticosa, Indigofera arrecta), legume trees (e.g., locust trees, Gleditsia spp., Robinia spp., Kentucky coffeetree, Gymnocladus dioicus, Acacia spp., Laburnum spp., Wisteria spp.), or hemp (e.g., cannabis, Cannabis sativa).

Further aspects of the present disclosure relate to methods of making the genetically modified plant of any of the preceding embodiments that have a genetically modified plant including a genetically modified cell including a TM receptor complex, including introducing a genetic alteration to the plant cell including a first nucleic acid sequence encoding a heterologous first subunit polypeptide including an affinity polypeptide; and/or introducing a genetic alteration to the plant cell including a second nucleic acid sequence encoding a heterologous second subunit polypeptide optionally including a tag polypeptide. In some embodiments, the methods include introducing a genetic alteration to the plant cell comprising a nucleic acid sequence encoding an affinity polypeptide and/or introducing a genetic alteration to the plant cell comprising a nucleic acid sequence encoding a tag polypeptide such that these polypeptides are linked to the endogenous first or second subunit polypeptide in the correct location. In an additional embodiment of this aspect, the first nucleic acid sequence is operably linked to a promoter and/or wherein the second nucleic acid sequence is operably linked to a promoter. In a further embodiment of this aspect, the promoter is a root specific promoter, an inducible promoter, a constitutive promoter, or a combination thereof. In yet another embodiment of this aspect, which may be combined with any preceding embodiment that has a promoter, the promoter is a root specific promoter, and wherein the promoter is selected from the group of a NFR1 promoter, a NFR5/NFP promoter, a LYK3 promoter, a CERK6 promoter, a NFR5/NFP promoter, a Lotus japonicus NFR5 promoter (SEQ ID NO: 27), a Lotus japonicus NFR1 promoter (SEQ ID NO: 69), a Lotus japonicus CERK6 promoter (SEQ ID NO: 46), a Medicago truncatula NFP promoter (SEQ ID NO: 29), a Medicago truncatula LYK3 promoter (SEQ ID NO: 28), a maize metallothionein promoter, a chitinase promoter, a maize ZRP2 promoter, a tomato LeExtl promoter, a glutamine synthetase soybean root promoter, a RCC3 promoter, a rice antiquitin promoter, a LRR receptor kinase promoter, or an Arabidopsis pCO2 promoter. In still another embodiment of this aspect, which may be combined with any preceding embodiment that has a promoter, the promoter is a constitutive promoter, and wherein the promoter is selected from the group of a CaMV35S promoter, a derivative of the CaMV35S promoter, a maize ubiquitin promoter, a polyubiquitin promoter, a vein mosaic cassava virus promoter, or an Arabidopsis UBQ10 promoter. In a further embodiment of this aspect, the first nucleic acid sequence is inserted into the genome of the plant so that the nucleic acid sequence is operably linked to an endogenous promoter, and/or wherein the second nucleic acid sequence is inserted into the genome of the plant so that the nucleic acid sequence is operably linked to an endogenous promoter. In an additional embodiment of this aspect, the endogenous promoter is a root specific promoter.

Additional aspects of the present disclosure relate to methods of making the genetically modified plant of any of the preceding embodiments that have a genetically modified plant including a genetically modified cell including a TM receptor complex, including genetically modifying the plant cell by transforming the plant cell with one or more gene editing components that target a first endogenous nuclear genome sequence encoding the first subunit polypeptide, wherein the first subunit polypeptide is genetically modified to include the affinity polypeptide; and/or genetically modifying the plant cell by transforming the plant cell with one or more gene editing components that target a second endogenous nuclear genome sequence encoding the second subunit polypeptide, wherein the endogenous second subunit polypeptide is genetically modified to include a tag polypeptide. In another embodiment of this aspect, the one or more gene editing components include a ribonucleoprotein complex that targets the first and/or second nuclear genome sequence; a vector including a TALEN protein encoding sequence, wherein the TALEN protein targets the first and/or second nuclear genome sequence; a vector including a ZFN protein encoding sequence, wherein the ZFN protein targets the first and/or second nuclear genome sequence; an oligonucleotide donor (OND), wherein the OND targets the first and/or second nuclear genome sequence; or a vector CRISPR/Cas enzyme encoding sequence and a targeting sequence, wherein the targeting sequence targets the first and/or second nuclear genome sequence. In a further embodiment of this aspect, genetically modifying the first subunit to include the affinity polypeptide includes inserting a first nucleic acid sequence encoding a heterologous first subunit polypeptide including an affinity polypeptide into the first endogenous nuclear genome sequence; and wherein genetically modifying the second subunit to include the tag polypeptide includes inserting a second nucleic acid sequence encoding a heterologous second subunit polypeptide including a tag polypeptide into the second endogenous nuclear genome sequence.

Further aspects of the present disclosure relate to methods of making the genetically modified plant or part thereof of any one of the preceding embodiments that have a genetically modified plant including a genetically modified cell including a bispecific affinity polypeptide, including introducing a genetic alteration to the plant cell including a first nucleic acid sequence encoding a heterologous first subunit polypeptide including an affinity polypeptide; and/or introducing a genetic alteration to the plant cell including a second nucleic acid sequence encoding a heterologous second subunit polypeptide including a tag polypeptide. In a further embodiment of this aspect, the first nucleic acid sequence is operably linked to a promoter and/or wherein the second nucleic acid sequence is operably linked to a promoter. In an additional embodiment of this aspect, the promoter is a root specific promoter, a constitutive promoter, or a combination thereof. In yet another embodiment of this aspect, which may be combined with any of the preceding embodiments that has a promoter, the promoter is a root specific promoter, and wherein the promoter is selected from the group of a NFR1 promoter, a NFR5 promoter, a LYK3 promoter, a CERK6 promoter, a NFP promoter, a Lotus japonicus NFR5 promoter (SEQ ID NO: 27), a Lotus japonicus NFR1 promoter (SEQ ID NO: 69), a Lotus japonicus CERK6 promoter (SEQ ID NO: 46), a Medicago truncatula NFP promoter (SEQ ID NO: 29), a Medicago truncatula LYK3 promoter (SEQ ID NO: 28), a maize metallothionein promoter, a chitinase promoter, a maize ZRP2 promoter, a tomato LeExtl promoter, a glutamine synthetase soybean root promoter, a RCC3 promoter, a rice antiquitin promoter, a LRR receptor kinase promoter, or an Arabidopsis pCO2 promoter. In still another embodiment of this aspect, which may be combined with any of the preceding embodiments that has a promoter, the promoter is a constitutive promoter, and wherein the promoter is selected from the group of a CaMV35S promoter, a derivative of the CaMV35S promoter, a maize ubiquitin promoter, a polyubiquitin promoter, a vein mosaic cassava virus promoter, or an Arabidopsis UBQ10 promoter. In another embodiment of this aspect, the first nucleic acid sequence is inserted into the genome of the plant so that the nucleic acid sequence is operably linked to an endogenous promoter, and/or wherein the second nucleic acid sequence is inserted into the genome of the plant so that the nucleic acid sequence is operably linked to an endogenous promoter. In a further embodiment of this aspect, the endogenous promoter is a root specific promoter.

Additional aspects of the present disclosure relate to methods of making the genetically modified plant or part thereof of any one of the preceding embodiments that have a genetically modified plant including a genetically modified cell including a bispecific affinity polypeptide, including genetically modifying the plant cell by transforming the plant cell with one or more gene editing components that target a first nuclear genome sequence encoding an endogenous first subunit polypeptide, wherein the endogenous first subunit polypeptide is genetically modified to include an affinity polypeptide; and/or genetically modifying the plant cell by transforming the plant cell with one or more gene editing components that target a second nuclear genome sequence encoding an endogenous second subunit polypeptide to include a tag polypeptide, wherein the endogenous second subunit polypeptide is genetically modified to include an affinity polypeptide. In a further embodiment of this aspect, the one or more gene editing components include a ribonucleoprotein complex that targets the first and/or second nuclear genome sequence; a vector including a TALEN protein encoding sequence, wherein the TALEN protein targets the first and/or second nuclear genome sequence; a vector including a ZFN protein encoding sequence, wherein the ZFN protein targets the first and/or second nuclear genome sequence; an oligonucleotide donor (OND), wherein the OND targets the first and/or second nuclear genome sequence; or a vector CRISPR/Cas enzyme encoding sequence and a targeting sequence, wherein the targeting sequence targets the first and/or second nuclear genome sequence.

Some aspects of the disclosure relate to a genetically modified plant or part thereof including the genetically modified plant cell of any of the preceding embodiments including a NFR1-NFR5 receptor complex. Another embodiment of this aspect further includes the NFR1-NFR5 receptor complex including the first subunit polypeptide and the second subunit polypeptide, wherein the first subunit polypeptide includes an affinity polypeptide that binds to the second subunit polypeptide inducing oligomerization; and wherein oligomerization of the first subunit polypeptide and the second subunit polypeptide activates NFR1-NFR5 receptor complex signaling. In an additional embodiment of this aspect, the affinity polypeptide binds directly to the second subunit polypeptide or wherein the second subunit polypeptide includes a tag polypeptide fused to an intracellular portion of the second subunit polypeptide, and wherein the affinity polypeptide binds to the tag polypeptide. In yet another embodiment of this aspect, which may be combined with any of the preceding embodiments, the genetically modified plant cell is selected from a root epidermal cell, a root cortex cell, a root endodermis cell, a root pericycle cell, a root primordia cell, a xylem cell, a phloem cell, a meristem cell, a leaf cell, a stem cell, a flower cell, or a fruit cell. In a further embodiment of this aspect, the genetically modified plant cell is a root epidermal cell, a root cortex cell, a root endodermis cell, a root pericycle cell, or a root primordia cell.

Further aspects of the disclosure relate to a genetically modified plant or part thereof including the bispecific affinity polypeptide of any of the preceding embodiments including a NFR1-NFR5 receptor complex. Another embodiment of this aspect further includes the first affinity polypeptide that binds to an intracellular portion of the NFR1 polypeptide and the second affinity polypeptide that binds to the intracellular portion of the NFR5 polypeptide, wherein binding of the first affinity polypeptide to the NFR1 polypeptide and of the second affinity polypeptide to the NFR5 polypeptide induces oligomerization; and wherein oligomerization of the NFR1 polypeptide and the NFR5 polypeptide activates NFR1-NFR5 receptor complex signaling. In a further embodiment of this aspect, the first affinity polypeptide binds directly to the NFR1 polypeptide and/or wherein the second affinity polypeptide binds directly to the NFR5 polypeptide; or wherein the NFR1 polypeptide includes a tag polypeptide fused to the intracellular portion of the NFR1 polypeptide, and wherein the first affinity polypeptide binds to the tag polypeptide, and/or wherein the NFR5 polypeptide includes a tag polypeptide fused to the intracellular portion of the NFR5 polypeptide, and wherein the second affinity polypeptide binds to the tag polypeptide. In yet another embodiment of this aspect, which may be combined with any of the preceding embodiments, the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, or a ubiquitin tag polypeptide. In a further embodiment of this aspect, the tag polypeptide is a fluorescent protein polypeptide selected from a green fluorescent protein (GFP) polypeptide, a red fluorescent polypeptide (RFP) (e.g., mCherry), or a blue fluorescent polypeptide (BFP); wherein the tag polypeptide is a luminescence polypeptide selected from a luciferase polypeptide; or wherein the tag polypeptide is a combination of a fluorescent protein polypeptide and a luminescence polypeptide. In still another embodiment of this aspect, which may be combined with any of the preceding embodiments, the affinity polypeptide is selected from a heavy-chain variable domain (VHH), a single-chain variable fragment (scFV), a designed ankyrin repeat protein (DARPins), a human fibronectin III domain 3 monobody, an Affibody, or an anticalin, or a synthetic version of any of the foregoing. In another embodiment of this aspect, the affinity polypeptide is a heavy-chain variable domain (VHH). In an additional embodiment of this aspect, the affinity polypeptide is a VHH_{LaG16}. In yet another embodiment of this aspect, which may be combined with any of the preceding embodiments, the Nfr1-nfr5 receptor complex is localized to a plant cell membrane. In a further embodiment of this aspect, which may be combined with any of the preceding embodiments, the plant part is a leaf, a stem, a root, a root primordia, a flower, a seed, a fruit, a kernel, a grain, a cell, or a portion thereof. In another embodiment of this aspect, which may be combined with any of the preceding embodiments, the plant is selected from the group of cassava (e.g., manioc, yucca, Manihot esculenta), corn (e.g., maize, Zea mays), rice (e.g., indica rice, japonica rice, aromatic rice, glutinous rice, Oryza sativa, Oryza glaberrima), wild rice (e.g., Zizania spp., Porteresia spp.), barley (e.g., Hordeum vulgare), sorghum (e.g., Sorghum bicolor), millet (e.g., finger millet, fonio millet, foxtail millet, pearl millet, barnyard millets, Eleusine coracana, Panicum sumatrense, Panicum milaceum, Setaria italica, Pennisetum glaucum, Digitaria spp., Echinocloa spp.), teff (e.g., Eragrostis tef), oat (e.g., Avena sativa), triticale (e.g., X Triticosecale Wittmack, Triticosecale schlanstedtense Wittm., Triticosecale neoblaringhemii A. Camus, Triticosecale neoblaringhemii A. Camus), rye (e.g., Secale cereale, Secale cereanum), wheat (e.g., common wheat, spelt, durum, einkorn, emmer, kamut, Triticum aestivum, Triticum spelta, Triticum durum, Triticum urartu, Triticum monococcum, Triticum turanicum, Triticum spp.), Trema spp. (e.g., Trema cannabina, Trema cubense, Trema discolor, Trema domingensis, Trema integerrima, Trema lamarckiana, Trema micrantha, Trema orientalis, Trema philippinensis, Trema strigilosa, Trema tomentosa, Trema levigata), apple (e.g., Malus domestica, Malus pumila, Pyrus malus), pear (e.g., Pyrus communis, Pyrus x bretschneideri, Pyrus pyrifolia, Pyrus sinkiangensis, Pyrus pashia, Pyrus spp.), plum (e.g., Mirabelle, greengage, damson, Prunus domestica, Prunus salicina, Prunus mume), apricot (e.g., Prunus armeniaca, Prunus brigantine, Prunus mandshurica), peach (e.g., Prunus persica), almond (e.g., Prunus dulcis, Prunus amygdalus), walnut (e.g., Persian walnut, English walnut, black walnut, Juglans regia, Juglans nigra, Juglans cinerea, Juglans californica), strawberry (e.g., Fragaria x ananassa, Fragaria chiloensis, Fragaria virginiana, Fragaria vesca), raspberry (e.g., European red raspberry, black raspberry, Rubus idaeus L., Rubus occidentalis, Rubus strigosus), blackberry (e.g., evergreen blackberry, Himalayan blackberry, Rubus fruticosus, Rubus ursinus, Rubus laciniatus, Rubus argutus, Rubus armeniacus, Rubus plicatus, Rubus ulmifolius, Rubus allegheniensis, Rubus subgenus Eubatus sect. Moriferi & Ursini), red currant (e.g., white currant, Ribes rubrum), black currant (e.g., cassis, Ribes nigrum), gooseberry (e.g., Ribes uva-crispa, Ribes grossulari, Ribes hirtellum), melon (e.g., watermelon, winter melon, casabas, cantaloupe, honeydew, muskmelon, Citrullus lanatus, Benincasa hispida, Cucumis melo, Cucumis melo cantalupensis, Cucumis melo inodorus, Cucumis melo reticulatus), cucumber (e.g., slicing cucumbers, pickling cucumbers, English cucumber, Cucumis sativus), pumpkin (e.g., Cucurbita pepo, Cucurbita maxima), squash (e.g., gourd, Cucurbita argyrosperma, Cucurbita ficifolia, Cucurbita maxima, Cucurbita moschata), grape (e.g., Vitis vinifera, Vitis amurensis, Vitis labrusca, Vitis mustangensis, Vitis riparia, Vitis rotundifolia), bean (e.g., Phaseolus vulgaris, Phaseolus lunatus, Vigna angularis, Vigna radiate, Vigna mungo, Phaseolus coccineus, Vigna umbellate, Vigna acontifolia, Phaseolus acutifolius, Vicia faba, Vicia faba equine, Phaseolus spp., Vigna spp.), soybean (e.g., soy, soya bean, Glycine max, Glycine soja), pea (e.g., Pisum spp., Pisum sativum var. sativum, Pisum sativum var. arvense), pea (e.g., Pisum spp., Pisum sativum var. sativum, Pisum sativum var. arvense), chickpea (e.g., garbanzo, Bengal gram, Cicer arietinum), cowpea (e.g., Vigna unguiculata), pigeon pea (e.g., Arhar/Toor, cajan pea, Congo bean, gandules, Caganus cajan), lentil (e.g., Lens culinaris), Bambara groundnut (e.g., earth pea, Vigna subterranea), lupin (e.g., Lupinus spp.), pulses (e.g., minor pulses, Lablab purpureaus, Canavalia ensiformis, Canavalia gladiate, Psophocarpus tetragonolobus, Mucuna pruriens var. utilis, Pachyrhizus erosus), Medicago spp. (e.g., Medicago sativa, Medicago truncatula, Medicago arborea), Lotus spp. (e.g., Lotus japonicus), forage legumes (e.g., Leucaena spp., Albizia spp., Cyamopsis spp., Sesbania spp., Stylosanthes spp., Trifolium spp., Vicia spp.), indigo (e.g., Indigofera spp., Indigofera tinctoria, Indigofera suffruticosa, Indigofera articulata, Indigofera oblongifolia, Indigofera aspalthoides, Indigofera suffruticosa, Indigofera arrecta), legume trees (e.g., locust trees, Gleditsia spp., Robinia spp., Kentucky coffeetree, Gymnocladus dioicus, Acacia spp., Laburnum spp., Wisteria spp.), or hemp (e.g., cannabis, Cannabis sativa).

Additional aspects of the disclosure relate to methods of making the genetically modified plant cell of any of the preceding embodiments including a NFR1-NFR5 receptor complex, including introducing a genetic alteration to the plant cell including a first nucleic acid sequence encoding a heterologous NFR1 polypeptide including an affinity polypeptide; and/or introducing a genetic alteration to the plant cell including a second nucleic acid sequence encoding a heterologous NFR5 polypeptide optionally including a tag polypeptide. In a further embodiment of this aspect, the NFR1 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 56, and wherein the NFR5 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 57. Additional suitable LysM receptor polypeptides may include RLK1, RLK2, RLK5, RLK7, CERK6, and SYMRK. In an additional embodiment of this aspect, which may be combined with any of the preceding embodiments, the first nucleic acid sequence is operably linked to a promoter and/or wherein the second nucleic acid sequence is operably linked to a promoter. In another embodiment of this aspect, the promoter is a root specific promoter, a constitutive promoter, or a combination thereof. In a further embodiment of this aspect, the promoter is a root specific promoter, and wherein the promoter is selected from the group of a NFR1 promoter, a NFR5/NFP promoter, a LYK3 promoter, a CERK6 promoter, a NFR5/NFP promoter, a Lotus japonicus NFR5 promoter (SEQ ID NO: 27), a Lotus japonicus NFR1 promoter (SEQ ID NO: 69), a Lotus japonicus CERK6 promoter (SEQ ID NO: 46), a Medicago truncatula NFP promoter (SEQ ID NO: 29), a Medicago truncatula LYK3 promoter (SEQ ID NO: 28), a maize metallothionein promoter, a chitinase promoter, a maize ZRP2 promoter, a tomato LeExtl promoter, a glutamine synthetase soybean root promoter, a RCC3 promoter, a rice antiquitin promoter, a LRR receptor kinase promoter, or an Arabidopsis pCO2 promoter. In yet another embodiment of this aspect, the promoter is a constitutive promoter, and wherein the promoter is selected from the group of a CaMV35S promoter, a derivative of the CaMV35S promoter, a maize ubiquitin promoter, a polyubiquitin promoter, a vein mosaic cassava virus promoter, or an Arabidopsis UBQ10 promoter. In still another embodiment of this aspect, the first nucleic acid sequence is inserted into the genome of the plant so that the nucleic acid sequence is operably linked to an endogenous promoter, and/or wherein the second nucleic acid sequence is inserted into the genome of the plant so that the nucleic acid sequence is operably linked to an endogenous promoter. In a further embodiment of this aspect, the endogenous promoter is a root specific promoter.

Further aspects of the disclosure relate to methods of making the genetically modified plant cell of any of the preceding embodiments including a NFR1-NFR5 receptor complex, including genetically modifying the plant cell by transforming the plant cell with one or more gene editing components that target a first nuclear genome sequence encoding an endogenous NFR1 polypeptide, wherein the endogenous NFR1 polypeptide is genetically modified to include an affinity polypeptide; and/or genetically modifying the plant cell by transforming the plant cell with one or more gene editing components that target a second nuclear genome sequence encoding an endogenous NFR5 polypeptide to include a tag polypeptide, wherein the endogenous NFR5 polypeptide is genetically modified to include a tag polypeptide. In a further embodiment of this aspect, the NFR1 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 56, and wherein the NFR5 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 57. Additional suitable LysM receptor polypeptides may include RLK1, RLK2, RLK5, RLK7, CERK6, and SYMRK. In an additional embodiment of this aspect, which may be combined with any of the preceding embodiments, the one or more gene editing components include a ribonucleoprotein complex that targets the first and/or second nuclear genome sequence; a vector including a TALEN protein encoding sequence, wherein the TALEN protein targets the first and/or second nuclear genome sequence; a vector including a ZFN protein encoding sequence, wherein the ZFN protein targets the first and/or second nuclear genome sequence; an oligonucleotide donor (OND), wherein the OND targets the first and/or second nuclear genome sequence; or a vector CRISPR/Cas enzyme encoding sequence and a targeting sequence, wherein the targeting sequence targets the first and/or second nuclear genome sequence. In yet another embodiment of this aspect, which may be combined with any of the preceding embodiments, genetically modifying the endogenous NFR1 polypeptide to include the affinity polypeptide includes inserting a first nucleic acid sequence encoding a heterologous NFR1 polypeptide including an affinity polypeptide into the endogenous NFR1 nuclear genome sequence; and wherein genetically modifying the endogenous NFR5 polypeptide to include the tag polypeptide includes inserting a second nucleic acid sequence encoding a heterologous NFR5 polypeptide including a tag polypeptide into the endogenous NFR5 nuclear genome sequence.

Yet further aspects of the disclosure relate to methods of making the genetically modified plant cell of any of the preceding embodiments including a NFR1-NFR5 receptor complex, including introducing a genetic alteration to the plant cell including a first nucleic acid sequence encoding a heterologous NFR1 polypeptide including an affinity polypeptide; and/or introducing a genetic alteration to the plant cell including a second nucleic acid sequence encoding a heterologous NFR5 polypeptide optionally including a tag polypeptide. In a further embodiment of this aspect, the NFR1 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 56, and wherein the NFR5 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 57. Additional suitable LysM receptor polypeptides may include RLK1, RLK2, RLK5, RLK7, CERK6, and SYMRK. In an additional embodiment of this aspect, which may be combined with any of the preceding embodiments, the first nucleic acid sequence is operably linked to a promoter and/or wherein the second nucleic acid sequence is operably linked to a promoter. In another embodiment of this aspect, the promoter is a root specific promoter, a constitutive promoter, or a combination thereof. In a further embodiment of this aspect, the promoter is a root specific promoter, and wherein the promoter is selected from the group of a NFR1 promoter, a NFR5/NFP promoter, a LYK3 promoter, a CERK6 promoter, a NFR5/NFP promoter, a Lotus japonicus NFR5 promoter (SEQ ID NO: 27), a Lotus japonicus NFR1 promoter (SEQ ID NO: 69), a Lotus japonicus CERK6 promoter (SEQ ID NO: 46), a Medicago truncatula NFP promoter (SEQ ID NO: 29), a Medicago truncatula LYK3 promoter (SEQ ID NO: 28), a maize metallothionein promoter, a chitinase promoter, a maize ZRP2 promoter, a tomato LeExtl promoter, a glutamine synthetase soybean root promoter, a RCC3 promoter, a rice antiquitin promoter, a LRR receptor kinase promoter, or an Arabidopsis pCO2 promoter. In yet another embodiment of this aspect, the promoter is a constitutive promoter, and wherein the promoter is selected from the group of a CaMV35S promoter, a derivative of the CaMV35S promoter, a maize ubiquitin promoter, a polyubiquitin promoter, a vein mosaic cassava virus promoter, or an Arabidopsis UBQ10 promoter. In still another embodiment of this aspect, the first nucleic acid sequence is inserted into the genome of the plant so that the nucleic acid sequence is operably linked to an endogenous promoter, and/or wherein the second nucleic acid sequence is inserted into the genome of the plant so that the nucleic acid sequence is operably linked to an endogenous promoter. In a further embodiment of this aspect, the endogenous promoter is a root specific promoter.

Further aspects of the present disclosure relate to methods of making the genetically modified plant of any of the preceding embodiments that have a genetically modified plant including the NFR1-NFR5 receptor complex and a bispecific affinity polypeptide, including introducing a genetic alteration to a plant cell including one or more nucleotide sequences encoding a bispecific affinity polypeptide including a first nucleic acid sequence encoding a first affinity polypeptide that binds to an intracellular portion of a NFR1 polypeptide, and a second nucleic acid sequence encoding a second affinity polypeptide that binds to an intracellular portion of a NFR5 polypeptide. In a further embodiment of this aspect, the first affinity polypeptide binds directly to the NFR1 polypeptide and/or wherein the second affinity polypeptide binds directly to the NFR5 polypeptide. In an additional embodiment of this aspect, the NFR1 polypeptide comprises a first tag polypeptide, and wherein the first affinity polypeptide binds to the first tag polypeptide, and/or wherein the NFR5 polypeptide comprises a second tag polypeptide, and wherein the second affinity polypeptide binds to the second tag polypeptide. In yet another embodiment of this aspect, the method further includes introducing a genetic alteration to a plant cell comprising a third nucleic acid sequence encoding a heterologous NFR1 polypeptide comprising a first tag polypeptide; and/or introducing a genetic alteration to the plant cell comprising a fourth nucleic acid sequence encoding a heterologous NFR5 polypeptide comprising a second tag polypeptide. In still another embodiment of this aspect, which may be combined with any preceding embodiment, the first nucleic acid, the second nucleic acid sequence, the third nucleic acid sequence, and/or the fourth nucleic acid sequence is operably linked to a promoter. In a further embodiment of this aspect, the promoter is a root specific promoter, an inducible promoter, a constitutive promoter, or a combination thereof. In an additional embodiment of this aspect, which may be combined with any preceding embodiment that has a promoter, the promoter is a root specific promoter, and wherein the promoter is selected from the group consisting of a NFR1 promoter, a NFR5 promoter, a LYK3 promoter, a CERK6 promoter, a NFP promoter, a Lotus japonicus NFR5 promoter (SEQ ID NO: 27), a Lotus japonicus NFR1 promoter (SEQ ID NO: 69), a Lotus japonicus CERK6 promoter (SEQ ID NO: 46), a Medicago truncatula NFP promoter (SEQ ID NO: 29), a Medicago truncatula LYK3 promoter (SEQ ID NO: 28), a maize metallothionein promoter, a chitinase promoter, a maize ZRP2 promoter, a tomato LeExtl promoter, a glutamine synthetase soybean root promoter, a RCC3 promoter, a rice antiquitin promoter, a LRR receptor kinase promoter, and an Arabidopsis pCO2 promoter. In yet another embodiment of this aspect, which may be combined with any preceding embodiment that has a promoter, the promoter is a constitutive promoter, and wherein the promoter is selected from the group consisting of a CaMV35S promoter, a derivative of the CaMV35S promoter, a maize ubiquitin promoter, a polyubiquitin promoter, a vein mosaic cassava virus promoter, and an Arabidopsis UBQ10 promoter. In still another embodiment of this aspect, the first nucleic acid sequence, the second nucleic acid sequence, the third nucleic acid sequence, and/or the fourth nucleic acid sequence is inserted into the genome of the plant so that the nucleic acid sequence is operably linked to an endogenous promoter. In a further embodiment of this aspect, the endogenous promoter is a root specific promoter.

Additional aspects of the present disclosure relate to methods of making the genetically modified plant of any of the preceding embodiments that have a genetically modified plant including the NFR1-NFR5 receptor complex and a bispecific affinity polypeptide, including genetically modifying the plant cell by transforming the plant cell with one or more gene editing components that target a first endogenous nuclear genome sequence, wherein the first endogenous nuclear genome sequence is genetically modified to insert bispecific affinity polypeptide comprising a first affinity polypeptide and a second affinity polypeptide, wherein the first affinity polypeptide binds to an intracellular portion of a NFR1 polypeptide, and wherein the second affinity polypeptide binds to an intracellular portion of a NFR5 polypeptide. In a further embodiment of this aspect, the first affinity polypeptide binds directly to the NFR1 polypeptide and/or wherein the second affinity polypeptide binds directly to the NFR5 polypeptide. In an additional embodiment of this aspect, the NFR1 polypeptide comprises a first tag polypeptide, and wherein the first affinity polypeptide binds to the first tag polypeptide, and/or wherein the NFR5 polypeptide comprises a second tag polypeptide, and wherein the second affinity polypeptide binds to the second tag polypeptide. In yet another embodiment of this aspect, the method further includes genetically modifying the plant cell by transforming the plant cell with one or more gene editing components that target a second endogenous nuclear genome sequence encoding the NFR1 polypeptide, wherein the NFR1 polypeptide is genetically modified to comprise the tag polypeptide; and/or genetically modifying the plant cell by transforming the plant cell with one or more gene editing components that target a third endogenous nuclear genome sequence encoding the NFR5 polypeptide, wherein the endogenous NFR5 polypeptide is genetically modified to comprise a tag polypeptide. In another embodiment of this aspect, the one or more gene editing components comprise a ribonucleoprotein complex that targets the first, second, and/or third nuclear genome sequence; a vector comprising a TALEN protein encoding sequence, wherein the TALEN protein targets the first, second, and/or third nuclear genome sequence; a vector comprising a ZFN protein encoding sequence, wherein the ZFN protein targets the first, second, and/or third nuclear genome sequence; an oligonucleotide donor (OND), wherein the OND targets the first, second, and/or third nuclear genome sequence; or a vector CRISPR/Cas enzyme encoding sequence and a targeting sequence, wherein the targeting sequence targets the first, second, and/or third nuclear genome sequence.

In some aspects, the present disclosure relates to a pollen grain or an ovule of the genetically altered plant of any of the above embodiments.

In some aspects, the present disclosure relates to a protoplast produced from the plant of any of the above embodiments.

In some aspects, the present disclosure relates to a tissue culture produced from protoplasts or cells from the plant of any of the above embodiments, wherein the cells or protoplasts are produced from a plant part selected from the group of leaf, anther, pistil, stem, petiole, root, root primordia, root tip, fruit, seed, flower, cotyledon, hypocotyl, embryo, or meristematic cell.

In certain embodiments, the plant part may be a seed, pod, fruit, leaf, flower, stem, root, any part of the foregoing or a cell thereof, or a non-regenerable part or cell of a genetically modified plant part. As used in this context, a “non-regenerable” part or cell of a genetically modified plant or part thereof is a part or cell that itself cannot be induced to form a whole plant or cannot be induced to form a whole plant capable of sexual and/or asexual reproduction. In certain embodiments, the non-regenerable part or cell of the plant part is a part of a transgenic seed, pod, fruit, leaf, flower, stem or root or is a cell thereof.

Processed plant products that contain a detectable amount of a nucleotide segment, expressed RNA, and/or protein comprising a genetic modification disclosed herein are also provided. Such processed products include, but are not limited to, plant biomass, oil, meal, animal feed, flour, flakes, bran, lint, hulls, and processed seed. The processed product may be non-regenerable. The plant product can comprise commodity or other products of commerce derived from a transgenic plant or transgenic plant part, where the commodity or other products can be tracked through commerce by detecting a nucleotide segment, expressed RNA, and/or protein that comprises distinguishing portions of a genetic modification disclosed herein.

A control as described herein can be a control sample or a reference sample from a wild-type, an azygous, or a null-segregant plant, species, or sample or from populations thereof. A reference value can be used in place of a control or reference sample, which was previously obtained from a wild-type, azygous, or null-segregant plant, species, or sample or from populations thereof or a group of a wild-type, azygous, or null-segregant plant, species, or sample. A control sample or a reference sample can also be a sample with a known amount of a detectable composition or a spiked sample.

### Expression Vectors or Isolated DNA Molecules, Cells or Kits Including the Same, and Related Methods

A further aspect of the present disclosure includes an expression vector or isolated DNA molecule including one or more nucleotide sequences encoding a first subunit polypeptide of a TM receptor including an affinity polypeptide, wherein the affinity polypeptide is heterologous to the first subunit polypeptide, and wherein the one or more nucleotide sequences are operably linked to at least one expression control sequence.

Yet another aspect of the present disclosure includes an expression vector or isolated DNA molecule including one or more nucleotide sequences encoding a second subunit polypeptide of a TM receptor optionally including a tag polypeptide, wherein the one or more nucleotide sequences are operably linked to at least one expression control sequence.

Still another aspect of the present disclosure includes an expression vector or isolated DNA molecule including one or more nucleotide sequences encoding: (a) a first subunit polypeptide of a transmembrane (TM) receptor complex including an affinity polypeptide, wherein the affinity polypeptide is heterologous to the first subunit polypeptide; and/or (b) a second subunit polypeptide of a TM receptor optionally including a tag polypeptide, wherein the one or more nucleotide sequences are operably linked to at least one expression control sequence.

Still another aspect of the present disclosure includes an expression vector or isolated DNA molecule including one or more nucleotide sequences encoding a bispecific affinity polypeptide including a first affinity polypeptide that binds to an intracellular portion of a first subunit polypeptide of a TM receptor and a second affinity polypeptide that binds to an intracellular portion of a second subunit polypeptide of a TM receptor, wherein the one or more nucleotide sequences are operably linked to at least one expression control sequence. In an additional embodiment of this aspect, the first affinity polypeptide binds directly to the first subunit polypeptide and/or wherein the second affinity polypeptide binds directly to the second subunit polypeptide. In yet another embodiment of this aspect, the first subunit polypeptide includes a tag polypeptide, and wherein the first affinity polypeptide binds to the tag polypeptide, and/or wherein the second polypeptide includes a tag polypeptide, and wherein the second affinity polypeptide binds to the tag polypeptide. In still another embodiment of this aspect, which may be combined with any preceding embodiments and aspects that have a tag polypeptide, the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, or a ubiquitin tag polypeptide. In a further embodiment of this aspect, the tag polypeptide is a fluorescent protein polypeptide selected from a green fluorescent protein (GFP) polypeptide, a red fluorescent polypeptide (RFP) (e.g., mCherry), or a blue fluorescent polypeptide (BFP); wherein the tag polypeptide is a luminescence polypeptide selected from a luciferase polypeptide; or wherein the tag polypeptide is a combination of a fluorescent protein polypeptide and a luminescence polypeptide. In yet another embodiment of this aspect, which may be combined with any preceding embodiments that have a first affinity polypeptide and a second affinity polypeptide, the first affinity polypeptide and the second affinity polypeptide are selected from a heavy-chain variable domain (VHH), a single-chain variable fragment (scFV), a designed ankyrin repeat protein (DARPins), a human fibronectin III domain 3 monobody, an Affibody, or an anticalin, or a synthetic version of any of the foregoing. In still another embodiment of this aspect, the first affinity polypeptide and/or the second affinity polypeptide is a heavy-chain variable domain (VHH). In a further embodiment of this aspect, the affinity polypeptide is a VHH_{LaG16}. In an additional embodiment of this aspect, which may be combined with any of the preceding embodiments and aspects that have an expression vector or isolated DNA molecule, the first subunit polypeptide and/or the second subunit polypeptide are selected from a LysM receptor, a Leucine rich repeat (LRR) receptor, a Malectin like receptor, a single-pass TM (SPTM) receptor, a plant TM receptor, a plant SPTM (pSPTM) receptor, a SPTM receptor including an intracellular kinase domain (SPTM-kinase), and a plant SPTM receptor including an intracellular kinase domain (pSPTM-kinase). In a further embodiment of this aspect, the first subunit polypeptide and/or the second subunit polypeptide is a LysM receptor. In another embodiment of this aspect, the first subunit polypeptide and/or the second subunit polypeptide lacks an ectodomain and/or a transmembrane domain. In still another embodiment of this aspect, which may be combined with any of the preceding embodiments and aspects that have a LysM receptor, the first subunit polypeptide is a NFR1 polypeptide and the second subunit polypeptide is a NFR5 polypeptide, wherein the first subunit polypeptide is a NFR5 polypeptide and the second subunit is a NFR1 polypeptide, wherein the first subunit polypeptide is a LYK3 polypeptide and the second subunit polypeptide is a NFP polypeptide, wherein the first subunit polypeptide is a NFP polypeptide and the second subunit polypeptide is a LYK3 polypeptide, wherein the first subunit polypeptide is a RLK4 receptor polypeptide and the second subunit polypeptide is a RLK10 receptor polypeptide, or wherein the first subunit polypeptide is a RLK10 polypeptide and the second subunit polypeptide is a RLK4 polypeptide. In yet another embodiment of this aspect, the NFR1, LYK3, or RLK4 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 56, and wherein the NFR5, NFP, or RLK10 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 57. Additional suitable LysM receptor polypeptides may include RLK1, RLK2, RLK5, RLK7, CERK6, and SYMRK. In an additional embodiment of this aspect, which may be combined with any of the preceding embodiments and aspects that have an expression vector or isolated DNA molecule, the at least one expression control sequence includes a promoter selected from the group of a root specific promoter, a constitutive promoter, or a combination thereof. In a further embodiment of this aspect, the promoter is a root specific promoter, and wherein the promoter is selected from the group of a NFR1 promoter, a NFR5/NFP promoter, a LYK3 promoter, a CERK6 promoter, a NFR5/NFP promoter, a Lotus japonicus NFR5 promoter (SEQ ID NO: 27), a Lotus japonicus NFR1 promoter (SEQ ID NO: 69), a Lotus japonicus CERK6 promoter (SEQ ID NO: 46), a Medicago truncatula NFP promoter (SEQ ID NO: 29), a Medicago truncatula LYK3 promoter (SEQ ID NO: 28), a maize metallothionein promoter, a chitinase promoter, a maize ZRP2 promoter, a tomato LeExtl promoter, a glutamine synthetase soybean root promoter, a RCC3 promoter, a rice antiquitin promoter, a LRR receptor kinase promoter, or an Arabidopsis pCO2 promoter. In another embodiment of this aspect, the promoter is a constitutive promoter, and wherein the promoter is selected from the group of a CaMV35S promoter, a derivative of the CaMV35S promoter, a maize ubiquitin promoter, a polyubiquitin promoter, a vein mosaic cassava virus promoter, or an Arabidopsis UBQ10 promoter.

Some aspects of the present disclosure relate to a bacterial cell or an Agrobacterium cell including the expression vector or isolated DNA molecule of any one of the preceding embodiments.

Additional aspects of the present disclosure relate to a genetically modified plant, plant part, plant cell, or seed including the expression vector or isolated DNA molecule of any one of the preceding embodiments.

Further aspects of the present disclosure relate to a kit including the expression vector or isolated DNA molecule of any one of the preceding embodiments of the bacterial cell or the Agrobacterium cell of the preceding embodiments.

Still further aspects of the present disclosure relate to methods of activating a target transmembrane (TM) receptor complex or inducing organogenesis including: introducing a genetic alteration via an expression vector or isolated DNA molecule of any one of the preceding embodiments to a cell. In an additional embodiment of this aspect, activating the target TM receptor complex or inducing organogenesis is in the absence of a native, an endogenous, or exogenous stimulus (e.g., Nod). In a further embodiment of this aspect, which may be combined with any preceding embodiments, the method further includes knocking out a native target TM receptor complex or subunits thereof in the cell. In still another embodiment of this aspect, which may be combined with any preceding embodiments, the cell is a plant cell.

A further aspect of the present disclosure includes an expression vector or isolated DNA molecule including one or more nucleotide sequences encoding a NFR1 polypeptide including an affinity polypeptide, wherein the affinity polypeptide is heterologous to the NFR1 polypeptide, wherein the one or more nucleotide sequences are operably linked to at least one expression control sequence.

Yet another aspect of the present disclosure includes an expression vector or isolated DNA molecule including one or more nucleotide sequences encoding a NFR5 polypeptide optionally including a tag polypeptide, wherein the one or more nucleotide sequences are operably linked to at least one expression control sequence.

Still another aspect of the present disclosure includes an expression vector or isolated DNA molecule including one or more nucleotide sequences encoding: (a) a NFR1 polypeptide including an affinity polypeptide, wherein the affinity polypeptide is heterologous to the first subunit polypeptide; and/or (b) a NFR5 polypeptide optionally including a tag polypeptide, wherein the one or more nucleotide sequences are operably linked to at least one expression control sequence.

An additional aspect of the present disclosure includes an expression vector or isolated DNA molecule including one or more nucleotide sequences encoding a bispecific affinity polypeptide including a first affinity polypeptide that binds to an intracellular portion of a NFR1 polypeptide and a second affinity polypeptide that binds to an intracellular portion of a NFR5 polypeptide, wherein the one or more nucleotide sequences are operably linked to at least one expression control sequence, and wherein the first affinity polypeptide is heterologous to the NFR1 polypeptide and the second affinity polypeptide is heterologous to the NFR5 polypeptide. In a further embodiment of this aspect, the first affinity polypeptide binds directly to the NFR1 polypeptide and/or wherein the second subunit polypeptide binds directly to the NFR5 polypeptide. In another embodiment of this aspect, which may be combined with any of the preceding embodiments, the NFR1 polypeptide includes a tag polypeptide, and wherein the first affinity polypeptide binds to the tag polypeptide and/or wherein the wherein the NFR5 polypeptide includes a tag polypeptide, and wherein second affinity polypeptide binds to the tag polypeptide. In a further embodiment of this aspect, which may be combined with any of the preceding embodiments and aspects, the NFR1 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 56, and wherein the NFR5 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 57. Additional suitable LysM receptor polypeptides may include RLK1, RLK2, RLK5, RLK7, CERK6, and SYMRK. In yet another embodiment of this aspect, which may be combined with any of the preceding embodiments and aspects including an expression vector or isolated DNA molecule, the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a luciferase tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, or a ubiquitin tag polypeptide. In a further embodiment of this aspect, the tag polypeptide is a fluorescent protein polypeptide selected from a green fluorescent protein (GFP) polypeptide, a red fluorescent polypeptide (RFP) (e.g., mCherry), or a blue fluorescent polypeptide (BFP); wherein the tag polypeptide is a luminescence polypeptide selected from a luciferase polypeptide; or wherein the tag polypeptide is a combination of a fluorescent protein polypeptide and a luminescence polypeptide. In still another embodiment of this aspect, which may be combined with any of the preceding embodiments, the affinity polypeptide is selected from a heavy-chain variable domain (VHH), a single-chain variable fragment (scFV), a designed ankyrin repeat protein (DARPins), a human fibronectin III domain 3 monobody, an Affibody, or an anticalin, or a synthetic version of any of the foregoing. In another embodiment of this aspect, the affinity polypeptide is a heavy-chain variable domain (VHH). In an additional embodiment of this aspect, the affinity polypeptide is a VHH_{LaG16}. In still another embodiment of this aspect, which may be combined with any of the preceding embodiments and aspects including an expression vector or isolated DNA molecule, the NFR1 polypeptide and/or the NFR5 polypeptide lacks an ectodomain and/or a transmembrane domain. In yet another embodiment of this aspect, which may be combined with any of the preceding embodiments and aspects including an expression vector or isolated DNA molecule, the at least one expression control sequence includes a promoter selected from the group of a root specific promoter, a constitutive promoter, or a combination thereof. In a further embodiment of this aspect, the promoter is a root specific promoter, and wherein the promoter is selected from the group of a NFR1 promoter, a NFR5/NFP promoter, a LYK3 promoter, a CERK6 promoter, a NFR5/NFP promoter, a Lotus japonicus NFR5 promoter (SEQ ID NO: 27), a Lotus japonicus NFR1 promoter (SEQ ID NO: 69), a Lotus japonicus CERK6 promoter (SEQ ID NO: 46), a Medicago truncatula NFP promoter (SEQ ID NO: 29), a Medicago truncatula LYK3 promoter (SEQ ID NO: 28), a maize metallothionein promoter, a chitinase promoter, a maize ZRP2 promoter, a tomato LeExtl promoter, a glutamine synthetase soybean root promoter, a RCC3 promoter, a rice antiquitin promoter, a LRR receptor kinase promoter, or an Arabidopsis pCO2 promoter. In yet another embodiment of this aspect, the promoter is a constitutive promoter, and wherein the promoter is selected from the group of a CaMV35S promoter, a derivative of the CaMV35S promoter, a maize ubiquitin promoter, a polyubiquitin promoter, a vein mosaic cassava virus promoter, or an Arabidopsis UBQ10 promoter.

Some aspects of the present disclosure relate to a bacterial cell or an Agrobacterium cell including the expression vector or isolated DNA molecule of any one of the preceding embodiments.

Additional aspects of the present disclosure relate to a genetically modified plant, plant part, plant cell, or seed including the expression vector or isolated DNA molecule of any one of the preceding embodiments.

Further aspects of the present disclosure relate to a kit including the expression vector or isolated DNA molecule of any one of the preceding embodiments of the bacterial cell or the Agrobacterium cell of the preceding embodiments.

Still further aspects of the present disclosure relate to methods of activating a target NFR1-NFR5 receptor complex or inducing organogenesis including: introducing a genetic alteration via an expression vector or isolated DNA molecule of any one of the preceding embodiments to a cell. In an additional embodiment of this aspect, activating the target NFR1-NFR5 receptor complex or inducing organogenesis is in the absence of a native, an endogenous, or exogenous stimulus (e.g., Nod). In a further embodiment of this aspect, which may be combined with any preceding embodiments, the method further includes knocking out a native target NFR1-NFR5 receptor complex or subunits thereof in the cell. In still another embodiment of this aspect, which may be combined with any preceding embodiments, the cell is a plant cell.

### Affinity Polypeptides

### Heavy-Chain Variable Domain (VHH)

A camelid heavy-chain variable domain (VHH) (also known as Nanobody®, Ablynx), also known as a single domain antibody (sdAb), single variable domain antibody or a single monomeric variable antibody domain, recombinant variable domain of a heavy-chain-only antibody, target-binding fragment of a monoclonal antibody, a polyclonal VHH fragment antibody, a miniature engineered antibody, or a multiple antigen-binding fragment, is an antibody fragment consisting of a single monomeric variable antibody domain. Like a whole antibody, the VHH is able to bind selectively to a specific antigen. With a molecular weight of only 12-15 kDa, VHHs are much smaller than common whole antibodies (150-160 kDa). A VHH is a peptide chain of about 110 amino acids in length, comprising one variable domain (VH) of a heavy chain antibody, or of a common IgG. Unlike whole antibodies, VHHs do not show complement system triggered cytotoxicity because they lack an Fc region. Camelid- and fish-derived VHHs are able to bind to hidden antigens that are not accessible to whole antibodies, for example to the active sites of enzymes. A VHH can be obtained by immunization of sharks or camelids with the desired antigen and subsequent isolation of the mRNA coding for heavy chain antibodies. Camelids are members of the biological family Camelidae, the only living family in the suborder Tylopoda. Camels, dromedaries, Bactrian Camels, llamas, alpacas, vicunas, and guanacos are in this group. Alternatively, VHHs can be synthesized or made by screening synthetic libraries.

“VHH domains”, also known as VHHs, VHH domains, VHH antibody fragments, and VHH antibodies, were originally described as the antigen binding immunoglobulin (variable) domain of “heavy chain antibodies” (i.e., of “antibodies devoid of light chains”; Hamers-Casterman et al. (1993) Nature 363: 446-448). The term “VHH domain” was chosen to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHHs, reference is made to the review article by Muyldermans (Reviews in J. Biotechnol. 74: 277-302, 2001), as well as to the following patent applications, which are mentioned as general background art: WO 94/04678, WO 95/04079, WO 96/34103; WO 94/25591, WO 99/37681, WO 00/40968, WO 00/43507, WO 00/65057, WO 01/40310, WO 01/44301, EP 1134231 and WO 02/48193; WO 97/49805, WO 01/21817, WO 03/035694, WO 03/054016 and WO 03/055527; WO 03/050531; WO 01/90190; WO 03/025020; WO 04/041867, WO 04/041862, WO 04/041865, WO 04/041863, WO 04/062551, WO 05/044858, WO 06/40153, WO 06/079372, WO 06/122786, WO 06/122787 and WO 06/122825. As described in these references, VHHs (in particular VHH sequences and partially humanized VHHs) can in particular be characterized by the presence of one or more “Hallmark residues” in one or more of the framework sequences. A further description of the VHHs, including humanization and/or camelization of VHHs, as well as other modifications, parts or fragments, derivatives or “VHH fusions”, multivalent constructs (including some non-limiting examples of linker sequences) and different modifications to increase the half-life of the VHHs and their preparations can be found, e.g., in WO 08/101985 and WO 08/142164.

### Protein-Protein Interaction Domains

An affinity polypeptide of the present disclosure may include any known and characterized protein-protein interaction domain. By way of example, the Protein Data Bank (PDB) includes thousands of domains that make stable dimers. One of these can be a coiled-coil domain

In addition, an AI-based pipeline may be used for developing artificial binders de novo (Watson J L, Juergens D, Bennett N R, Trippe B L, Yim J, Eisenach H E, Ahern W, Borst A J, Ragotte R J, Milles L F, Wicky B I M, Hanikel N, Pellock S J, Courbet A, Sheffler W, Wang J, Venkatesh P, Sappington I, Torres S V, Lauko A, De Bortoli V, Mathieu E, Ovchinnikov S, Barzilay R, Jaakkola T S, DiMaio F, Baek M, Baker D. De novo design of protein structure and function with RFdiffusion. Nature. 2023 August; 620(7976):1089-1100. doi: 10.1038/s41586-023-06415-8. Epub 2023 Jul. 11. PMID: 37433327; PMCID: PMC10468394).

As another example, an affinity polypeptide can be any polypeptide that enables any stable protein-protein dimerization (e.g., two proteins that bind). Protein dimerization by affinity polypeptides can be or can be derived from known dimers, known protein interactions, or proteins that bind in silico. As another example, an affinity polypeptide can covalently link two proteins.

### Tag Polypeptides

As used herein, the term “epitope tag” or “affinity tag” or “tag polypeptides” refers to a site on or fused to a target polypeptide (for example, an antigen, such as a protein, nucleic acid, carbohydrate or lipid) to which an affinity polypeptide (for example, a VHH, antibody, antibody fragment, or scaffold protein containing antibody binding regions) binds. As used herein, the term “paratope” refers to the region of an affinity polypeptide (for example, a VHH, antibody, antibody fragment, or scaffold protein containing antibody binding regions) which recognizes and binds to an epitope or epitope tag. Epitopes often include a chemically active surface grouping of molecules such as amino acids, polypeptides or sugar side chains and have specific three-dimensional structural characteristics as well as specific charge characteristics. Epitopes can be formed both from contiguous and/or juxtaposed noncontiguous residues (for example, amino acids, nucleotides, sugars, lipid moiety) of the target molecule. An epitope may include but is not limited to at least 3, at least 5 or 8-10 residues (for example, amino acids or nucleotides). An epitope may be less than 20 residues (for example, amino acids or nucleotides) in length, less than 15 residues or less than 12 residues. Two antibodies may bind the same epitope within an antigen if they exhibit competitive binding for the antigen. Unless explicitly denoted, a set of residues as an epitope does not exclude other residues from being part of the epitope for a particular antibody. Rather, the presence of such a set designates a minimal series (or set of species) of epitopes. Thus, a set of residues identified as an epitope designates a minimal epitope of relevance for the antigen, rather than an exclusive list of residues for an epitope on an antigen.

The term “tagged” refers to a target polypeptide fused to a tag polypeptide. The tag polypeptide has enough residues to provide an epitope against which an affinity polypeptide can be made, yet does not interfere with the activity of the polypeptide to which it is fused. The tag polypeptide is sufficiently unique so that the antibody or Nanobody® (Ablynx) there against does not substantially cross-react with other epitopes. Non-limiting examples of tag polypeptides include poly-histidine tag, poly-histidine-glycine tag, poly-arginine tag, poly-aspartate tag, poly-cysteine tag, poly-phenylalanine, c-myc tag, Herpes simplex virus glycoprotein D (gD) tag, FLAG tag, FLAG® tag, KT3 epitope tag, tubulin epitope tag, T7 gene 10 protein peptide tag, streptavidin tag, streptavidin binding peptide (SPB) tag, Strep-tag, Strep-tag II, albumin-binding protein (ABP) tag, alkaline phosphatase (AP) tag, bluetongue virus tag (B-tag), calmodulin binding peptide (CBP) tag, chloramphenicol acetyl transferase (CAT) tag, choline-binding domain (CBD) tag, chitin binding domain (CBD) tag, cellulose binding domain (CBP) tag, dihydrofolate reductase (DHFR) tag, galactose-binding protein (GBP) tag, maltose binding protein (MBP), glutathione-S-transferase (GST), Glu-Glu (EE) tag, the flu HA tag polypeptide, horseradish peroxidase (HRP) tag, NE-tag, HSV tag, ketosteroid isomerase (KSI) tag, KT3 tag, LacZ tag, luciferase tag, NusA tag, PDZ domain tag, AviTag, Calmodulin-tag, E-tag, S-tag, SBP-tag, Softag 1, Softag 3, TC tag, VSV-tag, Xpress tag, Isopeptag, SpyTag, SnoopTag, Profinity eXact tag, Protein C tag, 51-tag, S-tag, biotin-carboxy carrier protein (BCCP) tag, green fluorescent protein (GFP) tag, superfolder green fluorescent protein (sfGFP), mCherry, yellow fluorescent protein (YFP), red fluorescent protein (RFP), small ubiquitin-like modifier (SUMO) tag, tandem affinity purification (TAP) tag, HaloTag, Nus-tag, Thioredoxin-tag, Fc-tag, CYD tag, HPC tag, TrpE tag, ubiquitin tag, a VSV-G epitope tag derived from the Vesicular Stomatis viral glycoprotein, or a V5 tag derived from a small epitope (Pk) found on the P and V proteins of the paramyxovirus of simian virus 5 (SV5). Other tag polypeptides exist that are well known and routinely used in the art, and are embraced by the invention.

Existing synthetic libraries may be used to generate new tag polypeptides (McMahon C, Baier A S, Pascolutti R, Wegrecki M, Zheng S, Ong J X, Erlandson S C, Hilger D, Rasmussen S G F, Ring A M, Manglik A, Kruse A C. Yeast surface display platform for rapid discovery of conformationally selective nanobodies. Nat Struct Mol Biol. 2018 March; 25(3):289-296. doi: 10.1038/s41594-018-0028-6. Epub 2018 Feb. 12. PMID: 29434346; PMCID: PMC5839991; Binz H K, Stumpp M T, Forrer P, Amstutz P, Pluckthun A. Designing repeat proteins: well-expressed, soluble and stable proteins from combinatorial libraries of consensus ankyrin repeat proteins. J Mol Biol.
2003 Sep. 12; 332(2):489-503. doi: 10.1016/s0022-2836(03)00896-9. PMID: 12948497).

Those of ordinary skill in the art will recognize that the terms “tag polypeptide,” “affinity tag,” “epitope tag,” or “affinity acceptor tag” may be used interchangeably herein. As used herein, the term “tag polypeptide” refers to an amino acid sequence that permits the tagged polypeptide to be readily bound by an affinity polypeptide. A tag polypeptide is generally (but need not be) placed at or near the N- or C-terminus of a protein or polypeptide. In most cases, a tag polypeptide is joined to a protein or polypeptide using a genetically encoded translational fusion.

### Antibody or Antigen-Binding Fragment

An affinity polypeptide or tag polypeptide can be or comprise an antibody, an antigen-binding fragment, or a functional fragment or portion thereof (e.g., a VHH, a Fab, an scFv). In some embodiments, the antibody can be a monoclonal antibody, a chimeric antibody, a multivalent antibody, or a fragment or portion thereof. In some embodiments, the affinity polypeptide can be an agonist or an antagonist. In some embodiments, the affinity polypeptide can be synthetic.

Examples of antibodies or functional fragments thereof can be a multivalent antibody, a scFv-Fc (Sokolowska-Wedzina et al., Mol. Cancer Res. 15(8): 1040-1050, 2017), a VHH domain (Li et al., Immunol. Lett. 188:89-95, 2017), a VNAR domain (Hasler et al., Mol. Immunol. 75:28-37, 2016), a (scFv)2, a minibody, TriBi minibody, a monoclonal antibody, a humanized antibody, a chimeric antibody, a human or humanized monoclonal antibody (e.g., Hunter & Jones, Nat. Immunol. 16:448-457, 2015; Heo et al., Oncotarget 7(13): 15460-15473, 2016), a DVD-Ig (Wu et al., Nat. Biotechnolf. 25(11): 1290-1297, 2007; WO 08/024188; WO 07/024715), a dual-affinity re-targeting antibody (DART) (Tsai et al., Mol. Ther. Oncolytics 3: 15024, 2016), a triomab (Chelius et al., MAbs 2(3):309-3 19, 2010), a KiH IgG with a common LC (Kontermann et al., Drug Discovery Today 20(7):838-847, 2015), a crossmab (Regula et al., EMBO Mol. Med. 9(7):985, 2017), an ortho-Fab IgG (Kontermann et al., 2015), a 2-in-1-IgG (Kontermann et al., 2015), IgG-scFv (Cheal et al., Mol. Cancer Ther. 13(7): 1803-1812, 2014), an scFv2-Fc (Natsume et al., J. Biochem. 140(3):359-368, 2006), a bi-nanobody (Kontermann et al., 2015), a tandem antibody (Kontermann et al., 2015), a DART-Fe (Kontermann et al., 2015), a Legobody, a Fab-VHH, a scFv-HSA-scFv (Kontermann, 2015), a DNL-Fab3 (Kontermann, 2015), a DAF (two-in-one or four-in-one), a bispecific antibody, a triabody (Schoonooghe et al., BMC Biotechnol. 9:70, 2009), a tetrabody, scFv-Fc knobs-into-holes, a scFv-Fc-scFv, a (Fab′scFv)2, a V-IgG, a IvG-V, a dual V domain IgG, a heavy chain immunoglobulin or a camelid (Holt et al., Trends Biotechnol. 21(11):484-490, 2003), a heteroconjugate antibody (e.g., U.S. Pat. No. 4,676,980), a linear antibody (Zapata et al., Protein Eng. 8 (10: 1057-1062, 1995), a trispecific antibody (Tutt et al., J. Immunol. 147:60, 1991), a Fabs-in-Tandem immunoglobulin (WO 2015/103072), a humanized camelid antibody, a DutaMab, a DT-IgG, a knobs-in-holes common LC, a knobs-in-holes assembly, a charge pair antibody, a Fab-arm exchange antibody, a SEEDbody, a Triomab, a LUZ-Y, a Fcab, kk-body, an orthogonal Fab, a DVD-IgG, an IgG(H)-scFv, a scFv-(H)IgG, an IgG(L)-scFv, a scFv-(L)-IgG, an IgG (L,H)-Fe, an IgG(H)-V, a V(H)—IgG, an IgG(L)-V, a V(L)-IgG, a KIH IgG-scFab, a 2scFv-IgG, a IgG-2scFv, a scFv4-Ig, a zybody, a DVI-IgG, a nanobody (e.g., antibodies derived from Camelus bactriamus, Calelus dromaderius, Lama paccos) (U.S. Pat. No. 5,759,808; Stijlemans et al., J. Biol. Chem. 279:1256-1261, 2004; Dumoulin et al., Nature 424:783-788, 2003; Pleschberger et al., Bioconjugate Chem. 14:440-448, 2003), a nanobody-HSA, a bispecific diabody, a diabody (e.g., Poljak, Structure 2(12): 1121-1123, 1994; Hudson et al., J. Immunol. Methods 23(1-2): 177-189, 1999), a scDiabody (Cuesta et al., Trends in Biotechnol. 28(7):355-362, 2010), a scDiabody-CH3 (Sanz et al., Trends in Immunol. 25(2):85-91, 2004), a scDiabody-Fe, a diabody-Fe, a scDiabody-HSA, a Diabody-CH3, a TandAb (Reusch et al., mAbs 6(3):727-738, 2014), a Triple Body, a miniantibody, an scFv-CH3 KIH, a Fab-scFv, a scFv-CH-CL-scFv, a F(ab′)2-scFV2, a scFv-KIH, a Fab-scFv-Fe, a tetravalent HCAb, a tandem scFv-Fe, an intrabody (Huston et al., Human Antibodies 10(3-4): 127-142, 2001; Wheeler et al., Mol. Ther. 8(3):355-366, 2003; Stocks, Drug Discov. Today 9(22):960-966, 2004), a dock and lock bispecific antibody, an ImmTAC, an HSAbody, a tandem scFv, an IgG-IgG, a Cov-X-Body, a scFv1-PEG-scFv2, a synthetic polypeptide (e.g., DARPins) (Binz H K, Stumpp M T, Forrer P, Amstutz P, Pluckthun A. Designing repeat proteins: well-expressed, soluble and stable proteins from combinatorial libraries of consensus ankyrin repeat proteins. J Mol Biol. 2003 Sep. 12; 332(2):489-503. doi: 10.1016/s0022-2836(03)00896-9. PMID: 12948497), a fragment thereof, a portion thereof, or combinations thereof. An affinity polypeptide can be a synthetic polypeptide. For example, there are numerous synthetic polypeptide libraries that can be used for generating synthetic polypeptides (McMahon C, Baier A S, Pascolutti R, Wegrecki M, Zheng S, Ong J X, Erlandson S C, Hilger D, Rasmussen S G F, Ring A M, Manglik A, Kruse A C. Yeast surface display platform for rapid discovery of conformationally selective nanobodies. Nat Struct Mol Biol. 2018 March; 25(3):289-296. doi: 10.1038/s41594-018-0028-6. Epub 2018 Feb. 12. PMID: 29434346).

Additional examples of an antigen-binding fragment can be an Fv fragment, a Fab fragment, a F(ab′)2 fragment, or a Fab′ fragment. Additional examples of an antigen-binding fragment of an antibody can be an antigen-binding fragment of an IgG (e.g., an antigen-binding fragment of IgG1, IgG2, IgG3, or IgG4) (e.g., an antigen-binding fragment of a human, humanized IgG; human or humanized IgG1, IgG2, IgG3, or IgG4); an antigen-binding fragment of an IgA (e.g., an antigen-binding fragment of IgA1 or IgA2) (e.g., an antigen-binding fragment of a human or humanized IgA; a human or humanized IgA1 or IgA2); an antigen-binding fragment of an IgD (e.g., an antigen-binding fragment of a human, humanized IgD); an antigen-binding fragment of an IgE (e.g., an antigen-binding fragment of a human, humanized IgE); or an antigen-binding fragment of an IgM (e.g., an antigen-binding fragment of a human, humanized IgM). Additional examples of antibodies and antigen-binding fragments thereof are described in U.S. Pat. Nos. 8,440,196; 7,842,144; 8,034,344; and 8,529,895; US 2013/0317203; US 2014/0322239; US 2015/0166666; US 2016/0152714; and US 2017/0002082, each of which is incorporated by reference in its entirety.

### Affinity

An affinity polypeptide can have affinity for a polypeptide tag, a protein, such as a kinase or pseudokinase (e.g., NFR5), an intracellular domain of a protein, or an intracellular domain transmembrane receptor. A high-affinity can be any affinity value capable of activating the cognate receptor or having receptor-activating activity, e.g., via an intracellular domain interaction, wherein the activated receptor initiates nodule organogenesis. Increased nodule organogenesis can be measured by comparing organogenesis to a control or wild type cell, species, or plant.

In some embodiments, the affinity polypeptide has a dissociation constant (KD) of less than 1×10⁻⁵ M. In some embodiments, the affinity polypeptide has a dissociation constant of less than 0.5×10⁻⁵ M, less than 1×10⁻⁶ M, less than 0.5×10⁻⁶ M, less than 1×10⁻⁷ M, less than 0.5×10⁻⁷ M, less than 1×10⁻⁸ M, less than 0.5×10⁻⁸ M, less than 1×10⁻⁹ M, less than 0.5×10⁻⁹ M, less than 1×10⁻¹⁰ M, less than 0.5×10⁻¹⁰ M, less than 1×10⁻¹¹ M, less than 0.5×10⁻¹¹ M, less than 1×10⁻¹² M, less than 0.5×10⁻¹² M, less than 1×10⁻¹³ M, less than 0.5×10⁻¹³ M, less than 1×10⁻¹⁴ M, less than 0.5×10⁻¹⁴ M, or less than 1×10⁻¹⁵ M. Recitation of each of these discrete values is understood to include ranges between each value. Recitation of each range is understood to include discrete values within the range. In some embodiments, the affinity polypeptide has a KD of between about 1×10⁻¹⁵ M and about 1×10⁻⁵ M. In some embodiments, the affinity polypeptide has a KD of about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, about 0.5×10⁻⁹ M, about 1×10⁻¹⁰ M, about 0.5×10⁻¹⁰ M, about 1×10⁻¹¹ M, about 0.5×10⁻¹¹ M, about 1×10⁻¹² M, about 1×10⁻¹³M, about 1×10⁻¹⁴ M, or about 1×10⁻¹⁵ M. Recitation of each of these discrete values is understood to include ranges between each value. Recitation of each range is understood to include discrete values within the range.

In some embodiments, the affinity polypeptide has a K_{off} between about 1×10⁻⁶ s⁻¹ to about 1×10⁻³ s⁻¹. In some embodiments, the affinity polypeptide has a K_{off} of about 1×10⁻³ s⁻¹, about 0.5×10⁻³ s⁻¹, about 1×10⁻⁴ s⁻¹, about 0.5×10⁻⁴ s⁻¹, about 1×10⁻⁵ s⁻¹, about 0.5×10⁻⁵ s⁻¹, or about 1×10⁻⁶ s⁻¹. Recitation of each of these discrete values is understood to include ranges between each value. Recitation of each range is understood to include discrete values within the range.

In some embodiments, the affinity polypeptide has a Kₒₙ of between about 1×10⁻² M⁻¹s⁻¹ and about 1×10⁻⁶ M⁻¹s⁻¹. In some embodiments, the affinity polypeptide described herein has a Kₒₙ of about 1×10⁻⁶ M⁻¹s⁻¹, about 0.5×10⁻⁶ M⁻¹s⁻¹, about 1×10⁻⁵ M⁻¹s⁻¹, about 0.5×10⁻⁵ M⁻¹s⁻¹, about 1×10⁻⁴ M⁻¹s⁻¹, about 0.5×10⁻⁴ M⁻¹s⁻¹, about 1×10⁻³ M⁻¹s⁻¹, about 0.5×10⁻³ M⁻¹s⁻¹, or 1×10⁻² M⁻¹s⁻¹. Recitation of each of these discrete values is understood to include ranges between each value. Recitation of each range is understood to include discrete values within the range.

### Transmembrane Receptor Complexes

Transmembrane receptor complexes can be synthetically activated by methods described herein. For example, it was discovered that some transmembrane receptor complexes can be activated by intracellular domain interactions between two or more intracellular domains of transmembrane proteins. These intracellular domain interactions can be initiated by exogenous or endogenous signals. As shown herein, the intracellular domain interactions can be synthetically achieved by using affinity polypeptides to mimic an interaction induced by endogenous signaling (e.g., Nod) such that in the absence of endogenous signals (e.g., Nod), the receptor is activated to induce a response (e.g., nodule organogenesis).

Examples of transmembrane receptor complexes can comprise two or more integral membrane proteins. For example an integral membrane protein can be a single-pass transmembrane (SPTM) protein, a single-pass α-helix or a bitopic membrane protein, a polytopic transmembrane α-helical protein or helical bundle, or a polytopic transmembrane f-sheet protein or f-barrel. Integral polytopic proteins are transmembrane proteins that span across the membrane more than once. These proteins can have different transmembrane topology. These proteins can have one of two structural architectures: helix bundle proteins, which are present in all types of biological membranes or beta barrel proteins, which can be found in outer membranes of Gram-negative bacteria, or outer membranes of mitochondria or chloroplasts. Bitopic proteins are transmembrane proteins that span across the membrane only once. Transmembrane helices from these proteins have significantly different amino acid distributions to transmembrane helices from polytopic proteins. Integral monotopic proteins are integral membrane proteins that are attached to only one side of the membrane and do not span the whole way across.

### LysM Receptors and NFR1-NFR5

LysM receptors may be defined as proteins that contain an N terminal signal peptide followed by 3 tandem LysM domains. These LysM domains are flanked by conserved CXXXC or CXC motifs. They can be followed by a membrane anchor domain (LYM receptors), or by a transmembrane and an intracellular kinase domain (LysM-RLK). Most plant LysM receptors contain an intracellular kinase, while some, including NFR1 and NFR5, contain an intracellular pseudokinase.

Lohmann et al., 2010 presents in detail the characteristics of NFR1-type and NFR5-type receptors (Lohmann G V, Shimoda Y, Nielsen M W, Jergensen F G, Grossmann C, Sandal N, Serensen K, Thirup S, Madsen L H, Tabata S, Sato S, Stougaard J, Radutoiu S. Evolution and regulation of the Lotus japonicus LysM receptor gene family. Mol Plant Microbe Interact. 2010; 23(4):510-21). In short, NFR1-type receptors are translated from multi-exon genes, while NFR5-type receptors are translated from single-exon genes. NFR1-type receptors contain all the subdomains typical for an intracellular kinase, while NFR5-type receptors contain intracellular pseudokinases where subdomain I and VII/VIII are missing, or the conserved residues are substituted. NFR5-type receptors may also be referred to as NFP-type receptors or Lys11-type receptors, as each of Lotus japonicus NFR5, Medicago truncatula NFP, and Lotus japonicus Lys11 belong to this receptor type. NFR1-type receptors may also be referred to as LYK3-type receptors, as Lotus japonicus NFR1 and Medicago truncatula LYK3 both belong to this receptor type.

LysM receptors have three characteristic domains located in the ectodomain of the protein: LysM1, LysM2, and LysM3, which are present in this order on the protein sequence and separated by CxC motifs. The LysM1 domain is located toward the N-terminal end of the protein sequence, and is preceded by an N-terminal signal peptide.

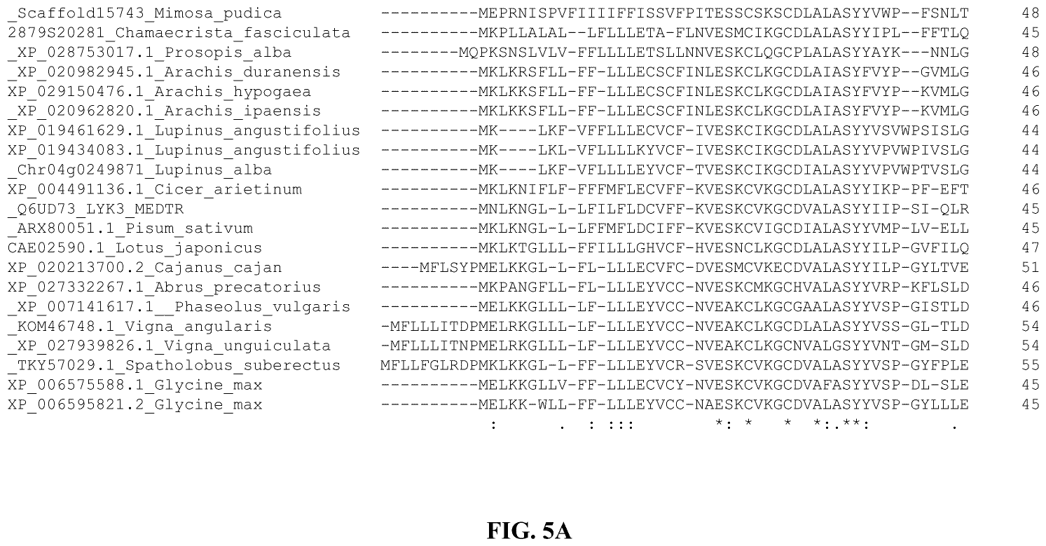

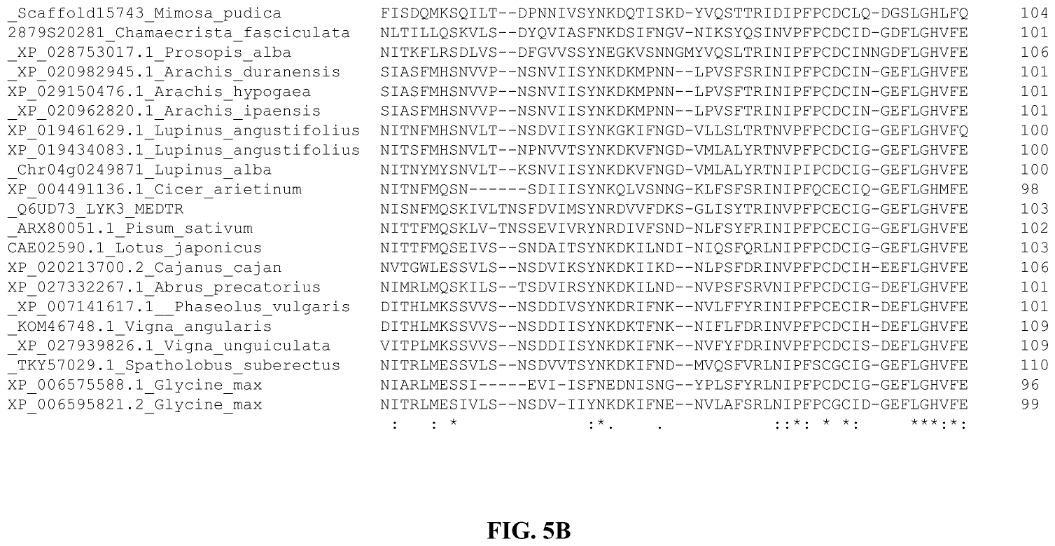

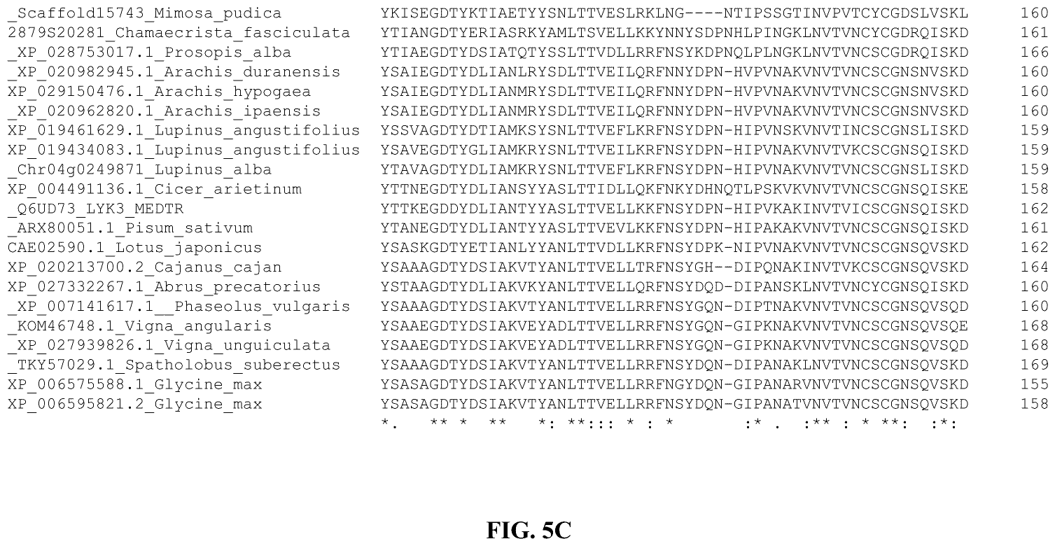

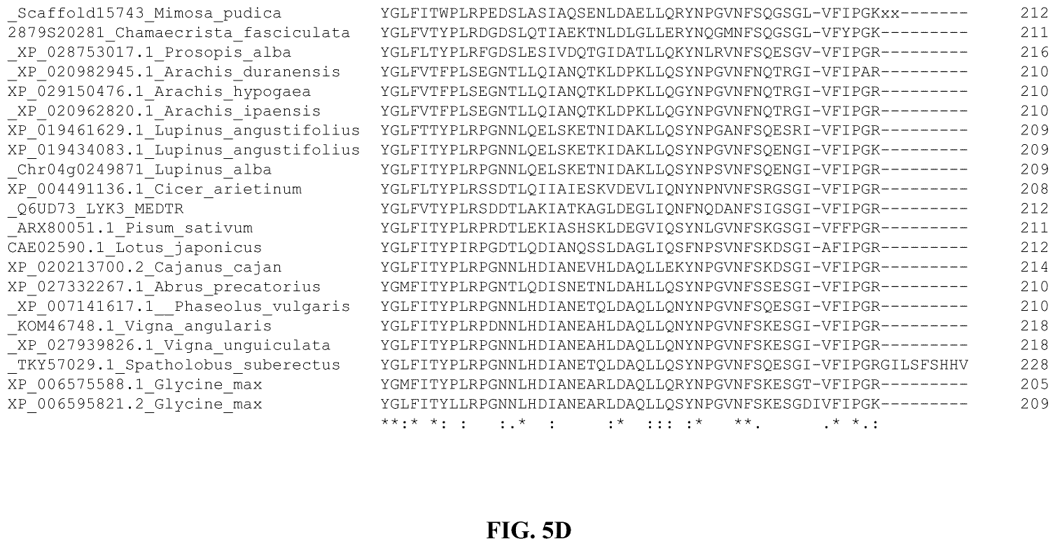

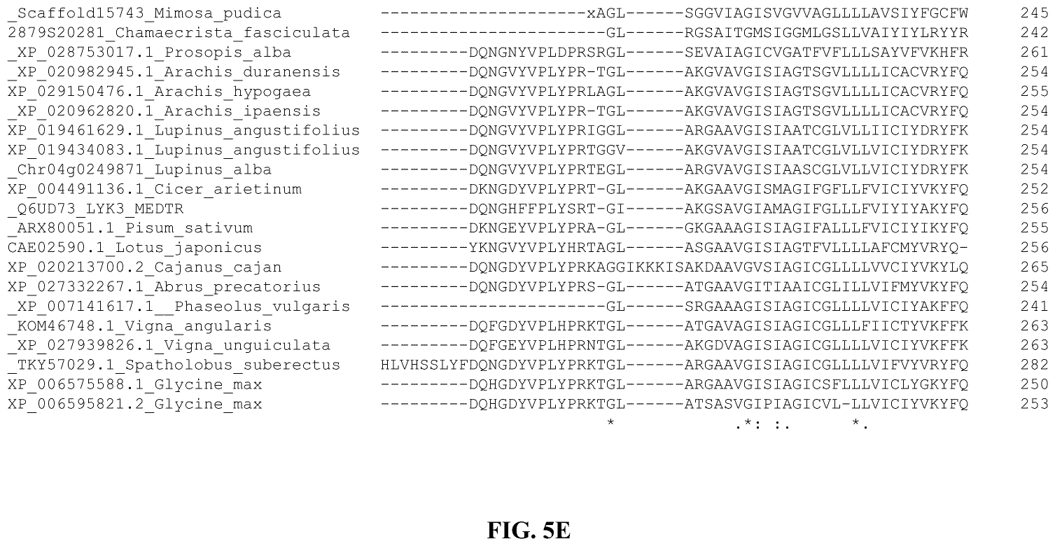

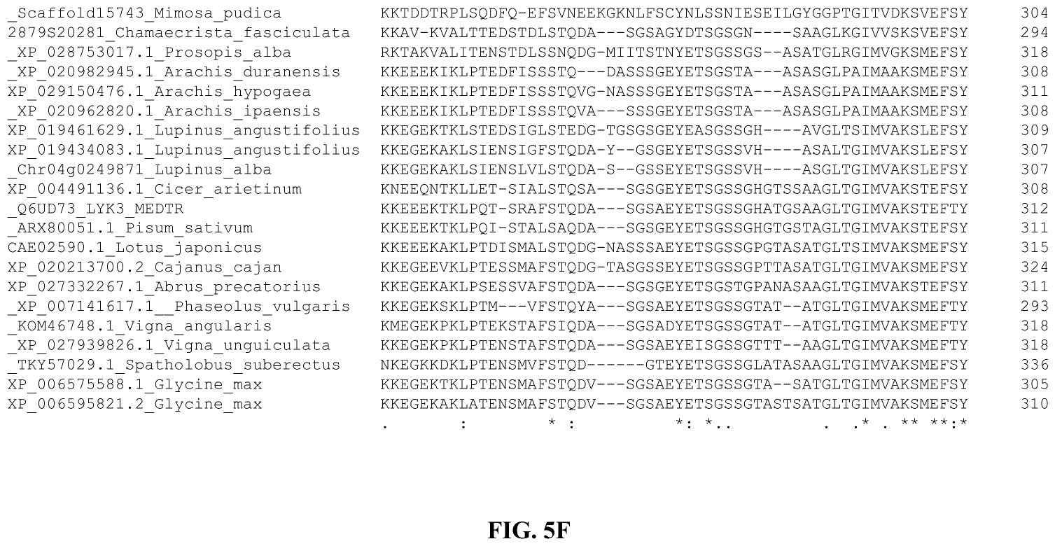

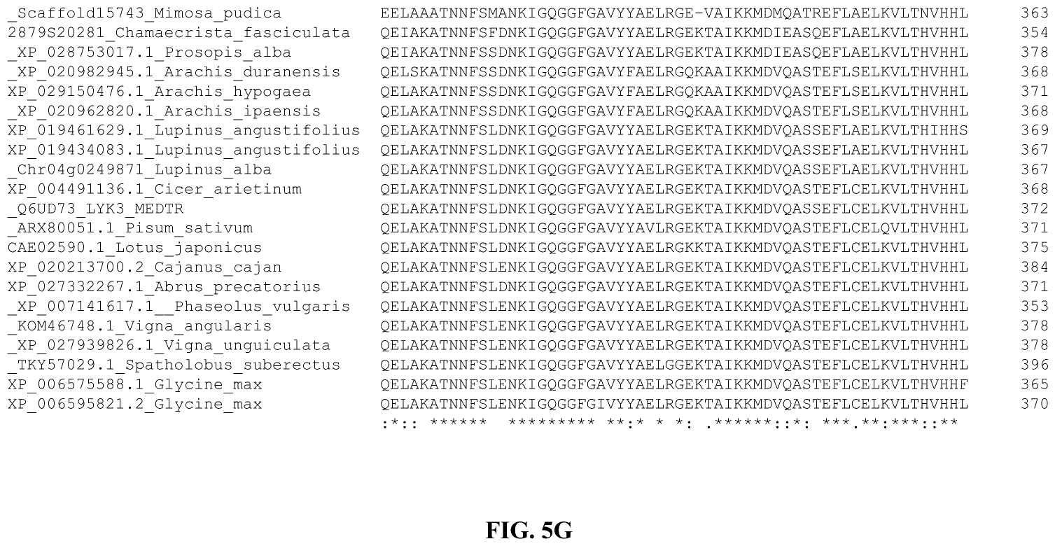

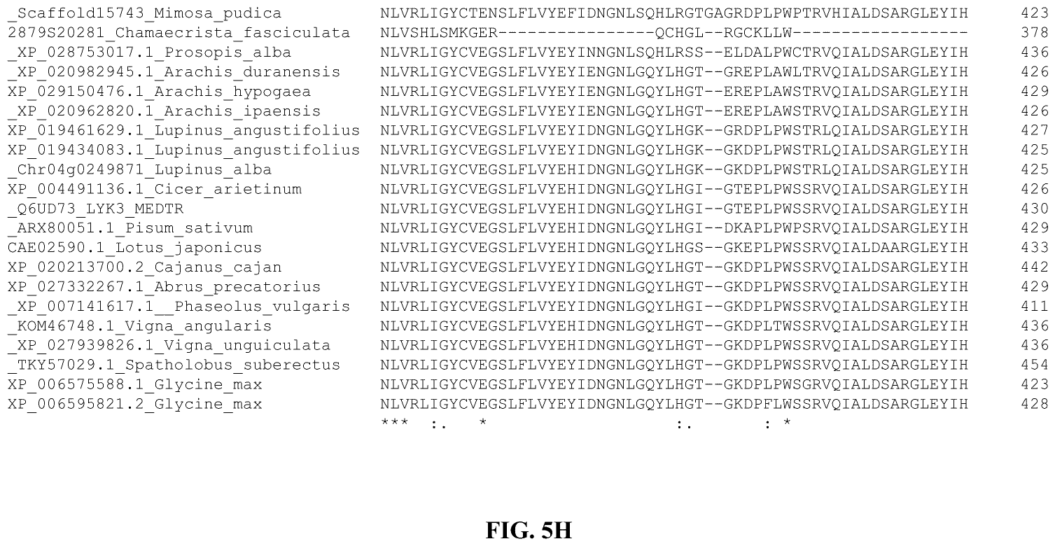

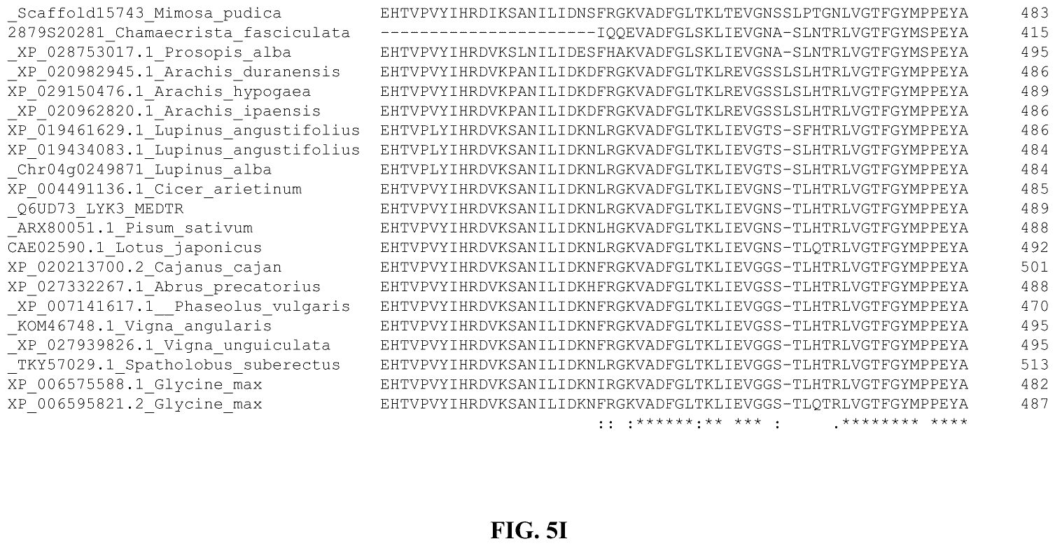

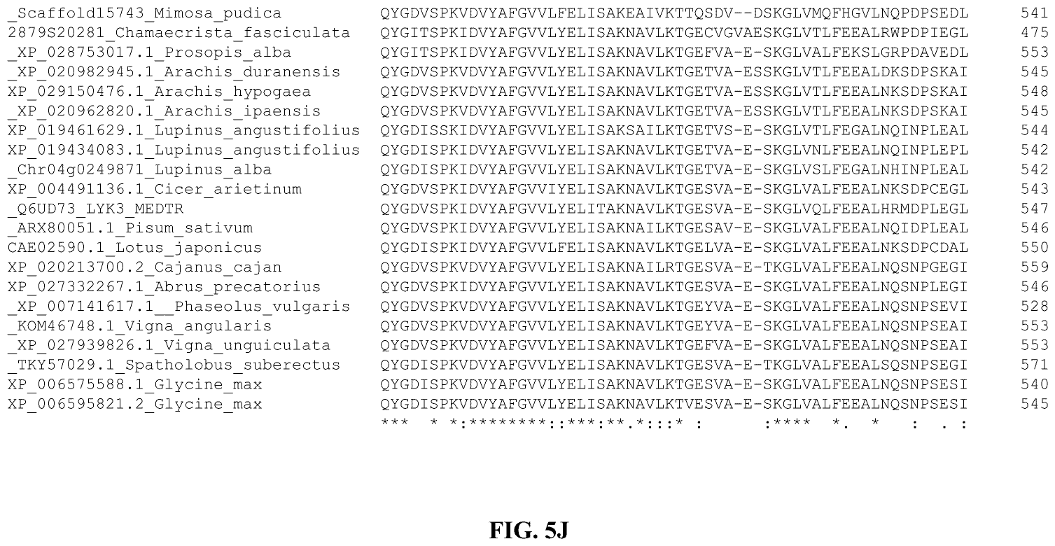

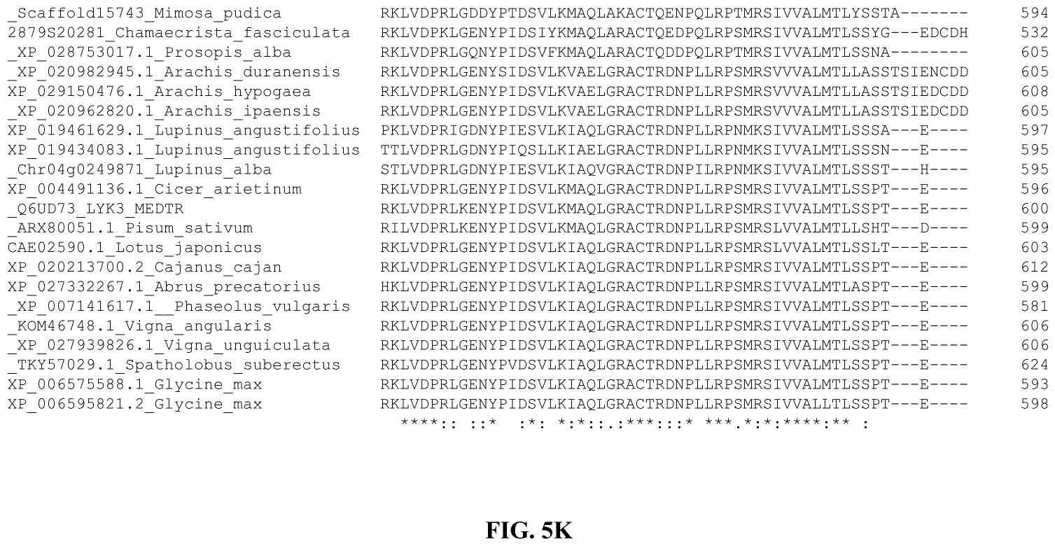

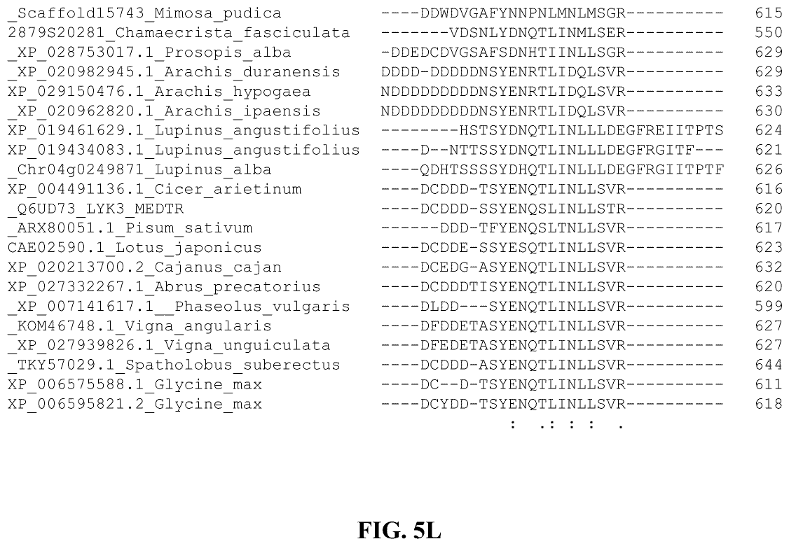

An alignment of NFR1-type LysM receptors is shown in FIGS. 5A-5L. Exemplary NFR1-type LysM Nod factor receptors include SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 56.

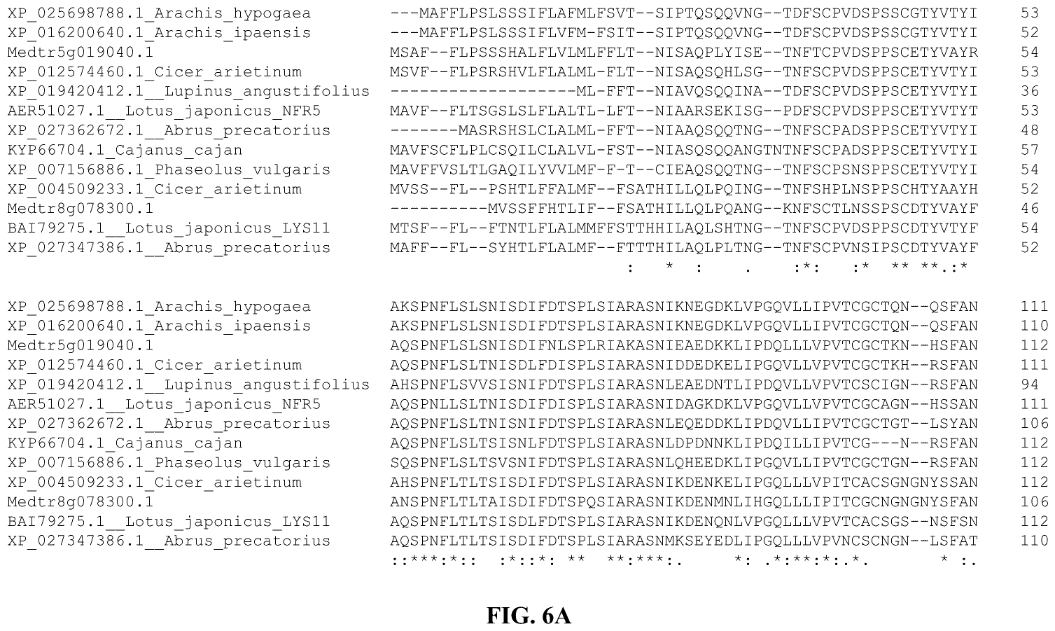

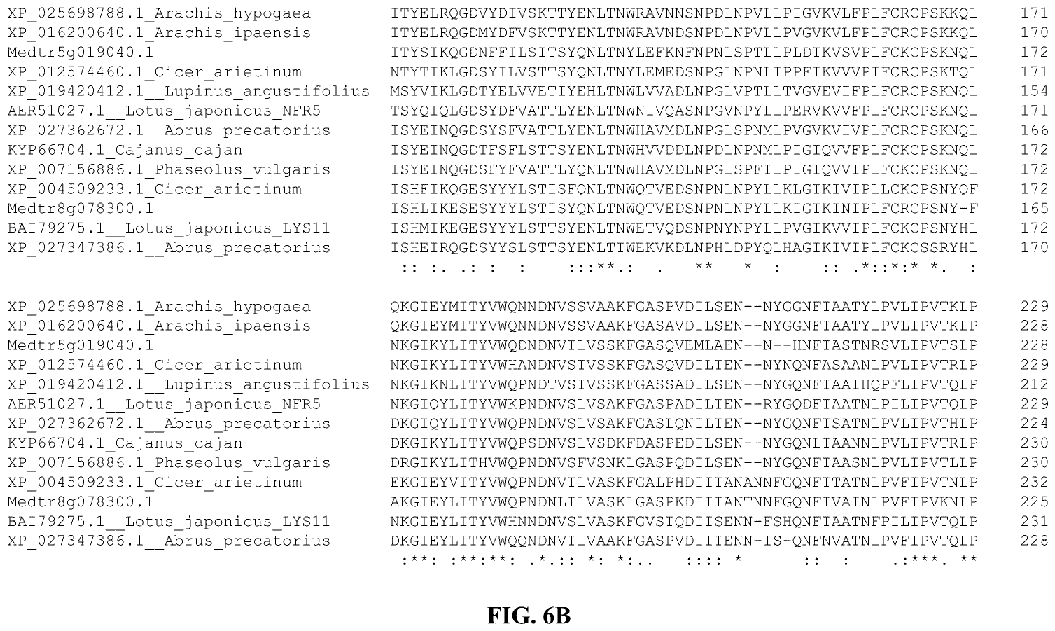

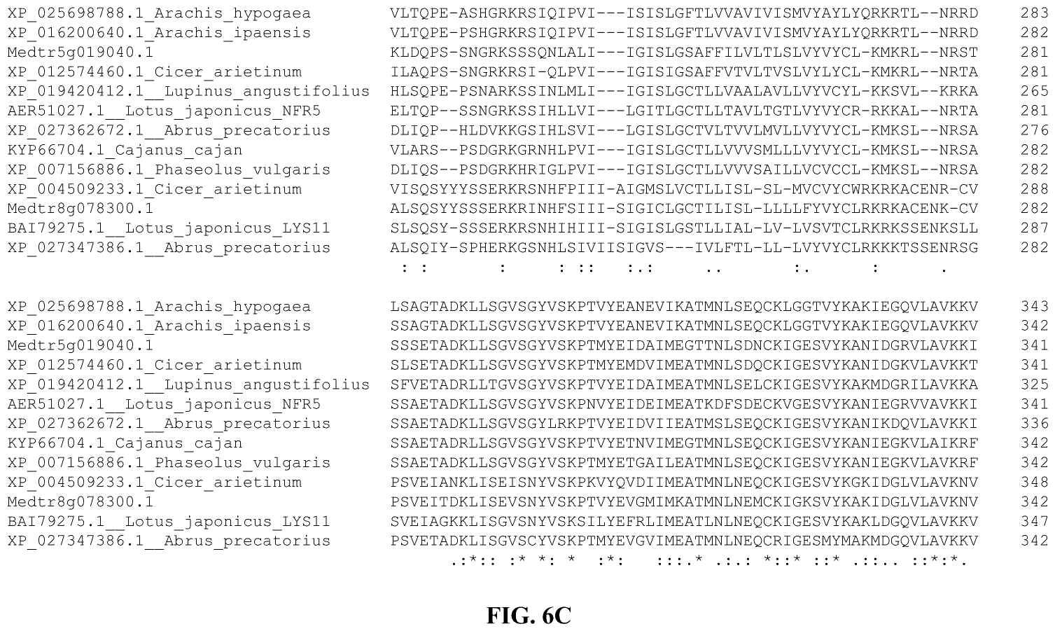

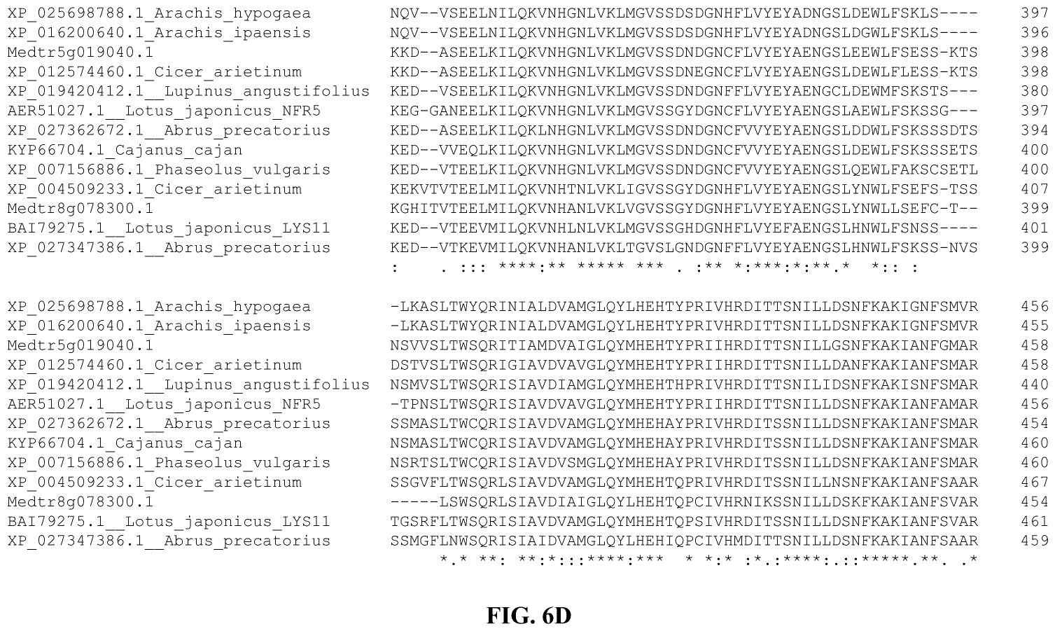

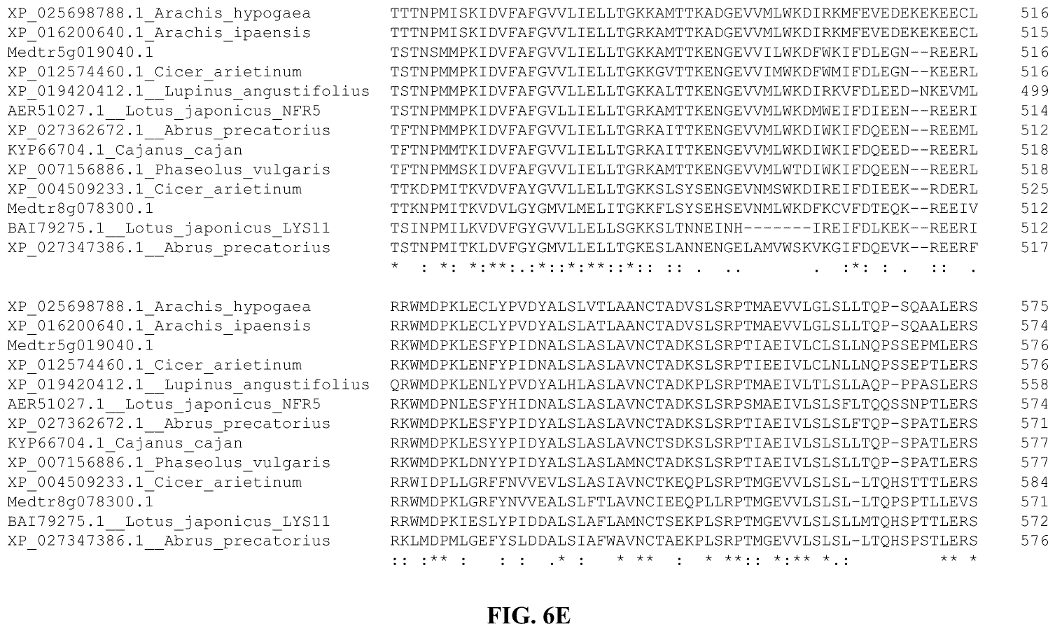

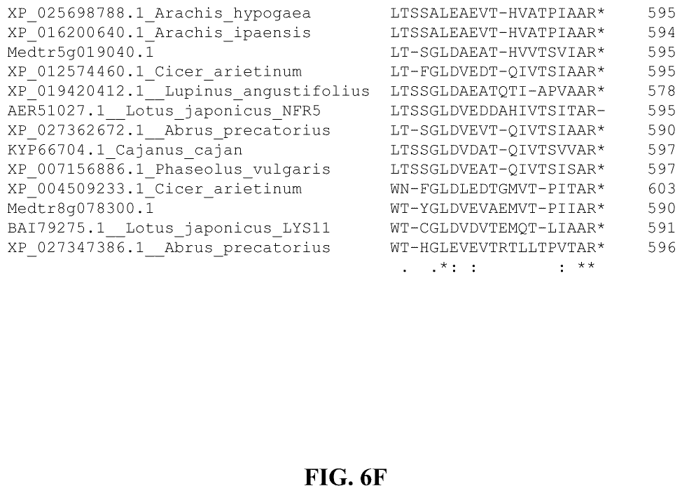

An alignment of NFR5-type LysM receptors is shown in FIGS. 6A-6F. Exemplary NFR5-type LysM Nod factor receptors include SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, and SEQ ID NO: 57. NFR5 receptors are also described in Gough et al., (2018) Evolutionary History of Plant LysM Receptor Proteins Related to Root Endosymbiosis, Front. Plant Sci. 9:923.

As described in Examples 1 through 5, the present disclosure provides VHH-based methods to dimerize the NFR1 and NFR5 receptors. These methods showed that the NFR1-NFR5 complex is essential for organogenesis signaling, and that VHH can be used to dimerize NFR1-NFR5 without impairing downstream signaling processes.

### Plant Breeding Methods

Plant breeding begins with the analysis of the current germplasm, the definition of problems and weaknesses of the current germplasm, the establishment of program goals, and the definition of specific breeding objectives. The next step is the selection of germplasm that possess the traits to meet the program goals. The selected germplasm is crossed in order to recombine the desired traits and through selection, varieties or parent lines are developed. The goal is to combine in a single variety or hybrid an improved combination of desirable traits from the parental germplasm. These important traits may include higher yield, field performance, improved fruit and agronomic quality, resistance to biological stresses, such as diseases and pests, and tolerance to environmental stresses, such as drought and heat.

Each breeding program should include a periodic, objective evaluation of the efficiency of the breeding procedure. Evaluation criteria vary depending on the goal and objectives, but should include gain from selection per year based on comparisons to an appropriate standard, overall value of the advanced breeding lines, and number of successful cultivars produced per unit of input (e.g., per year, per dollar expended, etc.). Promising advanced breeding lines are thoroughly tested and compared to appropriate standards in environments representative of the commercial target area(s) for three years at least. The best lines are candidates for new commercial cultivars; those still deficient in a few traits are used as parents to produce new populations for further selection. These processes, which lead to the final step of marketing and distribution, usually take five to ten years from the time the first cross or selection is made.

The choice of breeding or selection methods depends on the mode of plant reproduction, the heritability of the trait(s) being improved, and the type of cultivar used commercially (e.g., F₁ hybrid cultivar, inbred cultivar, etc.). For highly heritable traits, a choice of superior individual plants evaluated at a single location will be effective, whereas for traits with low heritability, selection should be based on mean values obtained from replicated evaluations of families of related plants. The complexity of inheritance also influences the choice of the breeding method. Backcross breeding is used to transfer one or a few genes for a highly heritable trait into a desirable cultivar (e.g., for breeding disease-resistant cultivars), while recurrent selection techniques are used for quantitatively inherited traits controlled by numerous genes, various recurrent selection techniques are used. Commonly used selection methods include pedigree selection, modified pedigree selection, mass selection, and recurrent selection.

Pedigree selection is generally used for the improvement of self-pollinating crops or inbred lines of cross-pollinating crops. Two parents which possess favorable, complementary traits are crossed to produce an F₁. An F₂ population is produced by selfing one or several F₁s or by intercrossing two F₁s (sib mating). Selection of the best individuals is usually begun in the F₂ population; then, beginning in the F₃, the best individuals in the best families are selected. Replicated testing of families, or hybrid combinations involving individuals of these families, often follows in the F₄ generation to improve the effectiveness of selection for traits with low heritability. At an advanced stage of inbreeding (i.e., F₆ and F₇), the best lines or mixtures of phenotypically similar lines are tested for potential release as new cultivars.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding (i.e., recurrent selection) may be used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In addition to phenotypic observations, the genotype of a plant can also be examined. There are many laboratory-based techniques available for the analysis, comparison and characterization of plant genotype; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs, which are also referred to as Microsatellites), Fluorescently Tagged Inter-simple Sequence Repeats (ISSRs), Single Nucleotide Polymorphisms (SNPs), Genotyping by Sequencing (GbS), and Next-generation Sequencing (NGS).

Molecular markers, or “markers”, can also be used during the breeding process for the selection of qualitative traits. For example, markers closely linked to alleles or markers containing sequences within the actual alleles of interest can be used to select plants that contain the alleles of interest. The use of markers in the selection process is often called genetic marker enhanced selection or marker-assisted selection. Methods of performing marker analysis are generally known to those of skill in the art.

Mutation breeding may also be used to introduce new traits into plant varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in Principles of Cultivar Development: Theory and Technique, Walter Fehr (1991), Agronomy Books, 1 (https://lib.dr.iastate.edu/agron_books/1).

The production of double haploids can also be used for the development of homozygous lines in a breeding program. Double haploids are produced by the doubling of a set of chromosomes from a heterozygous plant to produce a completely homozygous individual. For example, see Wan, et al., Theor. Appl. Genet., 77:889-892, 1989.

Additional non-limiting examples of breeding methods that may be used include, without limitation, those found in Principles of Plant Breeding, John Wiley and Son, pp. 115-161 (1960); Principles of Cultivar Development: Theory and Technique, Walter Fehr (1991), Agronomy Books, 1 (https://lib.dr.iastate.edu/agron_books/1), which are herewith incorporated by reference. Molecular biological methods to produce transgenic plant cells, plant parts, and plants

One aspect of the present disclosure provides transgenic plant cells, plant parts, or plants including an oligomeric single-pass transmembrane (SPTM) receptor including a first receptor subunit polypeptide and a second receptor subunit polypeptide, wherein the first receptor subunit polypeptide comprises an affinity polypeptide that binds to the second receptor subunit polypeptide intracellularly inducing oligomerization; and wherein oligomerization of the first receptor subunit polypeptide and the second receptor subunit polypeptide activates the SPTM receptor, wherein the affinity polypeptide is heterologous to the first receptor subunit polypeptide. Another aspect of the present disclosure provides transgenic plant cells, plant parts, or plants including a bispecific affinity polypeptide, including: a first affinity polypeptide that binds to an intracellular portion of a first receptor subunit polypeptide of a SPTM receptor; and a second affinity polypeptide that binds to an intracellular portion of a second receptor subunit polypeptide of the SPTM receptor; wherein binding of the first affinity polypeptide to the first receptor subunit polypeptide and of the second affinity polypeptide to the second receptor subunit polypeptide induces oligomerization; and wherein oligomerization of the first receptor subunit polypeptide and the second receptor subunit polypeptide activates the SPTM receptor. In additional embodiments of these aspects, the SPTM receptor is a NFR1-NFR5 receptor including a NFR1 polypeptide and a NFR5 polypeptide. In addition, the present disclosure provides isolated DNA molecules of vectors and gene editing components used to produce transgenic plants of the present disclosure.

In some embodiments, the TM receptor is engineered to link the first subunit polypeptide and the second subunit polypeptide (e.g., intracellularly or extracellularly) (e.g., via linker, sulfide bonding). In some embodiments, an endogenous transmembrane receptor can be knocked out for more efficient receptor subunit pairing. In some embodiments, the affinity polypeptide can comprise an enzyme-cleavable binding fragment (e.g., ubiquitin) for spatial- or location-dependent activation of the receptor.

As used in the present disclosure, the term “oligomerization” may mean covalently bound subunits or non-covalently bound subunits. TM receptor complexes of the present disclosure may be covalent complexes or non-covalent complexes.

A “subunit” of the present disclosure may be a receptor subunit polypeptide. Receptor subunit polypeptides may be transmembrane (TM) receptors. Two or more TM receptors can together form a TM receptor complex (or oligomeric receptor) that activates TM receptor signaling.

Transformation and generation of genetically altered monocotyledonous and dicotyledonous plant cells is well known in the art. See, e.g., Weising, et al., Ann. Rev. Genet. 22:421-477 (1988); U.S. Pat. No. 5,679,558; Agrobacterium Protocols, ed: Gartland, Humana Press Inc. (1995); Wang, et al. Acta Hort. 461:401-408 (1998), and Broothaerts, et al. Nature 433:629-633 (2005). The choice of method varies with the type of plant to be transformed, the particular application, and/or the desired result. The appropriate transformation technique is readily chosen by the skilled practitioner.

Any methodology known in the art to delete, insert or otherwise modify the cellular DNA (e.g., genomic DNA and organelle DNA) can be used in practicing the compositions, methods, and processes disclosed herein. As an example, the CRISPR/Cas-9 system and related systems (e.g., TALEN, ZFN, ODN, etc.) may be used to insert a heterologous gene to a targeted site in the genomic DNA or substantially edit an endogenous gene to express the heterologous gene or to modify the promoter to increase or otherwise alter expression of an endogenous gene through, for example, removal of repressor binding sites or introduction of enhancer binding sites. For example, a disarmed Ti plasmid, containing a genetic construct for deletion or insertion of a target gene, in Agrobacterium tumefaciens can be used to transform a plant cell, and thereafter, a transformed plant can be regenerated from the transformed plant cell using procedures described in the art, for example, in EP 0116718, EP 0270822, PCT publication WO 84/02913 and published European Patent application (“EP”) 0242246. Ti-plasmid vectors each contain the gene between the border sequences, or at least located to the left of the right border sequence, of the T-DNA of the Ti-plasmid. Of course, other types of vectors can be used to transform the plant cell, using procedures such as direct gene transfer (as described, for example in EP 0233247), pollen mediated transformation (as described, for example in EP 0270356, PCT publication WO 85/01856, and U.S. Pat. No. 4,684,611), plant RNA virus-mediated transformation (as described, for example in EP 0 067 553 and U.S. Pat. No. 4,407,956), liposome-mediated transformation (as described, for example in U.S. Pat. No. 4,536,475), and other methods such as the methods for transforming certain lines of corn (e.g., U.S. Pat. No. 6,140,553; Fromm et al., Bio/Technology (1990) 8, 833-839); Gordon-Kamm et al., The Plant Cell, (1990) 2, 603-618), rice (Shimamoto et al., Nature, (1989) 338, 274-276; Datta et al., Bio/Technology, (1990) 8, 736-740), and the method for transforming monocots generally (PCT publication WO 92/09696). For cotton transformation, the method described in PCT patent publication WO 00/71733 can be used. For soybean transformation, reference is made to methods known in the art, e.g., Hinchee et al. (Bio/Technology, (1988) 6, 915) and Christou et al. (Trends Biotech, (1990) 8, 145) or the method of WO 00/42207.

Genetically altered plants of the present disclosure can be used in a conventional plant breeding scheme to produce more genetically altered plants with the same characteristics, or to introduce the genetic alteration(s) in other varieties of the same or related plant species. Seeds, which are obtained from the altered plants, preferably contain the genetic alteration(s) as a stable insert in chromosomal DNA or as modifications to an endogenous gene or promoter. Plants including the genetic alteration(s) in accordance with this disclosure include plants including, or derived from, root stocks of plants including the genetic alteration(s) of this disclosure, e.g., fruit trees or ornamental plants. Hence, any non-transgenic grafted plant parts inserted on a transformed plant or plant part are included in this disclosure.

Genetic alterations of the disclosure, including in an expression vector or expression cassette, which result in the expression of an introduced gene or altered expression of an endogenous gene will typically utilize a plant-expressible promoter. A ‘plant-expressible promoter’ as used herein refers to a promoter that ensures expression of the genetic alteration(s) of this disclosure in a plant cell. Examples of constitutive promoters that are often used in plant cells are the cauliflower mosaic (CaMV) 35S promoter (Kay et al. Science, 236, 4805, 1987), the minimal CaMV 35S promoter (Benfey & Chua, Science, (1990) 250, 959-966), various other derivatives of the CaMV 35S promoter, the figwort mosaic virus (FMV) promoter (Richins, et al., Nucleic Acids Res. (1987) 15:8451-8466) the maize ubiquitin promoter (Christensen & Quail, Transgenic Res, 5, 213-8, 1996), the polyubiquitin promoter (Ljubql, Maekawa et al. Mol Plant Microbe Interact. 21, 375-82, 2008), the vein mosaic cassava virus promoter (International Application WO 97/48819), and the Arabidopsis UBQ10 promoter, Norris et al. Plant Mol. Biol. 21, 895-906, 1993).

Additional examples of promoters directing constitutive expression in plants are known in the art and include: the strong constitutive 35S promoters (the “35S promoters”) of the cauliflower mosaic virus (CaMV), e.g., of isolates CM 1841 (Gardner et al., Nucleic Acids Res, (1981) 9, 2871-2887), CabbB S (Franck et al., Cell (1980) 21, 285-294) and CabbB JI (Hull and Howell, Virology, (1987) 86, 482-493); promoters from the ubiquitin family (e.g., the maize ubiquitin promoter of Christensen et al., Plant Mol Biol, (1992) 18, 675-689), the gos2 promoter (de Pater et al., The Plant J (1992) 2, 834-844), the emu promoter (Last et al., Theor Appl Genet, (1990) 81, 581-588), actin promoters such as the promoter described by An et al. (The Plant J, (1996) 10, 107), the rice actin promoter described by Zhang et al. (The Plant Cell, (1991) 3, 1155-1165); promoters of the figwort mosaic virus (FMV) (Richins, et al., Nucleic Acids Res. (1987) 15:8451-8466), promoters of the Cassava vein mosaic virus (WO 97/48819; Verdaguer et al., Plant Mol Biol, (1998) 37, 1055-1067), the pPLEX series of promoters from Subterranean Clover Stunt Virus (WO 96/06932, particularly the S4 or S7 promoter), an alcohol dehydrogenase promoter, e.g., pAdhlS (GenBank accession numbers X04049, X00581), and the TR1′ promoter and the TR2′ promoter (the “TR1′ promoter” and “TR2′ promoter”, respectively) which drive the expression of the 1′ and 2′ genes, respectively, of the T DNA (Velten et al., EMBO J, (1984) 3, 2723-2730).

Alternatively, a plant-expressible promoter can be a tissue-specific promoter, i.e., a promoter directing a higher level of expression in some cells or tissues of the plant, e.g., in root epidermal cells or root cortex cells. In preferred embodiments, LysM receptor promoters will be used. Non-limiting examples include NFR1 promoters, NFR5 promoters, LYK3 promoters, NFP promoters, the Lotus japonicus NFR5 promoter (SEQ ID NO: 27), the Lotus japonicus NFR1 promoter (SEQ ID NO: 69), the Medicago truncatula NFP promoter (SEQ ID NO: 29), the Lotus japonicus CERK6 promoter (SEQ ID NO: 46), and the Medicago truncatula LYK3 promoter (SEQ ID NO: 28). In additional preferred embodiments, root specific promoters will be used. Non-limiting examples include the promoter of the maize metallothionein (De Framond et al, FEBS 290, 103-106, 1991 Application EP 452269), the chitinase promoter (Samac et al. Plant Physiol 93, 907-914, 1990), the glutamine synthetase soybean root promoter (Hirel et al. Plant Mol. Biol. 20, 207-218, 1992), the RCC3 promoter (PCT Application WO 2009/016104), the rice antiquitin promoter (PCT Application WO 2007/076115), the LRR receptor kinase promoter (PCT application WO 02/46439), the maize ZRP2 promoter (U.S. Pat. No. 5,633,363), the tomato LeExtl promoter (Bucher et al. Plant Physiol. 128, 911-923, 2002), and the Arabidopsis pCO2 promoter (Heidstra et al, Genes Dev. 18, 1964-1969, 2004). These plant promoters can be combined with enhancer elements, they can be combined with minimal promoter elements, or can comprise repeated elements to ensure the expression profile desired.

Examples of constitutive promoters that are often used in plant cells are the cauliflower mosaic (CaMV) 35S promoter (Kay et al. Science, 236, 4805, 1987), and various derivatives of the promoter, virus promoter vein mosaic cassava (International Application WO 97/48819), the maize ubiquitin promoter (Christensen & Quail, Transgenic Res, 5, 213-8, 1996), polyubiquitin (Ljubql, Maekawa et al. Mol Plant Microbe Interact. 21, 375-82, 2008) and Arabidopsis UBQ10 (Norris et al. Plant Mol. Biol. 21, 895-906, 1993).

In some embodiments, further genetic alterations to increase expression in plant cells can be utilized. For example, an intron at the 5′ end or 3′ end of an introduced gene, or in the coding sequence of the introduced gene, e.g., the hsp70 intron. Other such genetic elements can include, but are not limited to, promoter enhancer elements, duplicated or triplicated promoter regions, 5′ leader sequences different from another transgene or different from an endogenous (plant host) gene leader sequence, 3′ trailer sequences different from another transgene used in the same plant or different from an endogenous (plant host) trailer sequence.

An introduced gene of the present disclosure can be inserted in host cell DNA so that the inserted gene part is upstream (i.e., 5′) of suitable 3′ end transcription regulation signals (i.e., transcript formation and polyadenylation signals). This is preferably accomplished by inserting the gene in the plant cell genome (nuclear or chloroplast). Preferred polyadenylation and transcript formation signals include those of the nopaline synthase gene (Depicker et al., J. Molec Appl Gen, (1982) 1, 561-573), the octopine synthase gene (Gielen et al., EMBO J, (1984) 3:835-845), the SCSV or the Malic enzyme terminators (Schunmann et al., Plant Funct Biol, (2003) 30:453-460), and the T DNA gene 7 (Velten and Schell, Nucleic Acids Res, (1985) 13, 6981-6998), which act as 3′ untranslated DNA sequences in transformed plant cells. In some embodiments, one or more of the introduced genes are stably integrated into the nuclear genome. Stable integration is present when the nucleic acid sequence remains integrated into the nuclear genome and continues to be expressed (i.e., detectable mRNA transcript or protein is produced) throughout subsequent plant generations. Stable integration into the nuclear genome can be accomplished by any known method in the art (e.g., microparticle bombardment, Agrobacterium-mediated transformation, CRISPR/Cas9, electroporation of protoplasts, microinjection, etc.).

The term recombinant or modified nucleic acids refers to polynucleotides which are made by the combination of two otherwise separated segments of sequence accomplished by the artificial manipulation of isolated segments of polynucleotides by genetic engineering techniques or by chemical synthesis. In so doing one may join together polynucleotide segments of desired functions to generate a desired combination of functions.

As used herein, the term “overexpression” refers to increased expression (e.g., of mRNA, polypeptides, etc.) relative to expression in a wild type organism (e.g., plant) as a result of genetic modification and can refer to expression of heterologous genes at a sufficient level to achieve the desired result such as increased yield. In some embodiments, the increase in expression is a slight increase of about 10% more than expression in wild type. In some embodiments, the increase in expression is an increase of 50% or more (e.g., 60%, 70%, 80%, 100%, etc.) relative to expression in wild type. In some embodiments, an endogenous gene is upregulated. In some embodiments, an exogenous gene is upregulated by virtue of being expressed. Upregulation of a gene in plants can be achieved through any known method in the art, including but not limited to, the use of constitutive promoters with inducible response elements added, inducible promoters, high expression promoters (e.g., PsaD promoter) with inducible response elements added, enhancers, transcriptional and/or translational regulatory sequences, codon optimization, modified transcription factors, and/or mutant or modified genes that control expression of the gene to be upregulated in response to a stimulus such as cytokinin signaling.

Where a recombinant nucleic acid is intended for expression, cloning, or replication of a particular sequence, DNA constructs prepared for introduction into a host cell will typically include a replication system (e.g., vector) recognized by the host, including the intended DNA fragment encoding a desired polypeptide, and can also include transcription and translational initiation regulatory sequences operably linked to the polypeptide-encoding segment. Additionally, such constructs can include cellular localization signals (e.g., plasma membrane localization signals). In preferred embodiments, such DNA constructs are introduced into a host cell's genomic DNA, chloroplast DNA or mitochondrial DNA.

In some embodiments, a non-integrated expression system can be used to induce expression of one or more introduced genes. Expression systems (expression vectors) can include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides can also be included where appropriate from secreted polypeptides of the same or related species, which allow the protein to cross and/or lodge in cell membranes, cell wall, or be secreted from the cell.

Selectable markers useful in practicing the methodologies disclosed herein can be positive selectable markers. Typically, positive selection refers to the case in which a genetically altered cell can survive in the presence of a toxic substance only if the recombinant polynucleotide of interest is present within the cell. Negative selectable markers and screenable markers are also well known in the art and are contemplated by the present disclosure. One of skill in the art will recognize that any relevant markers available can be utilized in practicing the compositions, methods, and processes disclosed herein.

Screening and molecular analysis of recombinant strains of the present disclosure can be performed utilizing nucleic acid hybridization techniques. Hybridization procedures are useful for identifying polynucleotides, such as those modified using the techniques described herein, with sufficient homology to the subject regulatory sequences to be useful as taught herein. The particular hybridization techniques are not essential to this disclosure. As improvements are made in hybridization techniques, they can be readily applied by one of skill in the art. Hybridization probes can be labeled with any appropriate label known to those of skill in the art. Hybridization conditions and washing conditions, for example, temperature and salt concentration, can be altered to change the stringency of the detection threshold. See, e.g., Sambrook et al. (1989) vide infra or Ausubel et al. (1995) Current Protocols in Molecular Biology, John Wiley & Sons, NY, N.Y., for further guidance on hybridization conditions.

Additionally, screening and molecular analysis of genetically altered strains, as well as creation of desired isolated nucleic acids can be performed using Polymerase Chain Reaction (PCR). PCR is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki et al. (1985) Science 230:1350-1354). PCR is based on the enzymatic amplification of a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. Because the extension product of each primer can serve as a template for the other primer, each cycle essentially doubles the amount of DNA template produced in the previous cycle. This results in the exponential accumulation of the specific target fragment, up to several million-fold in a few hours. By using a thermostable DNA polymerase such as the Taq polymerase, which is isolated from the thermophilic bacterium Thermus aquaticus, the amplification process can be completely automated. Other enzymes which can be used are known to those skilled in the art.

Nucleic acids and proteins of the present disclosure can also encompass homologues of the specifically disclosed sequences. Homology (e.g., sequence identity) can be 50%-100%. In some instances, such homology is greater than 80%, greater than 85%, greater than 90%, or greater than 95%. The degree of homology or identity needed for any intended use of the sequence(s) is readily identified by one of skill in the art. As used herein percent sequence identity of two nucleic acids is determined using an algorithm known in the art, such as that disclosed by Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the BLASTN, BLASTP, and BLASTX, programs of Altschul et al. (1990) J. Mol. Biol. 215:402-410. BLAST nucleotide searches are performed with the BLASTN program, score=100, wordlength=12, to obtain nucleotide sequences with the desired percent sequence identity. To obtain gapped alignments for comparison purposes, Gapped BLAST is used as described in Altschul et al. (1997) Nucl. Acids. Res. 25:3389-3402. When utilizing BLAST and Gapped BLAST programs, the default parameters of the respective programs (BLASTN and BLASTX) are used. See www.ncbi.nih.gov. One of skill in the art can readily determine in a sequence of interest where a position corresponding to amino acid or nucleic acid in a reference sequence occurs by aligning the sequence of interest with the reference sequence using the suitable BLAST program with the default settings (e.g., for BLASTP: Gap opening penalty: 11, Gap extension penalty: 1, Expectation value: 10, Word size: 3, Max scores: 25, Max alignments: 15, and Matrix: blosum62; and for BLASTN: Gap opening penalty: 5, Gap extension penalty: 2, Nucleic match: 1, Nucleic mismatch −3, Expectation value: 10, Word size: 11, Max scores: 25, and Max alignments: 15).

Preferred host cells are plant cells. Recombinant host cells, in the present context, are those which have been genetically modified to contain an isolated nucleic molecule, contain one or more deleted or otherwise non-functional genes normally present and functional in the host cell, or contain one or more genes to produce at least one recombinant protein. The nucleic acid(s) encoding the protein(s) of the present disclosure can be introduced by any means known to the art which is appropriate for the particular type of cell, including without limitation, transformation, lipofection, electroporation or any other methodology known by those skilled in the art.

“Isolated”, “isolated DNA molecule” or an equivalent term or phrase is intended to mean that the DNA molecule or other moiety is one that is present alone or in combination with other compositions, but altered from or not within its natural environment. For example, nucleic acid elements such as a coding sequence, intron sequence, untranslated leader sequence, promoter sequence, transcriptional termination sequence, and the like, that are naturally found within the DNA of the genome of an organism are not considered to be “isolated” so long as the element is within the genome of the organism and at the location within the genome in which it is naturally found. However, each of these elements, and subparts of these elements, would be “isolated” from its natural setting within the scope of this disclosure so long as the element is not within the genome of the organism in which it is naturally found, the element is altered from its natural form, or the element is not at the location within the genome in which it is naturally found. Similarly, a nucleotide sequence encoding a protein or any naturally occurring variant of that protein would be an isolated nucleotide sequence so long as the nucleotide sequence was not within the DNA of the organism from which the sequence encoding the protein is naturally found in its natural location or if that nucleotide sequence was altered from its natural form. A synthetic nucleotide sequence encoding the amino acid sequence of the naturally occurring protein would be considered to be isolated for the purposes of this disclosure. For the purposes of this disclosure, any transgenic nucleotide sequence, i.e., the nucleotide sequence of the DNA inserted into the genome of the cells of a plant, alga, fungus, or bacterium, or present in an extrachromosomal vector, would be considered to be an isolated nucleotide sequence whether it is present within the plasmid or similar structure used to transform the cells, within the genome of the plant or bacterium, or present in detectable amounts in tissues, progeny, biological samples or commodity products derived from the plant or bacterium.

Having generally described the compositions, methods, and processes of this disclosure, the same will be better understood by reference to certain specific examples, which are included herein to further illustrate the disclosure and are not intended to limit the scope of the invention as defined by the claims.

### Examples

The present disclosure is described in further detail in the following examples which are not in any way intended to limit the scope of the disclosure as claimed. The attached figures are meant to be considered as integral parts of the specification and description of the disclosure. The following example is offered to illustrate, but not to limit the claimed disclosure.

### Example 1: Generation of a VHH to Drive In Vivo Receptor Assembly

The following example describes the generation of a VHH and the subsequent use of the antibody as a protein tag to enable controlled assembly of two receptors in living plant cells.

### Expression and Purification of the Lotus NFR5 Kinase

The NFR5 kinase (residues A276-T563) was cloned into pET-32 Ek/LIC (Merck) and fused at the N-terminal to a TEV-cleavable thioredoxin 6×-histidine tag. The plasmid was transformed into E. coli Rosetta 2 (Sigma-Aldrich, St. Louis, MO, USA). The expression culture was grown in LB containing ampicillin and chloramphenicol at 37° C., 150 RPM until OD₆₀₀=0.6 and incubated on ice for 20 minutes. Expression was induced by supplementing the culture with 0.2 mM IPTG and cells were incubated at 18° C. overnight in a shaking incubator at 100 RPM. Cells were pelleted by centrifugation (5000×g, 4° C. for 15 min), resuspended in lysis buffer (50 mM Tris-HCl pH 8.0, 500 mM NaCl, 20 mM Imidazole, 5 mM β-mercaptoethanol, 10% Glycerol, 1 mM Benzamidine), and lysed by sonication. The lysate was cleared by centrifugation (16,000×g, 4° C. for 30 minutes) and the NFR5 kinase was purified from the supernatant using a 1 mL FPLC Protino Ni-NTA column (Macherey-Nagel, Düren, Germany). The column was equilibrated in lysis buffer and NFR5 kinase was eluted in buffer B (50 mM Tris-HCl pH 8.0, 500 mM NaCl, 500 mM Imidazole, 5 mM β-mercaptoethanol, 5% Glycerol) after extensive washing with lysis buffer. Histidine-tagged tobacco etch virus (TEV) protease was added to the NFR5 kinase sample in a 1:50 molar ratio and the digestion mixture was dialysed overnight at 4° C. against SEC buffer (50 mM Tris-HCl pH 8.0, 500 mM NaCl, 5 mM β-mercaptoethanol). The TEV protease and digested thioredoxin-6× histidine tag was removed through a second nickel affinity chromatography purification step. The NFR5 kinase was purified in a final SEC step on a HiLoad® Superdex® 75 16/600 pg (Cytiva, Marlborough, MA, USA) in SEC buffer. All purification steps were analyzed by SDS-PAGE and elution fractions pooled accordingly.

### Generation of a NFR5 VHH

One Lama glama was immunized with 400 μg NFR5 kinase by Capralogics (Hardwick, USA). From a blood sample after 6-8 weeks, peripheral lymphocytes were isolated by Ficoll gradient centrifugation. Total RNA was purified using an RNeasy® Plus Mini Kit (Qiagen, Valencia, CA, USA) and cDNA was generated and amplified using a SuperScript TM III First-Strand Kit (Invitrogen) and random hexamer primers. The VHH/Nb regions were amplified by PCR and cloned into a phagemid pD-GFP vector as an E-tag pIII-fusion. E. coli ER2738 were transformed with VHH inserted phagemid and M13 phage-display libraries were produced using VCSM13 helper phages. A 96-well Nunc™ Maxisorp™ Immuno-Microplate (Thermo Fisher Scientific, Waltham, MA, USA) was coated with 1 μg/well NFR5 kinase overnight at 4° C. and blocked by addition of PBS, 2% w/v BSA for 1 hour at room temperature. 3×10¹² phages per selection were blocked in PBS, 2% w/v BSA for 1 hour before applying to antigen-coated wells for an additional 1 hour. Wells were washed 15 times in PBS supplemented with 0.1% Tween 20 (PBS-T) followed by 15 washes in PBS. Phages were eluted by addition of 100 μL 0.2 M glycine pH 2.2 and incubated on a shaking table for 10 minutes. Elutions were pH adjusted by addition of 15 μL 1 M Tris-HCl pH 9.1 and used for infecting 0.8 mL ER2738, OD₆₀₀=0.6. The phage-library was amplified for a second round of selection, using a more stringent 0.1 μg antigen-coating. After the second selection round, infected ER2738 were plated on Luria Agar, supplemented with 2% w/v glucose, 100 μg/mL ampicillin, and 10 μg/mL tetracycline and grown overnight at 37° C. Single colonies were picked and inoculated into a 96-well micro-titre plate filled with LB supplemented with 100 μg/mL ampicilin and 10 μg/mL tetracycline. The plate was covered with an AirPore sheet (Qiagen) and incubated for 6 hours at 37° C., 95 RPM at high humidity. E-tagged VHHs were expressed overnight at 30° C., 95 RPM by supplementing with 0.8 mM IPTG.

E. coli were pelleted at 1000×g, 4° C. for 15 minutes and the supernatant containing E-tagged VHHs were used to screen NFR5 kinase binding in an enzyme-linked immunosorbent assay (ELISA).

A 0.1 μg/well NFR5 kinase coated 96-well Nunc™ plate was generated as described above and 50 μL VHH supernatant was incubated in the ELISA plate for 1 hour at room temperature followed by 6×PBS-T wash. Secondary anti-E-tag HRP antibody (Bethyl laboratories, Montgomery, Texas, USA) was diluted 10000-fold in PBS, 2% w/v BSA and 100 μL was added to each ELISA well and incubated for 1 hour at room temperature. Wells were washed 3× in PBS-T and developed by addition of 100 μL TMB (Thermo Scientific™). Reactions were quenched with 100 μL 1 M HCl and ELISA plates were read at 450 nm using a Varioskan™ LUX Multimode Microplate Reader (Thermo Scientific™).

Positive clones were propagated in 5 mL LB supplemented with 100 μg/mL ampicillin and phagemid DNA was purified and sequenced. Nb_{NFR5} (VHH towards NFR5; SEQ ID NO: 2) was cloned into pET-22b(+) and fused at the C terminal to a non-cleavable 6×-histidine tag. Expression and purification were performed similarly as described for the NFR5 kinase. E. coli LOBSTR (Kerafast, Boston, MA, USA) (Andersen et al. (2013) Proteins 81, 1857-1861) was used as the expression strain, a single nickel affinity chromatography step was performed, and SEC was performed using a Superdex® 75 increase 10/300 GL column (Cytiva).

Analytical SEC was used to assay NFR5 kinase:Nb_{NFR5} complex formation. 100 μg NFR5 kinase was mixed in a 1:1.5 molar ratio with Nb_{NFR5} and incubated on ice for 30 minutes. Assays were performed using a Superdex® 200 increase 10/300 (Cytiva) in SEC buffer. Chromatograms were processed in Unicorn 6 (Cytiva) and analysed in GraphPad Prism 9 (GraphPad Software, San Diego, CA, USA).

### Generation of Plant Expression Vectors

For generation of the plant expression vector, the sequence of Nb_{NFR5} (SEQ ID NO: 2) was added in-frame as a C-terminal tag to the native NFR1 promoter and sequence into the pIV10_tYFP-NLS expression vector via Golden Gate cloning (Weber et al. (2011) PLoS One 6, e16765), where YFP fused to a nuclear localization signal (NLS) serves as a transformation control. Agrobacterium rhizogenes strain AR1193 was grown in LB medium at 28° C. and transformed with pIV10 vectors via conjugation. A construct overview is given in Table 1.

TABLE 1
Construct overview

| | |
|---|---|
| Standard | Back |

| | | | | |
|---|---|---|---|---|
| Short | ID | name | bone | Combined modules |

For hairy root transformations:

| | | | | | | | | | | |
|---|---|---|---|---|---|---|---|---|---|---|
| NFR1_ | EC31220 | EC31220_ | EC25164_ | PMC- | EC31213_ | EC25031_ | EC31217_ | EC49283 | | |
| NFR5 | | pL2V_pIV10- | pL2V-1 | 02596_ | pL1R2- | pL1V-F3- | pL1F4- | (ELB-4) | | |
| | | R1-pLjUbi- | pIV10 | pL1R1- | pLjNFR1- | buffer | pLjNFR5- | | | |
| | | tYFPnls- | | pLjUbi- | gLjNFR1- | | LjNFR5- | | | |
| | | tNOS-R2- | | tYFPnls- | 6xHis- | | 3xHA- | | | |
| | | pLjNFR1- | | tNOS | tLjNFR1 | | tLjNFR5 | | | |
| | | gLjNFR1- | | | | | | | | |
| | | 6xHis- | | | | | | | | |
| | | tLjNFR1- | | | | | | | | |
| | | F3- | | | | | | | | |
| | | PUSCT- | | | | | | | | |
| | | buffer-F4- | | | | | | | | |
| | | pLjNFR5- | | | | | | | | |
| | | LjNFR5- | | | | | | | | |
| | | 3xHA- | | | | | | | | |
| | | tLjNFR5- | | | | | | | | |
| | | ELE-4 | | | | | | | | |
| NFR1- | EC31959 | EC31959_ | EC25164_ | PMC- | EC31468_ | EC31914_ | | EC49283 | | |
| NB_{NFR5} | | pL2V_ | pL2V-1 | 02596_ | pL1V_ | pL1V-F4_ | | (ELB-4) | | |
| | | pIV10- | pIV10 | pL1R1- | pUC57G_ | pNFR1_ | | | | |
| | | R1_pLjUbi_ | | pLjUbi- | F2 + 3 | gNFR1_ | | | | |
| | | tYFPnls_ | | tYFPnls- | Buffer_ | 20linker _ | | | | |
| | | tNOS- | | tNOS | GCAA_ | KRA198- | | | | |
| | | F4_pNFR1_ | | | TTAC | HA_tNFR1 | | | | |
| | | gNFR1_20 | | | | | | | | |
| | | linker_NB | | | | | | | | |
| | | NFR5-HA- | | | | | | | | |
| | | tNFR1_ | | | | | | | | |
| | | ELB4 | | | | | | | | |
| NFR1Nb_ | EC31687 | EC31687_ | EC25164_ | PMC- | PMC- | EC25031 | EC31917_ | EC49283 | | |
| NFR5 | | pL2V_pIV10- | pL2V-1 | 02596_ | 03558_ | pL1M- | pL2V_ | (ELB-4) | | |
| sfGFP | | R1_pLjUbi_ | pIV10 | pL1R1- | L1V-F2_ | F3- | pIV10- | | | |
| | | tYFPnls_ | | pLjUbi- | PU- | Buffer- | R1_pLjUbi_ | | | |
| | | tNOS- | | tYFPnls- | pLjNfr5- | 25031 | tYFPnls | | | |
| | | F2_pNFR5_ | | tNOS | SC3- | | tNOS- | | | |
| | | NFR5_ | | | LjNfr5_ | | F4_pNFR1_ | | | |
| | | sfGFP_ | | | C4_ | | gNFR1_ | | | |
| | | tNFR5_ | | | sfGFP_ | | 20linker_ | | | |
| | | F4_pNFR1_ | | | T-LjNfr5 | | NbsfGFP- | | | |
| | | gNFR1_ | | | | | HA_tNFR1- | | | |
| | | 20linker_ | | | | | ELB4 | | | |
| | | NbsfGFP- | | | | | | | | |
| | | HA_tNFR1_ | | | | | | | | |
| | | ELB4 | | | | | | | | |
| NFR1 | EC31672 | EC31672_ | EC25164_ | PMC- | PMC- | EC25031 | EC31918_ | EC49283 | | |
| NbNb_ | | pL2V_ | pL2V-1 | 02596_ | 03558_ | pL1M-F3- | pL2V_ | (ELB-4) | | |
| NFR5 | | pIV10- | pIV10 | pL1R1- | L1V-F2_ | Buffer- | pIV10- | | | |
| sfGFP | | R1_pLjUbi_ | | pLjUbi- | PU- | 25031 | R1_pLjUbi_ | | | |
| | | tYFPnls_ | | tYFPnls- | pLjNfr5- | | tYFPnls_ | | | |
| | | tNOS- | | tNOS | SC3- | | tNOS- | | | |
| | | F2_pNFR5_ | | | LjNfr5_ | | F4_pNFR1_ | | | |
| | | NFR5_ | | | C4_ | | gNFR1_ | | | |
| | | sfGFP_tNFR5_ | | | sfG | | 20linker_ | | | |
| | | F4_pNFR1_ | | | FP_ | | NbsfGFP_ | | | |
| | | gNFR1_ | | | T- | | 40linker_ | | | |
| | | 20linker_ | | | LjNfr5 | | NbsfGFP- | | | |
| | | NbsfGFP- | | | | | HA_tNFR1- | | | |
| | | 40linker- | | | | | ELB4 | | | |
| | | NbsfGFP- | | | | | | | | |
| | | HA_tNFR1_ | | | | | | | | |
| | | ELB4 | | | | | | | | |
| NFR1 | EC31750 | EC31750_ | EC25164_ | PMC- | EC31650_ | EC31073_ | EC31655_ | EC49283 | | |
| NbNb_ | | pL2V_ | pL2V-1 | 02596_ | L1V-F2_ | pL1V- | L1V- | (ELB-4) | | |
| NFR5sfGFP | | pIV10_R1- | pIV10 | pL1R1- | pLjNfr1_ | R3_spacer1 | F4_pNFR5- | | | |
| | | pLjUbi- | | pLjUbi- | gLjNfr1- | | LjNFR5- | | | |
| | | tYFPnls- | | tYFPnls- | 20aa- | | sfGFP_T- | | | |
| | | tNOS_F2- | | tNOS | NbGFP- | | LjNFR5 | | | |
| | | pLjNfr1_ | | | 40aa- | | | | | |
| | | gLjNfr1- | | | NbGFP- | | | | | |
| | | 20aa- | | | HA | | | | | |
| | | NbGFP- | | | | | | | | |
| | | 40aa- | | | | | | | | |
| | | NbGFP- | | | | | | | | |
| | | HA_F3- | | | | | | | | |
| | | spacer_F4- | | | | | | | | |
| | | pNFR5- | | | | | | | | |
| | | LjNFR5- | | | | | | | | |
| | | sfGFP_T- | | | | | | | | |
| | | LjNFR5_ | | | | | | | | |
| | | ELB4 | | | | | | | | |
| NFR1 | EC31989 | EC31989_ | EC25164_ | PMC- | PMC- | EC25031 | EC31984_ | EC49283 | | |
| mutNb_ | | pL2V_pIV10- | pL2V-1 | 02596_ | 03558 | pL1M- | pL1V-F4_ | (ELB-4) | | |
| NFR5sfGFP | | R1_pLjUbi_ | pIV10 | pL1R1- | PU- | F3- | pNFR1_ | | | |
| | | tYFPnls_ | | pLjUbi- | pLjNfr5- | Buffer- | gNFR1_ | | | |
| | | tNOS- | | tYFPnls- | SC3- | 25031 | 20linker_ | | | |
| | | F2_pNFR5_ | | tNOS | LjNfr5_ | | mutNbsfGFP- | | | |
| | | NFR5_ | | | C4_ | | HA_tNFR1 | | | |
| | | sfGFP_ | | | sfGFP_ | | | | | |
| | | tNFR5_ | | | T-LjNfr5- | | | | | |
| | | F4_pNFR1_ | | | L1-V F2 | | | | | |
| | | gNFR1_ | | | | | | | | |
| | | 20linker_ | | | | | | | | |
| | | mutNbsfGFP- | | | | | | | | |
| | | HA_ | | | | | | | | |
| | | tNFR1_ELB4 | | | | | | | | |
| NFR1mut | EC31996 | EC31996_ | EC25164_ | PMC- | PMC- | EC25031 | EC31985_ | EC49283 | | |
| NbmutNb_ | | pL2V_pIV10- | pL2V-1 | 02596_ | 03558 | pL1M- | pL1V- | (ELB-4) | | |
| NFR5sfGFP | | R1_pLjUbi_ | pIV10 | pL1R1- | PU- | F3- | F4_pNFR1_ | | | |
| | | tYFPnls_ | | pLjUbi- | pLjNfr5- | Buffer- | gNFR1_ | | | |
| | | tNOS- | | tYFPnls- | SC3- | 25031 | 20linker_ | | | |
| | | F2_pNFR5_ | | tNOS | LjNfr5_ | | mutNbsfGFP- | | | |
| | | NFR5_sfGFP_ | | | C4_ | | 40linker- | | | |
| | | tNFR5_F4_ | | | sfGFP_ | | NbsfGFP- | | | |
| | | pNFR1_ | | | T-LjNfr5- | | HA_tNFR1 | | | |
| | | gNFR1_ | | | L1-V F2 | | | | | |
| | | 20linker_ | | | | | | | | |
| | | mutNbsfGFP- | | | | | | | | |
| | | 40linker- | | | | | | | | |
| | | mutNbsfGFP- | | | | | | | | |
| | | HA_tNFR1_ | | | | | | | | |
| | | ELB4 | | | | | | | | |
| NFR1 | EC31722 | EC31722_ | EC25164_ | PMC- | EC31072 | EC31693_ | EC31638_ | EC49283 | | |
| sfGFP_ | | pL2V_pIV10- | pL2V-1 | 02596_ | L1_V_ | pL1V-R3_ | pL1V- | (ELB-4) | | |
| NFR5Nb | | R1_pLjUbi_ | pIV10 | pL1R1- | F2_ | pNFR1_ | F4_pNFR5_ | | | |
| | | tYFPnls_ | | pLjUbi- | Buffer | gNFR1_ | NFR5_ | | | |
| | | tNOS- | | tYFPnls- | | sfGFP_ | 20linker_ | | | |
| | | R3_pNFR1_ | | tNOS | | tNFR1 | NbsfGFP- | | | |
| | | gNFR1_ | | | | | HA_tNFR5 | | | |
| | | sfGFP_ | | | | | | | | |
| | | tNFR1- | | | | | | | | |
| | | F4_pNFR5_ | | | | | | | | |
| | | NFR5_ | | | | | | | | |
| | | 20linker_ | | | | | | | | |
| | | NbsfGFP- | | | | | | | | |
| | | HA_tNFR5- | | | | | | | | |
| | | ELB4 | | | | | | | | |
| NFR1 | EC31724 | EC31724_ | EC25164_ | PMC- | EC31072 | EC31693_ | EC31640_ | EC49283 | | |
| sfGFP_ | | pL2V_pIV10- | pL2V-1 | 02596_ | L1_V_ | pL1V-R3_ | pL1V- | (ELB-4) | | |
| NFR5 | | R1_pLjUbi_ | pIV10 | pL1R1- | F2_Buffer | pNFR1_ | F4_pNFR5_ | | | |
| NbNb | | tYFPnls_ | | pLjUbi- | | gNFR1_ | NFR5_ | | | |
| | | tNOS- | | tYFPnls- | | sfGFP_ | 20linker_ | | | |
| | | R3_pNFR1_ | | tNOS | | tNFR1 | NbsfGFP- | | | |
| | | gNFR1_ | | | | | 40linker- | | | |
| | | sfGFP_ | | | | | NbsfGFP- | | | |
| | | tNFR1- | | | | | HA_tNFR5 | | | |
| | | F4_pNFR5_ | | | | | | | | |
| | | NFR5_ | | | | | | | | |
| | | 20linker_ | | | | | | | | |
| | | NbsfGFP- | | | | | | | | |
| | | 40linker- | | | | | | | | |
| | | NbsfGFP- | | | | | | | | |
| | | HA_tNFR5- | | | | | | | | |
| | | ELB4 | | | | | | | | |
| NFR1_ | EC55305 | EC55305_ | EC25164_ | PMC- | EC31213_ | | EC25031_ | EC31074- | EC31691_ | EC49299 |
| SYMRK | | pL2V_pIV10- | pL2V-1 | 02596_ | pL1R2- | | pL1V-F3- | L1_V_ | pL1V- | (ELB-5) |
| | | R1_pLjUbi_ | pIV10 | pL1R1- | pLjNFR1- | | buffer | F4_ | R5_ | |
| | | tYFPnls_ | | pLjUbi- | gLjNFR1- | | | spacer1 | pSymRK_ | |
| | | tNOS- | | tYFPnls- | 6xHis- | | | | LjSymRK_ | |
| | | R2_pNFR1_ | | tNOS | tLjNFR1 | | | | sfGFP_ | |
| | | gNFR1_ | | | | | | | tSymRK | |
| | | 6xHis_ | | | | | | | | |
| | | tNFR1- | | | | | | | | |
| | | R5_pSYMRK_ | | | | | | | | |
| | | SYRMK_ | | | | | | | | |
| | | HAtag_ | | | | | | | | |
| | | tSYMRK_ | | | | | | | | |
| | | ELB5 | | | | | | | | |
| NFR1Nb_ | EC55303 | EC55303_ | EC25164_ | PMC- | EC31468_ | EC31642_ | EC31691_ | EC49299 | | |
| SYMRK | | pL2V_pIV10- | pL2V-1 | 02596_ | pL1V_ | pL1V-F4_ | pL1V- | (ELB-5) | | |
| sfGFP | | R1_pLjUbi_ | pIV10 | pL1R1- | pUC57G_ | pNFR1_ | R5_ | | | |
| | | tYFPnls_ | | pLjUbi- | F2 + 3 | gNFR1_ | pSymRK_ | | | |
| | | tNOS- | | tYFPnls- | Buffer_ | 20linker_ | LjSymRK_ | | | |
| | | F4_pNFR1_ | | tNOS | GCAA_ | NbsfGFP- | sfGFP_ | | | |
| | | gNFR1_ | | | TTAC | HA_tNFR1 | tSymRK | | | |
| | | 20linker_ | | | | | | | | |
| | | NbsfGFP- | | | | | | | | |
| | | HA_tNFR1- | | | | | | | | |
| | | R5_pSYMRK_ | | | | | | | | |
| | | SYMRK_ | | | | | | | | |
| | | sfGFP_ | | | | | | | | |
| | | tSYMRK- | | | | | | | | |
| | | ELB5 | | | | | | | | |
| NFR5_ | EC31748 | EC31748_ | EC25164_ | PMC- | EC31468_ | EC31217_ | EC31841_ | EC49299 | | |
| SYMRK | | pL2V_pIV10- | pL2V-1 | 02596_ | pL1V_ | pL1F4- | pL2V_ | (ELB-5) | | |
| | | R1_pLjUbi_ | pIV10 | pL1R1- | pUC57G_ | pLjNFR5- | pIV10- | | | |
| | | tYFPnls_ | | pLjUbi- | F2 + 3 | LjNFR5- | R1_pLjUbi_ | | | |
| | | tNOS- | | tYFPnls- | Buffer_ | 3xHA- | tYFPnls_ | | | |
| | | F4_pNFR5_ | | tNOS | GCAA_ | tLjNFR5 | tNOS-R5_ | | | |
| | | NFR5_3xHA_ | | | TTAC | | pSYMRK_ | | | |
| | | tNFR5- | | | | | SYRMK_ | | | |
| | | R5_pSYMRK_ | | | | | HAtag_ | | | |
| | | SYRMK_ | | | | | tSYMRK_ | | | |
| | | HAtag_ | | | | | ELB5 | | | |
| | | tSYMRK_ | | | | | | | | |
| | | ELB5 | | | | | | | | |
| NFR5Nb_ | EC31726 | EC31726_ | EC25164_ | PMC- | EC31468_ | EC31638_ | EC31691_ | EC49299 | | |
| SYMRK | | pL2V_pIV10- | pL2V-1 | 02596_ | pL1V_ | pL1V-F4_ | pL1V-R5_ | (ELB-5) | | |
| sfGFP | | R1_pLjUbi_ | pIV10 | pL1R1- | pUC57G_ | pNFR5_ | pSymRK_ | | | |
| | | tYFPnls_ | | pLjUbi- | F2 + 3 | NFR5_ | LjSymRK_ | | | |
| | | tNOS- | | tYFPnls- | Buffer_ | 20linker_ | sfGFP_ | | | |
| | | F4_pNFR5_ | | tNOS | GCAA_ | NbsfGFP- | tSymRK | | | |
| | | NFR5_ | | | TTAC | HA_ | | | | |
| | | 20linker_ | | | | tNFR5 | | | | |
| | | NbsfGFP- | | | | | | | | |
| | | HA_tNFR5- | | | | | | | | |
| | | R5_pSYMRK_ | | | | | | | | |
| | | SYMRK_ | | | | | | | | |
| | | sfGFP_ | | | | | | | | |
| | | tSYMRK- | | | | | | | | |
| | | ELB5 | | | | | | | | |
| ΔLysM- | EC31944 | EC31944_ | EC25164_ | PMC- | PMC- | EC25031 | EC31908_ | EC49283 | | |
| NFR1Nb_ | | pL2V_pIV10- | pL2V-1 | 02596_ | 03558 | pL1M-F3- | pL1V- | (ELB-4) | | |
| NFR5sfGFP | | R1_pLjUbi_ | pIV10 | pL1R1- | PU- | Buffer- | F4_pNFR1_ | | | |
| | | tYFPnls_ | | pLjUbi- | pLjNfr5- | 25031 | ΔLysM- | | | |
| | | tNOS- | | tYFPnls- | SC3- | | gNFR1_ | | | |
| | | F2_pNFR5_ | | tNOS | LjNfr5_ | | 20linker_ | | | |
| | | NFR5_ | | | C4_ | | NbsfGFP- | | | |
| | | sfGFP_ | | | sfGFP_ | | HA_tNFR1 | | | |
| | | tNFR5_ | | | T-LjNfr5- | | | | | |
| | | F4_pNFR1_ | | | L1-V | | | | | |
| | | ΔLysM- | | | F2 | | | | | |
| | | gNFR1_ | | | | | | | | |
| | | 20linker_ | | | | | | | | |
| | | NbsfGFP- | | | | | | | | |
| | | HA_tNFR1_ | | | | | | | | |
| | | ELB4 | | | | | | | | |
| NFR1Nb_ | EC31922 | EC31922_ | EC25164_ | PMC- | EC31663_ | EC25031 | EC31642_ | EC49283 | | |
| ΔLysM- | | pL2V_pIV10- | pL2V-1 | 02596_ | pL1-F2_ | pL1M-F3- | pL1V- | (ELB-4) | | |
| NFR5sfGFP | | R1_pLjUbi_ | pIV10 | pL1R1- | pNFR5_ | Buffer- | F4_pNFR1_ | | | |
| | | tYFPnls_ | | pLjUbi- | ΔLySM- | 25031 | gNFR1_ | | | |
| | | tNOS- | | tYFPnls- | NFR5_ | | 20linker_ | | | |
| | | F2_pNFR5_ | | tNOS | sfGFP_ | | NbsfGFP- | | | |
| | | ΔLysM- | | | tNFR5 | | HA_tNFR1 | | | |
| | | NFR5_sfGFP_ | | | | | | | | |
| | | tNFR5-F4_ | | | | | | | | |
| | | pNFR1_ | | | | | | | | |
| | | gNFR1_ | | | | | | | | |
| | | 20linker_ | | | | | | | | |
| | | NbsfGFP- | | | | | | | | |
| | | HA_tNFR1- | | | | | | | | |
| | | ELB4 | | | | | | | | |
| ΔLysM- | EC31963 | EC31963_ | EC25164_ | PMC- | EC31663_ | EC25031 | EC31908_ | EC49283 | | |
| NFR1Nb_ | | pL2V_pIV10- | pL2V-1 | 02596_ | pL1-F2_ | pL1M-F3- | pL1V-F4_ | (ELB-4) | | |
| ΔLysM- | | R1_pLjUbi_ | pIV10 | pL1R1- | pNFR5_ | Buffer- | pNFR1_ | | | |
| NFR5sfGFP | | tYFPnls_ | | pLjUbi- | ΔLySM- | 25031 | ΔLysM- | | | |
| | | tNOS- | | tYFPnls- | NFR5_ | | gNFR1_ | | | |
| | | F2_pNFR5_ | | tNOS | sfGFP_ | | 20linker_ | | | |
| | | ΔLysM- | | | tNFR5 | | NbsfGFP- | | | |
| | | NFR5_sfGFP_ | | | | | HA_tNFR1 | | | |
| | | tNFR5 | | | | | | | | |
| | | F4_pNFR1_ | | | | | | | | |
| | | ΔLysM- | | | | | | | | |
| | | gNFR1_ | | | | | | | | |
| | | 20linker_ | | | | | | | | |
| | | NbsfGFP- | | | | | | | | |
| | | HA_tNFR1_ | | | | | | | | |
| | | ELB4 | | | | | | | | |
| NFR1 | EC31961 | EC31961_ | EC25164_ | PMC- | PMC- | EC25031 | EC31942_ | AE45 | | |
| T483ANb_ | | pL2V_pIV10- | pL2V-1 | 02596_ | 03558_ | pL1M-F3- | pL1V-F4_ | (ELB-4) | | |
| NFR5sfGFP | | R1_pLjUbi_ | pIV10 | pL1R1- | L1V-F2 | Buffer- | pNFR1_ | | | |
| | | tYFPnls_ | | pLjUbi- | PU- | 25031 | gNFR1- | | | |
| | | tNOS- | | tYFPnls- | pLjNfr5- | | kd- | | | |
| | | F2_pNFR5_ | | tNOS | SC3- | | T483A_ | | | |
| | | NFR5_ | | | LjNfr5_ | | 20linker_ | | | |
| | | sfGFP_ | | | C4_ | | NbsfGFP- | | | |
| | | tNFR5_ | | | sfGFP_ | | HA_tNFR1 | | | |
| | | F4_pNFR1_ | | | T- | | | | | |
| | | gNFR1-kd- | | | LjNfr5 | | | | | |
| | | T483A_ | | | | | | | | |
| | | 20linker_Nbsf | | | | | | | | |
| | | GFP- | | | | | | | | |
| | | HA_tNFR1_ | | | | | | | | |
| | | ELB4 | | | | | | | | |
| NFR5sfGFP | EC31916 | EC31916_ | EC25164_ | PMC- | PMC- | EC49266 | | | | |
| | | pL2V_pIV10- | pL2V-1 | 02596_ | 03558 | (ELB-2) | | | | |
| | | R1_pLjUbi_ | pIV10 | pL1R1- | PU- | | | | | |
| | | tYFPnls_ | | pLjUbi- | pLjNfr5- | | | | | |
| | | tNOS- | | tYFPnls- | SC3- | | | | | |
| | | F2_pNFR5_ | | tNOS | LjNfr5_ | | | | | |
| | | NFR5_ | | | C4_ | | | | | |
| | | sfGFP_tNFR5- | | | sfGFP_ | | | | | |
| | | ELB2 | | | T- | | | | | |
| | | | | | LjNfr5- | | | | | |
| | | | | | L1-V | | | | | |
| | | | | | F2 | | | | | |
| NFR1Nb | EC31917 | EC31917_ | EC25164_ | PMC- | EC31468_ | EC31642_ | EC49283 | | | |
| | | pL2V_pIV10- | pL2V-1 | 02596_ | pL1V_ | pL1V-F4_ | (ELB-4) | | | |
| | | R1_pLjUbi_ | pIV10 | pL1R1- | pUC57G_ | pNFR1_ | | | | |
| | | tYFPnls_ | | pLjUbi- | F2 + 3 | gNFR1_ | | | | |
| | | tNOS- | | tYFPnls- | Buffer_ | 20linker_ | | | | |
| | | F4_pNFR1_ | | tNOS | GCAA_ | NbsfGFP- | | | | |
| | | gNFR1_ | | | TTAC | HA_ | | | | |
| | | 20linker_ | | | | tNFR1 | | | | |
| | | NbsfGFP- | | | | | | | | |
| | | HA_tNFR1- | | | | | | | | |
| | | ELB4 | | | | | | | | |
| NFR1NbNb | EC31918 | EC31918_ | EC25164_ | PMC- | EC31468_ | EC31644_ | EC49283 | | | |
| | | pL2V_pIV10- | pL2V-1 | 02596_ | pL1V_ | pL1V-F4_ | (ELB-4) | | | |
| | | R1_pLjUbi_ | pIV10 | pL1R1- | pUC57G_ | pNFR1_ | | | | |
| | | tYFPnls_ | | pLjUbi- | F2 + 3 | gNFR1_ | | | | |
| | | tNOS- | | tYFPnls- | Buffer_ | 20linker_ | | | | |
| | | F4_pNFR1_ | | tNOS | GCAA_ | NbsfGFP- | | | | |
| | | gNFR1_ | | | TTAC | 40linker- | | | | |
| | | 20linker_ | | | | NbsfGFP- | | | | |
| | | NbsfGFP_ | | | | HA_tNFR1 | | | | |
| | | 40linker_ | | | | | | | | |
| | | NbsfGFP- | | | | | | | | |
| | | HA_tNFR1- | | | | | | | | |
| | | ELB4 | | | | | | | | |
| NFR1sfGFP | EC31919 | EC31919_ | EC25164_ | PMC- | EC31072 | EC31693_ | EC49277 | | | |
| | | pL2V_pIV10- | pL2V-1 | 02596_ | L1V_F2_ | pL1V-R3_ | (ELB3) | | | |
| | | R1_pLjUbi_ | pIV10 | pL1R1- | Buffer | pNFR1_ | | | | |
| | | tYFPnls_ | | pLjUbi- | | gNFR1_ | | | | |
| | | tNOS- | | tYFPnls- | | sfGFP_ | | | | |
| | | R3_pNFR1_ | | tNOS | | tNFR1 | | | | |
| | | gNFR1_ | | | | | | | | |
| | | sfGFP_tNFR1_ | | | | | | | | |
| | | ELB3 | | | | | | | | |
| NFR5Nb | EC31920 | EC31920_ | EC25164_ | PMC- | EC31468_ | EC31638_ | EC49283 | | | |
| | | pL2V_pIV10- | pL2V-1 | 02596_ | pL1V_ | pL1V-F4_ | (ELB-4) | | | |
| | | R1_pLjUbi_ | pIV10 | pL1R1- | pUC57G_ | pNFR5_ | | | | |
| | | tYFPnls_ | | pLjUbi- | F2 + 3 | NFR5_ | | | | |
| | | tNOS- | | tYFPnls- | Buffer_ | 20linker_ | | | | |
| | | F4_pNFR5_ | | tNOS | GCAA_ | NbsfGFP- | | | | |
| | | NFR5_ | | | TTAC | HA_tNFR5 | | | | |
| | | 20linker_ | | | | | | | | |
| | | NbsfGFP- | | | | | | | | |
| | | HA_tNFR5- | | | | | | | | |
| | | ELB4 | | | | | | | | |
| NFR5Nb | EC31921 | EC31921_ | EC25164_ | PMC- | EC31468_ | EC31640_ | EC49283 | | | |
| | | pL2V_pIV10- | pL2V-1 | 02596_ | pL1V_ | pL1V-F4_ | (ELB-4) | | | |
| | | R1_pLjUbi_ | pIV10 | pL1R1- | pUC57G_ | pNFR5_ | | | | |
| | | tYFPnls_ | | pLjUbi- | F2 + 3 | NFR5_ | | | | |
| | | tNOS- | | tYFPnls- | Buffer_ | 20linker_ | | | | |
| | | F4_pNFR5_ | | tNOS | GCAA_ | NbsfGFP- | | | | |
| | | NFR5_ | | | TTAC | 40linker- | | | | |
| | | 20linker_ | | | | NbsfGFP- | | | | |
| | | NbsfGFP_ | | | | HA_tNFR5 | | | | |
| | | 40linker_ | | | | | | | | |
| | | NbsfGFP- | | | | | | | | |
| | | HA_tNFR5- | | | | | | | | |
| | | ELB4 | | | | | | | | |
| NFP_ | EC55059 | EC55059_ | EC25164_ | PMC- | EC31972_ | EC31073_ | EC31968_ | EC49283 | | |
| LYK3Nb | | L2V- | pL2V-1 | 02596_ | L1V-F2_ | pL1V-R3_ | L1V-F4_ | (ELB-4) | | |
| | | pIV10_R1_ | pIV10 | pL1R1- | pMtNFP_ | spacer1 | pMLYK3_ | | | |
| | | pLjUbi- | | pLjUbi- | MtNFP- | | MtLyk3- | | | |
| | | tYFPnls- | | tYFPnls- | 6xhis_ | | 20aa- | | | |
| | | tNOS_F2_ | | tNOS | tNFP | | NbGFP_ | | | |
| | | pMtNFP_ | | | | | t35S | | | |
| | | MtNFP- | | | | | | | | |
| | | 6xhis_tNFP_ | | | | | | | | |
| | | R3_spacer_ | | | | | | | | |
| | | F4_pMLYK3_ | | | | | | | | |
| | | MtLyk3-20aa- | | | | | | | | |
| | | NbGFP_ | | | | | | | | |
| | | t35S_ELB4 | | | | | | | | |
| NFP_ | EC55060 | EC55060_ | EC25164_ | PMC- | EC31972_ | EC31073_ | EC31969_ | EC49283 | | |
| LYK3NbNb | | L2V- | pL2V-1 | 02596_ | L1V-F2_ | pL1V-R3_ | L1V-F4_ | (ELB-4) | | |
| | | pIV10_R1_ | pIV10 | pL1R1- | pMtNFP_ | spacer1 | pMLYK3_ | | | |
| | | pLjUbi- | | pLjUbi- | MtNFP- | | MtLyk3- | | | |
| | | tYFPnls- | | tYFPnls- | 6xhis_ | | 20aa- | | | |
| | | tNOS_F2_ | | tNOS | tNFP | | NbGFP- | | | |
| | | pMtNFP_ | | | | | 40aa- | | | |
| | | MtNFP- | | | | | NbGFP- | | | |
| | | 6xhis_tNFP_ | | | | | HA_t35S | | | |
| | | R3_spacer_ | | | | | | | | |
| | | F4_pMLYK3_ | | | | | | | | |
| | | MtLyk3- | | | | | | | | |
| | | 20aa-NbGFP- | | | | | | | | |
| | | 40aa- | | | | | | | | |
| | | NbGFP- | | | | | | | | |
| | | HA_t35S_ | | | | | | | | |
| | | ELB4 | | | | | | | | |
| NFPsfGFP_ | EC55051 | EC55051_ | EC25164_ | PMC- | EC31971_ | EC31073_ | EC31968_ | EC49283 | | |
| LYK3Nb | | L2V- | pL2V-1 | 02596_ | L1V-F2_ | pL1V-R3_ | L1V-F4_ | (ELB-4) | | |
| | | pIV10_R1_ | pIV10 | pL1R1- | pMtNFP_ | spacer1 | pMLYK3_ | | | |
| | | pLjUbi- | | pLjUbi- | MtNFP- | | MtLyk3- | | | |
| | | tYFPnls- | | tYFPnls- | sfGFP_ | | 20aa- | | | |
| | | tNOS_F2_ | | tNOS | tNFP | | NbGFP_t35S | | | |
| | | pMtNFP_ | | | | | | | | |
| | | MtNFP- | | | | | | | | |
| | | sfGFP_tNFP_ | | | | | | | | |
| | | R3_spacer_ | | | | | | | | |
| | | F4_pMLYK3_ | | | | | | | | |
| | | MtLyk3- | | | | | | | | |
| | | 20aa- | | | | | | | | |
| | | NbGFP_ | | | | | | | | |
| | | t35S_ELB4 | | | | | | | | |
| NFP | EC55056 | EC55056_ | EC25164_ | PMC- | EC31971_ | EC31073_ | EC31969_ | EC49283 | | |
| sfGFP_ | | L2V- | pL2V-1 | 02596_ | L1V-F2_ | pL1V-R3_ | L1V-F4_ | (ELB-4) | | |
| LYK3 | | pIV10_R1_ | pIV10 | pL1R1- | pMtNFP_ | spacer1 | pMLYK3_ | | | |
| NbNb | | pLjUbi- | | pLjUbi- | MtNFP- | | MtLyk3- | | | |
| | | tYFPnls- | | tYFPnls- | sfGFP_ | | 20aa- | | | |
| | | tNOS_F2_ | | tNOS | tNFP | | NbGFP- | | | |
| | | pMtNFP_ | | | | | 40aa- | | | |
| | | MtNFP- | | | | | NbGFP- | | | |
| | | sfGFP_tNFP_ | | | | | HA_t35S | | | |
| | | R3_spacer_ | | | | | | | | |
| | | F4_pMLYK3_ | | | | | | | | |
| | | MtLyk3- | | | | | | | | |
| | | 20aa- | | | | | | | | |
| | | NbGFP- | | | | | | | | |
| | | 40aa- | | | | | | | | |
| | | NbGFP- | | | | | | | | |
| | | HA_t35S_ | | | | | | | | |
| | | ELB4 | | | | | | | | |
| NFP | EC55057 | EC55057_ | EC25164_ | PMC- | EC31971_ | EC31073_ | EC32021_ | EC49283 | | |
| sfGFP_ | | L2V- | pL2V-1 | 02596_ | L1V-F2_ | pL1V-R3_ | L1V-F4_ | (ELB-4) | | |
| LYK3 | | pIV10_R1_ | pIV10 | pL1R1- | pMtNFP_ | spacer1 | pMLYK3_ | | | |
| mutNb | | pLjUbi- | | pLjUbi- | MtNFP- | | MtLyk3- | | | |
| | | tYFPnls- | | tYFPnls- | sfGFP_ | | 20aa- | | | |
| | | tNOS_F2_ | | tNOS | tNFP | | mutNbGFP_ | | | |
| | | pMtNFP_ | | | | | t35S | | | |
| | | MtNFP- | | | | | | | | |
| | | sfGFP_tNFP_ | | | | | | | | |
| | | R3_spacer_ | | | | | | | | |
| | | F4_pMLYK3_ | | | | | | | | |
| | | MtLyk3- | | | | | | | | |
| | | 20aa- | | | | | | | | |
| | | mutNbGFP_ | | | | | | | | |
| | | t35S_ELB4 | | | | | | | | |
| NFP | EC55058 | EC55058_ | EC25164_ | PMC- | EC31971_ | EC31073_ | EC32022_ | EC49283 | | |
| sfGFP_ | | L2V- | pL2V-1 | 02596_ | L1V-F2_ | pL1V-R3_ | L1V-F4_ | (ELB-4) | | |
| LYK3 | | pIV10_R1_ | pIV10 | pL1R1- | pMtNFP_ | spacer1 | pMLYK3_ | | | |
| mutNb | | pLjUbi- | | pLjUbi- | MtNFP- | | MtLyk3- | | | |
| mutNb | | tYFPnls- | | tYFPnls- | sfGFP_ | | 20aa- | | | |
| | | tNOS_F2_ | | tNOS | tNFP | | mutNbGFP- | | | |
| | | pMtNFP_ | | | | | 40aa- | | | |
| | | MtNFP- | | | | | mutNbGFP- | | | |
| | | sfGFP_tNFP_ | | | | | HA_t35S | | | |
| | | R3_spacer_ | | | | | | | | |
| | | F4_pMLYK3_ | | | | | | | | |
| | | MtLyk3- | | | | | | | | |
| | | 20aa- | | | | | | | | |
| | | mutNbGFP- | | | | | | | | |
| | | 40aa- | | | | | | | | |
| | | mutNbGFP- | | | | | | | | |
| | | HA_t35S_ | | | | | | | | |
| | | ELB4 | | | | | | | | |
| NFR5 | EC31730 | EC31730_ | EC25164_ | PMC- | PMC- | EC31073_ | EC31652_ | EC49283 | | |
| sfGFP_ | | pL2V_pIV10_ | pL2V-1 | 02596_ | 03558_ | pL1V-R3_ | L1V-F4_ | (ELB-4) | | |
| RLK4 | | R1-pLjUbi- | pIV10 | pL1R1- | L1V-F2_ | spacer1 | pLjNfr1_ | | | |
| NbNb | | tYFPnls- | | pLjUbi- | PU- | | HvRLK4cds- | | | |
| | | tNOS_F2- | | tYFPnls- | pLjNfr5- | | 20aa- | | | |
| | | pNFR5- | | tNOS | SC3- | | NbGFP- | | | |
| | | LjNFR5- | | | LjNfr5_ | | 40aa- | | | |
| | | sfGFP_T- | | | C4_ | | NbGFP-HA | | | |
| | | LjNFR5_F3- | | | sfGFP_ | | | | | |
| | | spacer_F4- | | | T- | | | | | |
| | | pLjNfr1_ | | | LjNfr5 | | | | | |
| | | HVRLK4cds- | | | | | | | | |
| | | 20aa- | | | | | | | | |
| | | NbGFP- | | | | | | | | |
| | | 40aa- | | | | | | | | |
| | | NbGFP- | | | | | | | | |
| | | HA_ELB4 | | | | | | | | |
| NFR1 | EC31792 | EC31792_ | EC25164_ | PMC- | EC31650_ | EC31073_ | EC31658_ | EC49283 | | |
| NbNb_ | | pL2V_pIV10_ | pL2V-1 | 02596_ | L1V-F2 | pL1V-R3_ | L1V-F4_ | (ELB-4) | | |
| RLK10 | | R1- | pIV10 | pL1R1- | pLjNfr1_ | spacer1 | pNFR5- | | | |
| sfGFP | | pLjUbi- | | pLjUbi- | gLjNfr1- | | LjNFR5- | | | |
| | | tYFPnls- | | tYFPnls- | 20aa- | | HvRLK10cds_ | | | |
| | | tNOS_F2- | | tNOS | NbGFP- | | T-LjNFR5 | | | |
| | | pLjNfr1_ | | | 40aa- | | | | | |
| | | gLjNfr1- | | | NbGFP- | | | | | |
| | | 20aa- | | | HA | | | | | |
| | | NbGFP- | | | | | | | | |
| | | 40aa- | | | | | | | | |
| | | NbGFP- | | | | | | | | |
| | | HA_F3- | | | | | | | | |
| | | spacer_F4- | | | | | | | | |
| | | pNFR5- | | | | | | | | |
| | | HvRLK10- | | | | | | | | |
| | | sfGFP_ | | | | | | | | |
| | | T-LjNFR5_ | | | | | | | | |
| | | ELB4 | | | | | | | | |
| RLK4 | EC31793 | EC31793_ | EC25164_ | PMC- | EC31652_ | EC31073_ | EC31658_ | EC49283 | | |
| NbNb_ | | pL2V_pIV10_ | pL2V-1 | 02596_ | L1V-F2_ | pL1V-R3_ | L1V-F4_ | (ELB-4) | | |
| RLK1 | | R1-pLjUbi- | pIV10 | pL1R1- | pLjNfr1_ | spacer1 | pNFR5- | | | |
| sfGFP | | tYFPnls- | | pLjUbi- | HvRLK4cds- | | LjNFR5- | | | |
| | | tNOS_F2- | | tYFPnls- | 20aa- | | HvRLK10cds_ | | | |
| | | pLjNfr1_ | | tNOS | NbGFP- | | T-LjNFR5 | | | |
| | | HVRLK4cds- | | | 40aa- | | | | | |
| | | 20aa- | | | NbGFP-HA | | | | | |
| | | NbGFP- | | | | | | | | |
| | | 40aa- | | | | | | | | |
| | | NbGFP- | | | | | | | | |
| | | HA_F3- | | | | | | | | |
| | | spacer_F4- | | | | | | | | |
| | | pNFR5- | | | | | | | | |
| | | HvRLK10- | | | | | | | | |
| | | sfGFP_T- | | | | | | | | |
| | | LjNFR5_ | | | | | | | | |
| | | ELB4 | | | | | | | | |
| RLK10 | EC55049 | EC55049_ | EC25164_ | PMC- | EC31973_ | EC31073_ | AE2017 | EC49283 | | |
| sfGFP_ | | L2V- | pL2V-1 | 02596_ | L1V-F2_ | pL1V-R3_ | EC31970_ | (ELB-4) | | |
| RLK4Nb | | pIV10_R1_ | pIV10 | pL1R1- | pNFR5- | spacer1 | L1V-F4_ | | | |
| | | pLjUbi- | | pLjUbi- | HvRLK10cds- | | pLjNfr1_ | | | |
| | | tYFPnls- | | tYFPnls- | sfGFP_ | | HvRLK4cds- | | | |
| | | tNOS_F2_ | | tNOS | T-LjNFR5 | | 20aa- | | | |
| | | pNFR5- | | | | | NbGFP-HA | | | |
| | | HvRLK10cds- | | | | | | | | |
| | | sfGFP_T- | | | | | | | | |
| | | LjNFR5_R3_ | | | | | | | | |
| | | spacer_F4_ | | | | | | | | |
| | | pLjNfr1_ | | | | | | | | |
| | | HvRLK4cds- | | | | | | | | |
| | | 20aa- | | | | | | | | |
| | | NbGFP- | | | | | | | | |
| | | HA_ELB4 | | | | | | | | |
| RLK10 | EC55050 | EC55050_ | EC25164_ | PMC- | EC31973_ | EC31073_ | EC32023_ | EC49283 | | |
| sfGFP_ | | L2V- | pL2V-1 | 02596_ | L1V-F2_ | pL1V-R3_ | L1V-F4_ | (ELB-4) | | |
| RLK4 | | pIV10_R1_ | pIV10 | pL1R1- | pNFR5- | spacer1 | pLjNfr1_ | | | |
| mutNb | | pLjUbi- | | pLjUbi- | HvRLK10cds- | | HvRLK4cds- | | | |
| | | tYFPnls- | | tYFPnls- | sfGFP_ | | 20aa- | | | |
| | | tNOS_F2_ | | tNOS | T-LjNFR5 | | mutNbGFP-HA | | | |
| | | pNFR5- | | | | | | | | |
| | | HvRLK10cds- | | | | | | | | |
| | | sfGFP_T- | | | | | | | | |
| | | LjNFR5_ | | | | | | | | |
| | | R3_spacer_ | | | | | | | | |
| | | F4_pLjNfr1_ | | | | | | | | |
| | | HvRLK4cds- | | | | | | | | |
| | | 20aa- | | | | | | | | |
| | | mutNbGFP- | | | | | | | | |
| | | HA_ELB4 | | | | | | | | |
| CERK6 | EC31751 | EC31751_ | EC25164_ | PMC- | EC31651_ | EC31073_ | EC31655_ | EC49283 | | |
| NbNb_ | | pL2V_pIV10_ | pL2V-1 | 02596_ | L1V-F2_ | pL1V-R3_ | L1V-F4_ | (ELB-4) | | |
| NFR5 | | R1- | pIV10 | pL1R1- | pLjNfr1_ | spacer1 | pNFR5- | | | |
| sfGFP | | pLjUbi- | | pLjUbi- | gCerk6- | | LjNFR5- | | | |
| | | tYFPnls- | | tYFPnls- | 20aa- | | sfGFP_ | | | |
| | | tNOS_F2- | | tNOS | NbGFP- | | T-LjNFR5 | | | |
| | | pLjNfr1_ | | | 40aa- | | | | | |
| | | gCerk6- | | | NbGFP-HA | | | | | |
| | | 20aa- | | | | | | | | |
| | | NbGFP- | | | | | | | | |
| | | 40aa- | | | | | | | | |
| | | NbGFP- | | | | | | | | |
| | | HA_F3- | | | | | | | | |
| | | spacer_F4- | | | | | | | | |
| | | pNFR5- | | | | | | | | |
| | | LjNFR5- | | | | | | | | |
| | | sfGFP_ | | | | | | | | |
| | | T-LjNFR5_ | | | | | | | | |
| | | ELB4 | | | | | | | | |

For tobacco transformation:

| | | | | | | | | |
|---|---|---|---|---|---|---|---|---|
| NFR5 | EC32013 | EC32013_ | EC50507_ | EC31048 | EC31888_ | EC25031 | EC31994_ | EC49283 |
| sfGFP_ | | pL2V_binary_ | pL2V | L1R1- | L1V-F2_ | pL1M- | L1V- | (ELB-4) |
| mCherryNb | | KAN- | | PUSCT- | pLjUbi_ | F3- | F4_p35S_ | |
| | | F2_pLjUbi_ | | buffer 1 | LjNFR5- | Buffer- | mCherry_ | |
| | | LjNFR5- | | | sfGFP_ | 25031 | 20linker_ | |
| | | sfGFP_T- | | | T-35S | | NbsfGFP- | |
| | | 35S- | | | | | HA_tNOS | |
| | | F4_p35S | | | | | | |
| | | mCherry_ | | | | | | |
| | | 20linker_ | | | | | | |
| | | NbsfGFP- | | | | | | |
| | | HA_tNOS_ | | | | | | |
| | | ELB4 | | | | | | |
| NFR5 | EC32015 | EC32015_ | EC50507_ | EC31048 | EC31888_ | EC25031 | EC31994_ | EC49283 |
| sfGFP_ | | pL2V_binary_ | pL2V | L1R1- | L1V-F2_ | pL1M-F3- | L1V- | (ELB-4) |
| mCherry | | KAN- | | PUSCT- | pLjUbi_ | Buffer- | F4_p35S_ | |
| mutNb | | F2_pLjUbi_ | | buffer 1 | LjNFR5- | 25031 | mCherry_ | |
| | | LjNFR5- | | | sfGFP_ | | 20linker_ | |
| | | sfGFP_T-35S- | | | T-35S | | NbsfGFP- | |
| | | F4_p35S_ | | | | | HA_tNOS | |
| | | mCherry_ | | | | | | |
| | | 20linker_ | | | | | | |
| | | mutNbsfGFP- | | | | | | |
| | | HA_tNOS_ | | | | | | |
| | | ELB4 | | | | | | |

For stable lines:

| | | | | | | | | |
|---|---|---|---|---|---|---|---|---|
| NFR5 | EC31974 | EC31974_ | EC50507_ | PMC- | PMC- | EC31073_ | EC31642_ | EC49283 |
| sfGFP_ | | pL2V_R1- | pL2V | 03816_ | 03558 | pL1V- | pL1V-F4_ | (ELB-4) |
| NFR1Nb | | pLjUbi_Hyg_ | | L1R1_ | PU-pLjNfr5- | R3_spacer1 | pNFR1_ | |
| | | t35S_F2- | | pLjUbi_ | SC3-LjNfr5 | | gNFR1_ | |
| | | pNFR5- | | Hyg_ | C4_sfGFP_ | | 20linker_ | |
| | | LjNFR5- | | t35S | T-LjNfr5- | | NbsfGFP- | |
| | | sfGFP_ | | | L1-VF2 | | HA_tNFR1 | |
| | | T-LjNFR5_ | | | | | | |
| | | F3-spacer_F4- | | | | | | |
| | | pLjNfr1_ | | | | | | |
| | | gLjNfr1- | | | | | | |
| | | 20aa- | | | | | | |
| | | NbGFP- | | | | | | |
| | | HA_tNFR1_ | | | | | | |
| | | ELB4 | | | | | | |

### Bacteria and Culture Conditions

E. coli TOP10 (ThermoFisher Scientific) were used for molecular cloning and grown in LB medium at 37° C. Agrobacterium rhizogenes strain AR1193 (32) was used for hairy root transformations. Agrobacterium strains were grown in LB medium at 28° C.

### Plant Materials

The Lotus japonicus ecotype Gifu pNin-gus wild type and mutant line nfr1-1 pNin-gus were used for nodulation assays. Plants were grown at 21° C. (16/8-hour light/dark conditions).

### Hairy Root Transformation

For scarification, Lotus seeds were either immersed in sulfuric acid for 15 minutes or treated with sandpaper. Seeds were then surface sterilized for 3 minutes with 3% sodium hypochlorite, washed 3 times in ddH₂O and dispersed on wet filter paper for germination. Three-day-old seedlings were transferred to square agar plates solidified with 0.8% Gelrite (Duchefa Biochemi) supplemented with ½ Gamborg's B35 nutrient solution (Duchefa Biochemi). A. rhizogenes AR1193 strains carrying the construct of interest were grown for three days on LB Agar containing Ampicillin, Rifampicin, and Spectinomycin (final concentration of each antibiotic was 100 μg/ml). For each construct the cells grown on one plate were resuspended in 4 mL YMB (5 g/L mannitol, 0.5 g/L yeast extract, 0.5 g/L K₂HPO₄, 0.2 g/L MgSO₄·7H₂O, 0.1 g NaCl, pH=6.8). The bacterial suspension was then used to transform 6-day-old seedlings using a 1 mL. syringe with a needle (Sterican® Ø 0.40×20 mm), punching the hypocotyl and placing a droplet on the root emerging from the wound. Square plates containing the transformed seedlings were sealed and left in the dark for one day and then moved to 21° C. under 16/8-hour light/dark conditions. After three weeks, primary roots were removed and seedlings with transformed roots were transferred to Magenta™ vessels (Sigma-Aldrich) filled with lightweight expanded clay aggregate (Leca®, 2-4 mm; Saint-Gobain Weber A/S) supplemented with 80 mL nitrogen-free ¼χ B&D nutrient solution.

### Nodulation Assay

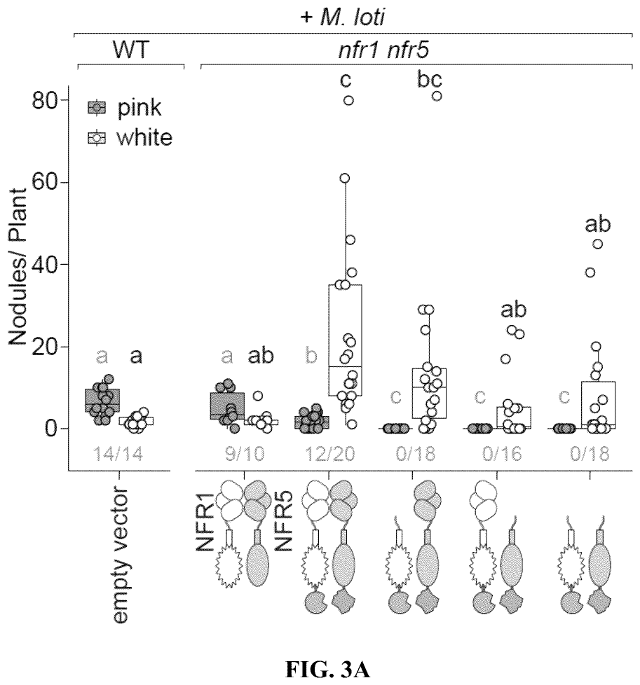

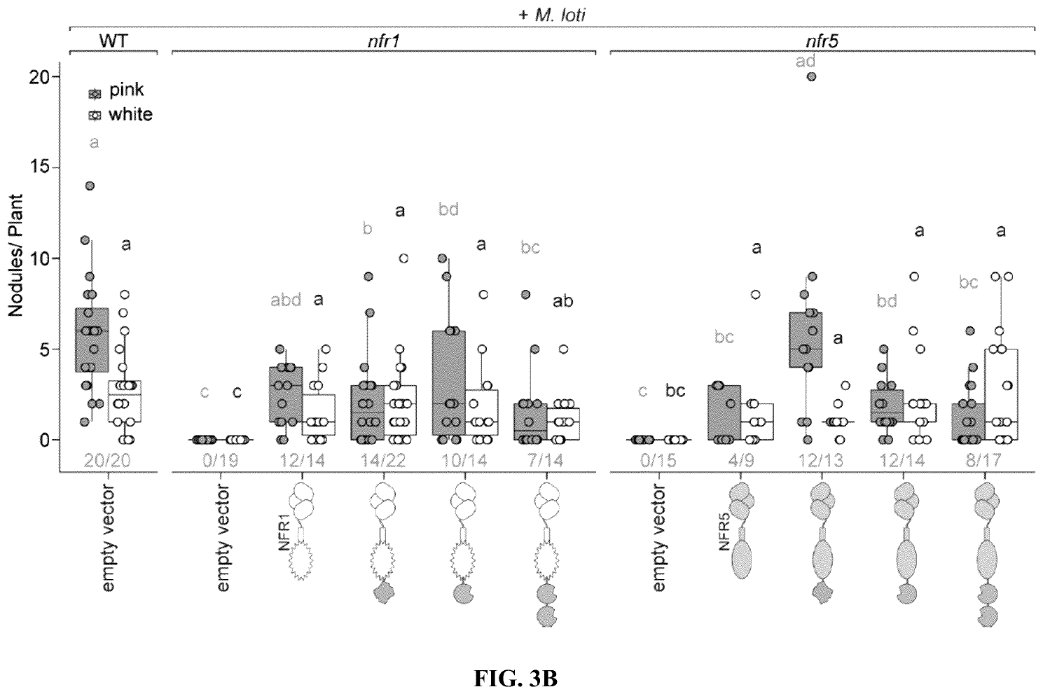

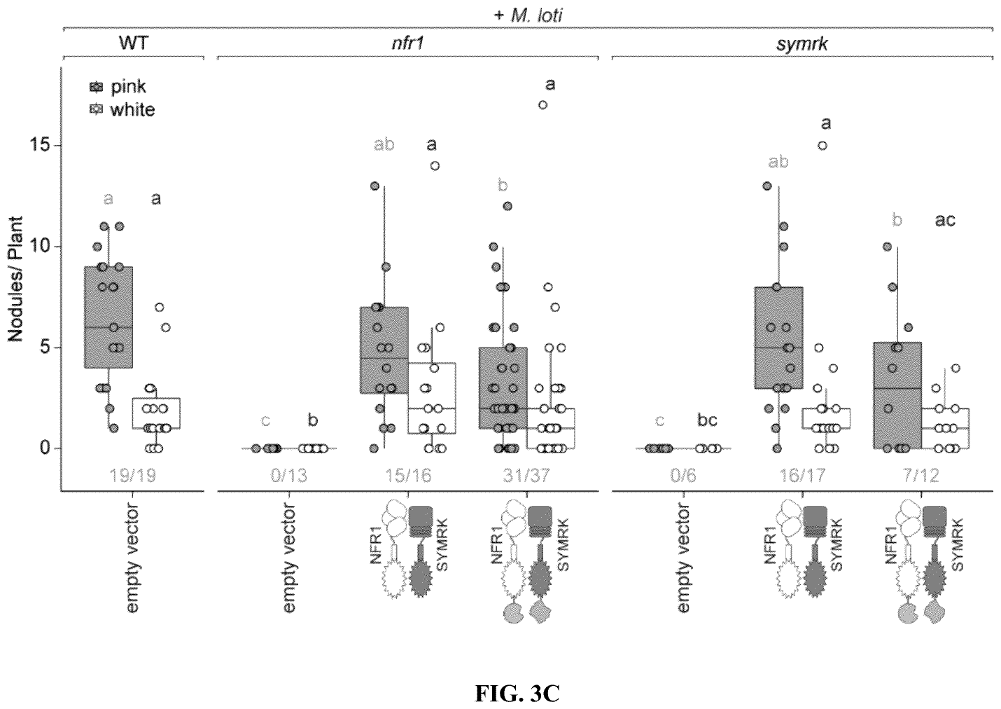

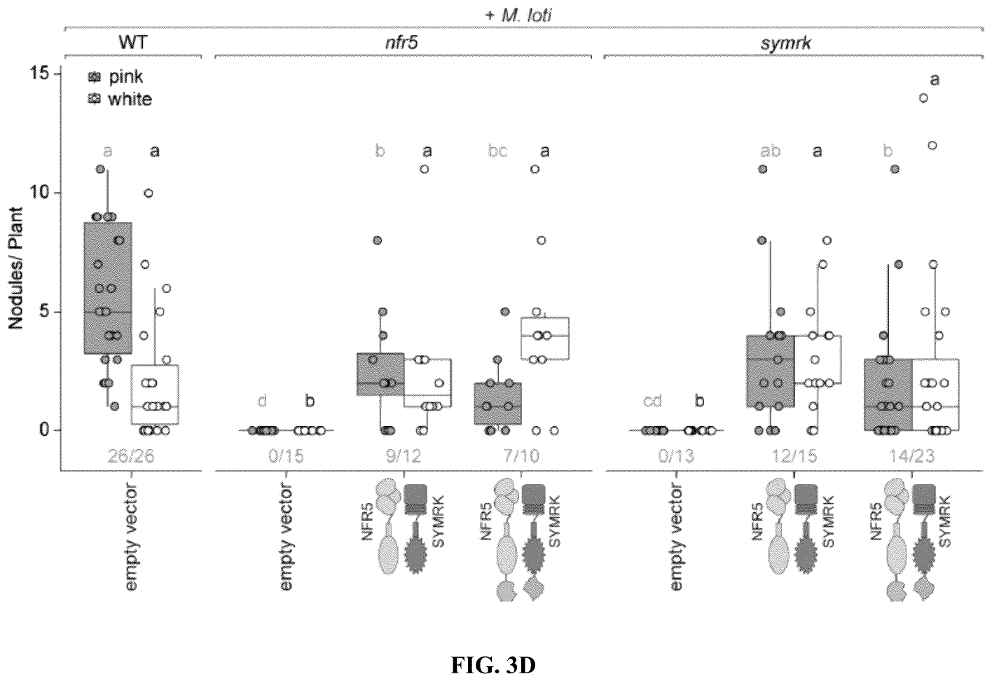

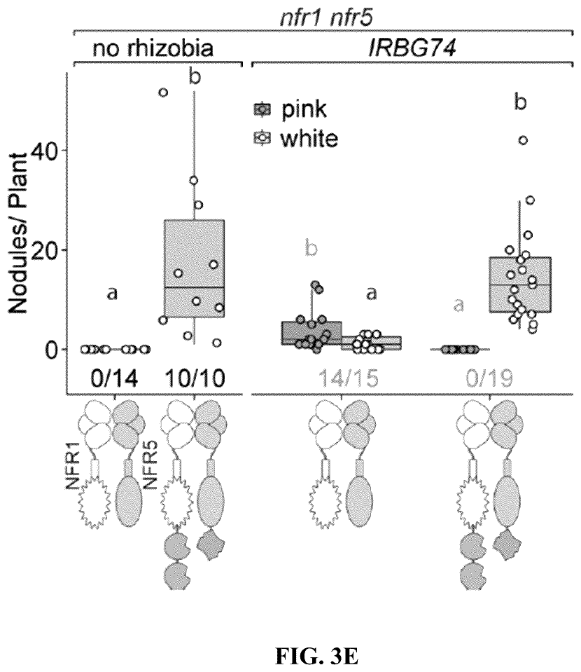

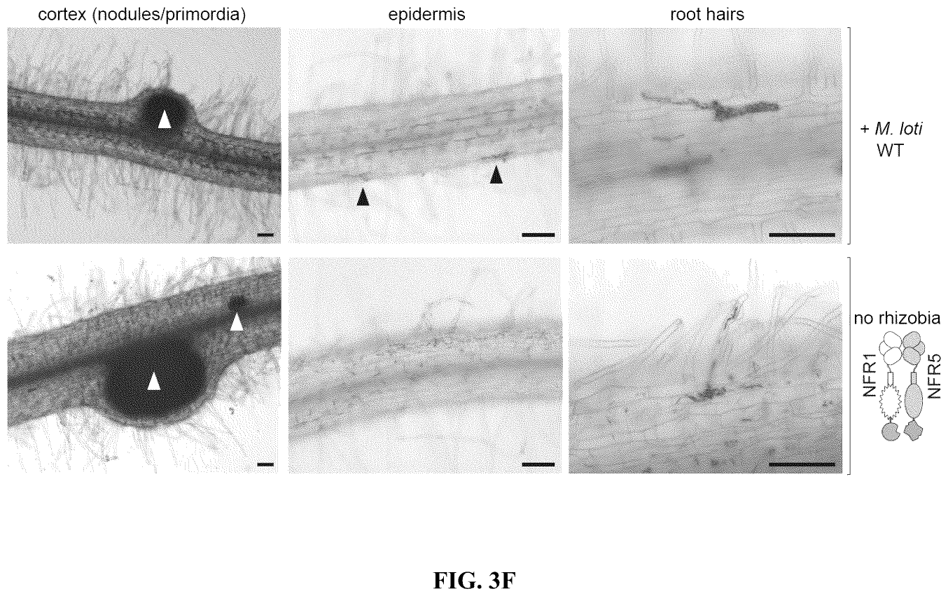

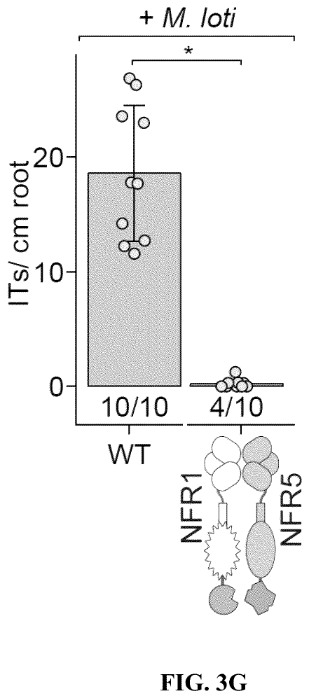

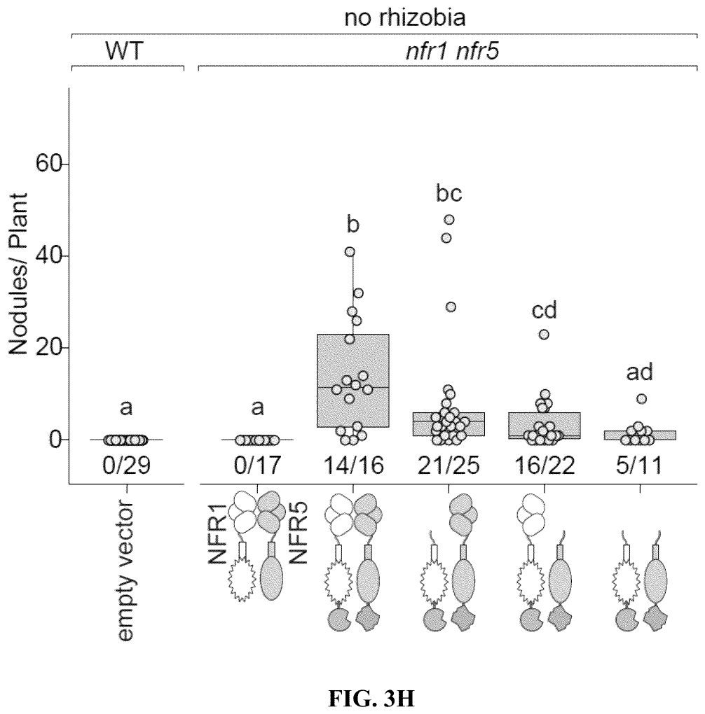

If the roots were inoculated, nodules were counted six weeks after the transfer to Magenta™ vessels (Sigma-Aldrich) and inoculation with Rhizobia. If the roots were not inoculated, nodules were counted six weeks after the transfer to Magenta™ vessels (Sigma-Aldrich) (equivalent to nine weeks after hairy root transformations). All nodulation assays in roots transformed using hairy root transformation occurred in Magenta™ vessels (Sigma-Aldrich). Nodulation assays in stable lines (see FIG. 3F) were done on plates. Pictures were acquired with a Leica M165FC Fluorescent Stereo Microscope equipped with the Leica DFC310 FX digital color camera.

A Kruskal-Wallis test (Kruskal et al. (1952) J Am Stat Assoc 47, 583-621) followed by Dunn's post-hoc test (Dunn (1964) Technometrics 6, 241-252) was used for statistical analysis of nodule counts.

### Results

### A VHH Tag was Able to Drive Specific Receptor Assembly

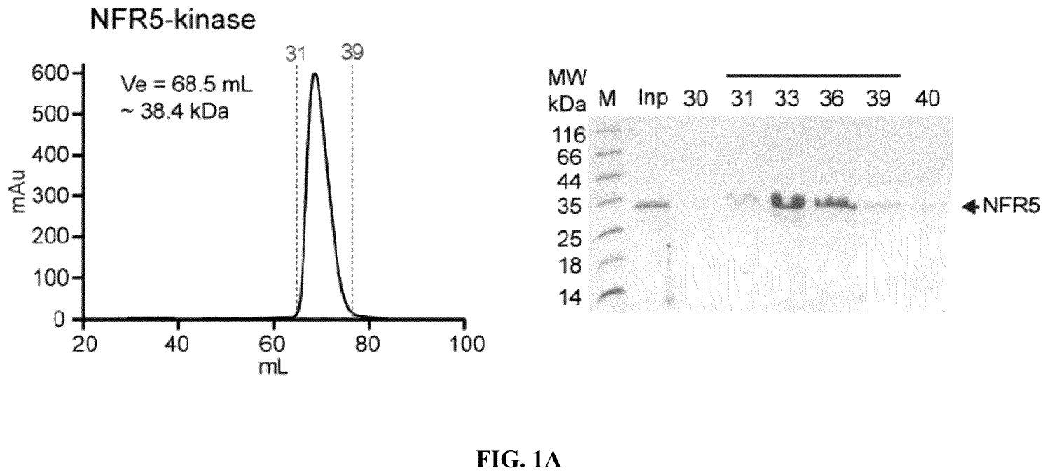

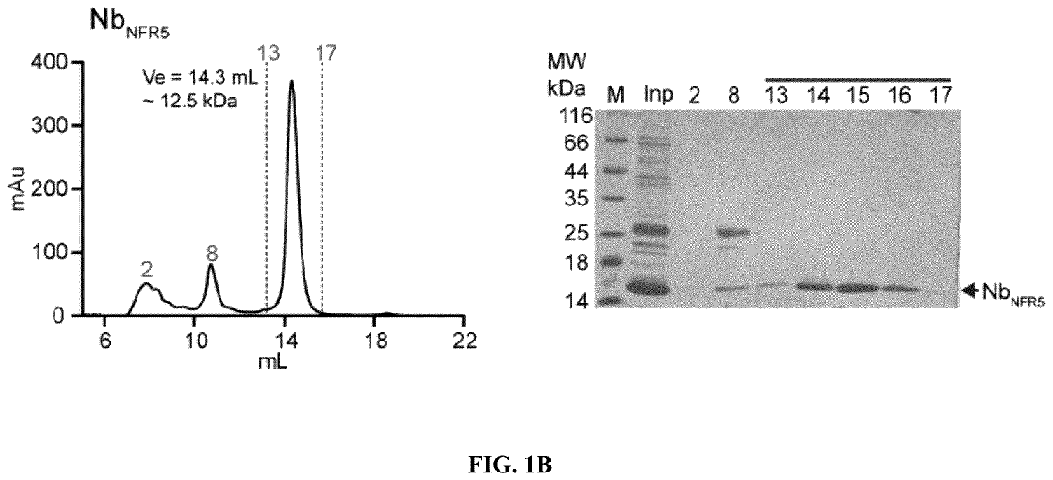

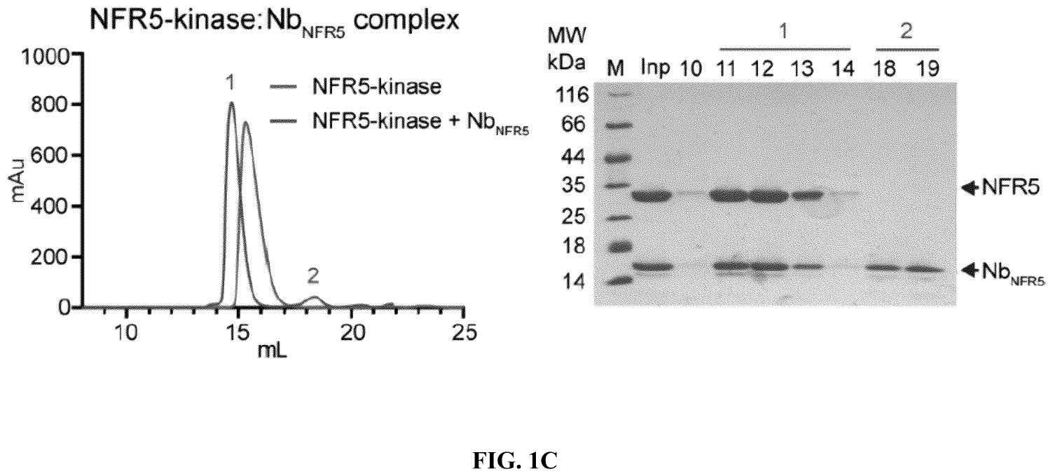

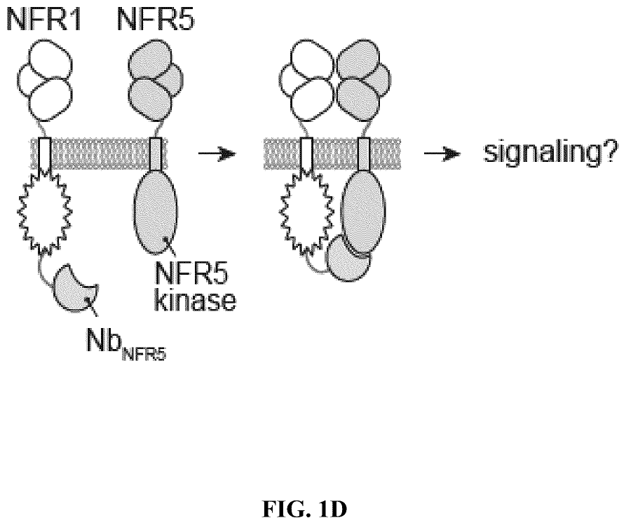

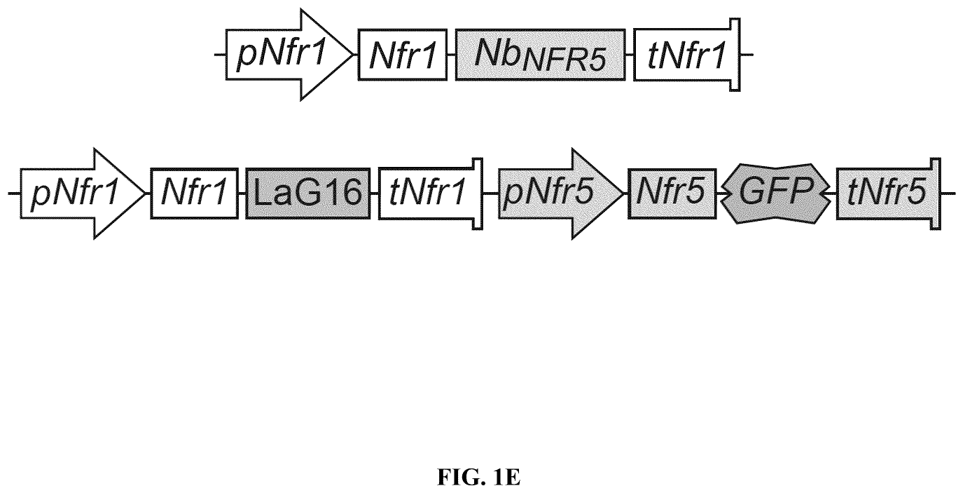

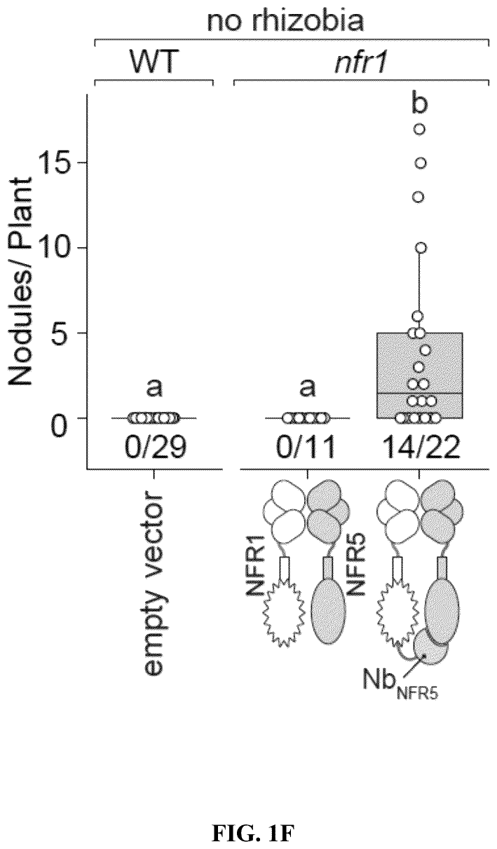

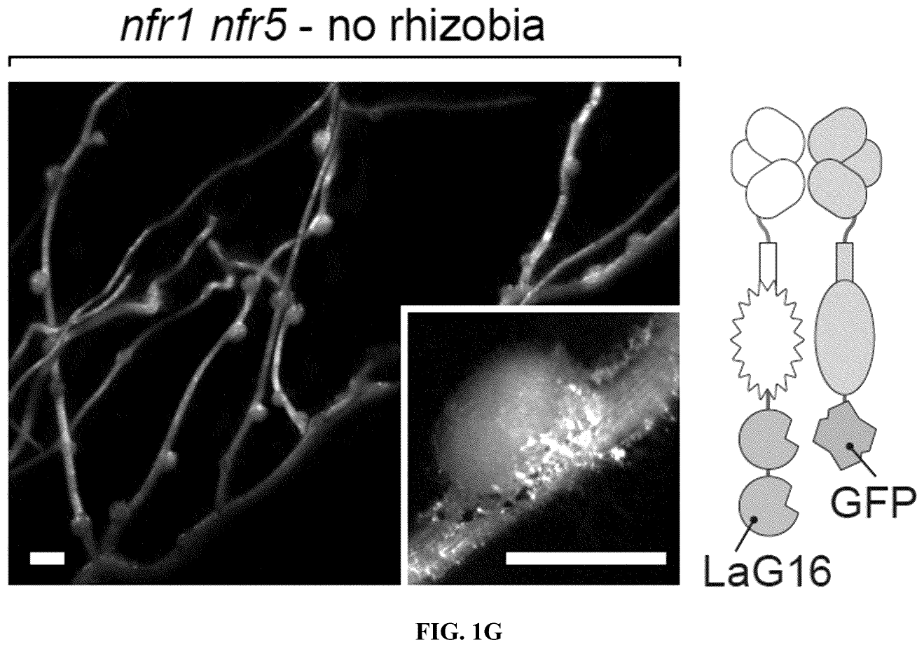

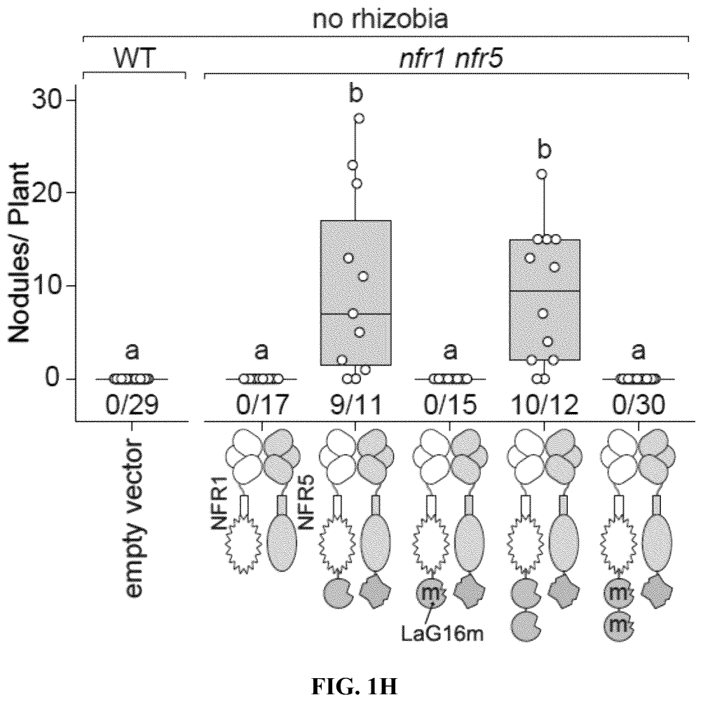

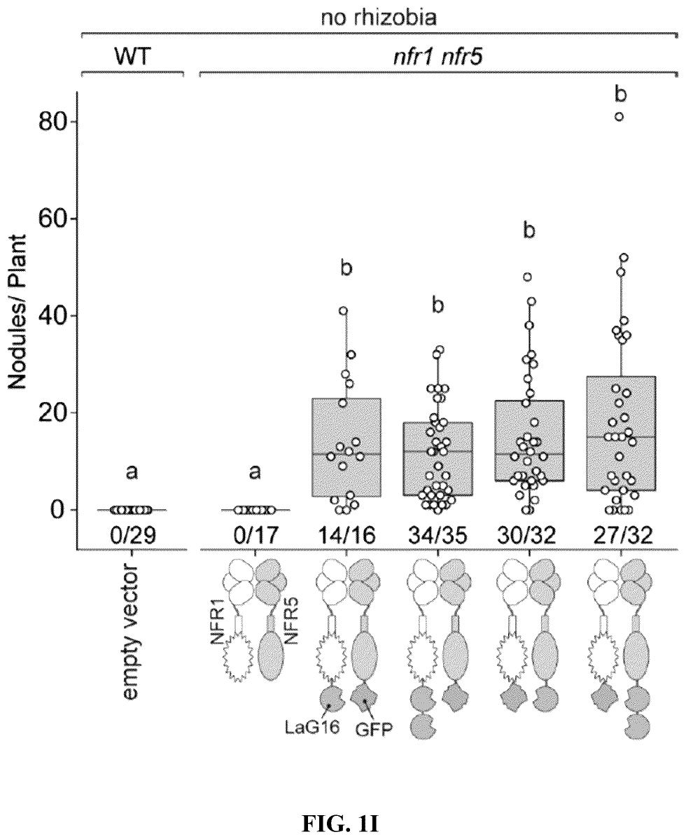

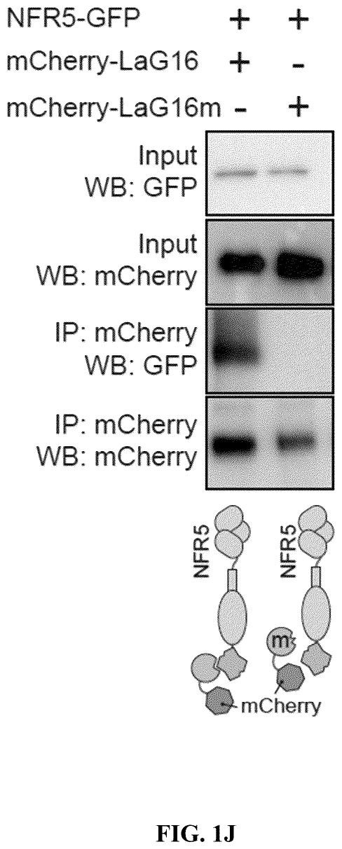

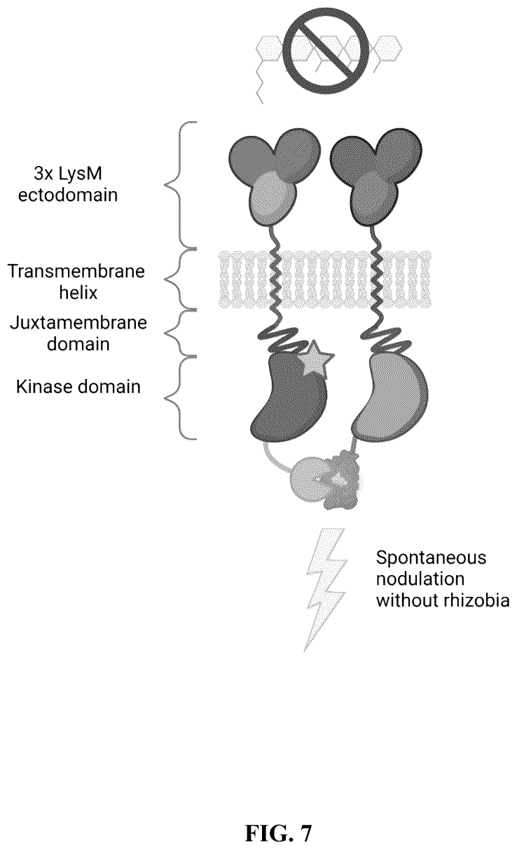

To investigate receptor complex activation, a technology that enables controlled assembly of receptors in living plant cells was developed. Symbiotic receptors NFR1 and NFR5 were chosen as proof-of-principle targets. To drive assembly, llama-derived VHHs were used as they are small, specific, and high-affinity binders that are active in the reducing environment of the cytosol (Ingram et al. (2018) Annual Review of Immunology 36, 695-715). A VHH with high affinity to the Lotus NFR5 kinase (Nb_{NFR5}) was generated (FIGS. 1A-1C, Table 2) and used as a C-terminal tag on the Lotus NFR1 receptor, with the goal that the VHH fused to the C-terminal tail of NFR1 would specifically recognize and bind the kinase domain of the native NFR5 receptor and drive the formation of a heteromeric complex (FIG. 1D). This was tested by expressing VHH-tagged NFR1 receptor constructs from the native Nfr1 promoter and terminator in Agrobacterium rhizogenes-induced nfr1 mutant plant roots (FIG. 1E, top). Nodule formation was observed independent of rhizobia or Nod factors (FIG. 1F, right), illustrating that receptor assembly is the key step in symbiotic signaling. In a control experiment, transforming Lotus nfr1 mutants with the same construct minus the VHH did not result in spontaneous nodulation (FIG. 1F, middle). A schematic of spontaneous nodulation without rhizobia is shown in FIG. 7.

This indicated that the presence of the VHH was necessary and sufficient for assembly of NFR1 and NFR5, which resulted in the formation of nodules. These data confirmed that VHHs were functional in planta and could serve as a tool to synthetically induce complex formation. It also showed that the formation of an NFR1-NFR5 complex mediated by VHHs binding to the NFR5 kinase domain induced symbiotic signaling, which led to organogenesis.

### Example 2: A VHH Towards GFP was Also Able to Drive In Vivo Receptor Assembly

The following example describes the use of a GFP tag and a VHH directed towards GFP to drive receptor assembly. The results presented in this example have widespread implications for uses in synthetic biology, as it is not always feasible to generate new customized VHHs for every experiment. As such, this general approach that does not rely on generating new customized VHHs each time provides a biological toolkit that can be used in many biological contexts.

### Materials and Methods

### Generation of Plant Expression Vector

For generation of the plant expression vector, the sequence of LaG16 (VHH directed toward GFP; SEQ ID NO: 3) was added in-frame as a C-terminal tag to the native NFR1 promoter and sequence into the pIV10_tYFP-NLS expression vector via Golden Gate cloning, and GFP (sfGFP; SEQ ID NO: 1) was added in-frame as a C-terminal tag to the native NFR5 promoter and sequence and cloned via Golden Gate cloning into PIV10_tYFP-NLS expression vector (Weber et al. (2011)PLoS One 6, e16765). The reverse, with GFP on NFR1 and LaG16 on NFR5, was also cloned. Agrobacterium rhizogenes strain AR1193 was grown in LB medium at 28° C. and transformed with pIV10 vectors via conjugation. For immunoprecipitation studies in N. benthamiana, plasmids expressing pLjUbi:Nfr5-sfGFP and p35S:mCherry-NbsfGFP were assembled by Golden Gate cloning in the pICH binary vector backbone. A construct overview is given in Table 1. VHH and fluorophore tag sequences are given in Table 2.

### Bacteria and Culture Conditions

E. coli TOP10 (ThermoFisher Scientific) were used for molecular cloning and grown in LB medium at 37° C. Agrobacterium rhizogenes strain AR1193 (32) was used for hairy root transformations and Agrobacterium tumefaciens strain GV3101 was used for transient transformation of N. benthamiana. Agrobacterium strains were grown in LB medium at 28° C.

### Plant Materials and Growth Conditions

L. japonicus ecotype Gifu wild type and mutant lines nfr1-1, nfr5-2, nfr1-1nfr5-2, symRK-3 and nfr1-1nfr5-2symRK-3 were used for nodulation assays. Nicotiana benthamiana was used for transient expression. All plants were grown at 21° C. under 16/8-h light/dark cycles.

### Hairy Root Transformation and Nodulation Assay

Hairy root transformation and nodulation assays were performed as in Example 1.

Transient Transformation of N. benthamiana Plants

The assay was performed as in Ochoa-Fernandez et al. (Nature Methods 17, 717-725 (2020)). In short, A. tumefaciens GV3101 bacteria carrying the indicated constructs in Table 2 were resuspended in infiltration solution (10 mM MgCl₂, 10 mM MES, 150 μM acetosyringone, pH 5.6) to an OD₆₀₀=0.025, incubated for two hours in the dark, and infiltrated into N. benthamiana leaves from the abaxial side with a blunt end syringe. Two days after infiltration, leaves were harvested and expression and localization were analyzed using microscopy.

TABLE 2
VHH and fluorophore tag sequences

| Construct | Sequence |
|---|---|
| Superfolder GFP | MVSKGEELFTGVVPILVELDGDVNGHKFSVSGEGEGDATYGKLTLKFICT |
| (GFP) | TGKLPVPWPTLVTTFTYGVQCFSRYPDHMKQHDFFKSAMPEGYVQERTI |
| | FFKDDGNYKTRAEVKFEGDTLVNRIELKGIDFKEDGNILGHKLEYNYNSHN |
| | VYIMADKQKNGIKVNFKIRHNIEDGSVQLADHYQQNTPIGDGPVLLPDNHY |
| | LSTQSALSKDPNEKRDHMVLLEFVTAAGITHGMDELYK (SEQ ID NO: 1) |
| Nb_{NFR5} (NFR5 | MQVQLVETGGGLVQDGDSLRLSCAASGFTFSNYYMSWVRQAPGKGLE |
| VHH) | WVSGITSGGTYTYDAVKDRFTISRDNAKNTLYLQMNSLKPEDTAVYYCAK |
| | GGTSVAVRGFYDYRGQGTQVTVSS (SEQ ID NO: 66) |
| LaG16 (GFP | MAQVQLVESGGRLVQAGDSLRLSCAASGRTFSTSAMAWFRQAPGRERE |
| VHH) | FVAAITWTVGNTILGDSVKGRFTISRDRAKNTVDLQMDNLEPEDTAVYYC |
| | SARSRGYVLSVLRSVDSYDYWGQGTQVTVSS (SEQ ID NO: 67) |
| LaG16m | MAQVQLVESGGRLVQAGDSLRLSCAASGRTWSTSAMAWFRQAPGRER |
| (mutated LaG16) | EFVAAITWWVGNTILGDSVKGRFTISRDRAKNTVDLQMDNLEPEDTAVYY |
| | CSARSRWYVLSVLRSVDSYDYWGQGTQVTVSS (SEQ ID NO: 68) |

### Co-Immunoprecipitation

The assay was performed as in Arora et al. (The Plant Cell 34, 7, 2806 (2022)). In short, N. benthamiana leaves were crushed to a powder in liquid nitrogen using a mortar and pestle, then incubated for 1 hour at 4° C. on a rotor in buffer containing 50 mM Tris-HCl (pH 7.5), 150 mM NaCl, 10% (v/v) glycerol, 2 mM EDTA, 5 mM DTT, 1 mM phenylmethylsulfonyl fluoride, Protease Inhibitor Cocktail (Sigma-Aldrich), and 1% (v/v) IGEPAL® CA-630 (Alfa Aesar) in a ratio of 2 mL/g tissue. Extracts were centrifuged at 4° C. and 4700 rpm for 30 minutes. Supernatants were directly incubated with RFP-Trap® Magnetic Agarose beads (ChromoTek®) overnight at 4° C. The beads were washed five times in buffer containing 50 mM Tris-HCl (pH 7.5), 150 mM NaCl, 10% (v/v) glycerol, 2 mM EDTA, Protease Inhibitor Cocktail (Sigma-Aldrich), and 0.5% (v/v) IGEPAL® CA-630 (Alfa Aesar) on ice. To release the proteins, 50 μL of SDS sample buffer (25 mM Tris-HCl, pH 6.8, 10% glycerol, 2% SDS, 0.2% 2-mercaptoethanol, 0.6 mg/mL Bromophenol blue) was added and samples were heated for 5 minutes at 95° C.

### Western Blot

Proteins were separated on 12% SDS-polyacrylamide gels at 120 V for 3 h. Blotting to PVDF membranes (Merck) was done by wet transfer for 2 hours at 90 V. Membranes were blocked with 5% milk for 1 hour. For detection of GFP, membranes were incubated over night at 4° C. with mouse anti-GFP (632592; TaKaRa; 1:5000) followed by anti-mouse HRP (A4416; Sigma-Aldrich; 1:10,000) for 2 hours. For detection of mCherry, membranes were incubated overnight at 4° C. with rabbit anti-mCherry (632496; TaKaRa; 1:5000) followed by anti-rabbit HRP (A6154; Sigma-Aldrich; 1:10,000) for 2 hours. Chemiluminescence was detected on a G-BOX (Syngene) using ECL Prime Western Blotting Detection Reagent (Cytiva).

### Results

### A GFP-Tag and a VHH-Tag Towards GFP on Separate Receptors Resulted in Receptor Association

The well-characterized LaG16 VHH that binds superfolder green fluorescent protein (GFP) (Fridy et al. (2014) Nature Methods 11, 1253-1260) was fused to NFR1, and GFP was fused to NFR5. These constructs were expressed from their native promoters and terminators in nfr1 nfr5 double mutant plants (FIG. 1E, bottom). When NFR1 was associated to NFR5 through VHH binding to GFP, nodules were formed independently of rhizobia (FIG. 1G and FIG. 1H, third column). Similarly, organogenesis was activated in the reverse situation with GFP fused to NFR1 and the VHH fused to NFR5, indicating that the VHH could be fused to either NFR1 or NFR5 (FIG. 1I, fifth column).

Receptor Association was Abolished when the VHH Towards GFP Lost GFP-Binding Ability

To test whether the observed phenotype was dependent on the direct binding between VHH and GFP, a mutant version of LaG16 was engineered to have three bulky tryptophan residues in the VHH paratope. In contrast to functional LaG16, mutated LaG16 (LaG16m) did not have the ability to bind GFP-tagged NFR5 in tobacco pull-down assays (FIG. 1J). Furthermore, when LaG16m was fused to NFR1 it failed to initiate organogenesis (FIG. 1H, fourth and sixth columns), demonstrating that NFR1-NFR5 receptor association is essential for signal transduction.

### A Single VHH was Sufficient to Induce Nodulation

To explore whether signaling was enhanced by assembling more receptors, two LaG16 VHHs were coupled in series to either NFR1 or NFR5, separated by a 40-residue long (˜130 Å) flexible linker which should sterically allow one receptor to associate with two co-receptors (FIG. 1H, fifth column and FIG. 1I, fourth and sixth columns). No difference in nodule formation was observed when one or two VHHs were used, indicating that a single association event between NFR1 and NFR5 receptors is sufficient to initiate nodule organogenesis (FIG. 1H, third and fifth columns and FIG. 1I, rightmost four columns).

### Example 3: Identification of a Conserved Core Receptor Complex in Legumes Through Use of VHH Activation of Dimeric Receptors

The following example describes the use of VHH activation of a heteromeric LYK3-NFP receptor state in Medicago truncatula, resulting in the identification of a conserved receptor complex in legumes.

### Materials and Methods

### Generation of Plant Expression Vector

For generation of Medicago expression vectors, sequences of NFP and LYK3 were assembled with NFP and LYK3 promoters, respectively, and with 35S terminators. These sequences were further assembled with VHH or fluorophore tags into the pIV10_tYFP-NLS expression vector via Golden Gate cloning (Weber et al. (2011) PLoS One 6, e16765). Agrobacterium rhizogenes strain AR1193 was grown in LB medium at 28° C. and transformed with pIV10 vectors via conjugation. A construct overview is given in Table 1.

### Plant Materials

The Medicago strains used were Medicago truncatula cv. wild type Jemalong A17 and lyk3nfp (hcl1-1nfp2). All plants were grown at 21° C. under 16/8-h light/dark cycles.

### Hairy Root Transformation and Nodulation Assay

Hairy root transformation and nodulation assays were performed as in Example 1, with the following exception: Medicago truncatula seeds were immersed in sulfuric acid for 3 minutes, instead of 15 minutes, and one-day old Medicago seedlings were transferred to agar plates, compared to 3-day-old Lotus seedlings.

### Results

### Conservation of the NRF1-NFR5 Dimeric Receptor Complex in the Process of Symbiotic Organogenesis Signaling is Observed in Medicago.

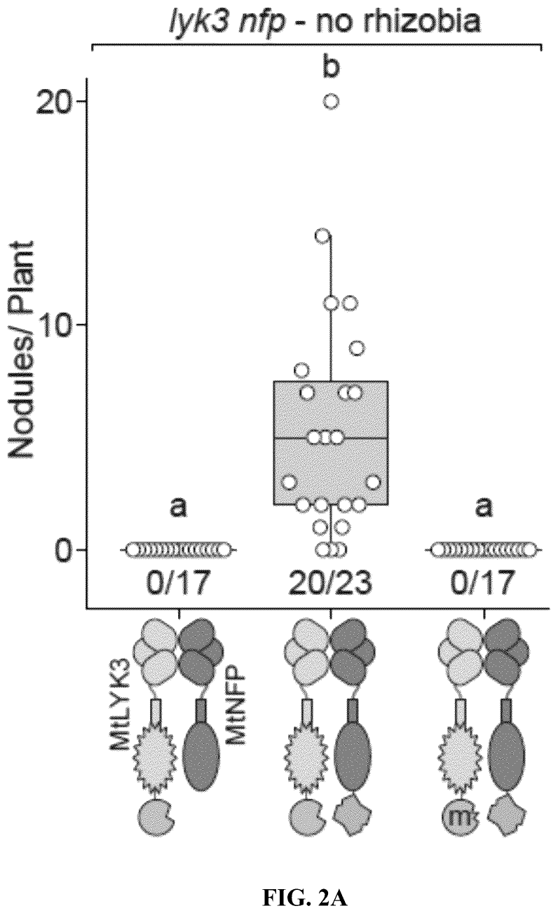

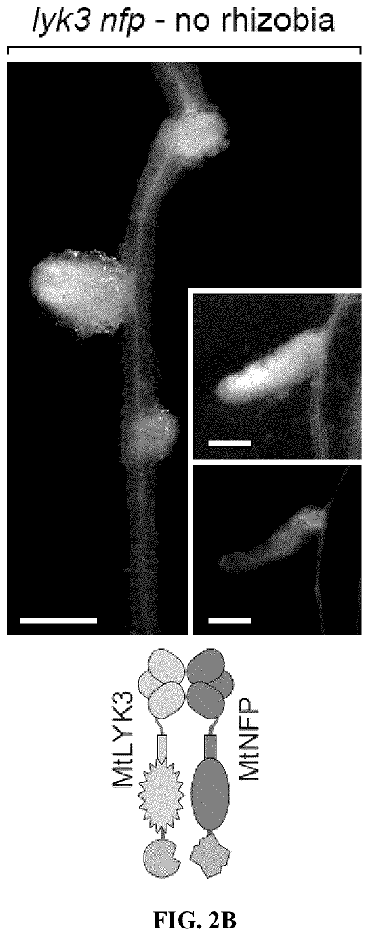

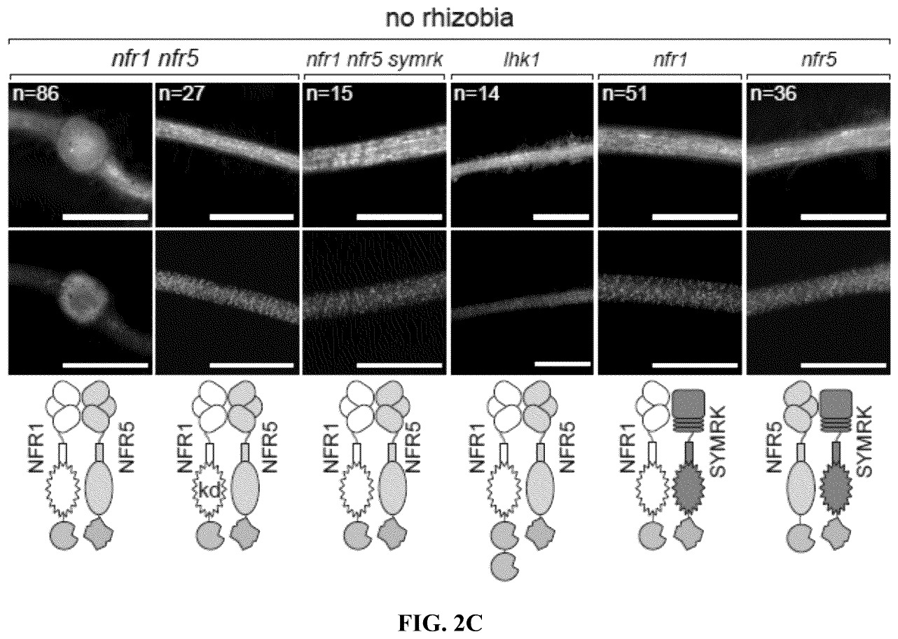

To understand if the heteromeric receptor complex corresponds to the activated receptor state in other legume-rhizobia symbioses, analogous experiments were performed in Medicago truncatula (Medicago), where the two Nod factor receptors LYK3 and NFP were associated via VHHs. A heteromeric LYK3-NFP receptor complex (i.e., VHH-driven dimerization of NFP and LYK3) was observed to be sufficient to initiate organogenesis in Medicago in the absence of external signals (FIG. 2A and FIG. 2B), demonstrating that the triggering event for signal activation leading to organogenesis is the associate of a conserved core receptor complex comprising a NFR1-type receptor and a NFR5-type receptor.

### Conserved Core Complex Activity is Mediated by Phosphorylation and Cytokinin Signaling

To understand if symbiotic signaling was exclusively dependent on the physical assembly of the receptors or if it also requires a phosphorylation event, a point mutation was introduced into NFR1, impairing its kinase function (Madsen et al. (2011) Plant Journal 65, 405-417). Plants that expressed the kinase-dead NFR1 failed to form nodules (FIG. 2C, second column), showing that the protein kinase activity mediates the downstream signaling pathway. The SYMRK receptor containing a malectin-like domain and leucine-rich repeats in the ectodomain has previously been shown to interact with NFR5 and to induce spontaneous nodules when overexpressed (Ried et al. (2014) eLife 3, e03891). To decipher the composition and functional requirements of the core complex, VHH-assembled NFR1-NFR5 signaling was tested in the nfr1 nfr5 symrk triple mutant plants. The transformed roots showed no nodulation (FIG. 2C, third column), verifying the dependence on the common symbiosis pathway and confirming the vital downstream function of SYMRK.

To investigate if it was possible to drive symbiotic signaling from SYMRK independently of the formed NFR1-NFR5 complex, VHHs were used to associate SYMRK to either NFR1 or NFR5. Neither the SYMRK-NFR1 nor the SYMRK-NFR5 complex drove organogenesis signaling (FIG. 2C, rightmost two columns), showing that the essential role of SYMRK in nodulation requires either a ligand- or VHH-induced NFR1-NFR5 complex.

To verify that the activated receptor complex operated through the known organogenesis pathways (Tirichine et al. (2007) Science 315, 104-107; Murray et al. (2007) Science 315, 101-104), its dependency on cytokinin signaling was explored. The activated NFR1-NFR5 complex was unable to initiate organogenesis in the lhk1 loss-of-function cytokinin receptor mutant (FIG. 2C, fourth column). Together, the data show that the NFR1-NFR5 complex is essential to initiate nodule organogenesis and that the downstream signaling cascade requires the symbiotic pathway.

### Example 4: VHH-Mediated Receptor Complex Initiates Both Cortical and Epidermal Programs

The following example describes the use of VHH-mediated NFR1-NFR5 receptor assembly to investigate the independent roles of the complex in infection and organogenesis signaling.

### Materials and Methods

### Generation of Plant Expression Vector

For generation of plant expression vectors, sequences of RLKs were assembled with the native NFR1 or NFR5 promoter- and terminator sequences and VHH or fluorophore tags into the pIV10_tYFP-NLS expression vector via Golden Gate cloning (Weber et al. (2011) PLoS One 6, e16765). A construct overview is given in Table 1.

### Bacteria and Culture Conditions

E. coli TOP10 (ThermoFisher Scientific) were used for molecular cloning and grown in LB medium at 37° C. Mesorhizobium loti strain R7A (Kelly et al. (2013) Molecular Plant-Microbe Interactions 26, 319-329) constitutively expressing the fluorescent protein DsRed or IRBG74 (Cummings et al. (2009) Environmental Microbiology 11, 2510-2525) was grown in YMB (5 g/L mannitol, 0.5 g/L yeast extract, 0.5 g/L K₂HPO₄, 0.2 g/L MgSO₄·7H₂O, 0.1 g NaCl, pH 6.8) medium at 28° C. Agrobacterium rhizogenes strain AR1193 was used for hairy root transformations. Agrobacterium strains were grown in LB medium at 28° C.

### Plant Materials

The following lines were used for nodulation assays: Lotus japonicus Gifu (Handberg et al. (1992) Plant Journal 2, 487-796), Gifu_pNin-gus, nfr1-1_pNin-gus, nfr5-2_pNin-gus, nfr1-1nfr5-2_pNin-gus (Madsen et al. (2003) Nature 425, 637-640; Gysel et al. (2021) PNAS 118, e2111031118), lhk1-1 (Murray et al. (2007) Science 315, 101-104), symRK-3 (Stracke et al. (2002) Nature 417, 959-962), and nfr1-1nfr5-2symRK-3. All plants were grown at 21° C. under 16/8-h light/dark cycles. Stable transformants of Lotus were generated as described in Thykjor et al. (Cell Biology: A Laboratory Manual, Academic Press Inc., Orlando Florida, 1997, pg. 518-525) using Agrobacterium-mediated hypocotyl transformation and regeneration with the phytohormone concentrations of Lombari et al. (Lotus japonicus Handbook, A. J. Marquez et al., Eds. Springer, Berlin, Germany, 2005, 251-259).

### Hairy Root Transformation and Nodulation Assay

Hairy root transformation was performed as in Example 1. Nodulation assays were done in Magenta™ vessels (Sigma-Aldrich). Mesorhizobium loti strain R7A constitutively expressing the fluorescent protein DsRed was grown in TY/YMB medium at 28° C. After transfer to magentas, plants were inoculated with 150 μl per plant of M. loti R7A DsRed strain, at a final concentration of OD₆₀₀=0.04. At six weeks post inoculation, nodules were counted and pictures were acquired with a Leica M165FC Fluorescent Stereo Microscope equipped with the Leica DFC310 FX camera. Nodulation assays were performed four weeks after inoculation with IRBG74.

A Kruskal-Wallis test (Kruskal et al. (1952) J Am Stat Assoc 47, 583-621) followed by Dunn's post-hoc test (Dunn (1964) Technometrics 6, 241-252) was used for statistical analysis of nodule counts. Statistical analyses for pink and white nodule counts were done separately.

### Nodulation and Infection Thread Assays on Plates

Three-day-old seedlings were transferred to square plates containing a slope of ¼ B&D medium (Broughton et al. (1971) Biochem J 125, 1075-1080) solidified with 1.4% Noble Agar (Difco™). The slope was covered with wet filter paper (AGF 651; Frisenette Aps) and a metal bar with 3 mm holes for roots was placed at the top of the agar slope. Plates were placed in boxes excluding light from the roots below the metal bar. Where needed, plants were inoculated with 75 μL of M. loti per plant at an OD₆₀₀ of 0.02. A Kruskal-Wallis test was used for statistical analysis of infection thread counts.

### GUS Staining

Roots of 15-day-old stable line seedlings were cut and incubated in GUS staining buffer (0.5 mg/mL 5-bromo-4-chloro-3-indolyl-β-D-glucuronic acid (X-Gluc), 100 mM potassium phosphate buffer (pH 7.0), 10 mM EDTA (pH 8.0), 1 mM potassium ferricyanide, 1 mM potassium ferrocyanide and 0.1% Triton X-100) at 37° C. overnight. Roots were washed with 70% ethanol. Pictures were taken using a Zeiss Axioplan 2 imaging microscope and processed using ImageJ (Schneider et al. (2012) Nature Methods 9, 671-675). The WT line used for GUS staining was a transformed line carrying pNIN:GUS (Gifu_pNin-gus).

### Results

Infection Occurred but was Reduced in Plants with VHH-Mediated NFR1-NFR5 Complexes

To investigate the effect of the VHH-induced NFR1-NFR5 complex on the infection program, plants with and without the VHH receptor fusion system were inoculated with Mesorhizobium loti (M. loti) bacteria, the symbiont of Lotus. Infection by M. loti was monitored via the appearance of pink, leghemoglobin-producing nodules on the roots. While an increased number of white nodules formed on plants expressing the VHH-associated NFR1-NFR5 complex, formation of infected pink nodules was reduced compared to plants expressing the native receptors (FIG. 3A and FIG. 3B). There was no impairment of infection when using a single receptor GFP or VHH as a tag (FIG. 3B). These results suggest that the activated NFR1-NFR5 complex initiates both parallel developmental programs of infection and organogenesis. Compared to organogenesis, however, infection was hampered by the presence of the VHH-driven heteromeric NFR1-NFR5 state. Interestingly, plants expressing the VHH-induced complexes of either NFR1-SYMRK or NFR5-SYMRK were efficiently infected by rhizobia (FIG. 3C and FIG. 3D). Infection was not impaired upon VHH-mediated association of NFR1-SYMRK (FIG. 3C), nor was it impaired upon VHH-mediated association of NFR5-SYMRK (FIG. 3D). These results suggest that only the activated NFR1-NFR5 receptor state affects the infection program.

It was considered possible that rhizobia that use an intercellular infection mechanism and circumvent the epidermal root hair infection program could more efficiently colonize the nodules induced by the VHH-mediated receptor complexes. To test this, infection of nodules was tested after inoculation with IRBG74, which uses an intercellular, crack-entry mechanism (Cummings et al. (2009) Environmental Microbiology 11, 2510-2525; Montiel et al. (2021) Plant Physiology 185, 1131-1147). The nfr1 nfr5 mutant roots only formed white, uninfected nodules (FIG. 3E, rightmost column), suggesting that both infection modes were impaired when NFR1 and NFR5 were part of a VHH-bound complex.

### VHHs Induce Constitutive Receptor Complex Formation in Roots

To understand how downstream processes are affected by the presence of the VHH-activated receptor complex, stable Lotus lines were generated that expressed GFP-tagged NFR5 and LaG16-tagged NFR1 in the nfr1 nfr5 double mutant background with a 0-glucuronidase (GUS) reporter gene driven by the NIN promoter. The effect of the activated receptor state on both infection and organogenesis was evaluated by looking at the expression of the transcription factor NIN, which orchestrates both the epidermal infection program and the cortical organogenesis program (Schauser et al. (1999) Nature 402, 191-195). Nodule organogenesis was observed independent of rhizobia in three independent stable lines at 7 to 10 days after germination. GUS staining revealed NIN activation in nodules as well as in dividing cortical cells which mark sites of emerging nodule primordia (FIG. 3F, bottom). This cortical NIN activation was very similar to the native condition for wild type plants inoculated with M. loti (FIG. 3F, top). In wild type, the epidermal infection program was tightly regulated, as NIN was only expressed in few epidermal root hair cells. In contrast, plants expressing the VHH-driven NFR1-NFR5 complex showed broad NIN expression in numerous epidermal cells along the root (FIG. 3F). This wider and less regulated epidermal NIN activation might interfere with the coordinated timing of organogenesis and infection (FIG. 3A). Infection efficiencies were quantified by comparing wild type plants with the stable Lotus lines expressing the NFR1-NFR5 complex 10 days after inoculation with M. loti. A strong reduction in the number of infection threads formed was observed when VHHs were used to constitutively activate NFR1-NFR5 (FIG. 3G). Infection and organogenesis signaling are independent functions of the NFR1-NF5 complex

Since transgenic roots expressing the VHH-mediated complex initiated both organogenesis and infection, the possibility that certain receptor domains contributed differently to the two developmental processes could be investigated. For this, VHH-mediated complexes were engineered with receptors lacking the ectodomains of NFR1, NFR5 or both. These complexes were expressed in transgenic roots. All these ectodomain-lacking versions of the complexes were still found to activate organogenesis, albeit with a reduced efficiency compared to full-length receptors. As shown in FIG. 3H, ectodomains of NFR1 and NFR5 were required for full efficiency of organogenesis activation. By contrast, removal of any ectodomain resulted in complete loss of infection by M. loti as no infected pink nodules appeared (FIG. 3A, rightmost three columns). These data show that the infection program was dependent on functional Nod factor-recognizing ectodomains, while organogenesis signaling could be mediated solely from the intracellular NFR1-NFR5 domains. Furthermore, this demonstrates that it is possible to bypass the ligand-induced signaling function conferred by the ectodomains to induce the cortical organogenesis program.

### Example 5: VHH Constructs Showed Conservation of Function of NFR1 and NFR5 Orthologs in Barley

The following example describes the use of VHHs in molecular biology to test the conservation of functionality of orthologs of NFR1 and NFR5 from Medicago and barley, and their role in infection and symbiosis-mediated organogenesis.

### Materials and Methods

### Generation of Plant Expression Vector

For generation of plant expression vectors, sequences of barley RLKs were assembled with the native NFR1 or NFR5 promoter- and terminator sequences and VHH or fluorophore tags into the pIV10_tYFP-NLS expression vector via Golden Gate cloning (Weber et al. (2011) PLoS One 6, e16765). Agrobacterium rhizogenes strain AR1193 was grown in LB medium at 28° C. and transformed with pIV10 vectors via conjugation. A construct overview is given in Table 1.

### Plant Materials

L. japonicus ecotype Gifu_pNin-gus wild type and mutant lines nfr1-1_pNin-gus, nfr5-2_pNin-gus, and nfr1-1nfr5-2_pNin-gus were utilized. All plants were grown at 21° C. under 16/8-h light/dark cycles.

### Hairy Root Transformation and Nodulation Assay

Hairy root transformation was performed as in Example 1. Nodulation assays were performed as in Example 4.

### Protein Modeling

Structure predictions were made using Alphafold v.2.2.0 (Jumper et al. (2021) Nature 596, 583-589; Varadi et al. (2022) Nucleic Acids Research 50, D439-D444) with default parameters. Since lipids are not included in the predictions the receptor ectodomains and kinases were not restricted to be on opposite sides of the membrane. To visualize a possible physical orientation of the receptors, the conformations of a few residues were manually rebuilt in the juxtamembrane parts in Coot (Crystallographic Object-Oriented Toolkit, Emsley et al. (2010) Acta Crystrallographica D. Biological Crystallography 66, 486-501). The models were aligned and visualized in PyMOL (The PyMOL Molecular Graphics System, Version 2.0, Schrödinger, LLC).

### Phylogenetic Analysis

Phylogenetic trees were constructed based on a multiple sequence alignment of full-length receptors created with the CLC Main Workbench 22.0.1 (QIAGEN). UPGMA and Jukes-Cantor were set as algorithm and distance measures, respectively. Amino acid sequence percent identities were compared with the CLC Main Workbench 22.0.1 (QIAGEN).

### Results

### Cereal Receptors can Activate Root Nodule Organogenesis

The ability to develop nitrogen-fixing root nodules is restricted to a single clade of flowering plants (Soltis et al. (1995) PNAS 92, 2647-2651). However, most plants, including cereals, can form arbuscular mycorrhizal symbiosis by activating the common symbiosis pathway that is shared with root nodule symbiosis in legumes (Kistner et al. (2005) Plant cell 17, 2217-2229; Radhakrishnan et al. (2020) Nature Plants 6, 280-289). This dichotomy indicates that the LysM receptors from cereals are unable to activate downstream signaling leading to nodulation.

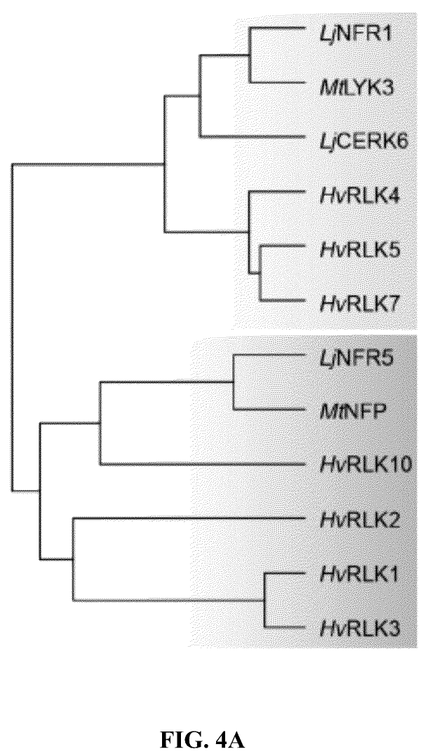

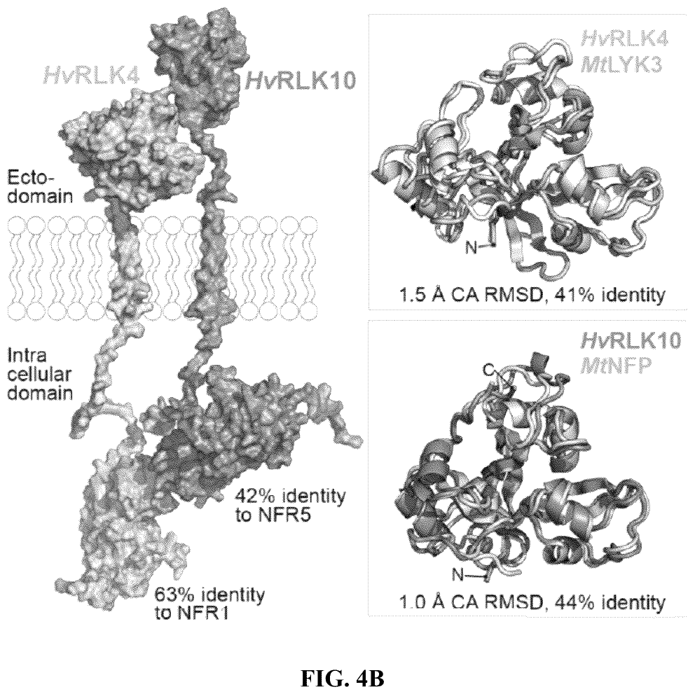

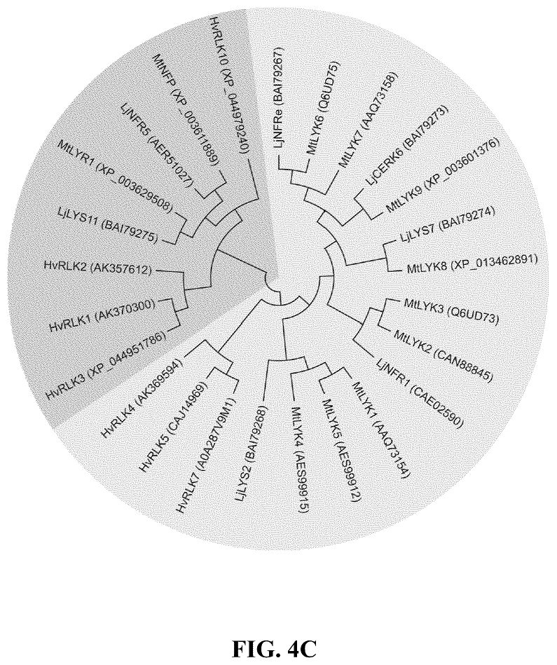

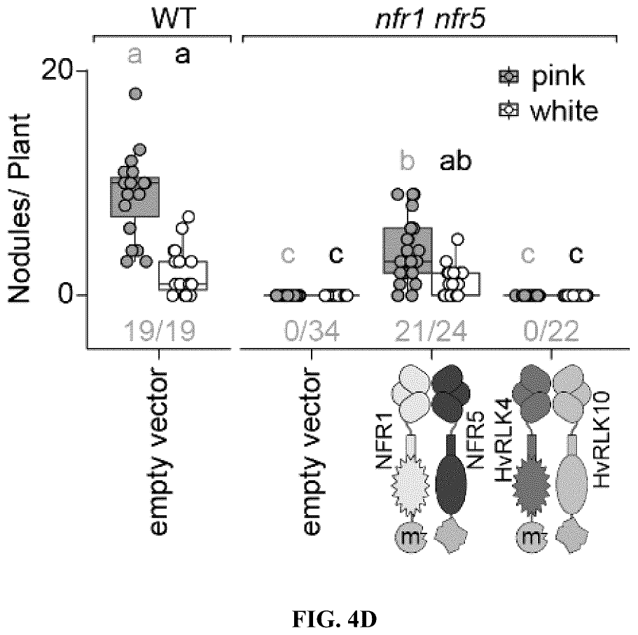

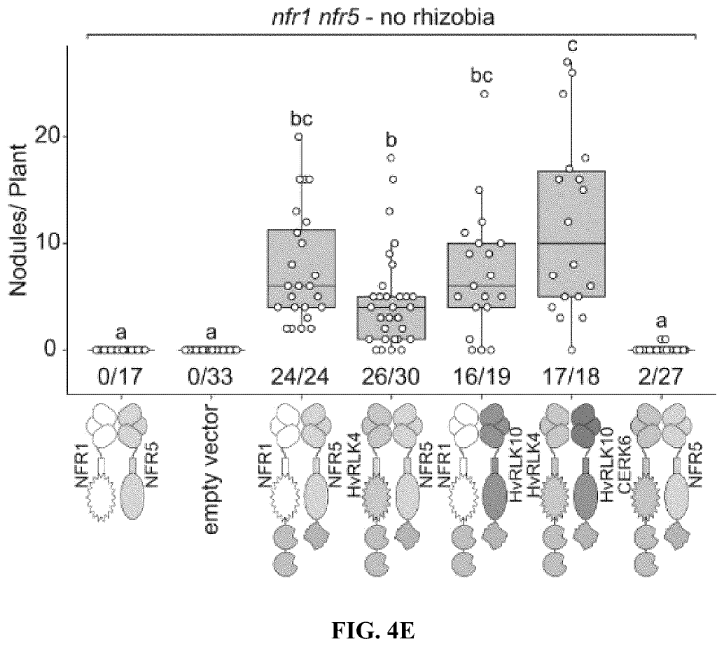

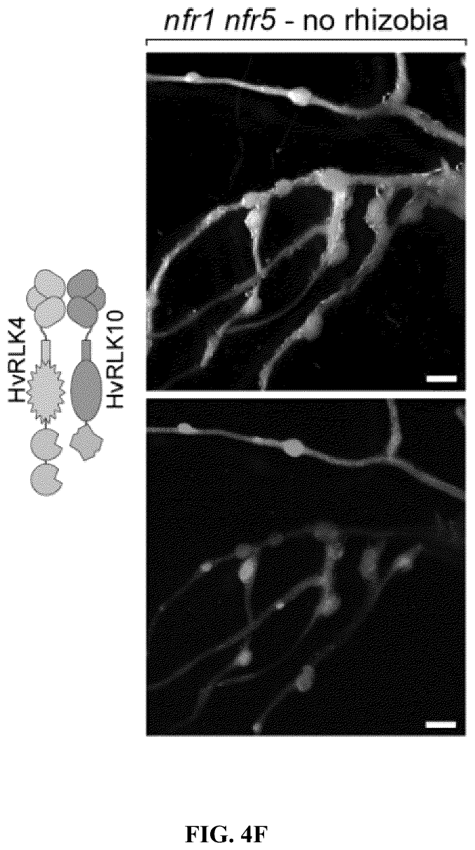

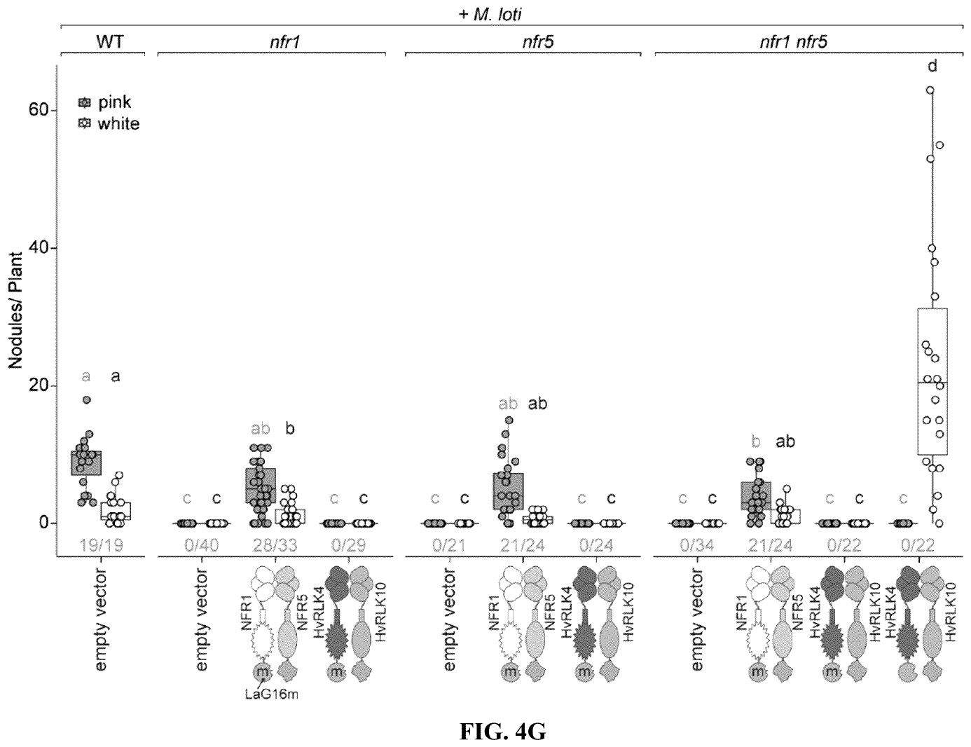

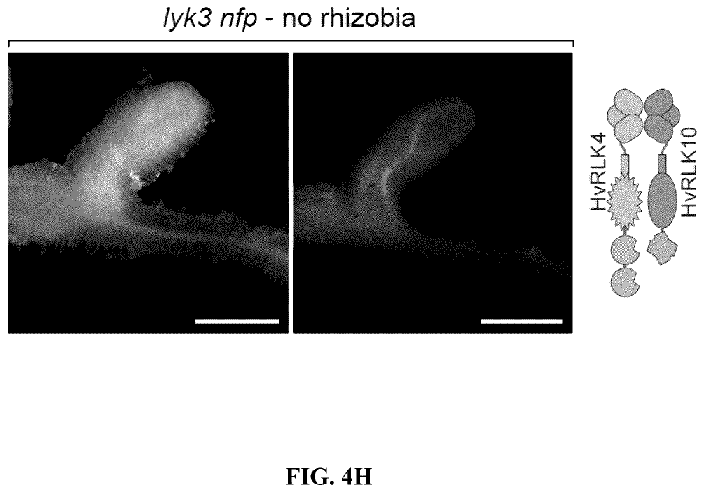

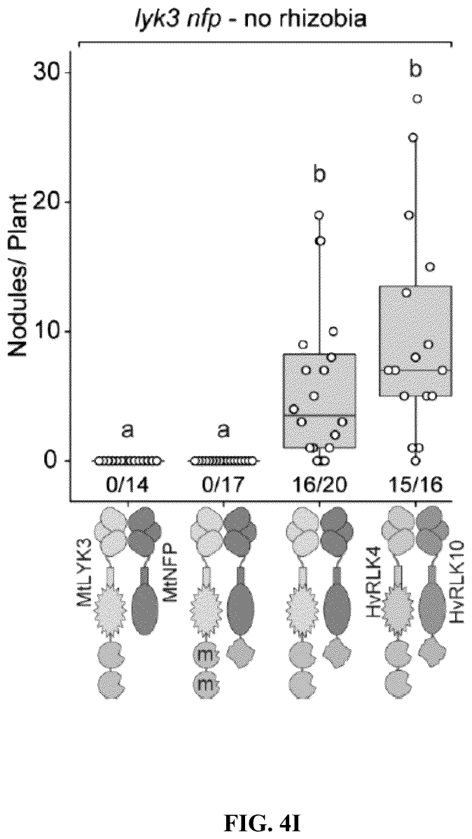

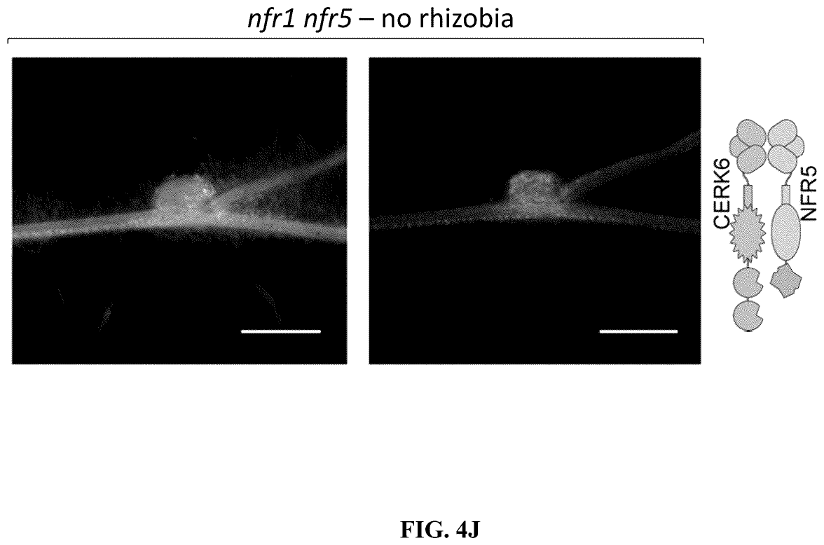

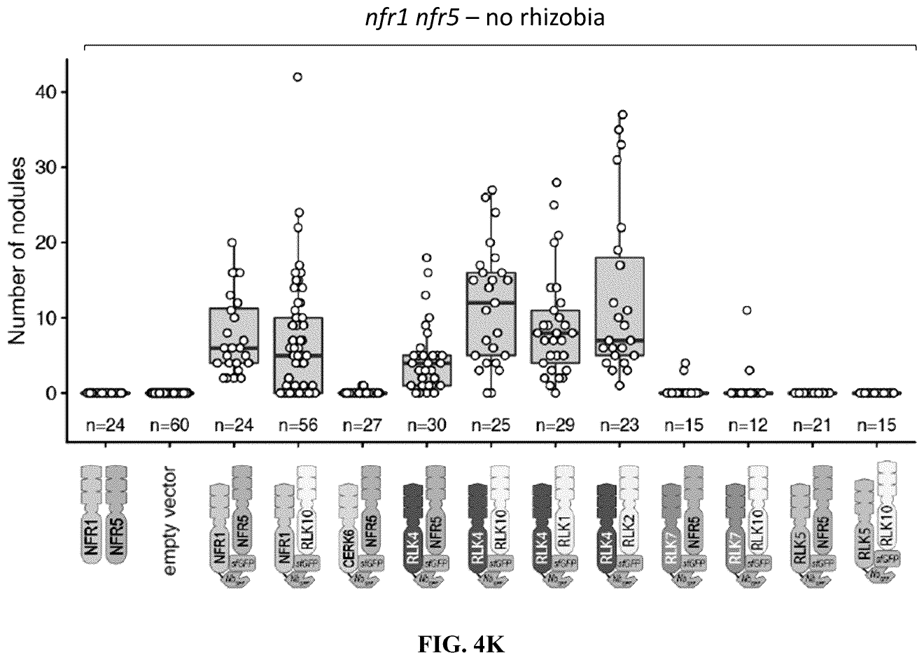

Genomic analysis was performed to identify receptors in barley with symbiotic signaling capacity, resulting in the identification of seven LysM receptor-like kinases (RLKs) belonging to the NFR1 and NFR5 families (FIG. 4A). Phylogenomic analysis and structural modeling of barley RLKs revealed that RLK4 and RLK10 showed the greatest similarity to the NFR1-type and NFR5-type classes of receptors, respectively (FIG. 4A, FIG. 4B, FIG. 4C) (Bozsoki et al. (2020) Science 369, 663-670; Gysel et al. (2021) PNAS 118, e211103111). The ability of the individual RLKs to functionally complement the nfr1 and nfr5 single or nfr1 nfr5 double mutants when expressed from the native Nfr1 and Nfr5 promoters and terminators was first tested. Despite the similarity, RLK4 and RLK10 were unable to functionally complement NFR1 and NFR5 (FIG. 4D, and FIG. 4G, third pink-and-white column pair of nfr1, nfr5, and nfr1 nfr5 genotypes, and Table 3). The possibility of VHH-driven association of Lotus NFR5 with barley RLK4 expressed from the native Nfr1 promoter and terminator was then tested in nfr1 nfr5 double mutant plants, resulting in nodules formed independently of rhizobia (FIG. 4E, fourth column). Likewise, nodules formed when barley RLK10 was in a VHH-mediated complex with Lotus NFR1 (FIG. 4E, fifth column). Moreover, associating barley RLK4 and RLK10 via VHHs led to nodule organogenesis independent of the Nod factor receptors in both Lotus and Medicago (FIG. 4E, sixth column; FIG. 4F; FIG. 4H; and FIG. 4I). Barley RLK4 and RLK10 failed to support infection as no pink nodules appeared when plants were inoculated with M. loti (FIG. 4G, third pink column of nfr1, nfr5, and nfr1 nfr5 genotypes).

Together, these surprising data demonstrated that LysM receptors from barley, which diverged from Lotus 200 million years ago (Wolfe et al. (1989) PNAS 86, 6201-6205), have retained the basic ability to activate the symbiotic pathway leading to root nodule organogenesis in legumes. Further investigation was performed to determine if the Lotus chitin receptor CERK6 (SEQ ID NO: 58; Bozsoki et al. (2017) PNAS 114, E8118-E8127), the closest homologue to Lotus NFR1 and in the same receptor family as barley RLK4, could support nodulation. Lotus CERK6 shares 62% sequence identity with Lotus NFR1 (Table 3), but only two out of 27 plants formed a single nodule when CERK6 and NFR5 were associated via VHHs in the nfr1 nfr5 mutants (FIG. 4E, rightmost column, FIG. 4J). This result shows that the ability to activate organogenesis was encoded within the individual receptors and that signaling specificity was ensured by receptor complex composition.

TABLE 3
Comparison of amino acid sequence identities
Percentage of amino acid identity between full-length receptors and intracellular domains
(in parenthesis) of receptors used in this application.

| | LjNFR1 | MtLYK3 | LjCERK6 | HvRLK4 | LjNFR5 | MtNFP | HvRLK10 | LjSYMRK |
|---|---|---|---|---|---|---|---|---|
| LjNFR1 | — | 78 (88) | 62 (70) | 51 (63) | 24 (29) | 25 (30) | 24 (24) | 18 (28) |
| MtLYK3 | | — | 62 (72) | 52 (64) | 25 (30) | 26 (30) | 23 (24) | 17 (28) |
| LjCERK6 | | | — | 58 (70) | 24 (28) | 23 (29) | 24 (26) | 18 (28) |
| HvRLK4 | | | | — | 24 (28) | 23 (28) | 24 (26) | 17 (29) |
| LjNFR5 | | | | | — | 72 (80) | 38 (42) | 15 (22) |
| MtNFP | | | | | | — | 37 (40) | 16 (20) |
| HvRLK10 | | | | | | | — | 14 (19) |
| LjSYMRK | | | | | | | | — |

### Distal Barley NFR5-Type Receptors Activate Root Nodule Organogenesis, but Distal Barley NFR1-Type Receptors do not

The barley genome contains four NFR5-type receptors, RLK1 (SEQ ID NO: 59; SEQ ID NO: 64), RLK2 (SEQ ID NO: 60; SEQ ID NO: 65), RLK3 (SEQ ID NO: 62), and RLK10 (SEQ ID NO: 57), of which RLK11 was the closest relative to the Lotus NFR5 (FIG. 4C, green shading). RLK1 and RLK2 were tested for their ability to induce spontaneous nodulation in the absence of rhizobia. The VHH-mediated association of RLK4 with NFR5, RLK10, RLK1, or RLK2 induced spontaneous nodulation in Lotus nfr1 nfr5 double mutant roots (FIG. 4K, sixth through ninth columns) and showed the conservation of functionality in NFR5-type receptors from Lotus to barley.

The barley genome contains three NFR1-type receptors, RLK4 (SEQ ID NO: 56), RLK5 (SEQ ID NO: 61), and RLK7 (SEQ ID NO: 63) (FIG. 4C. yellow shading). VHH-mediated association of RLK4 with Lotus NFR5 induced spontaneous nodulation in Lotus nfr1 nfr5 double mutant roots (FIG. 4E, fourth column, and FIG. 4K, sixth column). VHH-mediated association of RLK7 with NFR5 or with RLK10 rarely induced spontaneous nodulation (FIG. 4J, tenth and eleventh columns). The association of RLK5 with NFR5 or RLK10 failed to induce nodulation (FIG. 4K, rightmost two columns). This indicated that the conservation of function for activating organogenesis did not extend to all NFR1-type receptors in barley.

What is claimed is:

1. A genetically modified cell comprising:
a transmembrane (TM) receptor complex comprising a first subunit polypeptide and a second subunit polypeptide,
wherein the first subunit polypeptide comprises an affinity polypeptide that binds to the second subunit polypeptide intracellularly inducing oligomerization; and
wherein oligomerization of the first subunit polypeptide and the second subunit polypeptide activates TM receptor signaling, wherein the affinity polypeptide is heterologous to the first subunit polypeptide.

2. The genetically modified cell of claim 1, wherein the affinity polypeptide binds directly to the second subunit polypeptide, and/or wherein the second subunit polypeptide comprises a tag polypeptide, and wherein the affinity polypeptide binds to the tag polypeptide.

3. The genetically modified cell of claim 2, wherein the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, and a ubiquitin tag polypeptide.

4. The genetically modified cell of claim 1, wherein the affinity polypeptide is selected from a heavy-chain variable domain (VHH), a single-chain variable fragment (scFV), a designed ankyrin repeat protein (DARPins), a human fibronectin III domain 3 monobody, an Affibody, and an anticalin, and a synthetic version of any of the foregoing.

5. The genetically modified cell of claim 1, wherein the cell is a eubacterial cell, an archaeal cell, or a eukaryotic cell, optionally wherein the eukaryotic cell is a plant cell, an animal cell, or a fungal cell.

6. The genetically modified cell of claim 1, wherein the cell is a plant cell and the first subunit polypeptide and/or the second subunit polypeptide are selected from a LysM receptor, a Leucine rich repeat (LRR) receptor, a Malectin like receptor, a plant TM receptor, a plant SPTM (pSPTM) receptor, and a plant SPTM receptor comprising an intracellular kinase domain (pSPTM-kinase), optionally wherein the first subunit polypeptide and/or the second subunit polypeptide lacks an ectodomain and/or a transmembrane domain.

7. The genetically modified cell of claim 6, wherein the first subunit polypeptide is a NFR1 polypeptide and the second subunit polypeptide is a NFR5 polypeptide, wherein the first subunit polypeptide is a NFR5 polypeptide and the second subunit is a NFR1 polypeptide, wherein the first subunit polypeptide is a LYK3 polypeptide and the second subunit polypeptide is a NFP polypeptide, wherein the first subunit polypeptide is a NFP polypeptide and the second subunit polypeptide is a LYK3 polypeptide, wherein the first subunit polypeptide is a RLK4 receptor polypeptide and the second subunit polypeptide is a RLK10 receptor polypeptide, or wherein the first subunit polypeptide is a RLK10 polypeptide and the second subunit polypeptide is a RLK4 polypeptide.

8. The genetically modified cell of claim 7, wherein the NFR1, LYK3, or RLK4 polypeptide is selected from the group of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 56, and wherein the NFR5, NFP, or RLK10 polypeptide is selected from the group of SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, and SEQ ID NO: 57.

9. A bispecific affinity polypeptide comprising:
a first affinity polypeptide that binds to an intracellular portion of a first subunit polypeptide of a TM receptor; and
a second affinity polypeptide that binds to an intracellular portion of a second subunit polypeptide of the TM receptor;
wherein binding of the first affinity polypeptide to the first subunit polypeptide and of the second affinity polypeptide to the second subunit polypeptide induces oligomerization; and
wherein oligomerization of the first subunit polypeptide and the second subunit polypeptide activates TM receptor signaling.

10. The bispecific affinity polypeptide of claim 9, wherein the first affinity polypeptide binds directly to the first subunit polypeptide and/or wherein the second affinity polypeptide binds directly to the second subunit polypeptide; or wherein the first subunit polypeptide comprises a tag polypeptide, and wherein the first affinity polypeptide binds to the tag polypeptide, and/or wherein the second subunit polypeptide comprises a tag polypeptide, and wherein the second affinity polypeptide binds to the tag polypeptide.

11. The bispecific affinity polypeptide of claim 10, wherein the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, and a ubiquitin tag polypeptide.

12. The bispecific affinity polypeptide of claim 9, wherein the first affinity polypeptide and the second affinity polypeptide are selected from a heavy-chain variable domain (VHH), a single-chain variable fragment (scFV), a designed ankyrin repeat protein (DARPins), a human fibronectin III domain 3 monobody, an Affibody, and an anticalin, and a synthetic version of any of the foregoing.

13. A method of screening transmembrane (TM) receptor complexes comprising:
(a) providing a cell expressing a first subunit polypeptide of a TM receptor;
(b) expressing a second subunit polypeptide of a TM receptor in the cell; and
(c) assaying the cell for a TM receptor phenotype; and
wherein (i) the presence of the TM receptor phenotype indicates the first subunit polypeptide and the second subunit polypeptide oligomerize to form the TM receptor; or (ii) the absence of the TM receptor phenotype indicates the first subunit polypeptide and the second subunit polypeptide do not oligomerize to form a TM receptor, and wherein (1) the second subunit polypeptide comprises a tag polypeptide, the first subunit polypeptide comprises an affinity polypeptide that binds to the tag polypeptide, and the affinity polypeptide is heterologous to the first subunit polypeptide, or (2) the first subunit polypeptide comprises the tag polypeptide, the second subunit polypeptide comprises the affinity polypeptide that binds to the tag polypeptide, and the affinity polypeptide is heterologous to the second subunit polypeptide.

14. The method of claim 13, wherein the binding partner for the first subunit polypeptide is unknown and step (b) is repeated using two or more candidate second subunit polypeptides to identify the second subunit polypeptide that is the binding partner for the first subunit polypeptide.

15. The method of claim 13, wherein the tag polypeptide is selected from a fluorescent protein polypeptide, a luminescence polypeptide, a flu hemagglutin tag polypeptide, a c-myc tag polypeptide, a Herpes Simplex virus glycoprotein D (gD) tag polypeptide, a poly-histidine tag polypeptide, a FLAG™ tag polypeptide, a KT3 epitope tag polypeptide, a tubulin epitope tag polypeptide, a T7 gene 10 protein tag polypeptide, streptavidin tag polypeptide, a Vesicular Stomatis viral glycoprotein (VSV-G) epitope tag polypeptide, a small epitope (Pk) found on the P and V proteins of the paramyxovirus of simian virus 5 (V5) tag polypeptide, an alkaline phosphatase (AP) tag polypeptide, a bluetongue virus tag (B-tag) polypeptide, a calmodulin binding peptide (CalBP) tag polypeptide, a chloramphenicol acetyl transferase (CAT) tag polypeptide, a choline-binding domain (CholBD) tag polypeptide, a chitin binding domain (ChitBD) tag polypeptide, a cellulose binding domain (CellBP) tag polypeptide, a dihydrofolate reductase (DHFR) tag polypeptide, a galactose-binding protein (GBP) tag polypeptide, a maltose binding protein (MBP) polypeptide, a glutathione-S-transferase (GST) polypeptide, a Glu-Glu (EE) tag polypeptide, a human influenza hemagglutinin (HA) tag polypeptide, a horseradish peroxidase (HRP) tag polypeptide, a NE-tag polypeptide, a HSV tag polypeptide, a ketosteroid isomerase (KSI) tag, a LacZ tag polypeptide, a NusA tag polypeptide, a PDZ domain tag polypeptide, a AviTag polypeptide, a SBP-tag polypeptide, a Softag 1 polypeptide, a Softag 3 polypeptide, a TC tag polypeptide, a VSV-tag polypeptide, an Xpress tag polypeptide, an Isopeptag polypeptide, a SpyTag polypeptide, a SnoopTag polypeptide, a Profinity eXact tag polypeptide, a Protein C tag polypeptide, a 51-tag polypeptide, a S-tag polypeptide, a biotin-carboxy carrier protein (BCCP) tag polypeptide, a small ubiquitin-like modifier (SUMO) tag polypeptide, a tandem affinity purification (TAP) tag polypeptide, a HaloTag polypeptide, a Nus-tag polypeptide, a Thioredoxin-tag polypeptide, a CYD tag polypeptide, a HPC tag polypeptide, a TrpE tag polypeptide, and a ubiquitin tag polypeptide.

16. The method of claim 13, wherein the affinity polypeptide is selected from a heavy-chain variable domain (VHH), a single-chain variable fragment (scFV), a designed ankyrin repeat protein (DARPins), a human fibronectin III domain 3 monobody, an Affibody, and an anticalin, and a synthetic version of any of the foregoing.

17. The method of claim 13, wherein the cell is a eubacterial cell, an archaeal cell, or a eukaryotic cell, optionally wherein the eukaryotic cell is a plant cell, an animal cell, or a fungal cell.

18. A genetically modified plant or part thereof comprising the genetically modified plant cell of claim 1 and/or the bispecific affinity polypeptide of claim 9.

19. The genetically modified plant or part thereof of claim 18, wherein the plant is selected from the group consisting of cassava, corn, cowpea, rice, barley, wheat, Trema spp., apple, pear, plum, apricot, peach, almond, walnut, strawberry, raspberry, blackberry, red currant, black currant, melon, cucumber, pumpkin, squash, grape, bean, soybean, pea, chickpea, cowpea, pigeon pea, lentil, Bambara groundnut, lupin, pulses, Medicago spp., Lotus spp., forage legumes, indigo, legume trees, and hemp.

20. A method of making the genetically modified plant cell of claim 1, comprising introducing a genetic alteration to the plant cell comprising a first nucleic acid sequence encoding a heterologous first subunit polypeptide comprising an affinity polypeptide; and/or introducing a genetic alteration to the plant cell comprising a second nucleic acid sequence encoding a heterologous second subunit polypeptide optionally comprising a tag polypeptide; or comprising genetically modifying the plant cell by transforming the plant cell with one or more gene editing components that target a first endogenous nuclear genome sequence encoding the first subunit polypeptide, wherein the first subunit polypeptide is genetically modified to comprise the affinity polypeptide; and/or genetically modifying the plant cell by transforming the plant cell with one or more gene editing components that target a second endogenous nuclear genome sequence encoding the second subunit polypeptide, wherein the endogenous second subunit polypeptide is genetically modified to comprise a tag polypeptide.

21. An expression vector or isolated DNA molecule comprising one or more nucleotide sequences encoding a first subunit polypeptide of a TM receptor comprising an affinity polypeptide, wherein the affinity polypeptide is heterologous to the first subunit polypeptide, and wherein the one or more nucleotide sequences are operably linked to at least one expression control sequence; one or more nucleotide sequences encoding a second subunit polypeptide of a TM receptor optionally comprising a tag polypeptide, wherein the one or more nucleotide sequences are operably linked to at least one expression control sequence; one or more nucleotide sequences encoding: (a) a first subunit polypeptide of a transmembrane (TM) receptor complex comprising an affinity polypeptide, wherein the affinity polypeptide is heterologous to the first subunit polypeptide; and/or (b) a second subunit polypeptide of a TM receptor optionally comprising a tag polypeptide, wherein the one or more nucleotide sequences are operably linked to at least one expression control sequence; and/or one or more nucleotide sequences encoding a bispecific affinity polypeptide comprising a first affinity polypeptide that binds to an intracellular portion of a first subunit polypeptide of a TM receptor and a second affinity polypeptide that binds to an intracellular portion of a second subunit polypeptide of a TM receptor, wherein the one or more nucleotide sequences are operably linked to at least one expression control sequence.

22. A bacterial cell or an Agrobacterium cell comprising the expression vector or isolated DNA molecule of claim 21.

23. A genetically modified plant, plant part, plant cell, or seed comprising the expression vector or isolated DNA molecule of claim 21.

24. A kit comprising the expression vector or isolated DNA molecule of claim 21 or the bacterial cell or the Agrobacterium cell of claim 22.

25. A method of activating a target transmembrane (TM) receptor complex or inducing organogenesis comprising: introducing a genetic alteration via an expression vector or isolated DNA molecule of claim 21 to a cell.

26. A genetically modified plant cell comprising:
an NFR1-NFR5 receptor complex comprising a first subunit polypeptide and a second subunit polypeptide,
wherein the first subunit polypeptide comprises an affinity polypeptide that binds to the second subunit polypeptide inducing oligomerization, and wherein the affinity polypeptide is heterologous to the first subunit polypeptide; and
wherein oligomerization of the first subunit polypeptide and the second subunit polypeptide activates NFR1-NFR5 receptor complex signaling and wherein (i) the first subunit polypeptide is an NFR1 polypeptide and the second subunit polypeptide is an NFR5 polypeptide, or (ii) the first subunit polypeptide is the NFR5 polypeptide and the second subunit polypeptide is the NFR1 polypeptide.

27. A bispecific affinity polypeptide for activation of an NFR1-NFR5 receptor complex comprising:
a first affinity polypeptide that binds to an intracellular portion of an NFR1 polypeptide; and
a second affinity polypeptide that binds to an intracellular portion of an NFR5 polypeptide;
wherein binding of the first affinity polypeptide to the NFR1 polypeptide and of the second affinity to the NFR5 polypeptide induces dimerization; and wherein dimerization of the NFR1 polypeptide and the NFR5 polypeptide activates NFR1-NFR5 receptor complex signaling.

28. An expression vector or isolated DNA molecule comprising one or more nucleotide sequences encoding a NFR1 polypeptide comprising an affinity polypeptide, wherein the affinity polypeptide is heterologous to the NFR1 polypeptide, and wherein the one or more nucleotide sequences are operably linked to at least one expression control sequence; one or more nucleotide sequences encoding a NFR5 polypeptide optionally comprising a tag polypeptide, wherein the one or more nucleotide sequences are operably linked to at least one expression control sequence; one or more nucleotide sequences encoding: (a) a NFR1 polypeptide comprising an affinity polypeptide, wherein the affinity polypeptide is heterologous to the first subunit polypeptide; and/or (b) a NFR5 polypeptide optionally comprising a tag polypeptide, wherein the one or more nucleotide sequences are operably linked to at least one expression control sequence; and/or one or more nucleotide sequences encoding a bispecific affinity polypeptide comprising a first affinity polypeptide that binds to an intracellular portion of a NFR1 polypeptide and a second affinity polypeptide that binds to an intracellular portion of a NFR5 polypeptide, wherein the one or more nucleotide sequences are operably linked to at least one expression control sequence.
